(19) United States
(12) Patent Application Publication
(10) Pub. No.: US 2013/0237535 A1
(43) Pub. Date: Sep. 12, 2013

(54) FATTY ACID SYNTHASE INHIBITORS

(71) Applicants: Nicholas D. Adams, Collegeville, PA (US); Amita M. Chaudhari, Collegeville, PA (US); Terence John Kiesow, Collegeville, PA (US); Cynthia Ann Parrish, Collegeville, PA (US); Alexander Joseph Reif, Collegeville, PA (US); Lance Howard Ridgers, Collegeville, PA (US); Stanley J. Schmidt, Collegeville, PA (US); Kenneth Wiggall, Collegeville, PA (US)

(72) Inventors: Nicholas D. Adams, Collegeville, PA (US); Amita M. Chaudhari, Collegeville, PA (US); Terence John Kiesow, Collegeville, PA (US); Cynthia Ann Parrish, Collegeville, PA (US); Alexander Joseph Reif, Collegeville, PA (US); Lance Howard Ridgers, Collegeville, PA (US); Stanley J. Schmidt, Collegeville, PA (US); Kenneth Wiggall, Collegeville, PA (US)

(21) Appl. No.: 13/883,877

(22) PCT Filed: Nov. 7, 2011
(86) PCT No.: PCT/US11/59563

§ 371 Date: May 7, 2013

Related U.S. Application Data
(60) Provisional application No. 61/411,110, filed on Nov. 8, 2010.

(51) Int. Cl. C07D 498/10; A61K 45/06; A61K 31/499; A61K 31/4709; A61K 31/5386; C07D 471/10

(52) U.S. Cl. CPC C07D 498/10; A61K 31/5386; C07D 471/10; A61K 31/499; A61K 31/4709; A61K 45/06
USPC 514/230.8; 544/71; 544/231; 514/253.06; 546/16; 514/278

(57) ABSTRACT

This invention relates to the use of spirocyclic piperidine derivatives for the modulation, notably the inhibition of the activity or function of fatty acid synthase (FAS). Suitably, the present invention relates to the use of spirocyclic piperidines in the treatment of cancer.

### FIELD OF INVENTION

This invention relates to novel spirocyclic piperidines which are inhibitors of fatty acid synthase (FAS), to pharmaceutical compositions containing them, to processes for their preparation, and to their use in therapy for the treatment of cancers.

### BACKGROUND

Fatty acids have an essential role in a variety of cellular processes including building blocks for membranes, anchors for targeting membrane proteins, precursors in the synthesis of lipid second messengers and as a medium to store energy, Menendez J S and Lupu R, Fatty acid synthase and the lipogenic phenotype in cancer pathogenesis, Nature Reviews Cancer, 7: 763-777 (2007). Fatty acids can either be obtained from the diet or can be synthesized de novo from carbohydrate precursors. The biosynthesis of the latter is catalyzed by the multi-functional homodimeric FAS. FAS synthesizes long chain fatty acids by using acetyl-CoA as a primer and Malonyl Co-A as a 2 carbon donor, and NADPH as a reducing equivalents (Wakil S J, Lipids, Structure and function of animal fatty acid synthase, 39: 1045-1053 (2004), Asturias F J et al., Structure and molecular organization of mammalian fatty acid synthase, Nature Struct. Mol. Biol. 12:225-232 (2005), Maier T, et al., Architecture of Mammalian Fatty Acid Synthase at 4.5 Å Resolution, Science 311:1258-1262 (2006).

De novo fatty acid synthesis is active during embryogenesis and in fetal lungs where fatty acids are used for the production of lung surfactant. In adults, most normal human tissues preferentially acquire fatty acids from the diet. Therefore, the level of de novo lipogensis and expression of liopogenic enzymes is low, Weiss L, et al., Fatty-acid biosynthesis in man, a pathway of minor importance. Purification, optimal assay conditions, and organ distribution of fatty-acid synthase. Biological Chemistry Hoppe-Seyler 367(9):905-912 (1986). In contrast, many tumors have high rates of de novo fatty acid synthesis Medes G, et al., Metabolism of Neoplastic Tissue. IV. A Study of Lipid Synthesis in Neoplastic Tissue Slices in Vitro, Can Res, 13:27-29, (1953). FAS has now been shown to be overexpressed in numerous cancer types including prostate, ovary, colon, endometrium lung, bladder, stomach and kidney Kuhajda F P, Fatty-acid synthase and human cancer: new perspectives on its role in tumor biology, Nutrition; 16:202-208 (2000). This differential expression and function of FAS in tumors and normal cells provide an approach for cancer therapy with the potential of a substantial therapeutic window.

Pharmacological and small interference RNA mediated inhibition of FAS has demonstrated a preferential inhibition of cancer cell proliferation. Additionally these inhibitors induce apoptosis in cancers cells in vitro and retard growth in human tumors in murine xenograft models in vivo, Menendez J S and Lupu R, Nature Reviews Cancer, 7: 763-777 (2007). Based upon these findings, FAS is considered a major potential target of antineoplastic intervention.

### SUMMARY OF THE INVENTION

This invention relates to compounds of the Formula (I), as shown below

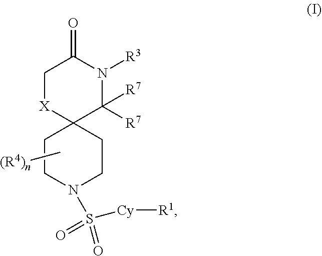

wherein

R³ is selected from the group consisting of: C₁-C₆alkyl, C₃-C₇cycloalkyl, and C₄-C₆heterocycloalkyl, wherein said C₁-C₆alkyl, C₃-C₇cycloalkyl or C₄-C₆heterocycloalkyl is optionally substituted with from 1 to 6 substituents independently selected from the group of: halogen, C₁-C₄alkyl, —C₁-C₄alkylhalogen, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹;

R⁵ is selected from the group consisting of: hydrogen, C₁-C₄alkyl, C₃-C₇cycloalkyl, —C₁-C₃alkylC₃-C₇cycloalkyl, phenyl, and —C₁-C₃alkylphenyl;

R⁶ is hydrogen, C₁-C₄alkyl, C₃-C₇cycloalkyl, or —C₁-C₃alkylC₃-C₇cycloalkyl;

or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl;

R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁵R⁶;

R⁴ is oxo, halogen or C₁-C₆alkyl;

Cy is selected from the group consisting of: phenyl, pyridinyl, and 5- or 6-membered heteroaryl wherein said phenyl, pyridinyl, and 5- or 6-membered heteroaryl are each optionally substituted with from one to three R² groups, wherein each R² is independently selected from C₁-C₆alkyl, cyano, C₁-C₄alkoxy, hydroxyl, —CF₃, or halogen;

R¹ is selected from the group consisting of: phenyl, 5- or 6-membered heteroaryl, napthyl, and 9- or 10-membered heterocyclyl, wherein said phenyl, 5- or 6-membered heteroaryl, napthyl, or 9- or 10-membered heterocyclyl, is optionally substituted with from 1 to 4 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹;

each R⁷ is independently H, C₁-C₃alkyl, —C₁-C₄alkylhalogen, halogen, cyano, —CONR⁵R⁶, —C(═O)OC₁-C₄alkyl, hydroxyC₁-C₄alkyl-, and —C(═O)OH;

### X is CH₂, NR₆ or O;

n is 0, 1, 2, 3, or 4;
or a pharmaceutically acceptable salt thereof.

This invention also relates to pharmaceutical compositions, which comprise compounds of Formula (I) and pharmaceutically acceptable carriers.

This invention also relates to methods of treating cancer which comprises administering an effective amount of a compound of formula (I) to a human in need thereof.

This invention also relates to methods of treating cancer which comprise co-administering an compound of Formula (I) and a second compound to a human in need thereof.

### DETAILED DESCRIPTION OF THE INVENTION

This invention also relates to compound of the Formula (I)(A), as shown below

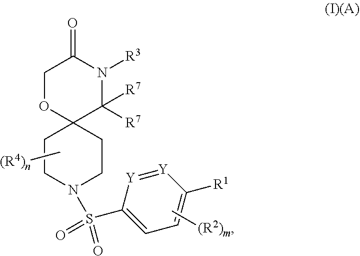

wherein

R³ is selected from the group consisting of: C₁-C₆alkyl, C₃-C₇cycloalkyl, and C₄-C₆heterocycloalkyl, wherein said C₁-C₆alkyl, C₃-C₇cycloalkyl or C₄-C₆heterocycloalkyl is optionally substituted with from 1 to 6 substituents independently selected from the group of: halogen, C₁-C₄alkyl, —C₁-C₄alkylhalogen, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄ alkoxyC₁-C₄ alkyl-, —OCF₃, —NR⁵R⁶, R⁵R⁶NC₁-C₄alkyl-, —NHC(O)C₁-C₄alkyl, —NH CONR⁵R⁶, —NHSO₂C₁-C₄alkyl, —NHSO₂NR⁵R⁶, and R⁹;

R⁵ is selected from the group consisting of: hydrogen, C₁-C₄alkyl, C₃-C₇cycloalkyl, —C₁-C₃alkylC₃-C₇cycloalkyl, phenyl, and —C₁-C₃alkylphenyl;

R⁶ is hydrogen, C₁-C₄alkyl, C₃-C₇cycloalkyl, or —C₁-C₃alkyl C₃-C₇cycloalkyl;

or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl;

R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁵R⁶;

R⁴ is oxo, halogen or C₁-C₆alkyl;

R¹ is selected from the group consisting of: phenyl, 5- or 6-membered heteroaryl, napthyl, and 9- or 10-membered heterocyclyl, wherein said phenyl, 5- or 6-membered heteroaryl, napthyl, or 9- or 10-membered heterocyclyl, is optionally substituted with from 1 to 4 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CON R⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹;

each R² is independently C₁-C₆alkyl, cyano, C₁-C₄alkoxy, hydroxyl, —CF₃, or halogen;

each R⁷ is independently H, C₁-C₃alkyl, —C₁-C₄alkylhalogen, halogen, cyano, —CONR⁵R⁶, —C(═O)OC₁-C₄alkyl, hydroxyC₁-C₄alkyl-, and —C(═O)OH;

n is 0, 1, 2, 3, or 4;
m is 0, 1, 2, or 3;
Y is C or N, provided that when one Y is N the other Y is C;
or a pharmaceutically acceptable salt thereof.

This invention also relates to compounds of Formula (I), wherein Cy is a phenyl, optionally substituted with from one to three groups selected from the group consisting of: C₁-C₆alkyl, cyano, C₁-C₄alkoxy, hydroxyl, —CF₃, and halogen; or a pharmaceutically acceptable salt thereof.

This invention also relates to compounds of Formula (I), wherein Cy is 5- or 6-membered heteroaryl, optionally substituted with one to two groups selected from the group consisting of: C₁-C₆alkyl, cyano, C₁-C₄alkoxy, hydroxyl, —CF₃, and halogen; or a pharmaceutically acceptable salt thereof.

This invention also relates to compounds of Formula (I), wherein Cy is 5-membered heteroaryl selected from the group consisting of:

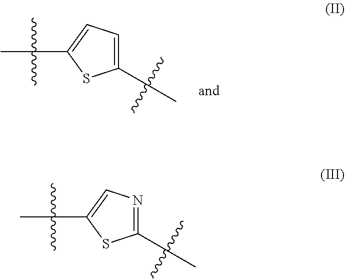 [SMILES: *c1ccc(*)s1 ; *c1cnc(*)s1]

which may be substituted with one to two groups selected from the group consisting of: C₁-C₆alkyl, cyano, C₁-C₄alkoxy, hydroxyl, —CF₃, and halogen; or a pharmaceutically acceptable salt thereof.

This invention also relates to compounds of Formula (I), wherein each R⁷ is H.

This invention also relates to compounds of Formula (I)(B),

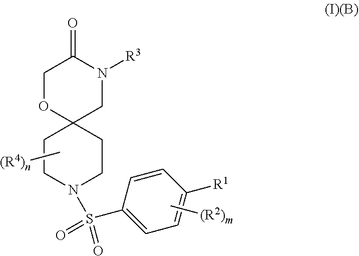

wherein

R³ is selected from the group consisting of: C₁-C₆alkyl, C₃-C₇cycloalkyl, and C₄-C₆heterocycloalkyl, wherein said C₁-C₆alkyl, C₃-C₇cycloalkyl or C₄-C₆heterocycloalkyl is optionally substituted with from 1 to 6 substituents independently selected from the group of: halogen, C₁-C₄alkyl, —C₁-C₄alkylhalogen, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —N HSO₂NR⁵R⁶, and R⁹;

R⁵ is selected from the group consisting of: hydrogen, C₁-C₄alkyl, C₃-C₇cycloalkyl, —C₁-C₃alkylC₃-C₇cycloalkyl, phenyl, and —C₁-C₃alkylphenyl;

R⁶ is hydrogen, C₁-C₄alkyl, C₃-C₇cycloalkyl, or —C₁-C₃alkyl C₃-C₇cycloalkyl;

or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl;

R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁵R⁶;

R⁴ is oxo, halogen or C₁-C₆alkyl;

R¹ is selected from the group consisting of: phenyl, 5- or 6-membered heteroaryl, napthyl, and 9- or 10-membered heterocyclyl, wherein said phenyl, 5- or 6-membered heteroaryl, napthyl, 9- or 10-membered heterocyclyl, is optionally substituted with from 1 to 4 substituents independently selected from halogen, C₁-C₄alkylhalogen, optionally substituted C₁-C₄alkyl, —CF₃, —C₃-C₇cyclo alkyl, —C(O)C₁-C₄alkyl, —C(O)C₃-C₇cyclo alkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹.

each R² is independently C₁-C₆alkyl, cyano, C₁-C₄alkoxy, hydroxyl, —CF₃, or halogen;

n is 0, 1, 2, 3, or 4;
m is 0, 1, 2, or 3;
or a pharmaceutically acceptable salt thereof.

This invention also relates to compounds of Formula (I) or pharmaceutically acceptable salt thereof, wherein R¹ is phenyl optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹, wherein R⁵, R⁶, and R⁹ are defined as for Formula (I).

This invention also relates to compounds of Formula (I) or pharmaceutically acceptable salt thereof, wherein R¹ is selected from furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl are each optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹, wherein R⁵, R⁶, and R⁹ are defined as for Formula (I).

This invention also relates to compounds of Formula (I) or pharmaceutically acceptable salt thereof, wherein R¹ is napthyl optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹, wherein R⁵, R⁶, and R⁹ are defined as for Formula (I).

This invention also relates to compounds of Formula (I) or pharmaceutically acceptable salt thereof, wherein R¹ is selected from benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl are each optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹, wherein R⁵, R⁶, and R⁹ are defined as for Formula (I).

This invention also relates to compounds of Formula (I) or pharmaceutically acceptable salt thereof, wherein R³ is C₁-C₆alkyl or C₃-C₇cycloalkyl.

This invention also relates to the following compounds:
4-methyl-9-{[4-(7-quinolinyl)phenyl]sulfonyl}-1-oxa-4,9-diazaspiro[5.5]undecan-3-one;
9-{[4-(1H-indol-6-yl)phenyl]sulfonyl}-4-methyl-1-oxa-4,9-diazaspiro[5.5]undecan-3-one;
9-{[4-(1-benzofuran-5-yl)phenyl]sulfonyl}-4-ethyl-1-oxa-4,9-diazaspiro[5.5]undecan-3-one;
4-ethyl-9-{[4-(7-quinolinyl)phenyl]sulfonyl}-1-oxa-4,9-diazaspiro[5.5]undecan-3-one;
4-(1-methylethyl)-9-{[4-(7-quinolinyl)phenyl]sulfonyl}-1-oxa-4,9-diazaspiro[5.5]undecan-3-one;
9-{[4-(7-quinolinyl)phenyl]sulfonyl}-4-(2,2,2-trifluoroethyl)-1-oxa-4,9-diazaspiro[5.5]undecan-3-one;
4-(2-furanylmethyl)-9-{[4-(7-quinolinyl)phenyl]sulfonyl}-1-oxa-4,9-diazaspiro[5.5]undecan-3-one;
4-[2-(methyloxy)ethyl]-9-{[4-(7-quinolinyl)phenyl]sulfonyl}-1-oxa-4,9-diazaspiro[5.5]undecan-3-one;
4-(phenylmethyl)-9-{[4-(7-quinolinyl)phenyl]sulfonyl}-1-oxa-4,9-diazaspiro[5.5]undecan-3-one;
4-(1,1-dimethylethyl)-9-{[4-(7-quinolinyl)phenyl]sulfonyl}-1-oxa-4,9-diazaspiro[5.5]undecan-3-one;
4-(1-methylcyclopropyl)-9-{[4-(7-quinolinyl)phenyl]sulfonyl}-1-oxa-4,9-diazaspiro[5.5]undecan-3-one;
4-cyclobutyl-9-{[4-(7-quinolinyl)phenyl]sulfonyl}-1-oxa-4,9-diazaspiro[5.5]undecan-3-one;
9-(4-biphenylylsulfonyl)-4-cyclopropyl-1-oxa-4,9-diazaspiro[5.5]undecan-3-one;
4-cyclopropyl-9-{[4-(1H-indol-6-yl)phenyl]sulfonyl}-1-oxa-4,9-diazaspiro[5.5]undecan-3-one;
4′-[(4-cyclopropyl-3-oxo-1-oxa-4,9-diazaspiro[5.5]undec-9-yl)sulfonyl]-4-biphenylcarbonitrile;
4-cyclopropyl-9-[(4′-fluoro-4-biphenylyl)sulfonyl]-1-oxa-4,9-diazaspiro[5.5]undecan-3-one;
4-cyclopropyl-9-{[4-(1H-indazol-6-yl)phenyl]sulfonyl}-1-oxa-4,9-diazaspiro[5.5]undecan-3-one;
4-cyclopropyl-9-{[4-(1H-indol-5-yl)phenyl]sulfonyl}-1-oxa-4,9-diazaspiro[5.5]undecan-3-one;
9-{[4-(1-benzofuran-5-yl)phenyl]sulfonyl}-4-cyclopropyl-1-oxa-4,9-diazaspiro[5.5]undecan-3-one;
4′-[(4-cyclopropyl-3-oxo-1-oxa-4,9-diazaspiro[5.5]undec-9-yl)sulfonyl]-3-methyl-4-biphenylcarbonitrile;
4-cyclopropyl-9-{[2-fluoro-4-(1H-indol-6-yl)phenyl]sulfonyl}-1-oxa-4,9-diazaspiro[5.5]undecan-3-one;
4′-[(4-cyclopropyl-3-oxo-1-oxa-4,9-diazaspiro[5.5]undec-9-yl)sulfonyl]-3′-fluoro-4-biphenylcarbonitrile;
4-cyclopropyl-9-[(3,4′-difluoro-4-biphenylyl)sulfonyl]-1-oxa-4,9-diazaspiro[5.5]undecan-3-one;
4-cyclopropyl-9-{[4-(1-methyl-2-oxo-1,2-dihydro-6-quinolinyl)phenyl]sulfonyl}-1-oxa-4,9-diazaspiro[5.5]undecan-3-one;
4-cyclopropyl-9-{[4-(1-methyl-2,3-dihydro-1H-indol-5-yl)phenyl]sulfonyl}-1-oxa-4,9-diazaspiro[5.5]undecan-3-one;
9-{[4-(1-benzofuran-2-yl)phenyl]sulfonyl}-4-cyclopropyl-1-oxa-4,9-diazaspiro[5.5]undecan-3-one;
9-{[4-(1-benzothien-2-yl)phenyl]sulfonyl}-4-cyclopropyl-1-oxa-4,9-diazaspiro[5.5]undecan-3-one;
9-{[4-(1,3-benzoxazol-2-yl)phenyl]sulfonyl}-4-cyclopropyl-1-oxa-4,9-diazaspiro[5.5]undecan-3-one;
4-cyclopropyl-9-{[4-(7-quinolinyl)phenyl]sulfonyl}-1-oxa-4,9-diazaspiro[5.5]undecan-3-one;
4-cyclopropyl-9-{[4-(7-quinolinyl)phenyl]sulfonyl}-1,4,9-triazaspiro[5.5]undecan-3-one;
4-cyclopropyl-1-methyl-9-{[4-(7-quinolinyl)phenyl]sulfonyl}-1,4,9-triazaspiro[5.5]undecan-3-one;
4-cyclopropyl-8-methyl-9-{[4-(7-quinolinyl)phenyl]sulfonyl}-1-oxa-4,9-diazaspiro[5.5]undecan-3-one;
4-cyclopropyl-7-methyl-9-{[4-(7-quinolinyl)phenyl]sulfonyl}-1-oxa-4,9-diazaspiro[5.5]undecan-3-one;
9-[(4-imidazo[1,2-a]pyridin-7-ylphenyl)sulfonyl]-4-(1-methylcyclopropyl)-1-oxa-4,9-diazaspiro[5.5]undecan-3-one;
4-cyclopropyl-7-fluoro-9-[(4-imidazo[1,2-a]pyridin-7-ylphenyl)sulfonyl]-1-oxa-4,9-diazaspiro[5.5]undecan-3-one;
4-cyclopropyl-7-fluoro-9-{[4-(7-quinolinyl)phenyl]sulfonyl}-1-oxa-4,9-diazaspiro[5.5]undecan-3-one;
4-cyclopropyl-9-{[3-fluoro-4-(1H-indol-6-yl)phenyl]sulfonyl}-1-oxa-4,9-diazaspiro[5.5]undecan-3-one;
4-cyclopropyl-9-{[3-fluoro-4-(7-quinolinyl)phenyl]sulfonyl}-1-oxa-4,9-diazaspiro[5.5]undecan-3-one;
9-{[3-chloro-4-(1H-indol-6-yl)phenyl]sulfonyl}-4-cyclopropyl-1-oxa-4,9-diazaspiro[5.5]undecan-3-one;
9-{[3-chloro-4-(7-quinolinyl)phenyl]sulfonyl}-4-cyclopropyl-1-oxa-4,9-diazaspiro[5.5]undecan-3-one;
4-cyclopropyl-9-{[4-(1H-indol-6-yl)-3-methylphenyl]sulfonyl}-1-oxa-4,9-diazaspiro[5.5]undecan-3-one;
4-cyclopropyl-9-{[3-methyl-4-(7-quinolinyl)phenyl]sulfonyl}-1-oxa-4,9-diazaspiro[5.5]undecan-3-one;
4-cyclopropyl-9-{[2,5-difluoro-4-(1H-indol-6-yl)phenyl]sulfonyl}-1-oxa-4,9-diazaspiro[5.5]undecan-3-one;
4-cyclopropyl-9-{[2,5-difluoro-4-(7-quinolinyl)phenyl]sulfonyl}-1-oxa-4,9-diazaspiro[5.5]undecan-3-one;
4-cyclopropyl-9-{[3-(methyloxy)-4-(7-quinolinyl)phenyl]sulfonyl}-1-oxa-4,9-diazaspiro[5.5]undecan-3-one;
4-cyclopropyl-9-{[4-(1H-indol-6-yl)-3-(methyloxy)phenyl]sulfonyl}-1-oxa-4,9-diazaspiro[5.5]undecan-3-one;
4-cyclopropyl-9-{[5-(7-quinolinyl)-2-pyridinyl]sulfonyl}-1-oxa-4,9-diazaspiro[5.5]undecan-3-one;
4-cyclopropyl-9-{[5-(1H-indol-6-yl)-2-pyridinyl]sulfonyl}-1-oxa-4,9-diazaspiro[5.5]undecan-3-one;
4-cyclopropyl-9-{[4-(7-quinolinyl)-3-(trifluoromethyl)phenyl]sulfonyl}-1-oxa-4,9-diazaspiro[5.5]undecan-3-one;
4-cyclopropyl-9-{[2-methyl-4-(7-quinolinyl)phenyl]sulfonyl}-1-oxa-4,9-diazaspiro[5.5]undecan-3-one;
4-cyclopropyl-9-{[4-(1H-indol-6-yl)-2-methylphenyl]sulfonyl}-1-oxa-4,9-diazaspiro[5.5]undecan-3-one;
9-{[2-chloro-4-(1H-indol-6-yl)phenyl]sulfonyl}-4-cyclopropyl-1-oxa-4,9-diazaspiro[5.5]undecan-3-one;
4-cyclopropyl-9-{[5-(1H-indol-6-yl)-2-thienyl]sulfonyl}-1-oxa-4,9-diazaspiro[5.5]undecan-3-one;
9-{[2-chloro-4-(7-quinolinyl)phenyl]sulfonyl}-4-cyclopropyl-1-oxa-4,9-diazaspiro[5.5]undecan-3-one;
4-cyclopropyl-9-{[2-fluoro-4-(7-quinolinyl)phenyl]sulfonyl}-1-oxa-4,9-diazaspiro[5.5]undecan-3-one;
4-cyclopropyl-9-{[5-(7-quinolinyl)-2-thienyl]sulfonyl}-1-oxa-4,9-diazaspiro[5.5]undecan-3-one;
4-cyclopropyl-9-{[6-(7-quinolinyl)-3-pyridinyl]sulfonyl}-1-oxa-4,9-diazaspiro[5.5]undecan-3-one;
4-cyclopropyl-9-{[6-(1H-indol-6-yl)-3-pyridinyl]sulfonyl}-1-oxa-4,9-diazaspiro[5.5]undecan-3-one;
4-cyclopropyl-9-{[2,3-dimethyl-4-(7-quinolinyl)phenyl]sulfonyl}-1-oxa-4,9-diazaspiro[5.5]undecan-3-one;
9-{[3-chloro-2-fluoro-4-(7-quinolinyl)phenyl]sulfonyl}-4-cyclopropyl-1-oxa-4,9-diazaspiro[5.5]undecan-3-one;
9-{[3-chloro-2-fluoro-4-(1H-indol-6-yl)phenyl]sulfonyl}-4-cyclopropyl-1-oxa-4,9-diazaspiro[5.5]undecan-3-one;
4-cyclopropyl-9-{[3,5-difluoro-4-(1H-indol-6-yl)phenyl]sulfonyl}-1-oxa-4,9-diazaspiro[5.5]undecan-3-one;
4-cyclopropyl-9-{[3,5-difluoro-4-(7-quinolinyl)phenyl]sulfonyl}-1-oxa-4,9-diazaspiro[5.5]undecan-3-one;
4-cyclopropyl-9-{[4-methyl-5-(7-quinolinyl)-2-thienyl]sulfonyl}-1-oxa-4,9-diazaspiro[5.5]undecan-3-one;
4-cyclopropyl-9-{[2-(7-quinolinyl)-1,3-thiazol-5-yl]sulfonyl}-1-oxa-4,9-diazaspiro[5.5]undecan-3-one;
4-cyclopropyl-9-{[2-fluoro-4-(1H-indol-5-yl)phenyl]sulfonyl}-1-oxa-4,9-diazaspiro[5.5]undecan-3-one;
9-{[4-(1-benzofuran-5-yl)-2-fluorophenyl]sulfonyl}-4-cyclopropyl-1-oxa-4,9-diazaspiro[5.5]undecan-3-one;
4-cyclopropyl-9-{[2-fluoro-4-(1H-indazol-5-yl)phenyl]sulfonyl}-1-oxa-4,9-diazaspiro[5.5]undecan-3-one;
4-cyclopropyl-9-{[2-fluoro-4-(6-quinolinyl)phenyl]sulfonyl}-1-oxa-4,9-diazaspiro[5.5]undecan-3-on;
4-cyclopropyl-9-[(2′,4′-dichloro-3-fluoro-4-biphenylyl)sulfonyl]-1-oxa-4,9-diazaspiro[5.5]undecan-3-one;
4-cyclopropyl-9-{[3-fluoro-4′-(methyloxy)-4-biphenylyl]sulfonyl}-1-oxa-4,9-diazaspiro[5.5]undecan-3-one;
9-{[4-(1,3-benzothiazol-5-yl)-2-fluorophenyl]sulfonyl}-4-cyclopropyl-1-oxa-4,9-diazaspiro[5.5]undecan-3-one;
{4′-[(4-cyclopropyl-3-oxo-1-oxa-4,9-diazaspiro[5.5]undec-9-yl)sulfonyl]-3′-fluoro-4-hydroxy-3-biphenylyl}formamide;
4-cyclopropyl-9-{[2-fluoro-4-(5-quinolinyl)phenyl]sulfonyl}-1-oxa-4,9-diazaspiro[5.5]undecan-3-one;
4-cyclopropyl-9-{[3-fluoro-4′-(1H-pyrazol-1-yl)-4-biphenylyl]sulfonyl}-1-oxa-4,9-diazaspiro[5.5]undecan-3-one;
9-{[4-(1,3-benzoxazol-5-yl)-2-fluorophenyl]sulfonyl}-4-cyclopropyl-1-oxa-4,9-diazaspiro[5.5]undecan-3-one;
4-cyclopropyl-9-{[3′-(1H-pyrazol-5-yl)-4-biphenylyl]sulfonyl}-1-oxa-4,9-diazaspiro[5.5]undecan-3-one;
4-cyclopropyl-9-{[4′-(1H-pyrazol-5-yl)-4-biphenylyl]sulfonyl}-1-oxa-4,9-diazaspiro[5.5]undecan-3-one;
4-cyclopropyl-9-{[4-(7-isoquinolinyl)phenyl]sulfonyl}-1-oxa-4,9-diazaspiro[5.5]undecan-3-one;
4-cyclopropyl-9-{[4-(7-quinazolinyl)phenyl]sulfonyl}-1-oxa-4,9-diazaspiro[5.5]undecan-3-one;
N′-{4′-[(4-cyclopropyl-3-oxo-1-oxa-4,9-diazaspiro[5.5]undec-9-yl)sulfonyl]-3-biphenylyl}-N,N-dimethylsulfamide;
4-cyclopropyl-9-{[4-(6-isoquinolinyl)phenyl]sulfonyl}-1-oxa-4,9-diazaspiro[5.5]undecan-3-one;
4-cyclopropyl-9-{[4-(3-quinolinyl)phenyl]sulfonyl}-1-oxa-4,9-diazaspiro[5.5]undecan-3-one;
4-cyclopropyl-9-{[4-(2-naphthalenyl)phenyl]sulfonyl}-1-oxa-4,9-diazaspiro[5.5]undecan-3-one;
4-cyclopropyl-9-{[4-(2-methyl-1,3-benzothiazol-5-yl)phenyl]sulfonyl}-1-oxa-4,9-diazaspiro[5.5]undecan-3-one;
4-cyclopropyl-9-({4-[4-(ethyloxy)-7-quinolinyl]phenyl}sulfonyl)-1-oxa-4,9-diazaspiro[5.5]undecan-3-one;
4-cyclopropyl-9-({4-[4-(methyloxy)-7-quinolinyl]phenyl}sulfonyl)-1-oxa-4,9-diazaspiro[5.5]undecan-3-one;
4-cyclopropyl-9-[(4-imidazo[1,2-c]pyridin-7-ylphenyl)sulfonyl]-1-oxa-4,9-diazaspiro[5.5]undecan-3-one;
9-{[4-(3-amino-1H-indazol-6-yl)phenyl]sulfonyl}-4-cyclopropyl-1-oxa-4,9-diazaspiro[5.5]undecan-3-one;
9-{[4-(3-amino-1H-indazol-5-yl)phenyl]sulfonyl}-4-cyclopropyl-1-oxa-4,9-diazaspiro[5.5]undecan-3-one;
9-{[4-(2-amino-4-pyridinyl)phenyl]sulfonyl}-4-cyclopropyl-1-oxa-4,9-diazaspiro[5.5]undecan-3-one;
4-cyclopropyl-9-{[4-(4-quinolinyl)phenyl]sulfonyl}-1-oxa-4,9-diazaspiro[5.5]undecan-3-one;
4-cyclopropyl-9-{[4-(1-methyl-1H-indol-6-yl)phenyl]sulfonyl}-1-oxa-4,9-diazaspiro[5.5]undecan-3-one;
4-cyclopropyl-9-{[4-(1-methyl-1H-indol-4-yl)phenyl]sulfonyl}-1-oxa-4,9-diazaspiro[5.5]undecan-3-one;
4-cyclopropyl-9-({4-[4-(methylamino)-7-quinolinyl]phenyl}sulfonyl)-1-oxa-4,9-diazaspiro[5.5]undecan-3-one;
4-cyclopropyl-9-{[4-(4-methyl-7-quinolinyl)phenyl]sulfonyl}-1-oxa-4,9-diazaspiro[5.5]undecan-3-one;
4-cyclopropyl-9-[(4-imidazo[1,2-c]pyridin-6-ylphenyl)sulfonyl]-1-oxa-4,9-diazaspiro[5.5]undecan-3-one;
9-{[4-(7-cinnolinyl)phenyl]sulfonyl}-4-cyclopropyl-1-oxa-4,9-diazaspiro[5.5]undecan-3-one;
4-cyclopropyl-9-[(4-imidazo[1,2-b]pyridazin-6-ylphenyl)sulfonyl]-1-oxa-4,9-diazaspiro[5.5]undecan-3-one;
9-{[4-(3-amino-1-methyl-1H-indazol-5-yl)phenyl]sulfonyl}-4-cyclopropyl-1-oxa-4,9-diazaspiro[5.5]undecan-3-one;
N-(5-{4-[(4-cyclopropyl-3-oxo-1-oxa-4,9-diazaspiro[5.5]undec-9-yl)sulfonyl]phenyl}-1-methyl-1H-indazol-3-yl)methanesulfonamide;
9-{[4-(3-amino-1-methyl-1H-indazol-6-yl)phenyl]sulfonyl}-4-cyclopropyl-1-oxa-4,9-diazaspiro[5.5]undecan-3-one;
N-(6-{4-[(4-cyclopropyl-3-oxo-1-oxa-4,9-diazaspiro[5.5]undec-9-yl)sulfonyl]phenyl}-1H-indazol-3-yl)-N′-methylurea;
4-cyclopropyl-9-((4-(8-methylquinolin-7-yl)phenyl)sulfonyl)-1-oxa-4,9-diazaspiro[5.5]undecan-3-one;
4-cyclopropyl-9-((4-(8-fluoroquinolin-7-yl)phenyl)sulfonyl)-1-oxa-4,9-diazaspiro[5.5]undecan-3-one;
1-(3-oxo-9-{[4-(7-quinolinyl)phenyl]sulfonyl}-1-oxa-4,9-diazaspiro[5.5]undec-4-yl)cyclopropanecarboxamide;
4-(1-methylcyclobutyl)-9-{[4-(7-quinolinyl)phenyl]sulfonyl}-1-oxa-4,9-diazaspiro[5.5]undecan-3-one;
1-(3-oxo-9-{[4-(7-quinolinyl)phenyl]sulfonyl}-1-oxa-4,9-diazaspiro[5.5]undec-4-yl)cyclopropanecarbonitrile;
4-(3-oxetanyl)-9-{[4-(7-quinolinyl)phenyl]sulfonyl}-1-oxa-4,9-diazaspiro[5.5]undecan-3-one;
4-[1-(hydroxymethyl)cyclopropyl]-9-{[4-(7-quinolinyl)phenyl]sulfonyl}-1-oxa-4,9-diazaspiro[5.5]undecan-3-one;
4-{1-[(methyloxy)methyl]cyclopropyl}-9-{[4-(7-quinolinyl)phenyl]sulfonyl}-1-oxa-4,9-diazaspiro[5.5]undecan-3-one;
4-[1-(hydroxymethyl)cyclopropyl]-9-{[4-(3-(methyloxy)-7-quinolinyl]phenyl}sulfonyl)-1-oxa-4,9-diazaspiro[5.5]undecan-3-one;
4-cyclopropyl-9-((4-(8-methoxyquinolin-7-yl)phenyl)sulfonyl)-1-oxa-4,9-diazaspiro[5.5]undecan-3-one;
4-cyclopropyl-9-((4-(8-hydroxyquinolin-7-yl)phenyl)sulfonyl)-1-oxa-4,9-diazaspiro[5.5]undecan-3-one;
4-cyclopropyl-9-{[4-(7-quinolinyl)phenyl]sulfonyl}-1-oxa-4,9-diazaspiro[5.5]undecan-3-one-d₄;
4-cyclopropyl-9-((4-(6-fluoronaphthalen-2-yl)phenyl)sulfonyl)-1-oxa-4,9-diazaspiro[5.5]undecan-3-one;
4-cyclopropyl-9-((4-(8-fluoronaphthalen-2-yl)phenyl)sulfonyl)-1-oxa-4,9-diazaspiro[5.5]undecan-3-one;
4-ethyl-9-((4-(3-methoxyquinolin-7-yl)phenyl)sulfonyl)-1-oxa-4,9-diazaspiro[5.5]undecan-3-one;
4-isopropyl-9-((4-(3-methoxyquinolin-7-yl)phenyl)sulfonyl)-1-oxa-4,9-diazaspiro[5.5]undecan-3-one;
4-ethyl-9-((4-(3-fluoroquinolin-7-yl)phenyl)sulfonyl)-1-oxa-4,9-diazaspiro[5.5]undecan-3-one;
9-((4-(3-fluoroquinolin-7-yl)phenyl)sulfonyl)-4-isopropyl-1-oxa-4,9-diazaspiro[5.5]undecan-3-one;
9-((4-(3-methoxyquinolin-7-yl)phenyl)sulfonyl)-4-(1-methylcyclopropyl)-1-oxa-4,9-diazaspiro[5.5]undecan-3-one;
9-((4-(3-fluoroquinolin-7-yl)phenyl)sulfonyl)-4-(1-methylcyclopropyl)-1-oxa-4,9-diazaspiro[5.5]undecan-3-one;
4′-[(4-cyclopropyl-3-oxo-1-oxa-4,9-diazaspiro[5.5]undec-9-yl)sulfonyl]-3-biphenylcarboxylic acid;
4-cyclopropyl-9-{[4′-(1H-tetrazol-5-yl)-4-biphenylyl]sulfonyl}-1-oxa-4,9-diazaspiro[5.5]undecan-3-one;
{4′-[(4-cyclopropyl-3-oxo-1-oxa-4,9-diazaspiro[5.5]undec-9-yl)sulfonyl]-3-biphenylyl}boronic acid;
4′-[(4-cyclopropyl-3-oxo-1-oxa-4,9-diazaspiro[5.5]undec-9-yl)sulfonyl]-3-biphenylsulfonic acid;
2-cyclopropyl-9-{[4-(7-quinolinyl)phenyl]sulfonyl}-2,9-diazaspiro[5.5]undecan-3-one;
ethyl 7-(4((4-cyclopropyl-3-oxo-1-oxa-4,9-diazaspiro[5.5]undecan-9-yl)sulfonyl)phenyl)quinoline-3-carboxylate;
7-(4-((4-cyclopropyl-3-oxo-1-oxa-4,9-diazaspiro[5.5]undecan-9-yl)sulfonyl)phenyl)quinoline-3-carboxylic acid;
9-((4-(3-aminoquinolin-7-yl)phenyl)sulfonyl)-4-cyclopropyl-1-oxa-4,9-diazaspiro[5.5]undecan-3-one;
N-(7-(4-((4-cyclopropyl-3-oxo-1-oxa-4,9-diazaspiro[5.5]undecan-9-yl)sulfonyl)phenyl)quinolin-3-yl)acetamide;
7-(4-((4-cyclopropyl-3-oxo-1-oxa-4,9-diazaspiro[5.5]undecan-9-yl)sulfonyl)phenyl)quinoline-3-carbonitrile;
4-cyclopropyl-9-((4-(3-methoxyquinolin-7-yl)phenyl)sulfonyl)-1-oxa-4,9-diazaspiro[5.5]undecan-3-one;
4-cyclopropyl-9-((4-(3-methylquinolin-7-yl)phenyl)sulfonyl)-1-oxa-4,9-diazaspiro[5.5]undecan-3-one;
9-((4-(3-chloroquinolin-7-yl)phenyl)sulfonyl)-4-cyclopropyl-1-oxa-4,9-diazaspiro[5.5]undecan-3-one;
4-cyclopropyl-9-((4-(3-hydroxyquinolin-7-yl)phenyl)sulfonyl)-1-oxa-4,9-diazaspiro[5.5]undecan-3-one;
7-(4-((4-cyclopropyl-3-oxo-1-oxa-4,9-diazaspiro[5.5]undecan-9-yl)sulfonyl)phenyl)quinoline-3-carboxamide;
N-(7-(4-((4-cyclopropyl-3-oxo-1-oxa-4,9-diazaspiro[5.5]undecan-9-yl)sulfonyl)phenyl)quinolin-3-yl)cyclopropanecarboxamide;
2-(4-((4-cyclopropyl-3-oxo-1-oxa-4,9-diazaspiro[5.5]undecan-9-yl)sulfonyl)phenyl)thieno[3,2-b]pyridine-6-carboxamide;
4-cyclopropyl-9-((4-(3-fluoroquinolin-7-yl)phenyl)sulfonyl)-1-oxa-4,9-diazaspiro[5.5]undecan-3-one; and
N-(7-(4-((4-cyclopropyl-3-oxo-1-oxa-4,9-diazaspiro[5.5]undecan-9-yl)sulfonyl)phenyl)quinolin-3-yl)methanesulfonamide
   or pharmaceutically acceptable salts thereof.

This invention also relates to a method of treating cancer, which comprise administering to a human in need thereof an effective amount of a compound of Formula (I), or a pharmaceutically acceptable salt thereof, as described above, in a pharmaceutical composition, wherein the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone and thyroid.

Typically, but not absolutely, the salts of the present invention are pharmaceutically acceptable salts. Salts encompassed within the term “pharmaceutically acceptable salts” refer to non-toxic salts of the compounds of this invention. Salts of the compounds of the present invention may comprise acid addition salts. In general, the salts are formed from pharmaceutically acceptable inorganic and organic acids. More specific examples of suitable acid salts include maleic, hydrochloric, hydrobromic, sulphuric, phosphoric, nitric, perchloric, fumic, acetic, propionic, succinic, glycolic, formic, lactic, aleic, tartaric, citric, palmoic, malonic, hydroxymaleic, phenylacetic, glutamic, benzoic, salicylic, fumaric, toluenesulfonic, methansulfonic (mesylate), naphthalene-2-sulfonic, benzenesulfonic, hydroxynaphthoic, hydroiodic, malic, teroic, tannic, and the like.

Other representative salts include acetate, benzenesulfonate, benzoate, bicarbonate, bisulfate, bitartrate, borate, calcium edetate, camsylate, carbonate, clavulanate, citrate, dihydrochloride, edisylate, estolate, esylate, fumarate, gluceptate, gluconate, glutamate, glycollylarsanilate, hexylresorcinate, hydrobromide, hydrochloride, hydroxynaphthoate, iodide, isethionate, lactate, lactobionate, laurate, malate, maleate, mandelate, mesylate, methylsulfate, monopotassium maleate, mucate, napsylate, nitrate, oxalate, pamoate (embonate), palmitate, pantothenate, phosphate/diphosphate, polygalacturonate, salicylate, stearate, subacetate, succinate, sulfate, tannate, tartrate, teoclate, tosylate, triethiodide, and valerate salts.

Other salts, which are not pharmaceutically acceptable, may be useful in the preparation of compounds of this invention and these should be considered to form a further aspect of the invention. These salts, such as oxalic or trifluoroacetate, while not in themselves pharmaceutically acceptable, may be useful in the preparation of salts useful as intermediates in obtaining the compounds of the invention and their pharmaceutically acceptable salts.

The compound of Formula (I) or a salt thereof may exist in stereoisomeric forms (e.g., it contains one or more asymmetric carbon atoms). The individual stereoisomers (enantiomers and diastereomers) and mixtures of these are included within the scope of the present invention. The present invention also includes deuterium forms of the present compounds, wherein at least one hydrogen atom of the molecule is replaced with deuterium and the quantity of the deuterium form of the molecular is substantially higher than its natural abundance. The invention also covers the individual isomers of the compound or salt represented by Formula (I) as mixtures with isomers thereof in which one or more chiral centers are inverted. Likewise, it is understood that a compound or salt of Formula (I) may exist in tautomeric forms other than that shown in the formula and these are also included within the scope of the present invention. It is to be understood that the present invention includes all combinations and subsets of the particular groups defined hereinabove. The scope of the present invention includes mixtures of stereoisomers as well as purified enantiomers or enantiomerically/diastereomerically enriched mixtures. Also included within the scope of the invention are individual isomers of the compound represented by Formula (I), as well as any wholly or partially equilibrated mixtures thereof. The present invention also includes the individual isomers of the compound or salt represented by the Formula (I) as well as mixtures with isomers thereof in which one or more chiral centers are inverted. It is to be understood that the present invention includes all combinations and subsets of the particular groups defined hereinabove.

### DEFINITIONS

Terms are used within their accepted meanings. The following definitions are meant to clarify, but not limit, the terms defined.

As used herein, the term “alkyl” refers to a straight or branched chain hydrocarbon radical, preferably having from one to twelve carbon atoms, which may be unsubstituted or substituted, saturated or unsaturated with multiple degrees of substitution included within the present invention. When optionally substituted, the alkyl group is unsubstituted or substituted with suitable substituents selected from the group consisting of: halogen, amino, substituted amino, cyano, hydroxyl, alkoxy, alkylthio, alkylsulfonyl, aminosulfonyl, carboxylic acid, carboxylic ester, carboxamide, aminocarbonyl, and heterocyclyl. Examples of “alkyl” as used herein include, but are not limited to, methyl, ethyl, propyl, isopropyl, isobutyl, n-butyl, t-butyl, isopentyl, n-pentyl, and the like, as well as substituted versions thereof.

As used herein, the term “cycloalkyl” refers to an unsubstituted or substituted mono- or polycyclic non-aromatic saturated ring, which optionally includes an alkylene linker through which the cycloalkyl may be attached. Exemplary “cycloalkyl” groups include, but are not limited to, cyclopropyl, cyclobutyl, cyclopentyl, cyclohexyl, cycloheptyl, and the like, as well as unsubstituted and substituted versions thereof.

As used herein, the term “alkoxy” refers to the group —OR^{a}, where R^{a} is C₁-C₄alkyl or C₃-C₇cycloalkyl as defined above.

As used herein, the term “substituted amino” is meant —NR′R″ wherein each R¹ and R″ is independently selected from a group including hydrogen, C₁-C₆alkyl, acyl, C₃-C₇cycloalkyl, methanesulfonyl, and N,N-dimethylaminosulfonyl, wherein at least one of R¹ and R″ is not hydrogen. Examples of substituted amino includes, but are not limited to alkylamino, dialkylamino, acylamino, and cycloalkylamino.

“Heterocyclic” groups and “heterocycl” may be heteroaryl or heterocycloalkyl groups.

“Heterocycloalkyl” represents a group or moiety comprising a non-aromatic, monovalent monocyclic or bicyclic radical, which is saturated or partially unsaturated, containing 3 to 10 ring atoms, which includes 1 to 4 heteroatoms selected from nitrogen, oxygen and sulfur, and which may be unsubstituted or substituted by one to three of the substituents defined herein. Illustrative examples of heterocycloalkyls include, but are not limited to, azetidinyl, pyrrolidyl (or pyrrolidinyl), piperidinyl, piperazinyl, morpholinyl, tetrahydro-2H-1,4-thiazinyl, tetrahydrofuryl (or tetrahydrofuranyl), dihydrofuryl, oxazolinyl, thiazolinyl, pyrazolinyl, tetrahydropyranyl, dihydropyranyl, 1,3-dioxolanyl, 1,3-dioxanyl, 1,4-dioxanyl, 1,3-oxathiolanyl, 1,3-oxathianyl, 1,3-dithianyl, azabicylo[3.2.1]octyl, azabicylo[3.3.1]nonyl, azabicylo[4.3.0]nonyl, oxabicylo[2.2.1]heptyl and 1,5,9-triazacyclododecyl.

As used herein, the term “heteroaryl”, unless otherwise defined, is meant an aromatic ring system containing carbon(s) and at least one heteroatom. Heteroaryl may be monocyclic or polycyclic, substituted or unsubstituted. A monocyclic heteroaryl group may have 1 to 4 heteroatoms in the ring, while a polycyclic heteroaryl may contain 1 to 8 hetero atoms. A polycyclic heteroaryl ring may contain fused, spiro or bridged ring junctions, for example, bicyclic heteroaryl is a polycyclic heteroaryl. Bicyclic heteroaryl rings may contain from 8 to 12 member atoms. Monocyclic heteroaryl rings may contain from 5 to 8 member atoms (carbons and heteroatoms). Exemplary monocyclic heteroaryl include, but are not limited to furyl (or furanyl), thienyl, pyrrolyl, imidazolyl, pyrazolyl, triazolyl, tetrazinyl, tetrazolyl, thiazolyl, isothiazolyl, oxazolyl, isoxazolyl, furazanyl, oxadiazolyl, thiadiazolyl, isothiazolyl, pyridyl (or pyridinyl), pyridazinyl, pyrazinyl, pyrimidinyl, and triazinyl. Exemplary polycyclic heteroaryl groups include, but are not limited to benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl. Suitable substituents for heteroaryl are described in the definition of “optionally substituted”.

As used herein, the term “heterocyclyl” and grammatical variations thereof refer to an unsubstituted or substituted mono- or polycyclic ring system containing one to four heteroatoms. Heteroatoms include nitrogen, oxygen, and sulfur, including N-oxides, sulfur oxides, and dioxides. A heterocyclic ring may be, but is not limited to, three to eight-membered and is either fully saturated or has one or more degrees of unsaturation. Multiple degrees of substitution are included within the present definition. Examples of “heterocyclic” groups include, but are not limited to tetrahydrofuranyl, pyranyl, 1,4-dioxanyl, 1,3-dioxanyl, piperidinyl, pyrrolidinyl, morpholinyl, azetidinyl, piperazinyl, pyrrolidinonyl, piperazinonyl, pyrazolidinyl, and their various tautomers, as well as unsubstituted and substituted versions thereof. The term “9- or 10-membered heterocyclyl” represents a fully unsaturated or partially unsaturated, bicyclic group, containing 9 or 10 ring atoms, including 1 to 5 heteroatoms independently selected from nitrogen, oxygen and sulfur, which group may be unsubstituted or substituted by one to four of the substituents defined herein. Selected 9- or 10-membered heterocycyl groups contain one nitrogen, oxygen or sulfur ring heteroatom, and optionally contain 1, 2, 3, or 4 additional nitrogen ring atoms and/or 1 additional oxygen or sulfur atom. Examples of 9- or 10-membered heterocyclyl groups include, but are not limited to benzofuranyl, isobenzofuryl, 2,3-dihydrobenzo furyl, 1,3-benzodioxolyl, dihydrobenzodioxinyl, benzothienyl, indolizinyl, indolyl, isoindolyl, indolinyl, isoindolinyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl.

As used herein, the term “aryl”, unless otherwise defined, is meant aromatic, hydrocarbon, ring system. The ring system may be monocyclic or fused polycyclic (e.g., bicyclic, tricyclic, etc.), substituted or unsubstituted. In various embodiments, the monocyclic aryl ring is C₅-C₁₀, or C₅-C₇, or C₅-C₆, where these carbon numbers refer to the number of carbon atoms that form the ring system. A 6-membered ring system, i.e. a phenyl ring, is a suitable aryl group. In various embodiments, the polycyclic ring is a bicyclic aryl group, where suitable bicyclic aryl groups are C₈-C₁₂, or C₉-C₁₀. A naphthyl ring, which has 10 carbon atoms, is a suitable polycyclic aryl group. Suitable substituents for aryl are described in the definition of “optionally substituted”. Examples of aryl radicals include, but are not limited to, phenyl, naphthyl, indenyl, azulenyl, fluorenyl, anthracenyl, phenanthrenyl, tetrahydronaphthyl, indanyl, phenanthridinyl and the like. Unless otherwise indicated, the term “aryl” also includes each possible positional isomer of an aromatic hydrocarbon radical, such as in 1-naphthyl, 2-naphthyl, 5-tetrahydronaphthyl, 6-tetrahydronaphthyl, 1-phenanthridinyl, 2-phenanthridinyl, 3-phenanthridinyl, 4-phenanthridinyl, 7-phenanthridinyl, 8-phenanthridinyl, 9-phenanthridinyl and 10-phenanthridinyl.

As used herein, the term “cyano” refers to the group —CN.

As used herein, the term “optionally” means that the subsequently described event(s) may or may not occur, and includes both event(s) that occur and event(s) that do not occur.

As used herein, unless otherwise defined, the phrase “optionally substituted” or variations thereof denote an optional substitution, including multiple degrees of substitution, suitably with one to four substitutent group. The phrase should not be interpreted as duplicative of the substitutions herein described and depicted. Exemplary optional substituent groups include acyl, C₁-C₆alkyl, alkylsulfonyl, alkoxy, cyano, carboxylic acid, ester, halogen, C₁-C₄alkylhalogen, —CF₃, hydroxyl, oxo, amide, amino, substituted amino, alkylthio, sulfonamide, sulfamide, urea, thiourea and nitro.

The invention further provides a pharmaceutical composition (also referred to as pharmaceutical formulation) comprising a compound of Formula (I) or pharmaceutically acceptable salt, thereof and one or more excipients (also referred to as carriers and/or diluents in the pharmaceutical arts). The excipients are acceptable in the sense of being compatible with the other ingredients of the formulation and not deleterious to the recipient thereof (i.e., the patient).

“Enantiomerically enriched” refers to products whose enantiomeric excess is greater than zero. For example, enantiomerically enriched refers to products whose enantiomeric excess is greater than about 50% ee, greater than about 75% ee, and greater than about 90% ee.

“Enantiomeric excess” or “ee” is the excess of one enantiomer over the other expressed as a percentage. As a result, since both enantiomers are present in equal amounts in a racemic mixture, the enantiomeric excess is zero (0% ee). However, if one enantiomer was enriched such that it constitutes 95% of the product, then the enantiomeric excess would be 90% ee (the amount of the enriched enantiomer, 95%, minus the amount of the other enantiomer, 5%).

“Enantiomerically pure” refers to products whose enantiomeric excess is 100% ee.

“Diasteriomer” refers to a compound having at least two chiral centers.

“Diasteriomer excess” or “de” is the excess of one diasteriomer over the others expressed as a percentage.

“Diasteriomerically pure” refers to products whose diasteriomeric excess is 100% de.

“Half-life” (or “half-lives”) refers to the time required for half of a quantity of a substance to be converted to another chemically distinct specie in vitro or in vivo.

“Halo” or “halogen” refers to fluoro, chloro, bromo, or iodo.

“Heteroatom” refers to a nitrogen, sulphur, or oxygen atom.

“Member atoms” refers to the atom or atoms that form a chain or ring. Where more than one member atom is present in a chain and within a ring, each member atom is covalently bound to an adjacent member atom in the chain or ring. Atoms that make up a substituent group on a chain or ring are not member atoms in the chain or ring.

“Oxo” refers to the substituent group ═O.

As used herein, the term “physiologically functional derivative” refers to any pharmaceutically acceptable derivative of a compound of the present invention, for example, an ester or an amide, which upon administration to a mammal is capable of providing (directly or indirectly) a compound of the present invention or an active metabolite thereof. Such derivatives are clear to those skilled in the art, without undue experimentation, and with reference to the teaching of Burger's Medicinal Chemistry And Drug Discovery, 5th Edition, Vol 1: Principles and Practice, which is incorporated herein by reference to the extent that it teaches physiologically functional derivatives.

“Pharmaceutically acceptable” refers to those compounds, materials, compositions, and dosage forms which are, within the scope of sound medical judgment, suitable for use in contact with the tissues of human beings and animals without excessive toxicity, irritation, or other problem or complication, commensurate with a reasonable benefit/risk ratio.

Compounds within the invention may occur in two or more tautometric forms; all such tautomeric forms are included within the scope of the invention.

In accordance with another aspect of the invention there is provided a process for the preparation of a pharmaceutical composition comprising mixing (or admixing) a compound of Formula (I) or salt thereof with at least one excipient.

### Pharmaceutical Compositions

Pharmaceutical compositions may be in unit dose form containing a predetermined amount of active ingredient per unit dose. Such a unit may contain a therapeutically effective dose of the compound of Formula (I) or salt thereof or a fraction of a therapeutically effective dose such that multiple unit dosage forms might be administered at a given time to achieve the desired therapeutically effective dose. Preferred unit dosage formulations are those containing a daily dose or sub-dose, as herein above recited, or an appropriate fraction thereof, of an active ingredient. Furthermore, such pharmaceutical compositions may be prepared by any of the methods well-known in the pharmacy art.

Pharmaceutical compositions may be adapted for administration by any appropriate route, for example, by oral (including buccal or sublingual), rectal, nasal, topical (including buccal, sublingual, or transdermal), vaginal, or parenteral (including subcutaneous, intramuscular, intravenous, or intradermal) routes. Such compositions may be prepared by any method known in the art of pharmacy, for example, by bringing into association the active ingredient with the excipient(s).

When adapted for oral administration, pharmaceutical compositions may be in discrete units such as tablets or capsules; powders or granules; solutions or suspensions in aqueous or non-aqueous liquids; edible foams or whips; oil-in-water liquid emulsions or water-in-oil liquid emulsions. The compound or salt thereof of the invention or the pharmaceutical composition of the invention may also be incorporated into a candy, a wafer, and/or tongue tape formulation for administration as a “quick-dissolve” medicine.

For instance, for oral administration in the form of a tablet or capsule, the active drug component can be combined with an oral, non-toxic pharmaceutically acceptable inert carrier such as ethanol, glycerol, water, and the like. Powders or granules are prepared by comminuting the compound to a suitable fine size and mixing with a similarly comminuted pharmaceutical carrier such as an edible carbohydrate, as, for example, starch or mannitol. Flavoring, preservative, dispersing, and coloring agents can also be present.

Capsules are made by preparing a powder mixture, as described above, and filling formed gelatin or non-gelatinous sheaths. Glidants and lubricants such as colloidal silica, talc, magnesium stearate, calcium stearate, solid polyethylene glycol can be added to the powder mixture before the filling operation. A disintegrating or solubilizing agent such as agar-agar, calcium carbonate, or sodium carbonate can also be added to improve the availability of the medicine when the capsule is ingested.

Moreover, when desired or necessary, suitable binders, lubricants, disintegrating agents, and coloring agents can also be incorporated into the mixture. Suitable binders include starch, gelatin, natural sugars, such as glucose or beta-lactose, corn sweeteners, natural and synthetic gums such as acacia, tragacanth, sodium alginate, carboxymethylcellulose, polyethylene glycol, waxes, and the like. Lubricants used in these dosage forms include sodium oleate, sodium stearate, magnesium stearate, sodium benzoate, sodium acetate, sodium chloride, and the like. Disintegrators include, without limitation, starch, methylcellulose, agar, bentonite, xanthan gum, and the like.

Tablets are formulated, for example, by preparing a powder mixture, granulating or slugging, adding a lubricant and disintegrant, and pressing into tablets. A powder mixture is prepared by mixing the compound, suitably comminuted, with a diluent or base as described above, and optionally, with a binder such as carboxymethylcellulose, and aliginate, gelatin, or polyvinyl pyrrolidone, a solution retardant such as paraffin, a resorption accelerator such as a quaternary salt, and/or an absorption agent such as bentonite, kaolin, or dicalcium phosphate. The powder mixture can be granulated by wetting a binder such as syrup, starch paste, acadia mucilage, or solutions of cellulosic or polymeric materials and forcing through a screen. As an alternative to granulating, the powder mixture can be run through the tablet machine and the result is imperfectly formed slugs broken into granules. The granules can be lubricated to prevent sticking to the tablet forming dies by means of the addition of stearic acid, a stearate salt, talc, or mineral oil. The lubricated mixture is then compressed into tablets. The compound or salt of the present invention can also be combined with a free-flowing inert carrier and compressed into tablets directly without going through the granulating or slugging steps. A clear opaque protective coating consisting of a sealing coat of shellac, a coating of sugar, or polymeric material, and a polish coating of wax can be provided. Dyestuffs can be added to these coatings to distinguish different dosages.

Oral fluids such as solutions, syrups, and elixirs can be prepared in dosage unit form so that a given quantity contains a predetermined amount of active ingredient. Syrups can be prepared by dissolving the compound or salt thereof of the invention in a suitably flavoured aqueous solution, while elixirs are prepared through the use of a non-toxic alcoholic vehicle. Suspensions can be formulated by dispersing the compound or salt of the invention in a non-toxic vehicle. Solubilizers and emulsifiers, such as ethoxylated isostearyl alcohols and polyoxyethylene sorbitol ethers, preservatives, flavor additives such as peppermint oil, natural sweeteners, saccharin, or other artificial sweeteners, and the like, can also be added.

Where appropriate, dosage unit formulations for oral administration can be microencapsulated. The formulation can also be prepared to prolong or sustain the release as, for example, by coating or embedding particulate material in polymers, wax, or the like.

In the present invention, tablets and capsules are preferred for delivery of the pharmaceutical composition.

As used herein, the term “treatment” includes prophylaxis and refers to alleviating the specified condition, eliminating or reducing one or more symptoms of the condition, slowing or eliminating the progression of the condition, and preventing or delaying the reoccurrence of the condition in a previously afflicted or diagnosed patient or subject. Prophylaxis (or prevention or delay of disease onset) is typically accomplished by administering a drug in the same or similar manner as one would to a patient with the developed disease or condition.

The present invention provides a method of treatment in a mammal, especially a human, suffering from disease conditions targeted by the present compounds. Such treatment comprises the step of administering a therapeutically effective amount of a compound of Formula (I) or salt thereof to said mammal, particularly a human. Treatment can also comprise the step of administering a therapeutically effective amount of a pharmaceutical composition containing a compound of Formula (I) or salt thereof to said mammal, particularly a human.

As used herein, the term “effective amount” means that amount of a drug or pharmaceutical agent that will elicit the biological or medical response of a tissue, system, animal, or human that is being sought, for instance, by a researcher or clinician.

The term “therapeutically effective amount” means any amount which, as compared to a corresponding subject who has not received such amount, results in improved treatment, healing, prevention, or amelioration of a disease, disorder, or side effect, or a decrease in the rate of advancement of a disease or disorder. The term also includes within its scope amounts effective to enhance normal physiological function. For use in therapy, therapeutically effective amounts of a compound of Formula (I), as well as salts thereof, may be administered as the raw chemical. Additionally, the active ingredient may be presented as a pharmaceutical composition.

While it is possible that, for use in therapy, a therapeutically effective amount of a compound of Formula (I) or salt thereof may be administered as the raw chemical, it is typically presented as the active ingredient of a pharmaceutical composition or formulation.

The precise therapeutically effective amount of a compound or salt thereof of the invention will depend on a number of factors, including, but not limited to, the age and weight of the subject (patient) being treated, the precise disorder requiring treatment and its severity, the nature of the pharmaceutical formulation/composition, and route of administration, and will ultimately be at the discretion of the attending physician or veterinarian. Typically, a compound of Formula (I) or salt thereof will be given for the treatment in the range of about 0.1 to 100 mg/kg body weight of recipient (patient, mammal) per day and more usually in the range of 0.1 to 10 mg/kg body weight per day. Acceptable daily dosages may be from about 1 to about 1000 mg/day, and preferably from about 1 to about 100 mg/day. This amount may be given in a single dose per day or in a number (such as two, three, four, five, or more) of sub-doses per day such that the total daily dose is the same. An effective amount of a salt thereof may be determined as a proportion of the effective amount of the compound of Formula (I) per se. Similar dosages should be appropriate for treatment (including prophylaxis) of the other conditions referred herein for treatment. In general, determination of appropriate dosing can be readily arrived at by one skilled in medicine or the pharmacy art.

### Combinations

When a compound of Formula (I) is administered for the treatment of cancer, the term “co-administering” and derivatives thereof as used herein is meant either simultaneous administration or any manner of separate sequential administration of a FAS inhibiting compound, as described herein, and a further active ingredient or ingredients, known to be useful in the treatment of cancer, including chemotherapy and radiation treatment. The term further active ingredient or ingredients, as used herein, includes any compound or therapeutic agent known to or that demonstrates advantageous properties when administered to a patient in need of treatment for cancer. Preferably, if the administration is not simultaneous, the compounds are administered in a close time proximity to each other. Furthermore, it does not matter if the compounds are administered in the same dosage form, e.g. one compound may be administered topically and another compound may be administered orally.

Typically, any anti-neoplastic agent that has activity versus a susceptible tumor being treated may be co-administered in the treatment of cancer in the present invention. Examples of such agents can be found in Cancer Principles and Practice f Oncology by V. T. Devita and S. Hellman (editors), 6^{th} edition (Feb. 15, 2001), Lippincott Williams & Wilkins Publishers. A person of ordinary skill in the art would be able to discern which combinations of agents would be useful based on the particular characteristics of the drugs and the cancer involved. Typical anti-neoplastic agents useful in the present invention include, but are not limited to, anti-microtubule agents such as diterpenoids and vinca alkaloids; platinum coordination complexes; alkylating agents such as nitrogen mustards, oxazaphosphorines, alkylsulfonates, nitrosoureas, and triazenes; antibiotic agents such as anthracycline, actinomycins and bleomycins; topoisomerase II inhibitors such as epipodophyllotoxins; antimetabolites such as purine and pyrimidine analogues and anti-folate compounds; topoisomerase I inhibitors such as camptothecins; hormones and hormonal analogues; signal transduction pathway inhibitors; non-receptor tyrosine kinase angiogenesis inhibitors; immunotherapeutic agents; proapoptotic agents; and cell cycle signaling inhibitors.

Examples of a further active ingredient or ingredients for use in combination or co-administered with the present FAS inhibiting compounds are chemotherapeutic agents.

Anti-microtubule or anti-mitotic agents are phase specific agents active against the microtubules of tumor cells during M or the mitosis phase of the cell cycle. Examples of anti-microtubule agents include, but are not limited to, diterpenoids and vinca alkaloids.

Diterpenoids, which are derived from natural sources, are phase specific anti-cancer agents that operate at the G₂/M phases of the cell cycle. It is believed that the diterpenoids stabilize the β-tubulin subunit of the microtubules, by binding with this protein. Disassembly of the protein appears then to be inhibited with mitosis being arrested and cell death following. Examples of diterpenoids include, but are not limited to, paclitaxel and its analog docetaxel.

Paclitaxel, 5β,20-epoxy-1,2α,4,7β,10β,13α-hexa-hydroxytax-11-en-9-one 4,10-diacetate 2-benzoate 13-ester with (2R,3S)—N-benzoyl-3-phenylisoserine; is a natural diterpene product isolated from the Pacific yew tree Taxus brevifolia and is commercially available as an injectable solution TAXOL®. It is a member of the taxane family of terpenes. It was first isolated in 1971 by Wani et al. J. Am. Chem, Soc., 93:2325. 1971), who characterized its structure by chemical and X-ray crystallographic methods. One mechanism for its activity relates to paclitaxel's capacity to bind tubulin, thereby inhibiting cancer cell growth. Schiff et al., Proc. Natl, Acad, Sci. USA, 77:1561-1565 (1980); Schiff et al., Nature, 277:665-667 (1979); Kumar, J. Biol, Chem, 256: 10435-10441 (1981). For a review of synthesis and anticancer activity of some paclitaxel derivatives see: D. G. I. Kingston et al., Studies in Organic Chemistry vol. 26, entitled “New trends in Natural Products Chemistry 1986”, Attaur-Rahman, P. W. Le Quesne, Eds. (Elsevier, Amsterdam, 1986) pp 219-235.

Paclitaxel has been approved for clinical use in the treatment of refractory ovarian cancer in the United States (Markman et al., Yale Journal of Biology and Medicine, 64:583, 1991; McGuire et al., Ann. Intem, Med., 111:273, 1989) and for the treatment of breast cancer (Holmes et al., J. Nat. Cancer Inst., 83:1797, 1991.) It is a potential candidate for treatment of neoplasms in the skin (Einzig et. al., Proc. Am. Soc. Clin. Oncol., 20:46) and head and neck carcinomas (Forastire et. al., Sem. Oncol., 20:56, 1990). The compound also shows potential for the treatment of polycystic kidney disease (Woo et. al., Nature, 368:750. 1994), lung cancer and malaria. Treatment of patients with paclitaxel results in bone marrow suppression (multiple cell lineages, Ignoff, R. J. et. al, Cancer Chemotherapy Pocket Guid; 1998) related to the duration of dosing above a threshold concentration (50 nM) (Kearns, C. M. et. al., Seminars in Oncology, 3(6) p. 16-23, 1995).

Docetaxel, (2R,3S)—N-carboxy-3-phenylisoserine, N-tert-butyl ester, 13-ester with 5β-20-epoxy-1,2α,4,7β,10β,13α-hexahydroxytax-11-en-9-one 4-acetate 2-benzoate, trihydrate; is commercially available as an injectable solution as TAXOTERE®. Docetaxel is indicated for the treatment of breast cancer. Docetaxel is a semisynthetic derivative of paclitaxel q.v., prepared using a natural precursor, 10-deacetyl-baccatin III, extracted from the needle of the European Yew tree. The dose limiting toxicity of docetaxel is neutropenia.

Vinca alkaloids are phase specific anti-neoplastic agents derived from the periwinkle plant. Vinca alkaloids act at the M phase (mitosis) of the cell cycle by binding specifically to tubulin. Consequently, the bound tubulin molecule is unable to polymerize into microtubules. Mitosis is believed to be arrested in metaphase with cell death following. Examples of vinca alkaloids include, but are not limited to, vinblastine, vincristine, and vinorelbine.

Vinblastine, vincaleukoblastine sulfate, is commercially available as VELBAN® as an injectable solution. Although, it has possible indication as a second line therapy of various solid tumors, it is primarily indicated in the treatment of testicular cancer and various lymphomas including Hodgkin's Disease; and lymphocytic and histiocytic lymphomas. Myelosuppression is the dose limiting side effect of vinblastine.

Vincristine, vincaleukoblastine, 22-oxo-, sulfate, is commercially available as ONCOVIN® as an injectable solution. Vincristine is indicated for the treatment of acute leukemias and has also found use in treatment regimens for Hodgkin's and non-Hodgkin's malignant lymphomas. Alopecia and neurologic effects are the most common side effect of vincristine and to a lesser extent myelosupression and gastrointestinal mucositis effects occur.

Vinorelbine, 3′,4′-didehydro-4′-deoxy-C′-norvincaleukoblastine [R—(R*,R*)-2,3-dihydroxybutanedioate (1:2)(salt)], commercially available as an injectable solution of vinorelbine tartrate (NAVELBINE®), is a semisynthetic vinca alkaloid. Vinorelbine is indicated as a single agent or in combination with other chemotherapeutic agents, such as cisplatin, in the treatment of various solid tumors, particularly non-small cell lung, advanced breast, and hormone refractory prostate cancers. Myelosuppression is the most common dose limiting side effect of vinorelbine.

Platinum coordination complexes are non-phase specific anti-cancer agents, which are interactive with DNA. The platinum complexes enter tumor cells, undergo, aquation and form intra- and interstrand crosslinks with DNA causing adverse biological effects to the tumor. Examples of platinum coordination complexes include, but are not limited to, cisplatin and carboplatin.

Cisplatin, cis-diamminedichloroplatinum, is commercially available as PLATINOL® as an injectable solution. Cisplatin is primarily indicated in the treatment of metastatic testicular and ovarian cancer and advanced bladder cancer. The primary dose limiting side effects of cisplatin are nephrotoxicity, which may be controlled by hydration and diuresis, and ototoxicity.

Carboplatin, platinum, diammine [1,1-cyclobutane-dicarboxylate(2-)-O,O′], is commercially available as PARAPLATIN® as an injectable solution. Carboplatin is primarily indicated in the first and second line treatment of advanced ovarian carcinoma. Bone marrow suppression is the dose limiting toxicity of carboplatin.

Alkylating agents are non-phase anti-cancer specific agents and strong electrophiles. Typically, alkylating agents form covalent linkages, by alkylation, to DNA through nucleophilic moieties of the DNA molecule such as phosphate, amino, sulfhydryl, hydroxyl, carboxyl, and imidazole groups. Such alkylation disrupts nucleic acid function leading to cell death. Examples of alkylating agents include, but are not limited to, nitrogen mustards such as cyclophosphamide, melphalan, and chlorambucil; alkyl sulfonates such as busulfan; nitrosoureas such as carmustine; and triazenes such as dacarbazine.

Cyclophosphamide, 2-[bis(2-chloroethyl)amino]tetrahydro-2H-1,3,2-oxazaphosphorine 2-oxide monohydrate, is commercially available as an injectable solution or tablets as CYTOXAN®. Cyclophosphamide is indicated as a single agent or in combination with other chemotherapeutic agents, in the treatment of malignant lymphomas, multiple myeloma, and leukemias. Alopecia, nausea, vomiting and leukopenia are the most common dose limiting side effects of cyclophosphamide.

Melphalan, 4-[bis(2-chloroethyl)amino]-L-phenylalanine, is commercially available as an injectable solution or tablets as ALKERAN®. Melphalan is indicated for the palliative treatment of multiple myeloma and non-resectable epithelial carcinoma of the ovary. Bone marrow suppression is the most common dose limiting side effect of melphalan.

Chlorambucil, 4-[bis(2-chloroethyl)amino]benzenebutanoic acid, is commercially available as LEUKERAN® tablets. Chlorambucil is indicated for the palliative treatment of chronic lymphatic leukemia, and malignant lymphomas such as lymphosarcoma, giant follicular lymphoma, and Hodgkin's disease. Bone marrow suppression is the most common dose limiting side effect of chlorambucil.

Busulfan, 1,4-butanediol dimethanesulfonate, is commercially available as MYLERAN® TABLETS. Busulfan is indicated for the palliative treatment of chronic myelogenous leukemia. Bone marrow suppression is the most common dose limiting side effects of busulfan.

Carmustine, 1,3-[bis(2-chloroethyl)-1-nitrosourea, is commercially available as single vials of lyophilized material as BiCNU®. Carmustine is indicated for the palliative treatment as a single agent or in combination with other agents for brain tumors, multiple myeloma, Hodgkin's disease, and non-Hodgkin's lymphomas. Delayed myelosuppression is the most common dose limiting side effects of carmustine.

Dacarbazine, 5-(3,3-dimethyl-1-triazeno)-imidazole-4-carboxamide, is commercially available as single vials of material as DTIC-Dome®. Dacarbazine is indicated for the treatment of metastatic malignant melanoma and in combination with other agents for the second line treatment of Hodgkin's Disease. Nausea, vomiting, and anorexia are the most common dose limiting side effects of dacarbazine.

Antibiotic anti-neoplastics are non-phase specific agents, which bind or intercalate with DNA. Typically, such action results in stable DNA complexes or strand breakage, which disrupts ordinary function of the nucleic acids leading to cell death. Examples of antibiotic anti-neoplastic agents include, but are not limited to, actinomycins such as dactinomycin, anthrocyclins such as daunorubicin and doxorubicin; and bleomycins.

Dactinomycin, also know as Actinomycin D, is commercially available in injectable form as COSMEGEN®. Dactinomycin is indicated for the treatment of Wilm's tumor and rhabdomyosarcoma. Nausea, vomiting, and anorexia are the most common dose limiting side effects of dactinomycin.

Daunorubicin, (8S-cis-)-8-acetyl-10-[(3-amino-2,3,6-trideoxy-α-L-lyxo-hexopyranosyl)oxy]-7,8,9,10-tetrahydro-6,8,11-trihydroxy-1-methoxy-5,12 naphthacenedione hydrochloride, is commercially available as a liposomal injectable form as DAUNOXOME@ or as an injectable as CERUBIDINE®. Daunorubicin is indicated for remission induction in the treatment of acute nonlymphocytic leukemia and advanced HIV associated Kaposi's sarcoma. Myelosuppression is the most common dose limiting side effect of daunorubicin.

Doxorubicin, (8S,10S)-10-[(3-amino-2,3,6-trideoxy-α-L-lyxo-hexopyranosyl)oxy]-8-glycoloyl, 7,8,9,10-tetrahydro-6,8,11-trihydroxy-1-methoxy-5,12 naphthacenedione hydrochloride, is commercially available as an injectable form as RUBEX® or ADRIAMYCIN RDF®. Doxorubicin is primarily indicated for the treatment of acute lymphoblastic leukemia and acute myeloblastic leukemia, but is also a useful component in the treatment of some solid tumors and lymphomas. Myelosuppression is the most common dose limiting side effect of doxorubicin.

Bleomycin, a mixture of cytotoxic glycopeptide antibiotics isolated from a strain of Streptomyces verticillus, is commercially available as BLENOXANE®. Bleomycin is indicated as a palliative treatment, as a single agent or in combination with other agents, of squamous cell carcinoma, lymphomas, and testicular carcinomas. Pulmonary and cutaneous toxicities are the most common dose limiting side effects of bleomycin.

Topoisomerase II inhibitors include, but are not limited to, epipodophyllotoxins.

Epipodophyllotoxins are phase specific anti-neoplastic agents derived from the mandrake plant. Epipodophyllotoxins typically affect cells in the S and G₂ phases of the cell cycle by forming a ternary complex with topoisomerase II and DNA causing DNA strand breaks. The strand breaks accumulate and cell death follows. Examples of epipodophyllotoxins include, but are not limited to, etoposide and teniposide.

Etoposide, 4′-demethyl-epipodophyllotoxin 9[4,6-0-(R)-ethylidene-β-D-glucopyranoside], is commercially available as an injectable solution or capsules as VePESID® and is commonly known as VP-16. Etoposide is indicated as a single agent or in combination with other chemotherapy agents in the treatment of testicular and non-small cell lung cancers. Myelosuppression is the most common side effect of etoposide. The incidence of leucopenia tends to be more severe than thrombocytopenia.

Teniposide, 4′-demethyl-epipodophyllotoxin 9[4,6-0-(R)-thenylidene-β-D-glucopyranoside], is commercially available as an injectable solution as VUMON® and is commonly known as VM-26. Teniposide is indicated as a single agent or in combination with other chemotherapy agents in the treatment of acute leukemia in children. Myelosuppression is the most common dose limiting side effect of teniposide. Teniposide can induce both leucopenia and thrombocytopenia.

Antimetabolite neoplastic agents are phase specific anti-neoplastic agents that act at S phase (DNA synthesis) of the cell cycle by inhibiting DNA synthesis or by inhibiting purine or pyrimidine base synthesis and thereby limiting DNA synthesis. Consequently, S phase does not proceed and cell death follows. Examples of antimetabolite anti-neoplastic agents include, but are not limited to, fluorouracil, methotrexate, cytarabine, mercaptopurine, thioguanine, and gemcitabine.

5-fluorouracil, 5-fluoro-2,4-(1H,3H) pyrimidinedione, is commercially available as fluorouracil. Administration of 5-fluorouracil leads to inhibition of thymidylate synthesis and is also incorporated into both RNA and DNA. The result typically is cell death. 5-fluorouracil is indicated as a single agent or in combination with other chemotherapy agents in the treatment of carcinomas of the breast, colon, rectum, stomach and pancreas. Myelosuppression and mucositis are dose limiting side effects of 5-fluorouracil. Other fluoropyrimidine analogs include 5-fluoro deoxyuridine (floxuridine) and 5-fluorodeoxyuridine monophosphate.

Cytarabine, 4-amino-1-β-D-arabinofuranosyl-2 (1H)-pyrimidinone, is commercially available as CYTOSAR-U® and is commonly known as Ara-C. It is believed that cytarabine exhibits cell phase specificity at S-phase by inhibiting DNA chain elongation by terminal incorporation of cytarabine into the growing DNA chain. Cytarabine is indicated as a single agent or in combination with other chemotherapy agents in the treatment of acute leukemia. Other cytidine analogs include 5-azacytidine and 2′,2′-difluorodeoxycytidine (gemcitabine). Cytarabine induces leucopenia, thrombocytopenia, and mucositis.

Mercaptopurine, 1,7-dihydro-6H-purine-6-thione monohydrate, is commercially available as PURINETHOL®. Mercaptopurine exhibits cell phase specificity at S-phase by inhibiting DNA synthesis by an as of yet unspecified mechanism. Mercaptopurine is indicated as a single agent or in combination with other chemotherapy agents in the treatment of acute leukemia. Myelosuppression and gastrointestinal mucositis are expected side effects of mercaptopurine at high doses. A useful mercaptopurine analog is azathioprine.

Thioguanine, 2-amino-1,7-dihydro-6H-purine-6-thione, is commercially available as TABLOID®. Thioguanine exhibits cell phase specificity at S-phase by inhibiting DNA synthesis by an as of yet unspecified mechanism. Thioguanine is indicated as a single agent or in combination with other chemotherapy agents in the treatment of acute leukemia. Myelosuppression, including leucopenia, thrombocytopenia, and anemia, is the most common dose limiting side effect of thioguanine administration. However, gastrointestinal side effects occur and can be dose limiting. Other purine analogs include pentostatin, erythrohydroxynonyladenine, fludarabine phosphate, and cladribine.

Gemcitabine, 2′-deoxy-2′,2′-difluorocytidine monohydrochloride (β-isomer), is commercially available as GEMZAR®. Gemcitabine exhibits cell phase specificity at S-phase and by blocking progression of cells through the G1/S boundary. Gemcitabine is indicated in combination with cisplatin in the treatment of locally advanced non-small cell lung cancer and alone in the treatment of locally advanced pancreatic cancer. Myelosuppression, including leucopenia, thrombocytopenia, and anemia, is the most common dose limiting side effect of gemcitabine administration.

Methotrexate, N-[4[[(2,4-diamino-6-pteridinyl)methyl]methylamino]benzoyl]-L-glutamic acid, is commercially available as methotrexate sodium. Methotrexate exhibits cell phase effects specifically at S-phase by inhibiting DNA synthesis, repair and/or replication through the inhibition of dyhydrofolic acid reductase which is required for synthesis of purine nucleotides and thymidylate. Methotrexate is indicated as a single agent or in combination with other chemotherapy agents in the treatment of choriocarcinoma, meningeal leukemia, non-Hodgkin's lymphoma, and carcinomas of the breast, head, neck, ovary and bladder. Myelosuppression (leucopenia, thrombocytopenia, and anemia) and mucositis are expected side effect of methotrexate administration.

Camptothecins, including, camptothecin and camptothecin derivatives are available or under development as Topoisomerase I inhibitors. Camptothecins cytotoxic activity is believed to be related to its Topoisomerase I inhibitory activity. Examples of camptothecins include, but are not limited to irinotecan, topotecan, and the various optical forms of 7-(4-methylpiperazino-methylene)-10,11-ethylenedioxy-20-camptothecin described below.

Irinotecan HCl, (4S)-4,11-diethyl-4-hydroxy-9-[(4-piperidinopiperidino) carbonyloxy]-1H-pyrano[3′,4′,6,7]indolizino[1,2-b]quinoline-3,14(4H,12H)-dione hydrochloride, is commercially available as the injectable solution CAMPTOSAR®.

Irinotecan is a derivative of camptothecin which binds, along with its active metabolite SN-38, to the topoisomerase I-DNA complex. It is believed that cytotoxicity occurs as a result of irreparable double strand breaks caused by interaction of the topoisomerase I:DNA:irintecan or SN-38 ternary complex with replication enzymes.

Irinotecan is indicated for treatment of metastatic cancer of the colon or rectum. The dose limiting side effects of irinotecan HCl are myelosuppression, including neutropenia, and GI effects, including diarrhea.

Topotecan HCl, (S)-10-[(dimethylamino)methyl]-4-ethyl-4,9-dihydroxy-1H-pyrano[3′,4′,6,7]indolizino[1,2-b]quinoline-3,14-(4H,12H)-dione monohydrochloride, is commercially available as the injectable solution HYCAMTIN®. Topotecan is a derivative of camptothecin which binds to the topoisomerase I-DNA complex and prevents religation of singles strand breaks caused by Topoisomerase I in response to torsional strain of the DNA molecule. Topotecan is indicated for second line treatment of metastatic carcinoma of the ovary and small cell lung cancer. The dose limiting side effect of topotecan HCl is myelosuppression, primarily neutropenia.

Also of interest, is the camptothecin derivative of formula A following, currently under development, including the racemic mixture (R,S) form as well as the R and S enantiomers:

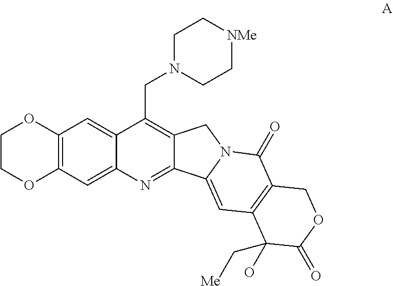 [SMILES: CCC1(O)C(=O)OCc2c1cc1n(c2=O)Cc2c-1nc1cc3c(cc1c2CN1CCN(C)CC1)OCCO3]

known by the chemical name “7-(4-methylpiperazino-methylene)-10,11-ethylenedioxy-20(R,S)-camptothecin (racemic mixture) or “7-(4-methylpiperazino-methylene)-10,11-ethylenedioxy-20(R)-camptothecin (R enantiomer) or “7-(4-methylpiperazino-methylene)-10,11-ethylenedioxy-20(S)-camptothecin (S enantiomer). Such compound as well as related compounds are described, including methods of making, in U.S. Pat. Nos. 6,063,923; 5,342,947; 5,559,235; 5,491,237 and pending U.S. patent application Ser. No. 08/977,217 filed Nov. 24, 1997.

Hormones and hormonal analogues are useful compounds for treating cancers in which there is a relationship between the hormone(s) and growth and/or lack of growth of the cancer. Examples of hormones and hormonal analogues useful in cancer treatment include, but are not limited to, adrenocorticosteroids such as prednisone and prednisolone which are useful in the treatment of malignant lymphoma and acute leukemia in children; aminoglutethimide and other aromatase inhibitors such as anastrozole, letrazole, vorazole, and exemestane useful in the treatment of adrenocortical carcinoma and hormone dependent breast carcinoma containing estrogen receptors; progestrins such as megestrol acetate useful in the treatment of hormone dependent breast cancer and endometrial carcinoma; estrogens, androgens, and anti-androgens such as flutamide, nilutamide, bicalutamide, cyproterone acetate and 5α-reductases such as finasteride and dutasteride, useful in the treatment of prostatic carcinoma and benign prostatic hypertrophy; anti-estrogens such as tamoxifen, toremifene, raloxifene, droloxifene, iodoxyfene, as well as selective estrogen receptor modulators (SERMS) such those described in U.S. Pat. Nos. 5,681,835, 5,877,219, and 6,207,716, useful in the treatment of hormone dependent breast carcinoma and other susceptible cancers; and gonadotropin-releasing hormone (GnRH) and analogues thereof which stimulate the release of leutinizing hormone (LH) and/or follicle stimulating hormone (FSH) for the treatment prostatic carcinoma, for instance, LHRH agonists and antagagonists such as goserelin acetate and luprolide.

Signal transduction pathway inhibitors are those inhibitors, which block or inhibit a chemical process which evokes an intracellular change. As used herein this change is cell proliferation or differentiation. Signal transduction inhibitors useful in the present invention include inhibitors of receptor tyrosine kinases, non-receptor tyrosine kinases, SH2/SH3domain blockers, serine/threonine kinases, phosphotidyl inositol-3 kinases, myo-inositol signaling, and Ras oncogenes.

Several protein tyrosine kinases catalyse the phosphorylation of specific tyrosyl residues in various proteins involved in the regulation of cell growth. Such protein tyrosine kinases can be broadly classified as receptor or non-receptor kinases.

Receptor tyrosine kinases are transmembrane proteins having an extracellular ligand binding domain, a transmembrane domain, and a tyrosine kinase domain. Receptor tyrosine kinases are involved in the regulation of cell growth and are generally termed growth factor receptors. Inappropriate or uncontrolled activation of many of these kinases, i.e. aberrant kinase growth factor receptor activity, for example by over-expression or mutation, has been shown to result in uncontrolled cell growth. Accordingly, the aberrant activity of such kinases has been linked to malignant tissue growth. Consequently, inhibitors of such kinases could provide cancer treatment methods. Growth factor receptors include, for example, epidermal growth factor receptor (EGFr), platelet derived growth factor receptor (PDGFr), erbB2, erbB4, vascular endothelial growth factor receptor (VEGFr), tyrosine kinase with immunoglobulin-like and epidermal growth factor homology domains (TIE-2), insulin growth factor-I (IGFI) receptor, macrophage colony stimulating factor (cfms), BTK, ckit, cmet, fibroblast growth factor (FGF) receptors, Trk receptors (TrkA, TrkB, and TrkC), ephrin (eph) receptors, and the RET protooncogene. Several inhibitors of growth receptors are under development and include ligand antagonists, antibodies, tyrosine kinase inhibitors and anti-sense oligonucleotides. Growth factor receptors and agents that inhibit growth factor receptor function are described, for instance, in Kath, John C., Exp. Opin. Ther. Patents (2000) 10(6):803-818; Shawver et al DDT Vol 2, No. 2 Feb. 1997; and Lofts, F. J. et al, “Growth factor receptors as targets”, New Molecular Targets for Cancer Chemotherapy, ed. Workman, Paul and Kerr, David, CRC press 1994, London.

Tyrosine kinases, which are not growth factor receptor kinases are termed non-receptor tyrosine kinases. Non-receptor tyrosine kinases useful in the present invention, which are targets or potential targets of anti-cancer drugs, include cSrc, Lck, Fyn, Yes, Jak, cAbl, FAK (Focal adhesion kinase), Brutons tyrosine kinase, and Bcr-Abl. Such non-receptor kinases and agents which inhibit non-receptor tyrosine kinase function are described in Sinh, S, and Corey, S. J., (1999) Journal of Hematotherapy and Stem Cell Research 8 (5): 465-80; and Bolen, J. B., Brugge, J. S., (1997) Annual review of Immunology. 15: 371-404.

SH2/SH3 domain blockers are agents that disrupt SH2 or SH3 domain binding in a variety of enzymes or adaptor proteins including, PI3-K p85 subunit, Src family kinases, adaptor molecules (Shc, Crk, Nck, Grb2) and Ras-GAP. SH2/SH3 domains as targets for anti-cancer drugs are discussed in Smithgall, T. E. (1995), Journal of Pharmacological and Toxicological Methods. 34(3) 125-32.

Inhibitors of Serine/Threonine Kinases including MAP kinase cascade blockers which include blockers of Raf kinases (rafk), Mitogen or Extracellular Regulated Kinase (MEKs), and Extracellular Regulated Kinases (ERKs); and Protein kinase C family member blockers including blockers of PKCs (alpha, beta, gamma, epsilon, mu, lambda, iota, zeta) IkB kinase family (IKKa, IKKb), PKB family kinases, AKT kinase family members, and TGF beta receptor kinases. Such Serine/Threonine kinases and inhibitors thereof are described in Yamamoto, T., Taya, S., Kaibuchi, K., (1999), Journal of Biochemistry. 126 (5) 799-803; Brodt, P, Samani, A., and Navab, R. (2000), Biochemical Pharmacology, 60. 1101-1107; Massague, J., Weis-Garcia, F. (1996) Cancer Surveys. 27:41-64; Philip, P. A., and Harris, A. L. (1995), Cancer Treatment and Research. 78: 3-27, Lackey, K. et al Bioorganic and Medicinal Chemistry Letters, (10), 2000, 223-226; U.S. Pat. No. 6,268,391; and Martinez-Iacaci, L., et al, Int. J. Cancer (2000), 88(1), 44-52.

Inhibitors of Phosphotidyl inositol-3 Kinase family members including blockers of PI3-kinase, ATM, DNA-PK, and Ku are also useful in the present invention. Such kinases are discussed in Abraham, R. T. (1996), Current Opinion in Immunology. 8 (3) 412-8; Canman, C. E., Lim, D. S. (1998), Oncogene 17 (25) 3301-3308; Jackson, S. P. (1997), International Journal of Biochemistry and Cell Biology. 29 (7):935-8; and Zhong, H. et al, Cancer res, (2000) 60(6), 1541-1545.

Also useful in the present invention are Myo-inositol signaling inhibitors such as phospholipase C blockers and Myoinositol analogues. Such signal inhibitors are described in Powis, G., and Kozikowski A., (1994) New Molecular Targets for Cancer Chemotherapy ed., Paul Workman and David Kerr, CRC press 1994, London.

Another group of signal transduction pathway inhibitors are inhibitors of Ras Oncogene. Such inhibitors include inhibitors of farnesyltransferase, geranyl-geranyl transferase, and CAAX proteases as well as anti-sense oligonucleotides, ribozymes and immunotherapy. Such inhibitors have been shown to block ras activation in cells containing wild type mutant ras, thereby acting as antiproliferation agents. Ras oncogene inhibition is discussed in Scharovsky, O. G., Rozados, V. R., Gervasoni, S. I. Matar, P. (2000), Journal of Biomedical Science. 7(4) 292-8; Ashby, M. N. (1998), Current Opinion in Lipidology. 9 (2) 99-102; and BioChim. Biophys. Acta, (19899) 1423(3):19-30.

As mentioned above, antibody antagonists to receptor kinase ligand binding may also serve as signal transduction inhibitors. This group of signal transduction pathway inhibitors includes the use of humanized antibodies to the extracellular ligand binding domain of receptor tyrosine kinases. For example Imclone C225 EGFR specific antibody (see Green, M. C. et al, Monoclonal Antibody Therapy for Solid Tumors, Cancer Treat. Rev., (2000), 26(4), 269-286); Herceptin® erbB2 antibody (see Tyrosine Kinase Signalling in Breast cancer:erbB Family Receptor Tyrosine Kniases, Breast cancer Res., 2000, 2(3), 176-183); and 2CB VEGFR2 specific antibody (see Brekken, R. A. et al, Selective Inhibition of VEGFR2Activity by a monoclonal Anti-VEGF antibody blocks tumor growth in mice, Cancer Res. (2000) 60, 5117-5124).

Non-receptor kinase angiogenesis inhibitors may also find use in the present invention. Inhibitors of angiogenesis related VEGFR and TIE2 are discussed above in regard to signal transduction inhibitors (both receptors are receptor tyrosine kinases). Angiogenesis in general is linked to erbB2/EGFR signaling since inhibitors of erbB2 and EGFR have been shown to inhibit angiogenesis, primarily VEGF expression. Thus, the combination of an erbB2/EGFR inhibitor with an inhibitor of angiogenesis makes sense. Accordingly, non-receptor tyrosine kinase inhibitors may be used in combination with the EGFR/erbB2 inhibitors of the present invention. For example, anti-VEGF antibodies, which do not recognize VEGFR (the receptor tyrosine kinase), but bind to the ligand; small molecule inhibitors of integrin (alpha beta₃) that will inhibit angiogenesis; endostatin and angiostatin (non-RTK) may also prove useful in combination with the disclosed erb family inhibitors. (See Bruns C J et al (2000), Cancer Res., 60: 2926-2935; Schreiber A B, Winkler M E, and Derynck R. (1986), Science, 232: 1250-1253; Yen L et al. (2000), Oncogene 19: 3460-3469).

Agents used in immunotherapeutic regimens may also be useful in combination with the compounds of formula (I). There are a number of immunologic strategies to generate an immune response against erbB2 or EGFR. These strategies are generally in the realm of tumor vaccinations. The efficacy of immunologic approaches may be greatly enhanced through combined inhibition of erbB2/EGFR signaling pathways using a small molecule inhibitor. Discussion of the immunologic/tumor vaccine approach against erbB2/EGFR are found in Reilly R T et al. (2000), Cancer Res. 60: 3569-3576; and Chen Y, Hu D, Eling D J, Robbins J, and Kipps T J. (1998), Cancer Res. 58: 1965-1971.

Agents used in proapoptotic regimens (e.g., bcl-2 antisense oligonucleotides) may also be used in the combination of the present invention. Members of the Bcl-2 family of proteins block apoptosis. Upregulation of bcl-2 has therefore been linked to chemoresistance. Studies have shown that the epidermal growth factor (EGF) stimulates anti-apoptotic members of the bcl-2 family (i.e., mcl-1). Therefore, strategies designed to downregulate the expression of bcl-2 in tumors have demonstrated clinical benefit and are now in Phase II/III trials, namely Genta's G3139 bcl-2 antisense oligonucleotide. Such proapoptotic strategies using the antisense oligonucleotide strategy for bcl-2 are discussed in Water J S et al. (2000), J. Clin. Oncol. 18: 1812-1823; and Kitada S et al. (1994), Antisense Res. Dev. 4: 71-79.

Cell cycle signalling inhibitors inhibit molecules involved in the control of the cell cycle. A family of protein kinases called cyclin dependent kinases (CDKs) and their interaction with a family of proteins termed cyclins controls progression through the eukaryotic cell cycle. The coordinate activation and inactivation of different cyclin/CDK complexes is necessary for normal progression through the cell cycle. Several inhibitors of cell cycle signalling are under development. For instance, examples of cyclin dependent kinases, including CDK2, CDK4, and CDK6 and inhibitors for the same are described in, for instance, Rosania et al, Exp. Opin. Ther. Patents (2000) 10(2):215-230.

Rituximab is a chimeric monoclonal antibody which is sold as RITUXAN® and MABTHERA®. Rituximab binds to CD20 on B cells and causes cell apoptosis. Rituximab is administered intravenously and is approved for treatment of rheumatoid arthritis and B-cell non-Hodgkin's lymphoma.

Ofatumumab is a fully human monoclonal antibody which is sold as ARZERRA®. Ofatumumab binds to CD20 on B cells and is used to treat chronic lymphocytic leukemia (CLL; a type of cancer of the white blood cells) in adults who are refractory to treatment with fludarabine (Fludara) and alemtuzumab (Campath).

mTOR inhibitors include but are not limited to rapamycin and rapalogs, RAD001 or everolimus (Afinitor), CCl-779 or temsirolimus, AP23573, AZD8055, WYE-354, WYE-600, WYE-687 and Pp121.

Bexarotene is sold as Targretin® and is a member of a subclass of retinoids that selectively activate retinoid X receptors (RXRs). These retinoid receptors have biologic activity distinct from that of retinoic acid receptors (RARs). The chemical name is 4-[1-(5,6,7,8-tetrahydro-3,5,5,8,8-pentamethyl-2-naphthalenyl)ethenyl]benzoic acid. Bexarotene is used to treat cutaneous T-cell lymphoma (CTCL, a type of skin cancer) in people whose disease could not be treated successfully with at least one other medication.

Sorafenib marketed as Nexavar® is in a class of medications called multikinase inhibitors. Its chemical name is 4-[4-[[4-chloro-3-(trifluoromethyl)phenyl]carbamoylamino]phenoxy]-N-methyl-pyridine-2-carboxamide. Sorafenib is used to treat advanced renal cell carcinoma (a type of cancer that begins in the kidneys). Sorafenib is also used to treat unresectable hepatocellular carcinoma (a type of liver cancer that cannot be treated with surgery).

In one embodiment, the cancer treatment method of the claimed invention includes the co-administration a compound of Formula (I) and/or a pharmaceutically acceptable salt, hydrate, solvate or pro-drug thereof and at least one anti-neoplastic agent, such as one selected from the group consisting of anti-microtubule agents, platinum coordination complexes, alkylating agents, antibiotic agents, topoisomerase II inhibitors, antimetabolites, topoisomerase I inhibitors, hormones and hormonal analogues, signal transduction pathway inhibitors, non-receptor tyrosine kinase angiogenesis inhibitors, immunotherapeutic agents, proapoptotic agents, and cell cycle signaling inhibitors.

In another embodiments the present invention provides uses of any of the compounds of Formula I from the treatment of cancer. In one aspect, the present invention provides uses of the compounds of Formula I for the manufacture of a medicament for the treatment of cancer.

### Experimentals

### Preparation

The derivatives described herein were prepared by the general methods described below. Formulas and R group designations used in the schemes below are meant to be used for this section only, and they may be inconsistent with those in the claims.

### Schemes/Experimentals

A piperidine aryl sulfonamide can be prepared by condensation of an aryl sulfonyl chloride with 4-piperidinone (Scheme I). The intermediate ketone can be readily converted to an epoxide, which can be opened with various amines to give an amino alcohol intermediate. Cyclization to the spirocyclic lactam can be accomplished in two steps with a reagent such as chloroacetyl chloride. Final products can then be prepared by Suzuki cross-coupling with various aryl or heteroaryl boronates or boronic acids.

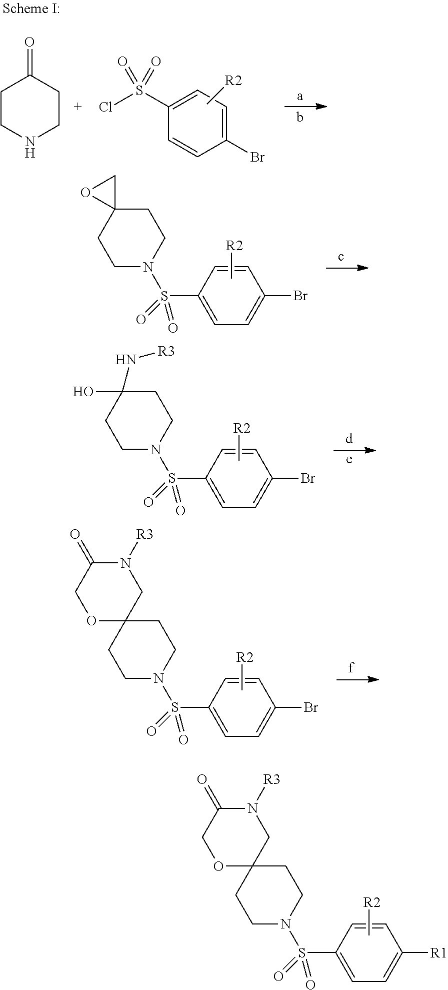

Conditions: a) pyridine, CH₂Cl₂; b) trimethylsulfoxonium iodide, NaH, DMSO; c) R3-NH₂, MeOH or EtOH, reflux; d) chloroacetyl chloride, Et₃N, CH₂Cl₂; e) NaH, DMSO, THF, rt to reflux; f) R1-B(OR)₂, PdCl₂(dppf)-CH₂Cl₂ (cat.), aq K₂CO₃, dioxane, 100-150° C.

Alternatively, the core spirocycle can be prepared first (Scheme II), starting from a protected piperidinone and following a synthetic route similar to that outlined for Scheme I.

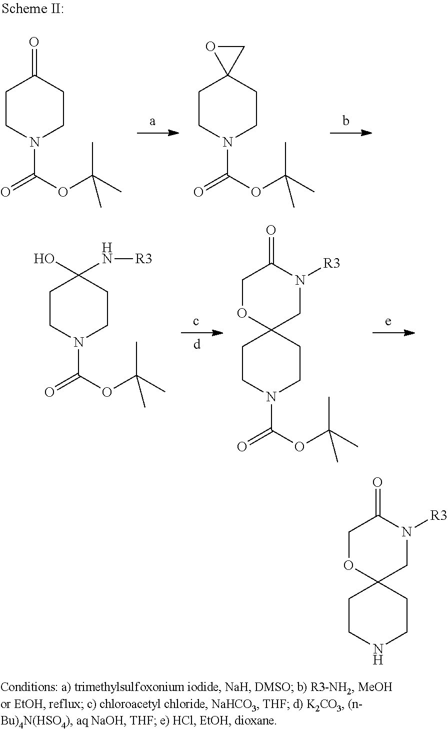

The spirocyclic core can then be elaborated to the final products through condensation with an aryl sulfonyl chloride and Suzuki cross-coupling with various aryl or heteroaryl boronates or boronic acids (Scheme III).

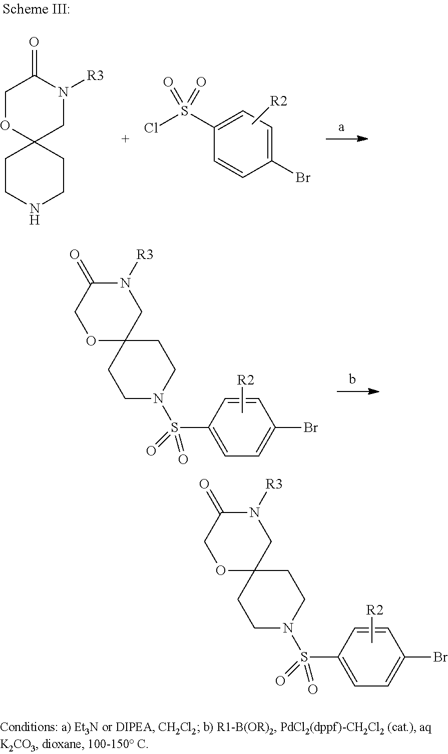

To allow for greater flexibility in the Suzuki cross-coupling reaction, the spirocyclic aryl bromide can also be converted to the intermediate boronate and then coupled with various aryl or heteroaryl halides to prepare the target compounds (Scheme IV).

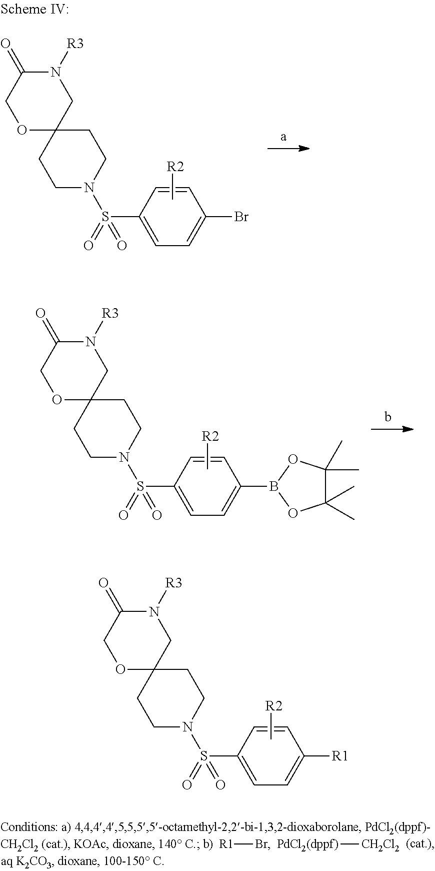

Modifications to the lactam can be made from functionalized piperidines that are protected or that are already condensed with an aryl sulfonyl chloride (Scheme V). Standard manipulations to build up the lactam ring are typically employed before Suzuki cross-coupling with various aryl or heteroaryl boronic esters or acids to yield the target analogs. Additional examples are described in the experimental section.

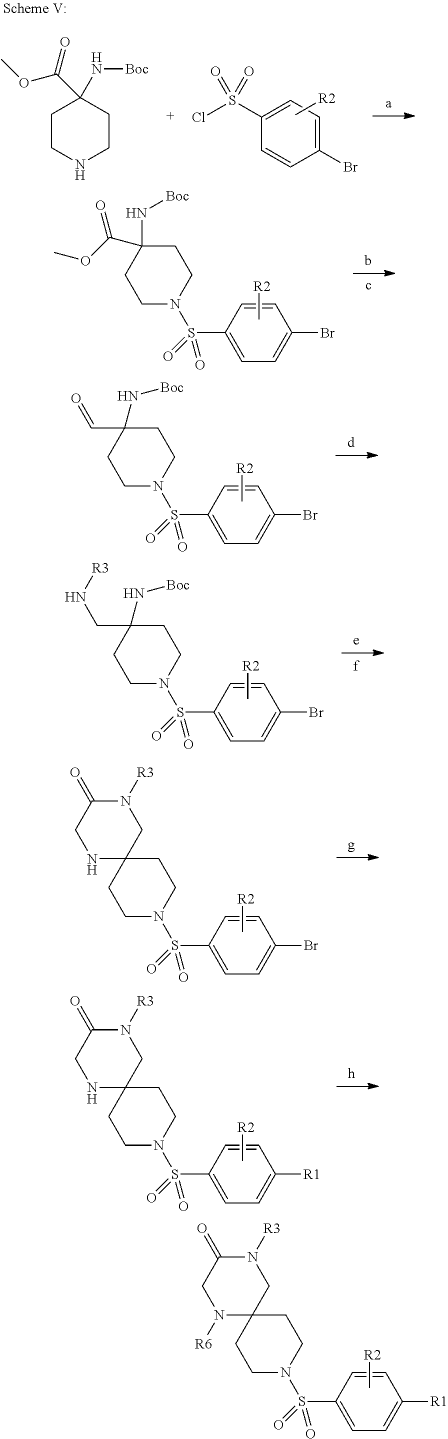

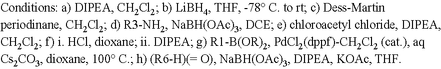

Analogs containing substitution on the piperidine can be made from commercially available piperidinones or by enolate chemistry via a metal enolate or by reaction of a silyl enol ether with a suitable electrophile (Scheme VI). The functionalized piperidinones can then be elaborated to the spirocyclic products using methodology described above. Additional examples are also described in the experimental section.

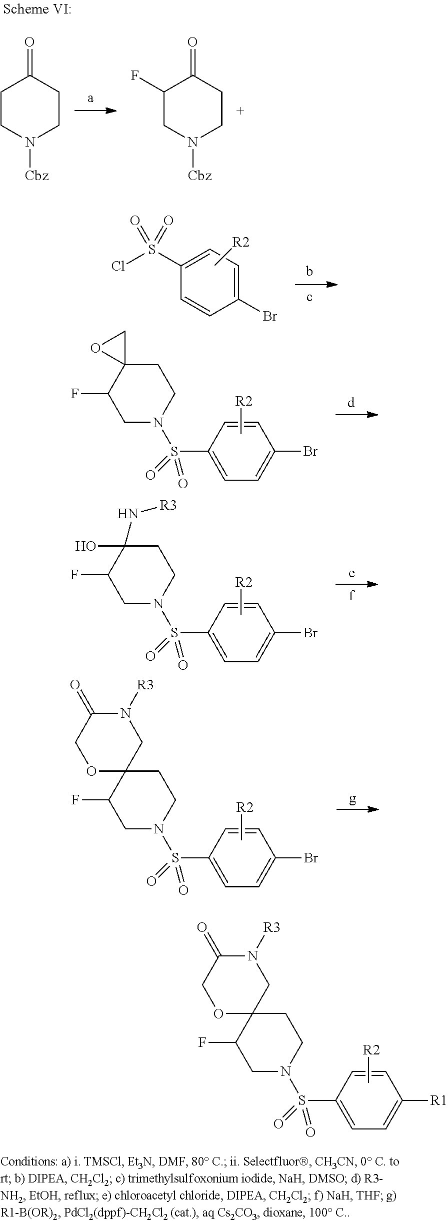

Analogs where the Cy group is pyridinyl or 5-membered heteroaromatic can be prepared following similar procedures as outlined in the above Schemes using appropriate starting materials, as described in the experimentals below.

### EXPERIMENTAL SECTION

### Example 1

### 4-methyl-9-{[4-(7-quinolinyl)phenyl]sulfonyl}-1-oxa-4,9diazaspiro[5.5]undecan-3-one

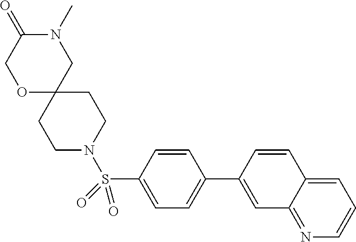 [SMILES: CN1CC2(CCN(S(=O)(=O)c3ccc(-c4ccc5cccnc5c4)cc3)CC2)OCC1=O]

### a) 1-[(4-bromophenyl)sulfonyl]-4-piperidinone

A suspension of 4-piperidinone hydrochloride (36.9 mmol) and pyridine (10 mL) in anhydrous dichloromethane (DCM) (100 mL) was stirred at room temperature for 3 h. The resulting cloudy solution was treated with 4-bromobenzenesulfonyl chloride (40.6 mmol) in one portion and stirring continued for 20 h. The resulting slurry was concentrated under reduced pressure to a thick mass that was triturated with water (400 mL). The slurry was stirred with cooling to room temperature, then the solids were collected, rinsed well with water and suction dried overnight to afford the title product (8.9 g, 75% yield) as a yellow-tan solid. MS(ES)+ m/e 317.9, 320.0 [M+H]⁺.

### b) 6-[(4-bromophenyl)sulfonyl]-1-oxa-6-azaspiro[2.5]octane

To an ice bath cooled suspension of trimethylsulfoxonium iodide (27.8 mmol) in anhydrous dimethyl sulfoxide (DMSO) (28 mL) was added 60% sodium hydride in mineral oil (33.4 mmol) portionwise over several minutes. The resulting white slurry was stirred at room temperature for 3 h then briefly cooled again to 0° C. and treated with 1-[(4-bromophenyl)sulfonyl]-4-piperidinone (27.8 mmol). The reaction was stirred at room temperature for 2 h. The brown suspension was diluted with ice cold water and the resulting slurry was extracted with dichloromethane. The extracts were washed with water and brine then dried (sodium sulfate) and evaporated to a light orange solid. This was adsorbed onto silica then placed on a short pad of silica and eluted with 40% ethyl acetate in hexanes. The filtrate was evaporated under reduced pressure to give a white residue that was triturated from 5:1 hexanes/ethyl acetate, collected by filtration, and then dried via suction to afford the title product (4.23 g, 45.3% yield) as a white solid. MS(ES)+ m/e 332.0, 334.1 [M+H]⁺. ¹H NMR (400 MHz, DMSO-d₆) δ ppm 1.49 (ddd, J=13.33, 6.25, 3.66 Hz, 2H) 1.80 (ddd, J=13.26, 8.97, 4.04 Hz, 2H) 2.61 (s, 2H) 2.92 (ddd, J=11.81, 8.65, 3.54 Hz, 2H) 3.15-3.28 (m, 2H) 7.62-7.75 (m, 2H) 7.83-7.92 (m, 2H).

### c) 1-[(4-bromophenyl)sulfonyl]-4-[(methylamino)methyl]-4-piperidinol

A 25 mL microwave vial was charged with a slurry of 6-[(4-bromophenyl)sulfonyl]-1-oxa-6-azaspiro[2.5]octane (1.505 mmol) in 2.0M methylamine in methanol (5.0 mL, 10.00 mmol) then sealed with a standard aluminum crimp cap. The reaction was heated on an aluminum block at 75° C. for 2 h. The resulting clear solution was cooled then treated with silica powder (1g) and evaporated under reduced pressure to dryness. This was then purified via flash chromatography (7% methanol/ethyl acetate) and evaporated to an oil that was evaporated from dichloromethane (2×) to afford the title product (489 mg, 82% yield) as a clear, colorless residue. MS(ES)+ m/e 363.0, 365.1 [M+H]⁺.

### d) 9-[(4-bromophenyl)sulfonyl]-4-methyl-1-oxa-4,9-diazaspiro[5.5]undecan-3-one

To an ice bath cooled solution of 1-[(4-bromophenyl)sulfonyl]-4-[(methylamino)methyl]-4-piperidinol (1.321 mmol) and triethylamine (0.460 mL, 3.30 mmol) in anhydrous dichloromethane (DCM) (20.0 mL) was added neat chloroacetyl chloride (0.159 mL, 1.982 mmol). The cold bath was removed and stirring continued for 2 h whereupon LCMS indicated complete conversion to the intermediate chloroacetamide. The resulting brown solution was quenched with brine, extracted with dichloromethane, and then the extracts were dried (sodium sulfate) and evaporated under reduced pressure to give crude N-({1-[(4-bromophenyl)sulfonyl]-4-hydroxy-4-piperidinyl}methyl)-2-chloro-N-methylacetamide (635 mg, 89% yield) as a brown solid. MS(ES)+ m/e 439.0, 440.9 [M+H]⁺.

To a stirring solution of crude N-({1-[(4-bromophenyl)sulfonyl]-4-hydroxy-4-piperidinyl}methyl)-2-chloro-N-methylacetamide (1.170 mmol) in anhydrous tetrahydrofuran (THF) (20 mL) and anhydrous dimethyl sulfoxide (5.0 mL) was added 60% sodium hydride in mineral oil (211 mg, 5.26 mmol). The reaction was heated at 75° C. for 1 h, then the resulting dark brown suspension was cooled and concentrated under reduced pressure to an oil that was taken into brine and extracted into dichloromethane. The extracts were washed with brine, dried (sodium sulfate), and then evaporated to a crude oil. Purification of the residue by flash chromatography (ethyl acetate) afforded the title product (329 mg, 63% yield) as a clear residue that solidified on standing. MS(ES)+ m/e 402.9, 405.0 [M+H]⁺.

### e) 7-quinolinyl trifluoromethanesulfonate

To an ice-bath cooled suspension of 7-quinolinol (9.44 mmol) and pyridine (12.27 mmol) in anhydrous dichloromethane (DCM) (25.0 mL) was slowly added triflic anhydride (10.38 mmol) and the resulting dark solution was stirred at room temperature overnight. The mixture was washed with water, brine, and saturated aqueous sodium bicarbonate, and then dried over sodium sulfate, filtered, and evaporated to give a tan solid. Purification by flash chromatography (50% hexanes in ethyl acetate) gave the title product (2.43 g, 92% yield) as a white solid. MS(ES)+ m/e 277.9 [M+H]⁺. ¹H NMR (400 MHz, DMSO-d₆) δ ppm 7.68 (dd, J=8.34, 4.29 Hz, 1H) 7.76 (dd, J=8.97, 2.65 Hz, 1H) 8.14 (d, J=2.78 Hz, 1H) 8.24 (d, J=9.09 Hz, 1H) 8.49-8.55 (m, 1H) 9.04 (dd, J=4.30, 1.77 Hz, 1H).

### f) 7-(4,4,5,5-tetramethyl-1,3,2-dioxaborolan-2-yl)quinoline

A flask was charged with a suspension of 7-quinolinyl trifluoromethanesulfonate (8.66 mmol), bis(pinacolato)diboron (10.39 mmol), PdCl2(dppf)-CH₂Cl₂ adduct (0.432 mmol), 1,1′-bis(diphenylphosphino)ferrocene (0.433 mmol) and potassium acetate (26.0 mmol) in 1,4-dioxane (40 mL) and heated at 100° C. for 2 h. The resulting dark suspension was cooled to room temperature, taken up into ethyl acetate, washed with water (2×) and brine, dried (sodium sulfate), and evaporated to an oil (2.25 g). The oil was purified by flash chromatography (10-60% ethyl acetate in hexanes). The desired fractions were combined and evaporated to an oil that was taken up in dichloromethane and evaporated again in vacuo to afford the title product (1.72 g, 74% yield) as a pale yellow oil that solidified upon standing. MS(ES)+ m/e 256.2 [M+H]⁺. ¹H NMR (400 MHz, DMSO-d₆) δ ppm 1.34 (s, 12H) 7.58 (dd, J=8.34, 4.04 Hz, 1H) 7.80 (dd, J=8.08, 1.01 Hz, 1H) 7.93-8.00 (m, 1H) 8.34 (s, 1H) 8.36-8.41 (m, 1H) 8.95 (dd, J=4.29, 1.77 Hz, 1H).

### g) 4-methyl-9-{[4-(7-quinolinyl)phenyl]sulfonyl}-1-oxa-4,9-diazaspiro[5.5]undecan-3-one

A flask was charged with a suspension of 9-[(4-bromophenyl)sulfonyl]-4-methyl-1-oxa-4,9-diazaspiro[5.5]undecan-3-one (0.397 mmol), 7-(4,4,5,5-tetramethyl-1,3,2-dioxaborolan-2-yl)quinoline (0.595 mmol), PdCl₂(dppf)-CH₂Cl₂ adduct (0.020 mmol) and 2.0M aq potassium carbonate (0.793 mL) in 1,4-dioxane (5.00 mL) and then heated at 100° C. for 3 h. The resulting dark slurry was treated with silica gel (2 g) then diluted with methanol and evaporated to dryness. Purification of this by flash chromatography (0-5% methanol in ethyl acetate) gave the product as a tan foam, which was then further purified by reverse phase HPLC (10-60% acetonitrile w/0.1% TFA/water w/0.1% TFA). The combined desired fractions were treated with saturated aq sodium bicarbonate (10 mL) and then extracted with dichloromethane. The dried extracts (sodium sulfate) were evaporated to a foam that was taken into acetonitrile (2 mL) and water (2 mL), frozen, and lyophilized to afford the title product (96 mg, 53% yield) as a white solid. MS(ES)+m/e 451.9 [M+H]⁺. ¹H NMR (400 MHz, DMSO-d₆) δ ppm 1.58-1.79 (m, 2H) 1.91 (d, J=13.39 Hz, 2H) 2.51-2.59 (m, 2H) 2.82 (s, 3H) 3.21 (s, 2H) 3.52 (d, J=11.62 Hz, 2H) 3.88 (s, 2H) 7.59 (dd, J=8.21, 4.17 Hz, 1H) 7.87 (d, J=8.34 Hz, 2H) 8.05 (dd, J=8.59, 1.52 Hz, 1H) 8.11-8.21 (m, 3H) 8.41 (s, 1H) 8.44 (d, J=8.08 Hz, 1H) 8.98 (d, J=2.53 Hz, 1H).

### Example 2

### 9-{[4-(1H-indol-6-yl)phenyl]sulfonyl}-4-methyl-1-oxa-4,9-diazaspiro[5.5]undecan-3-one

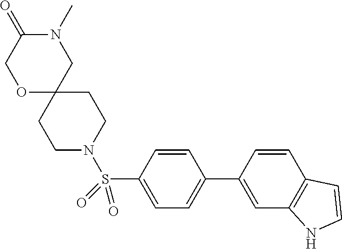 [SMILES: CN1CC2(CCN(S(=O)(=O)c3ccc(-c4ccc5cc[nH]c5c4)cc3)CC2)OCC1=O]

a) Following the procedure described in Example 1g with 6-indole boronic acid afforded the title product (54%). MS(ES)+ m/e 440.1 [M+H]⁺. ¹H NMR (400 MHz, DMSO-d₆) δ ppm 1.70 (dd, J=13.14, 3.79 Hz, 2H) 1.90 (d, J=13.39 Hz, 2H) 2.51-2.55 (m, 2H) 2.82 (s, 3H) 3.21 (s, 2H) 3.50 (d, J=11.62 Hz, 2H) 3.88 (s, 2H) 6.49 (t, J=2.02 Hz, 1H) 7.41 (dd, J=8.34, 1.77 Hz, 1H) 7.43-7.47 (m, 1H) 7.67 (d, J=8.34 Hz, 1H) 7.76 (s, 1H) 7.79 (d, J=8.34 Hz, 2H) 7.96 (d, J=8.59 Hz, 2H) 11.31 (br s, 1H).

### Example 3

### 9-{[4-(1-benzofuran-5-yl)phenyl]sulfonyl}-4-ethyl-1-oxa-4,9-diazaspiro[5.5]undecan-3-one

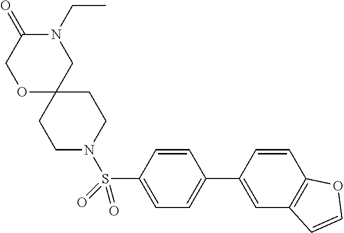 [SMILES: CCN1CC2(CCN(S(=O)(=O)c3ccc(-c4ccc5occc5c4)cc3)CC2)OCC1=O]

### a) 1-[(4-bromophenyl)sulfonyl]-4-[(ethylamino)methyl]-4-piperidinol

A 25 mL microwave vial was charged with a slurry of 6-[(4-bromophenyl)sulfonyl]-1-oxa-6-azaspiro[2.5]octane (1.511 mmol) in 2.0-Methylamine in methanol (5.0 mL, 10.00 mmol) and then sealed with a standard aluminum crimp cap. The reaction was heated on an aluminum block at 75° C. for 2 h. The clear yellow solution was evaporated under reduced pressure to give the crude title product (577 mg, 91% yield) as an off-white solid. MS(ES)+ m/e 377.1, 380.3 [M+H]⁺.

### b) 9-[(4-bromophenyl)sulfonyl]-4-ethyl-1-oxa-4,9-diazaspiro[5.5]undecan-3-one

To an ice-bath cooled solution of 1-[(4-bromophenyl)sulfonyl]-4-[(ethylamino)methyl]-4-piperidinol (1.511 mmol) and triethylamine (3.78 mmol) in anhydrous dichloromethane (DCM) (20 mL) was added neat chloroacetyl chloride (2.266 mmol). The ice bath was removed and stirring continued for 2 h whereupon analysis by LCMS indicated the complete conversion to the intermediate chloroacetamide. The resulting brown suspension was quenched with water, diluted with saturated aqueous sodium bicarbonate and extracted with dichloromethane. The dried extracts (sodium sulfate) were evaporated in vacuo to afford crude N-({1-[(4-bromophenyl)sulfonyl]-4-hydroxy-4-piperidinyl}methyl)-2-chloro-N-ethylacetamide as a brown solid. MS(ES)+m/e 454.9 [M+H]⁺.

To a solution of N-({1-[(4-bromophenyl)sulfonyl]-4-hydroxy-4-piperidinyl}methyl)-2-chloro-N-ethylacetamide (1.382 mmol) in anhydrous dimethyl sulfoxide (DMSO) (3.0 mL) was added 60% sodium hydride in mineral oil (6.22 mmol). The sides of the flask were rinsed down with anhydrous tetrahydrofuran (THF) (3.00 mL) and then the reaction was allowed to stir at room temperature for 18 h. The resulting brown suspension was quenched with water, diluted further with saturated aqueous sodium bicarbonate, and then extracted with dichloromethane The dried extracts (sodium sulfate) were treated with silica powder (˜2 g) then evaporated under reduced pressure to dryness. This was purified by flash chromatography (20% hexanes in ethyl acetate) to afford the title product (331 mg, 56% yield) as a white solid. MS(ES)+ m/e 417.0, 419.1 [M+H]⁺. ¹H NMR (400 MHz, DMSO-d₆) δ ppm 1.00 (t, J=7.20 Hz, 3H) 1.59-1.73 (m, 2H) 1.85 (d, J=12.88 Hz, 2H) 2.45 (td, J=11.94, 2.40 Hz, 2H) 3.21 (s, 2H) 3.28 (q, J=7.07 Hz, 2H) 3.45 (d, J=11.87 Hz, 2H) 3.87 (s, 2H) 7.62-7.72 (m, 2H) 7.82-7.91 (m, 2H).

### c) 9-{[4-(1-benzofuran-5-yl)phenyl]sulfonyl}-4-ethyl-1-oxa-4,9-diazaspiro[5.5]undecan-3-one

A suspension of 9-[(4-bromophenyl)sulfonyl]-4-ethyl-1-oxa-4,9-diazaspiro[5.5]undecan-3-one (0.383 mmol), 5-(4,4,5,5-tetramethyl-1,3,2-dioxaborolan-2-yl)-1-benzofuran (0.575 mmol), 2.0M aq potassium carbonate (0.767 mL) and PdCl₂(dppf)-CH₂Cl₂ adduct (0.019 mmol) in 1,4-dioxane (6.136 mL) was heated at reflux for 4 h. The resulting dark slurry was treated with silica powder (2 g) then diluted with methanol and evaporated to dryness. This was purified by flash chromatography (10% hexanes in ethyl acetate) to give the product as a tan oily residue. This was purified again by reverse phase HPLC (15-80% acetonitrile w/0.1% TFA/water w/0.1% TFA). The combined, desired fractions were treated with saturated aq sodium bicarbonate (10 mL) and then extracted with dichloromethane. The dried extracts (sodium sulfate) were evaporated under reduced pressure to give the title product (115 mg, 65% yield) as a white solid. MS(ES)+ m/e 455.0 [M+H]⁺. ¹H NMR (400 MHz, DMSO-d₆) δ ppm 1.00 (t, J=7.20 Hz, 3H) 1.61-1.77 (m, 2H) 1.81-1.95 (m, 2H) 2.43-2.49 (m, 2H) 3.21 (s, 2H) 3.28 (q, J=7.07 Hz, 2H) 3.45-3.57 (m, 2H) 3.87 (s, 2H) 7.05 (d, J=1.52 Hz, 1H) 7.71 (dd, J=8.59, 1.77 Hz, 1H) 7.74 (d, J=8.59 Hz, 1H) 7.81 (d, J=8.59 Hz, 2H) 7.98 (d, J=8.59 Hz, 2H) 8.06 (s, 1H) 8.08 (d, J=2.27 Hz, 1H).

### Example 4

### 4-ethyl-9-{[4-(7-quinolinyl)phenyl]sulfonyl}-1-oxa-4,9-diazaspiro[5.5]undecan-3-one

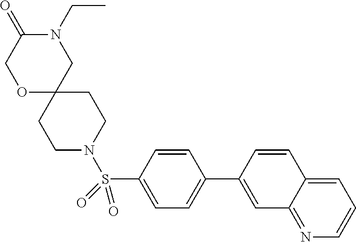 [SMILES: CCN1CC2(CCN(S(=O)(=O)c3ccc(-c4ccc5cccnc5c4)cc3)CC2)OCC1=O]

a) Following the procedure described in Example 3c with 7-(4,4,5,5-tetramethyl-1,3,2-dioxaborolan-2-yl)quinoline afforded the title product (53%). MS(ES)+ m/e 466.1 [M+H]⁺. ¹H NMR (400 MHz, DMSO-d₆) δ ppm 1.00 (t, J=7.20 Hz, 3H) 1.62-1.77 (m, 2H) 1.89 (d, J=13.14 Hz, 2H) 2.51-2.58 (m, 2H) 3.22 (s, 2H) 3.28 (q, J=7.07 Hz, 2H) 3.54 (d, J=11.62 Hz, 2H) 3.88 (s, 2H) 7.59 (dd, J=8.34, 4.29 Hz, 1H) 7.87 (d, J=8.34 Hz, 2H) 8.05 (dd, J=8.46, 1.89 Hz, 1H) 8.15 (d, J=8.34 Hz, 1H) 8.15-8.19 (m, 2H) 8.41 (d, J=1.77 Hz, 1H) 8.44 (dd, J=8.72, 1.39 Hz, 1H) 8.98 (dd, J=4.29, 1.77 Hz, 1H).

### Example 5

### 4-(1-methylethyl)-9-{[4-(7-quinolinyl)phenyl]sulfonyl}-1-oxa-4,9-diazaspiro[5.5]undecan-3-one

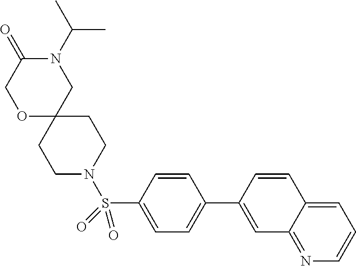 [SMILES: CC(C)N1CC2(CCN(S(=O)(=O)c3ccc(-c4ccc5cccnc5c4)cc3)CC2)OCC1=O]

### a) 1-[(4-bromophenyl)sulfonyl]-4-{[(1-methylethyl)amino]methyl}-4-piperidinol

A suspension of 6-[(4-bromophenyl)sulfonyl]-1-oxa-6-azaspiro[2.5]octane (0.707 mmol) and isopropylamine (2.83 mmol) in absolute ethanol (5.0 mL) was heated at 75° C. for 2 h whereupon analysis by LCMS indicated complete conversion. The solvents were removed under reduced pressure to afford the title product (277 mg, 98% yield) as a white solid. MS(ES)+ m/e 391.0, 393.0 [M+H]⁺, bromine pattern. ¹H NMR (400 MHz, DMSO-d₆) δ ppm 0.94 (d, J=6.32 Hz, 6H) 1.23-1.40 (m, 1H) 1.43-1.63 (m, 4H) 2.36 (s, 2H) 2.43-2.55 (m, 5H) 2.60 (s, 1H) 3.38 (d, J=11.62 Hz, 2H) 4.17 (s, 1H) 7.62-7.72 (m, 2H) 7.84-7.92 (m, 2H).

### b) 9-[(4-bromophenyl)sulfonyl]-4-(1-methylethyl)-1-oxa-4,9-diazaspiro[5.5]undecan-3-one

A solution of 1-[(4-bromophenyl)sulfonyl]-4-{[(1-methylethyl)amino]methyl}-4-piperidinol (0.698 mmol) and triethylamine (1.744 mmol) in anhydrous dichloromethane (DCM) (10.0 mL) was cooled to 0° C. then treated with neat chloroacetyl chloride (1.046 mmol). The ice bath was removed and the reaction stirred for 30 min, whereupon analysis by LCMS indicated conversion to intermediate chloroacetamide. The brown solution was diluted with dichloromethane, washed with brine, and then dried (sodium sulfate) and evaporated to a foam. MS(ES)+ m/e 466.9 [M+H]⁺.

The intermediate was taken into anhydrous dimethyl sulfoxide (DMSO) (3.0 mL) and tetrahydrofuran (THF) (3.00 mL) then treated with 60% sodium hydride in mineral oil (3.14 mmol) and stirred at 60° C. for 2 h. The reaction was cooled and quenched with water (500 μl), then evaporated to a residue that was purified directly by reverse phase HPLC (15-80% acetonitrile w/0.1% TFA/water w/0.1% TFA). The combined desired fractions were treated with saturated aq sodium bicarbonate (10 mL) and then extracted into dichloromethane. The extracts were dried (sodium sulfate) and evaporated to afford the title product (102 mg, 34% yield) as a pale yellow solid. MS(ES)+ m/e 431.0, 433.1 [M+H]⁺. ¹H NMR (400 MHz, DMSO-d₆) δ ppm 1.01 (d, J=6.82 Hz, 6H) 1.54-1.73 (m, 2H) 1.82 (d, J=12.88 Hz, 2H) 2.37-2.48 (m, 2H) 3.10 (s, 2H) 3.47 (d, J=11.87 Hz, 2H) 3.88 (s, 2H) 4.56-4.71 (m, 1H) 7.61-7.73 (m, 2H) 7.83-7.91 (m, 2H).

### c) 4-(1-methylethyl)-9-{[4-(7-quinolinyl)phenyl]sulfonyl}-1-oxa-4,9-diazaspiro[5.5]undecan-3-one

A flask was charged with a suspension of 9-[(4-bromophenyl)sulfonyl]-4-(1-methylethyl)-1-oxa-4,9-diazaspiro[5.5]undecan-3-one (0.232 mmol), 7-(4,4,5,5-tetramethyl-1,3,2-dioxaborolan-2-yl)quinoline (0.348 mmol), PdCl₂(dppf)-CH₂Cl₂ adduct (0.012 mmol) and 2.0M aq potassium carbonate (0.464 mL) in 1,4-dioxane (3.71 mL) and heated at 95° C. for 2 h. The resulting dark solution was cooled to room temperature and then diluted with water and extracted into dichloromethane. The extracts were dried (sodium sulfate) and evaporated to a crude solid, which was purified by reverse phase HPLC (15-55% acetonitrile w/0.1% TFA/water w/0.1% TFA). The combined desired fractions were treated with saturated aq sodium bicarbonate (10 mL) and concentrated under reduced pressure to remove organic solvents. The resulting slurry was filtered and the solids were rinsed well with water then suction and vacuum dried to afford the title product (66 mg, 59% yield) as a white solid. MS(ES)+ m/e 480.0 [M+H]⁺. ¹H NMR (400 MHz, DMSO-d₆) δ ppm 1.01 (d, J=6.82 Hz, 6H) 1.59-1.78 (m, 2H) 1.86 (d, J=12.88 Hz, 2H) 2.51-2.55 (m, 2H) 3.12 (s, 2H) 3.56 (d, J=11.62 Hz, 2H) 3.88 (s, 2H) 4.56-4.70 (m, 1H) 7.59 (dd, J=8.21, 4.17 Hz, 1H) 7.87 (d, J=8.34 Hz, 2H) 8.05 (dd, J=8.46, 1.89 Hz, 1H) 8.15 (d, J=8.34 Hz, 1H) 8.16 (d, J=8.59 Hz, 2H) 8.41 (d, J=1.77 Hz, 1H) 8.44 (dd, J=8.72, 1.39 Hz, 1H) 8.98 (dd, J=4.29, 1.77 Hz, 1H).

### Example 6

### 9-{[4-(7-quinolinyl)phenyl]sulfonyl}-4-(2,2,2-trifluoroethyl)-1-oxa-4,9-diazaspiro[5.5]undecan-3-one

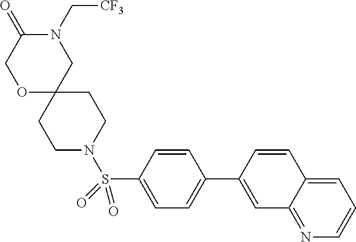 [SMILES: O=C1COC2(CCN(S(=O)(=O)c3ccc(-c4ccc5cccnc5c4)cc3)CC2)CN1CC(F)(F)F]

### a) 1-[(4-bromophenyl)sulfonyl]-4-{[(2,2,2-trifluoroethyl)amino]methyl}-4-piperidinol

A 25 mL microwave vessel was charged with a suspension of 6-[(4-bromophenyl)sulfonyl]-1-oxa-6-azaspiro[2.5]octane (1.355 mmol) and 2,2,2-trifluoroethanamine (6.77 mmol) in absolute ethanol (5.0 mL) then sealed with an aluminum crimp cap. The reaction was heated on an aluminum block at 85° C. for 24 h. The reaction was cooled and evaporated under reduced pressure to afford the crude title product (594 mg, 98% yield) as a white solid. MS(ES)+ m/e 431.0, 433.1 [M+H]⁺. ¹H NMR (400 MHz, DMSO-d₆) δ ppm 1.43-1.64 (m, 4H) 2.12-2.27 (m, 1H) 2.51-2.57 (m, 2H) 3.15-3.31 (m, 2H) 3.34-3.43 (m, 2H) 4.27 (s, 1H) 7.63-7.72 (m, 2H) 7.83-7.92 (m, 2H).

### b) 9-[(4-bromophenyl)sulfonyl]-4-(2,2,2-trifluoroethyl)-1-oxa-4,9-diazaspiro[5.5]undecan-3-one

To an ice-bath cooled solution of 1-[(4-bromophenyl)sulfonyl]-4-{[(2,2,2-trifluoroethyl)amino]methyl}-4-piperidinol (1.363 mmol) and triethylamine (3.41 mmol) in anhydrous dichloromethane (DCM) (25 mL) was added neat chloroacetyl chloride (2.045 mmol). The ice bath was removed and stirring continued at room temperature for 3 h. The resulting brown solution was diluted with brine then extracted further with dichloromethane. The extracts were dried (sodium sulfate) and evaporated under reduced pressure to give the intermediate chloroacetamide as a tan solid. MS(ES)+ m/e 508.9 [M+H]⁺.

The intermediate was taken into anhydrous tetrahydrofuran (THF) (25 mL) then treated with 60% sodium hydride in mineral oil (6.14 mmol). The reaction was then heated at reflux for 20 h whereupon analysis by LCMS showed approximately 25% of the starting material remaining DMSO (˜1 mL) was added to aid solubility and the reaction was heated a further 3 h. The resulting dark solution was cooled to room temperature then quenched with water and extracted with dichloromethane. The extracts were dried (sodium sulfate), treated with silica powder (2 g) and decolorizing charcoal (100 mg) and then evaporated to dryness. This was purified by flash chromatography (30% hexanes in ethyl acetate) to afford the title product (463 mg, 65% yield) as a white solid. MS(ES)+m/e 471.1, 472.9 [M+H]⁺.

### c) 9-{[4-(7-quinolinyl)phenyl]sulfonyl}-4(2,2,2-trifluoroethyl)-1-oxa-4,9-diazaspiro[5.5]undecan-3-one

A flask was charged with a solution of 9-[(4-bromophenyl)sulfonyl]-4(2,2,2-trifluoroethyl)-1-oxa-4,9-diazaspiro[5.5]undecan-3-one (0.296 mmol), 7-(4,4,5,5-tetramethyl-1,3,2-dioxaborolan-2-yl)quinoline (0.444 mmol), PdCl₂(dppf)-CH₂Cl₂ adduct (0.015 mmol) and 2.0M aq potassium carbonate (0.592 mL) in 1,4-dioxane (10.06 mL) and heated at 95° C. for 18 h. The resulting dark solution was cooled then diluted with water and extracted with dichloromethane. The extracts were dried (sodium sulfate) and then treated with silica powder (2 g) and evaporated under reduced pressure to dryness. This was purified by flash chromatography (60-100% ethyl acetate in hexanes) and then reverse phase HPLC (10-60% acetonitrile w/0.1% TFA/water w/0.1% TFA). The desired fractions were combined, treated with saturated aq sodium bicarbonate (10 mL) and extracted with dichloromethane. The extracts were dried (sodium sulfate) and then evaporated to a white foam that was taken into acetonitrile (1 mL) and water (3 mL), frozen, and lyophilized to afford the title product (93 mg, 60% yield) as a white solid. MS(ES)+ m/e 520.1 [M+H]⁺. ¹H NMR (400 MHz, DMSO-d₆) δ ppm 1.63-1.78 (m, 2H) 1.92 (br. s., 2H) 2.52-2.60 (m, 2H) 3.39 (s, 2H) 3.48-3.59 (m, 2H) 4.05 (s, 2H) 4.18 (q, J=9.60 Hz, 2H) 7.59 (dd, J=8.08, 4.04 Hz, 1H) 7.88 (d, J=8.59 Hz, 2H) 8.05 (dd, J=8.59, 2.02 Hz, 1H) 8.15 (d, J=7.07 Hz, 1H) 8.17 (d, J=7.07 Hz, 2H) 8.41 (d, J=1.77 Hz, 1H) 8.44 (dd, J=8.34, 1.01 Hz, 1H) 8.98 (dd, J=4.29, 1.77 Hz, 1H).

### Example 7

### 4-(2-furanylmethyl)-9-{[4-(7-quinolinyl)phenyl]sulfonyl}-1-oxa-4,9-diazaspiro[5.5]undecan-3-one

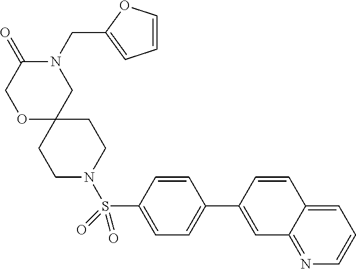 [SMILES: O=C1COC2(CCN(S(=O)(=O)c3ccc(-c4ccc5cccnc5c4)cc3)CC2)CN1Cc1ccco1]

### a) 1-[(4-bromophenyl)sulfonyl]-4-{[(2-furanylmethyl)amino]methyl}-4-piperidinol

A 25 mL microwave vessel was charged with a suspension of 6-[(4-bromophenyl)sulfonyl]-1-oxa-6-azaspiro[2.5]octane (0.762 mmol) and furfurylamine (3.05 mmol) in absolute ethanol (5.0 mL) then sealed with an aluminum crimp cap. The reaction was heated on an aluminum block at 85° C. for 3 h. The resulting yellow solution was evaporated under reduced pressure to an oil that was purified via flash chromatography (20% hexanes in ethyl acetate). The desired fractions were combined and evaporated in vacuo to afford the title product (309 mg, 90% yield) as an off-white solid. MS(ES)+ m/e 428.9, 431.1 [M+H]⁺.

### b) 9-[(4-bromophenyl)sulfonyl]-4-(2-furanylmethyl)-1-oxa-4,9-diazaspiro[5.5]undecan-3-one

To an ice-bath cooled solution of 1-[(4-bromophenyl)sulfonyl]-4-{[(2-furanylmethyl)amino]methyl}-4-piperidinol (0.699 mmol) and triethylamine (1.747 mmol) in anhydrous dichloromethane (DCM) (20 mL) was added neat chloroacetyl chloride (1.049 mmol) in one portion. The ice bath was removed and the solution stirred for 30 min, whereupon analysis by LCMS indicated complete conversion to the intermediate chloroacetamide. The reaction was diluted with dichloromethane and washed with water, and then the organic phase was dried (sodium sulfate) and evaporated under reduced pressure to afford the intermediate as a tan foam. MS(ES)+ m/e 504.8, 506.8, 472.9 [M+H]⁺.

To a solution of the chloroacetamide intermediate in anhydrous tetrahydrofuran (THF) (20 mL) was added 60% sodium hydride in mineral oil (3.14 mmol). DMSO (2 mL) was added and the reaction was heated at 75° C. for 6 h. The reaction was extracted with ethyl acetate then the extracts were dried (sodium sulfate) and evaporated onto silica powder (2 g). This was purified by flash chromatography (40-60% ethyl acetate in hexanes) and then by reverse phase HPLC (20-80% acetonitrile w/0.1% TFA/water w/0.1% TFA). The desired fractions were combined, treated with saturated aq sodium bicarbonate, and extracted with dichloromethane. The organic layer was dried (sodium sulfate) and evaporated to afford the title product (93 mg, 28% yield) as a colorless residue. MS(ES)+ m/e 469.0, 471.1 [M+H]⁺.

### c) 4-(2-furanylmethyl)-9-{[4-(7-quinolinyl)phenyl]sulfonyl}-1-oxa-4,9-diazaspiro[5.5]undecan-3-one

A suspension of 9-[(4-bromophenyl)sulfonyl]-4-(2-furanylmethyl)-1-oxa-4,9-diazaspiro[5.5]undecan-3-one (0.192 mmol), 7-(4,4,5,5-tetramethyl-1,3,2-dioxaborolan-2-yl)quinoline (0.288 mmol), PdCl₂(dppf)-CH₂Cl₂ adduct (9.59 μmol) and 2.0M aq potassium carbonate (0.384 mL) in 1,4-dioxane (8.0 mL) was heated at 100° C. for 4 h. The resulting dark solution was cooled then diluted with water and extracted with dichloromethane. The extracts were dried (sodium sulfate), treated with silica powder (2 g), and evaporated under reduced pressure to dryness. This was purified by flash chromatography (50-100% ethyl acetate in hexanes) and then by reverse phase HPLC (10-60% acetonitrile w/0.1% TFA/water w/0.1% TFA). The desired fractions were combined, treated with saturated aq sodium bicarbonate (30 mL), and extracted with dichloromethane. The extracts were dried (sodium sulfate) and evaporated to a colorless residue. This was dissolved into acetonitrile (1 mL) and water (2 mL), frozen, and lyophilized to afford the title product (57.4 mg, 57% yield) as a white solid. MS(ES)+m/e 518.2 [M+H]⁺. ¹H NMR (400 MHz, DMSO-d₆) δ ppm 1.52-1.71 (m, 2H) 1.79-1.93 (m, 2H) 2.53-2.60 (m, 2H) 3.17 (s, 2H) 3.37-3.49 (m, 2H) 3.96 (s, 2H) 4.51 (s, 2H) 6.32 (d, J=3.03 Hz, 1H) 6.41 (dd, J=3.16, 1.89 Hz, 1H) 7.56-7.62 (m, 2H) 7.86 (d, J=8.59 Hz, 2H) 8.05 (dd, J=8.59, 1.77 Hz, 1H) 8.12-8.19 (m, 3H) 8.41 (d, J=1.77 Hz, 1H) 8.42-8.48 (m, 1H) 8.98 (dd, J=4.04, 1.52 Hz, 1H).

### Example 8

### 4-[2-(methyloxy)ethyl]-9-{[4-(7-quinolinyl)phenyl]sulfonyl}-1-oxa-4,9-diazaspiro[5.5]undecan-3-one

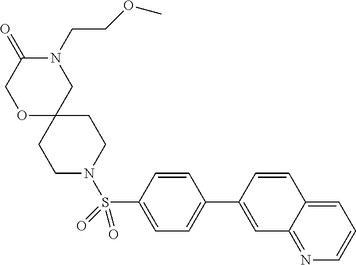 [SMILES: COCCN1CC2(CCN(S(=O)(=O)c3ccc(-c4ccc5cccnc5c4)cc3)CC2)OCC1=O]

### a) 1-[(4-bromophenyl)sulfonyl]-4-({[2-(methyloxy)ethyl]amino}methyl)-4-piperidinol

A 25 mL microwave vessel was charged with a suspension of 6-[(4-bromophenyl)sulfonyl]-1-oxa-6-azaspiro[2.5]octane (0.960 mmol) and 2-(methyloxy)ethanamine (3.84 mmol) in absolute ethanol (5.0 mL) then sealed with an aluminum crimp cap. The reaction was heated on an aluminum block at 85° C. for 3 h, whereupon analysis by LCMS indicated the complete conversion of the epoxide. The reaction was cooled and evaporated to an oil that was purified via flash chromatography (5% methanol in dichloromethane) to afford the title product (375 mg, 95% yield) as a colorless residue that solidified on standing. MS(ES)+ m/e 407.3, 409.2 [M+H]⁺.

### b) 9-[(4-bromophenyl)sulfonyl]-4-[2-(methyloxy)ethyl]-1-oxa-4,9-diazaspiro[5.5]undecan-3-one

To an ice-bath cooled solution of 1-[(4-bromophenyl)sulfonyl]-4-({[2-(methyloxy)ethyl]amino}methyl)-4-piperidinol (0.906 mmol) and triethylamine (2.265 mmol) in anhydrous dichloromethane (DCM) (20 mL) was added neat chloroacetyl chloride (1.361 mmol). The ice bath was removed and stirring continued for 30 min, whereupon analysis by LCMS indicated complete conversion to the intermediate chloroacetamide. The reaction was diluted with dichloromethane, washed with water and brine, then dried (sodium sulfate) and evaporated to afford the intermediate as a tan residue. MS(ES)+ m/e 483.0, 485.0 [M+H]⁺.

To a solution of the chloroacetamide intermediate in anhydrous tetrahydrofuran (THF) (20 mL) was added 60% sodium hydride in mineral oil (4.08 mmol). The reaction was heated at reflux for 3 h. The cooled reaction was diluted with water and extracted into ethyl acetate. The extracts were dried (sodium sulfate), treated with silica powder (2 g), and evaporated to dryness. This was purified by flash chromatography (ethyl acetate) to afford the title product (214 mg, 49% yield) as a white solid. MS(ES)+ m/e 447.0, 449.0 [M+H]⁺.

### c) 4-[2-(methyloxy)ethyl]-9-{[4-(7-quinolinyl)phenyl]sulfonyl}-1-oxa-4,9-diazaspiro[5.5]undecan-3-one

A suspension of 9-[(4-bromophenyl)sulfonyl]-4-[2-(methyloxy)ethyl]-1-oxa-4,9-diazaspiro[5.5]undecan-3-one (0.311 mmol), 7-(4,4,5,5-tetramethyl-1,3,2-dioxaborolan-2-yl)quinoline (0.466 mmol), PdCl₂(dppf)-CH₂Cl₂ adduct (0.016 mmol) and 2.0M aq potassium carbonate (0.621 mL) in 1,4-dioxane (10 mL) was heated at 95° C. for 20 h. The dark suspension was cooled, diluted with water, and extracted with dichloromethane. The extracts were dried (sodium sulfate), treated with silica powder (2 g), and evaporated under reduced pressure to dryness. This was purified by flash chromatography (3% methanol in ethyl acetate) and then by reverse phase HPLC (10-55% acetonitrile w/0.1% TFA/water w/0.1% TFA). The desired fractions were combined, diluted with saturated aq sodium bicarbonate and brine, and extracted with dichloromethane. The extracts were dried (sodium sulfate) and evaporated under reduced pressure. The resulting colorless residue was taken into acetonitrile (1 mL) and water (2 mL), frozen, and lyophilized to afford the title product (97 mg, 62% yield) as a white solid. MS(ES)+ m/e 496.2 [M+H]⁺. ¹H NMR (400 MHz, DMSO-d₆) δ ppm 1.60-1.76 (m, 2H) 1.81-1.95 (m, 2H) 2.52-2.63 (m, 2H) 3.22 (s, 3H) 3.28 (s, 2H) 3.42 (s, 4H) 3.45-3.53 (m, 2H) 3.91 (s, 2H) 7.59 (dd, J=8.34, 4.29 Hz, 1H) 7.88 (d, J=8.59 Hz, 2H) 8.05 (dd, J=8.46, 1.89 Hz, 1H) 8.15 (d, J=6.57 Hz, 1H) 8.17 (d, J=6.57 Hz, 2H) 8.41 (d, J=1.77 Hz, 1H) 8.44 (dd, J=8.59, 1.26 Hz, 1H) 8.98 (dd, J=4.04, 1.77 Hz, 1H).

### Example 9

### 4-(phenylmethyl)-9-{[4-(7-quinolinyl)phenyl]sulfonyl}-1-oxa-4,9-diazaspiro[5.5]undecan-3-one

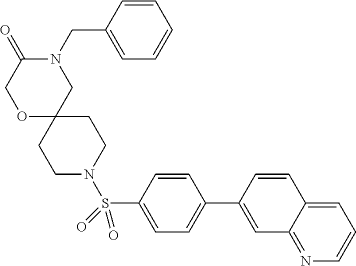 [SMILES: O=C1COC2(CCN(S(=O)(=O)c3ccc(-c4ccc5cccnc5c4)cc3)CC2)CN1Cc1ccccc1]

### a) 1-[(4-bromophenyl)sulfonyl]-4-{[(phenylmethyl)amino]methyl}-4-piperidinol

A 25 mL microwave vessel was charged with a suspension of 6-[(4-bromophenyl)sulfonyl]-1-oxa-6-azaspiro[2.5]octane (0.951 mmol) and benzylamine (2.378 mmol) in absolute ethanol (5.0 mL). The vessel was sealed with an aluminum crimp cap then heated on an aluminum block at 85° C. for 3 h. The reaction was cooled then evaporated under reduced pressure and the residue was taken into dichloromethane and washed with water and brine. The organic phase was dried (sodium sulfate) and evaporated to a yellow oil. This was purified via flash chromatography (75-100% ethyl acetate in hexanes) to afford the title product (365 mg, 86% yield) as a white solid. MS(ES)+ m/e 439.1, 441.0 [M+H]⁺, bromine pattern. ¹H NMR (400 MHz, DMSO-d₆) δ ppm 1.44-1.67 (m, 4H) 1.97 (br. s., 1H) 2.36 (s, 2H) 2.51-2.57 (m, 2H) 3.27-3.41 (m, 2H) 3.68 (s, 2H) 4.23 (s, 1H) 7.15-7.25 (m, 1H) 7.29 (d, J=4.29 Hz, 4H) 7.61-7.72 (m, 2H) 7.81-7.91 (m, 2H).

### b) 9-[(4-bromophenyl)sulfonyl]-4-(phenylmethyl)-1-oxa-4,9-diazaspiro[5.5]undecan-3-one

To an ice-bath cooled solution of 1-[(4-bromophenyl)sulfonyl]-4-{[(phenylmethyl)amino]methyl}-4-piperidinol (0.808 mmol) and triethylamine (2.020 mmol) in anhydrous dichloromethane (DCM) (20 mL) was added neat chloroacetyl chloride (1.211 mmol). The ice bath was removed and stirring continued for 30 min whereupon analysis by LCMS indicated complete conversion to the intermediate chloroacetamide. The resulting light brown solution was diluted with dichloromethane, washed with water and brine then dried (sodium sulfate) and evaporated under reduced pressure to a tan foam. MS(ES)+ m/e 517.1 [M+H]⁺.

To a solution of the intermediated chloroacetamide in anhydrous tetrahydrofuran (THF) (20 mL) was added 60% sodium hydride in mineral oil (3.64 mmol). The reaction was heated at reflux for 18 h. To resulting dark suspension was cooled, quenched with water, and extracted with dichloromethane. The extracts were washed with brine, dried over sodium sulfate, and evaporated onto silica powder (2 g). This was then purified by flash chromatography (40% hexanes in ethyl acetate) to afford the title product (315 mg, 79% yield) as a white solid. MS(ES)+ m/e 478.7, 481.0 [M+H]⁺. ¹H NMR (400 MHz, DMSO-d₆) δ ppm 1.47-1.64 (m, 2H) 1.84 (d, J=13.39 Hz, 2H) 2.40-2.49 (m, 2H) 3.12 (s, 2H) 3.29-3.33 (m, 2H) 4.01 (s, 2H) 4.50 (s, 2H) 7.16-7.26 (m, 2H) 7.26-7.39 (m, 3H) 7.59-7.69 (m, 2H) 7.84-7.91 (m, 2H).

### c) 4-(phenylmethyl)-9-{[4-(7-quinolinyl)phenyl]sulfonyl}-1-oxa-4,9-diazaspiro[5.5]undecan-3-one

A suspension of 9-[(4-bromophenyl)sulfonyl]-4-(phenylmethyl)-1-oxa-4,9-diazaspiro[5.5]undecan-3-one (0.280 mmol), 7-(4,4,5,5-tetramethyl-1,3,2-dioxaborolan-2-yl)quinoline (0.419 mmol), PdCl₂(dppf)-CH₂Cl₂ adduct (0.014 mmol) and 2.0M aq potassium carbonate (0.559 mL) in 1,4-dioxane (10 mL) was heated at 95° C. for 20 h. The dark suspension was cooled, diluted with water, and extracted with dichloromethane. The extracts were dried (sodium sulfate), treated with silica powder (2 g), and evaporated under reduced pressure to dryness. This was purified by flash chromatography (ethyl acetate) and then by reverse phase HPLC (10-60% acetonitrile w/0.1% TFA/water w/0.1% TFA). The desired fractions were combined and diluted with saturated aq sodium bicarbonate and brine then extracted with dichloromethane. The extracts were dried (sodium sulfate) and evaporated under reduced pressure. The resulting colorless residue was taken into acetonitrile (1 mL) and water (2 mL), frozen, and lyophilized to afford the title product (78.8 mg, 53% yield) as a white solid. MS(ES)+ m/e 528.2 [M+H]⁺. ¹H NMR (400 MHz, DMSO-d₆) δ ppm 1.50-1.68 (m, 2H) 1.81-1.97 (m, 2H) 2.51-2.60 (m, 2H) 3.13 (s, 2H) 3.36-3.47 (m, 2H) 4.01 (s, 2H) 4.50 (s, 2H) 7.19-7.25 (m, 2H) 7.25-7.39 (m, 3H) 7.59 (dd, J=8.21, 4.17 Hz, 1H) 7.85 (d, J=8.59 Hz, 2H) 8.05 (dd, J=8.59, 2.02 Hz, 1H) 8.15 (d, J=8.59 Hz, 1H) 8.16 (d, J=8.59 Hz, 2H) 8.41 (d, J=1.77 Hz, 1H) 8.42-8.48 (m, 1H) 8.98 (dd, J=4.17, 1.64 Hz, 1H).

### Example 10

### 4-(1,1-dimethylethyl)-9-{[4-(7-quinolinyl)phenyl]sulfonyl}-1-oxa-4,9-diazaspiro[5.5]undecan-3-one

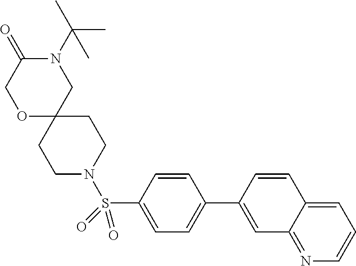 [SMILES: CC(C)(C)N1CC2(CCN(S(=O)(=O)c3ccc(-c4ccc5cccnc5c4)cc3)CC2)OCC1=O]

### a) 1-[(4-bromophenyl)sulfonyl]-4-{[(1,1-dimethylethyl)amino]methyl}-4-piperidinol

A 25 mL microwave reaction vial was charged with a suspension of 6-[(4-bromophenyl)sulfonyl]-1-oxa-6-azaspiro[2.5]octane (0.626 mmol) and tert-butylamine (2.191 mmol) in absolute ethanol (5.0 mL) then sealed with a standard aluminum crimp cap. The vessel was heated on an aluminum block at 85° C. for 4 h. The resulting suspension was cooled to room temperature and the solids were collected by filtration, rinsed with hexanes, then suction and vacuum dried to afford the title product (230 mg, 90% yield) as a white crystalline solid. MS(ES)+ m/e 405.3, 407.4 [M+H]⁺. ¹H NMR (400 MHz, DMSO-d₆) δ ppm 0.97 (s, 9H) 1.26 (br. s., 1H) 1.41-1.61 (m, 4H) 2.32 (br. s., 2H) 2.50-2.55 (m, 2H) 3.34-3.45 (m, 2H) 4.10 (s, 1H) 7.67 (d, J=8.59 Hz, 2H) 7.86 (d, J=8.59 Hz, 2H).

### b) 9-[(4-bromophenyl)sulfonyl]-4-(1,1-dimethylethyl)-1-oxa-4,9-diazaspiro[5.5]undecan-3-one

To a solution of 1-[(4-bromophenyl)sulfonyl]-4-{[(1,1-dimethylethyl)amino]methyl}-4-piperidinol (0.558 mmol) and triethylamine (1.394 mmol) in anhydrous dichloromethane (DCM) (20 mL) cooled to 0° C. was added neat chloroacetyl chloride (0.836 mmol). The ice bath was removed and stirring continued for 30 min, whereupon analysis by LCMS indicated complete conversion to the intermediate chloroacetamide. The solution was diluted with dichloromethane, washed with water and brine, dried (sodium sulfate), and evaporated under reduced pressure to give the intermediate as a tan solid. MS(ES)+ m/e 481.0, 482.9 [M+H]⁺.

The crude intermediate was taken into anhydrous tetrahydrofuran (THF) (20.00 mL) then treated with 60% sodium hydride in mineral oil (2.509 mmol). The resulting brown suspension was heated at reflux for 3 h then cooled to room temperature and slowly quenched with water. The reaction was diluted further with water, extracted with ethyl acetate, and the extracts were dried (sodium sulfate), treated with silica powder (2 g) and evaporated under reduced pressure to dryness. This was purified by flash chromatography (50% hexanes in ethyl acetate) to afford the title product (175 mg, 67% yield) as a white solid. MS(ES)+ m/e 445.1, 446.9 [M+H]⁺. ¹H NMR (400 MHz, DMSO-d₆) δ ppm 1.34 (s, 9H) 1.58-1.72 (m, 2H) 1.73-1.85 (m, 2H) 2.32-2.46 (m, 2H) 3.23 (s, 2H) 3.42-3.55 (m, 2H) 3.79 (s, 2H) 7.66 (d, J=8.59 Hz, 2H) 7.87 (d, J=8.84 Hz, 2H).

### c) 4-(1,1-dimethylethyl)-9-{[4-(7-quinolinyl)phenyl]sulfonyl}-1-oxa-4,9-diazaspiro[5.5]undecan-3-one

A suspension of 9-[(4-bromophenyl)sulfonyl]-4-(1,1-dimethylethyl)-1-oxa-4,9-diazaspiro[5.5]undecan-3-one (0.377 mmol), 7-(4,4,5,5-tetramethyl-1,3,2-dioxaborolan-2-yl)quinoline (0.566 mmol), PdCl₂(dppf)-CH₂Cl₂ adduct (0.019 mmol) and 2.0M aq potassium carbonate (754 μL) in 1,4-dioxane (10.0 ml) was heated at 100° C. for 2 h. The resulting dark suspension was cooled to room temperature then diluted with water and extracted with dichloromethane. The extracts were dried (sodium sulfate), treated with silica powder (2 g), and evaporated under reduced pressure to dryness. This was purified by flash chromatography (ethyl acetate). The desired fractions were combined and evaporated in vacuo to a light yellow solid that was recrystallized from acetonitrile/water to afford the title product (90 mg, 48% yield) as a white crystalline solid. MS(ES)+ m/e 494.2 [M+H]⁺. ¹H NMR (400 MHz, DMSO-d₆) δ ppm 1.34 (s, 9H) 1.60-1.75 (m, 2H) 1.77-1.89 (m, 2H) 2.40-2.48 (m, 2H) 3.24 (s, 2H) 3.50-3.64 (m, 2H) 3.79 (s, 2H) 7.59 (dd, J=8.34, 4.29 Hz, 1H) 7.87 (d, J=8.59 Hz, 2H) 8.04 (dd, J=8.59, 2.02 Hz, 1H) 8.11-8.19 (m, 3H) 8.40 (s, 1H) 8.42-8.48 (m, 1H) 8.98 (dd, J=4.29, 1.77 Hz, 1H).

### Example 11

### 4-(1-methylcyclopropyl)-9-{[4-(7-quinolinyl)phenyl]sulfonyl}-1-oxa-4,9-diazaspiro[5.5]undecan-3-one

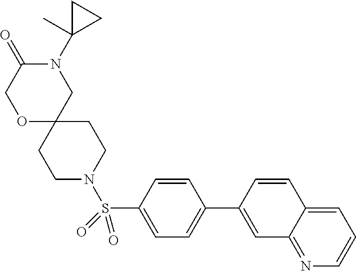 [SMILES: CC1(N2CC3(CCN(S(=O)(=O)c4ccc(-c5ccc6cccnc6c5)cc4)CC3)OCC2=O)CC1]

### a) 1-[(4-bromophenyl)sulfonyl]-4-{[(1-methylcyclopropyl)amino]methyl}-4-piperidinol trifluoroacetic acid salt

A 25 mL microwave reaction vial was charged with a suspension of 6-[(4-bromophenyl)sulfonyl]-1-oxa-6-azaspiro[2.5]octane (0.725 mmol), 1-methylcyclopropanamine hydrochloride 1.451 mmol) and N,N-diisopropylethylamine (1.451 mmol) in absolute ethanol (5.0 mL). The vessel was sealed with a standard aluminum crimp cap then heated on an aluminum block at 85° C. for 16 h. The reaction was evaporated under reduced pressure to a crude oil that was purified by reverse phase HPLC (10-60% acetonitrile w/0.1% TFA/water w/0.1% TFA) to give the title product (152 mg, 39% yield) as a white solid. MS(ES)+ m/e 402.9, 404.8 [M+H]⁺. ¹H NMR (400 MHz, DMSO-d₆) δ ppm 0.57-0.69 (m, 2H) 0.97-1.10 (m, 2H) 1.35 (s, 3H) 1.56-1.73 (m, 4H) 2.52-2.63 (m, 2H) 3.01 (br. s., 2H) 3.39-3.56 (m, 2H) 5.17 (br. s., 1H) 7.69 (d, J=8.34 Hz, 2H) 7.88 (d, J=8.34 Hz, 2H) 8.40 (br. s., 2H).

### b) 9-[(4-bromophenyl)sulfonyl]-4-(1-methylcyclopropyl)-1-oxa-4,9-diazaspiro[5.5]undecan-3-one

To a solution of 1-[(4-bromophenyl)sulfonyl]-4-{[(1-methylcyclopropyl)amino]methyl}-4-piperidinoltrifluoroacetic acid salt (0.286 mmol) and triethylamine (1.144 mmol) in anhydrous dichloromethane (DCM) (10.0 mL) cooled to 0° C. was added neat chloroacetyl chloride (0.429 mmol). The reaction stirred at 0° C. for 30 min then at room temperature for 2 h. The resulting brown solution was diluted with dichloromethane then washed with water and brine. The organic phase was collected, dried (sodium sulfate), and evaporated under reduced pressure to afford the chloroacetamide intermediate as a tan foam. MS(ES)+ m/e 481.0 [M+H]⁺.

To a solution of the intermediate in anhydrous tetrahydrofuran (THF) (10.0 mL) was added 60% sodium hydride in mineral oil (1.287 mmol). Anhydrous dimethyl sulfoxide (DMSO) (3.0 mL) was added and stirring continued at room temperature for 4 days. The resulting brown solution was quenched with water and extracted with ethyl acetate. The extracts were washed with brine, dried (sodium sulfate), treated with silica powder (2 g), and evaporated under reduced pressure to dryness. This was purified via flash chromatography (40% hexanes in ethyl acetate) to afford the title product (90 mg, 70% yield) as a white solid. MS(ES)+ m/e 443.2, 445.2 [M+H]⁺. ¹H NMR (400 MHz, DMSO-d₆) δ ppm 0.54-0.64 (m, 2H) 0.68-0.78 (m, 2H) 1.20 (s, 3H) 1.59-1.75 (m, 2H) 1.74-1.86 (m, 2H) 2.34-2.46 (m, 2H) 3.20 (s, 2H) 3.41-3.53 (m, 2H) 3.82 (s, 2H) 7.66 (d, J=8.59 Hz, 2H) 7.87 (d, J=8.34 Hz, 2H).

### c) 4-(1-methylcyclopropyl)-9-{[4-(7-quinolinyl)phenyl]sulfonyl}-1-oxa-4,9-diazaspiro[5.5]undecan-3-one

A suspension of 9-[(4-bromophenyl)sulfonyl]-4-(1-methylcyclopropyl)-1-oxa-4,9-diazaspiro[5.5]undecan-3-one (0.192 mmol), 7-(4,4,5,5-tetramethyl-1,3,2-dioxaborolan-2-yl)quinoline (0.288 mmol), PdCl₂(dppf)-CH₂Cl₂ adduct (9.59 μmol) and 2.0M aq potassium carbonate (383 μL) in 1,4-dioxane (8.0 mL) was heated at 100° C. for 3 h. The resulting dark suspension was cooled to room temperature, diluted with water, and extracted with ethyl acetate. The extracts were dried (sodium sulfate), treated with silica gel (2 g), and evaporated under reduced pressure to dryness. This was purified by flash chromatography (ethyl acetate) and then by reverse phase HPLC (10-60% acetonitrile w/0.1% TFA/water w/0.1% TFA). The desired fractions were combined with saturated aq sodium bicarbonate and extracted with dichloromethane. The extracts were dried (sodium sulfate) and evaporated to a colorless residue that was taken into acetonitrile (1 mL) and water (1 mL), frozen, and lyophilized to afford the title product (41.6 mg, 44% yield) as a white solid. MS(ES)+ m/e 492.3 [M+H]⁺. ¹H NMR (400 MHz, DMSO-d₆) δ ppm 0.54-0.63 (m, 2H) 0.70-0.75 (m, 2H) 1.20 (s, 3H) 1.64-1.77 (m, 2H) 1.78-1.88 (m, 2H) 2.43-2.48 (m, 2H) 3.21 (s, 2H) 3.50-3.60 (m, 2H) 3.82 (s, 2H) 7.59 (dd, J=8.34, 4.29 Hz, 1H) 7.87 (d, J=8.34 Hz, 2H) 8.05 (dd, J=8.59, 1.77 Hz, 1H) 8.11-8.19 (m, 3H) 8.39-8.42 (m, 1H) 8.42-8.47 (m, 1H) 8.98 (dd, J=4.29, 1.77 Hz, 1H).

### Example 12

### 4-cyclobutyl-9-{[4-(7-quinolinyl)phenyl]sulfonyl}-1-oxa-4,9-diazaspiro[5.5]undecan-3-one

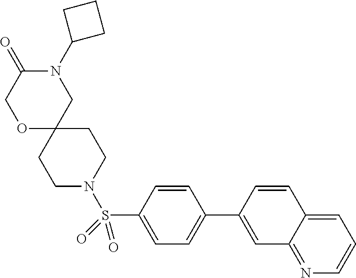 [SMILES: O=C1COC2(CCN(S(=O)(=O)c3ccc(-c4ccc5cccnc5c4)cc3)CC2)CN1C1CCC1]

### a) 1-[(4-bromophenyl)sulfonyl]-4-[(cyclobutylamino)methyl]-4-piperidinol

A 25 mL microwave reaction vial was charged with a suspension of 6-[(4-bromophenyl)sulfonyl]-1-oxa-6-azaspiro[2.5]octane (0.563 mmol) and cyclobutylamine (1.970 mmol) in absolute ethanol (15.0 mL). The vessel was sealed with a standard aluminum crimp cap then heated at 85° C. for 18 h. The resulting clear solution was adsorbed onto silica powder (1 g) and evaporated to dryness. This was purified via flash chromatography (1% methanol in ethyl acetate) to afford the title product (146 mg, 61% yield) as a white solid. MS(ES)+ m/e 402.8, 405.0 [M+H]⁺. ¹H NMR (400 MHz, DMSO-d₆) δ ppm 1.43-1.55 (m, 5H) 1.56-1.65 (m, 4H) 2.02-2.10 (m, 2H) 2.28 (s, 2H) 2.44-2.49 (m, 2H) 3.02-3.14 (m, 1H) 3.37 (d, J=11.37 Hz, 2H) 4.15 (s, 1H) 7.67 (d, J=8.08 Hz, 2H) 7.86 (d, J=8.08 Hz, 2H).

### b) 9-[(4-bromophenyl)sulfonyl]-4-cyclobutyl-1-oxa-4,9-diazaspiro[5.5]undecan-3-one

To a stirring solution of 1-[(4-bromophenyl)sulfonyl]-4-[(cyclobutylamino)methyl]-4-piperidinol (0.362 mmol) and triethylamine (0.905 mmol) in anhydrous dichloromethane (DCM) (15.0 mL) cooled to 0° C. was slowly added neat chloroacetyl chloride (0.537 mmol). The resulting light-brown solution stirred at 0° C. for 30 min, whereupon analysis by LCMS indicated complete conversion to the intermediate chloroacetamide. The reaction was diluted with dichloromethane, washed with water and brine, dried (sodium sulfate), and evaporated under reduced pressure to give the intermediate as a crude oil. MS(ES)+ m/e 480.9 [M+H]⁺.

This intermediate was taken into anhydrous tetrahydrofuran (THF) (15.0 mL) and dimethyl sulfoxide (DMSO) (3.0 ml) then treated with 60% sodium hydride in mineral oil (1.629 mmol) and heated at 60° C. for 18 h. The reaction was quenched with water, extracted with dichloromethane, and the extracts were dried (sodium sulfate), treated with silica gel (1 g) and evaporated under reduced pressure to dryness. This was purified by flash chromatography (40% hexanes in ethyl acetate) and then by reverse phase HPLC (10-85% acetonitrile w/0.1% TFA/water w/0.1% TFA). The fractions of interest were combined, treated with saturated aq sodium bicarbonate, and extracted with dichloromethane. The extracts were dried (sodium sulfate) and evaporated under reduced pressure to afford the title product (55 mg, 34% yield) as a white solid. MS(ES)+ m/e 443.0, 445.1 [M+H]⁺. ¹H NMR (400 MHz, DMSO-d₆) δ ppm 1.60 (dt, J=9.73, 4.99 Hz, 2H) 1.64-1.75 (m, 2H) 1.77-1.86 (m, 2H) 1.89-1.99 (m, 2H) 2.03-2.18 (m, 2H) 2.36-2.46 (m, 2H) 3.25 (s, 2H) 3.48 (d, J=11.87 Hz, 2H) 3.87 (s, 2H) 4.80-4.95 (m, 1H) 7.67 (d, J=8.59 Hz, 2H) 7.87 (d, J=8.34 Hz, 2H).

### c) 4-cyclobutyl-9-{[4-(7-quinolinyl)phenyl]sulfonyl}-1-oxa-4,9-diazaspiro[5.5]undecan-3-one

A suspension of 9-[(4-bromophenyl)sulfonyl]-4-cyclobutyl-1-oxa-4,9-diazaspiro[5.5]undecan-3-one (0.113 mmol), 7-(4,4,5,5-tetramethyl-1,3,2-dioxaborolan-2-yl)quinoline (0.169 mmol), PdCl₂(dppf)-CH₂Cl₂ adduct (5.64 μmol) and 2.0M aq potassium carbonate (226 μL) in 1,4-dioxane (8.0 ml) was heated at 100° C. for 3 h. The resulting dark suspension was cooled to room temperature, diluted with water, and extracted with ethyl acetate. The extracts were dried (sodium sulfate) and evaporated under reduced pressure to a brown residue that was purified by reverse phase HPLC (10-60% acetonitrile w/0.1% TFA/water w/0.1% TFA). The desired fractions were combined, treated with saturated aqueous sodium bicarbonate, and extracted with dichloromethane. The dried extracts (sodium sulfate) were evaporated to a residue that was recrystallized from absolute ethanol to afford the title product (30 mg, 51% yield) as an off-white crystalline solid (half ethanol solvate). MS(ES)+ m/e 492.2 [M+H]⁺. ¹H NMR (400 MHz, DMSO-d₆) δ ppm 1.05 (t, J=6.95 Hz, 3H) 1.60 (s, 2H) 1.66-1.78 (m, 2H) 1.81-1.88 (m, 2H) 1.89-1.98 (m, 2H) 2.03-2.18 (m, 2H) 2.43-2.49 (m, 2H) 3.27 (s, 2H) 3.44 (dd, J=7.07, 5.05 Hz, 1H) 3.52-3.62 (m, 2H) 3.87 (s, 2H) 4.35 (s, 0H) 4.80-4.95 (m, 1H) 7.59 (dd, J=8.21, 4.17 Hz, 1H) 7.87 (d, J=8.59 Hz, 2H) 8.05 (dd, J=8.59, 1.77 Hz, 1H) 8.15 (d, J=8.34 Hz, 1H) 8.16 (d, J=8.34 Hz, 2H) 8.41 (d, J=1.52 Hz, 1H) 8.42-8.47 (m, 1H) 8.98 (dd, J=4.29, 1.77 Hz, 1H).

### Example 13

### 9-(4-biphenylylsulfonyl)-4-cyclopropyl-1-oxa-4,9-diazaspiro[5.5]undecan-3-one

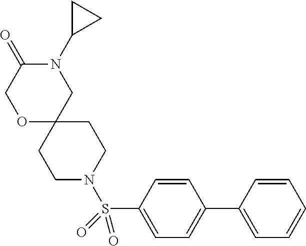 [SMILES: O=C1COC2(CCN(S(=O)(=O)c3ccc(-c4ccccc4)cc3)CC2)CN1C1CC1]

### a) 1,1-Dimethylethyl 1-oxa-6-azaspiro[2.5]octane-6-carboxylate

A mixture of trimethylsulfoxonium iodide (50.2 mmol) and anhydrous dimethyl sulfoxide (DMSO) (50 mL) was stirred at room temperature for 1 h. The reaction was then cooled to 0° C. and 60% sodium hydride in mineral oil (60.2 mmol) was added in small portions over several minutes. The reaction was allowed to warm to room temperature and stirred for 2 h. The resulting white slurry was cooled to 0° C. then treated with solid 1,1-dimethylethyl 4-oxo-1-piperidinecarboxylate (50.2 mmol) in one portion. The ice bath was removed and stirring continued at room temperature for 18 h. Ice cold water (150 mL) was added and the mixture was extracted into diethyl ether (3×). The extracts were washed with brine, dried (sodium sulfate) then evaporated under reduced pressure to a yellow oil. The oil was dissolved in ethyl acetate, treated with silica powder (˜20 g), and evaporated to dryness. This was placed on a short pad of silica in a sintered glass funnel and washed with hexanes (500 mL; the filtrate was discarded). The silica pad was then washed with 2:1 hexanes/ethyl acetate. The filtrate was evaporated in vacuo to give the title product (6.40 g, 57% yield) as a pale yellow oil that solidified upon standing. MS(ES)+ m/e 214.1 [M+H]⁺. ¹H NMR (400 MHz, CDCl₃) δ ppm 1.47 (s, 11H) 1.72-1.87 (m, 2H) 2.69 (s, 2H) 3.36-3.50 (m, 2H) 3.63-3.83 (m, 2H).

### b) 1,1-dimethylethyl 4-[(cyclopropylamino)methyl]-4-hydroxy-1-piperidine carboxylate

A sealable reaction vessel was charged with 1,1-dimethylethyl 1-oxa-6-azaspiro[2.5]octane-6-carboxylate (14.07 mmol), ethanol (70 mL) and cyclopropylamine (42.2 mmol). The vessel was purged with nitrogen, sealed and placed in a 75° C. oil bath for 20 h. The reaction mixture was cooled to room temperature and concentrated under reduced pressure. The resulting oil was purified by silica gel chromatography (5% methanol in ethyl acetate). The appropriate fractions were concentrated under reduced pressure and dried to afford the title product (3.56 g, 94% yield) as a viscous colorless oil. MS(ES)+ m/e 271.4 [M+H]⁺. ¹H NMR (400 MHz, DMSO-d₆) δ 4.20 (s, 1H), 3.59 (d, J=12.6 Hz, 2H), 3.05 (br. s., 2H), 2.16-2.00 (m, 2H), 1.49-1.31 (m, 14H), 0.42-0.29 (m, 2H), 0.24-0.13 (m, 2H).

### c) 1,1-dimethylethyl 4-{[(chloroacetyl)(cyclopropyl)amino]methyl}-4-hydroxy-1-piperidinecarboxylate

A solution of 1,1-dimethylethyl 4-[(cyclopropylamino)methyl]-4-hydroxy-1-piperidinecarboxylate (329 mmol) in tetrahydrofuran (500 mL) was added to a vigorously stirred suspension of sodium hydrogen carbonate (3193 mmol) in tetrahydrofuran (500 mL) at 0° C. Chloroacetyl chloride (332 mmol) was added dropwise over 10 min, maintaining the temperature at 0° C. The ice bath was removed and the mixture was stirred for 2 h, at which point a further aliquot of chloroacetyl chloride (41.1 mmol) was added. The mixture was stirred for 72 h then was filtered to remove the sodium hydrogen carbonate and the filter bed washed with tetrahydrofuran (300 mL) to afford the crude title product. MS(ES)+ m/e 347.1 [M+H+].

### d) 1,1-dimethylethyl 4-cyclopropyl-3-oxo-1-oxa-4,9-diazaspiro[5.5]undecane-9-carboxylate

The solution of 1,1-dimethylethyl 4-{[(chloroacetyl)(cyclopropyl)amino]methyl}-4-hydroxy-1-piperidinecarboxylate from Example 13c in tetrahydrofuran (1300 mL) was treated with potassium carbonate (28.8 mmol) and tetrabutylammonium hydrogensulfate (11.72 mmol), and a 15% w/w solution of sodium hydroxide (1195 mmol) was added over 4 h. The mixture was stirred overnight and transferred to a separating flask. The aqueous layer was drained and the organic layer diluted with t-butyl methyl ether (1.5 L) and washed with a mixture of brine and saturated aq ammonium chloride (250 mL). The organic layer was dried (Na₂SO₄) and evaporated to afford the crude title product as a gel. MS(ES)+ m/e 311.3 [M+H+]; ¹H NMR (400 MHz, CDCl₃) δ 4.14 (s, 2H), 3.86 (br s, 2H), 3.26-3.01 (m, 4H), 2.84-2.70 (m, 1H), 1.83 (d, J=12.1 Hz, 2H), 1.58-1.42 (m, 11H), 0.92-0.80 (m, 2H), 0.74-0.58 (m, 2H).

### e) 4-cyclopropyl-1-oxa-4,9-diazaspiro[5.5]undecan-3-one hydrochloride

The 1,1-dimethylethyl 4-cyclopropyl-3-oxo-1-oxa-4,9-diazaspiro[5.5]undecane-9-carboxylate from Example 13d was dissolved in ethanol (300 mL) and cooled over an ice bath. A 4M solution of hydrogen chloride in dioxane (300 mL) was added, such that the temperature remained low. The ice bath was removed and the mixture was stirred at ambient temperature overnight. The solid was collected, washed with a little ethanol and diethyl ether to give the title product (47.1 g, 58%). The mother liquors were treated with diethyl ether (1.2 L), stirred for 30 min, and the solid was collected and dried to give a total combined yield of 78% of the title product (63.69 g) over the three steps. MS (ES)+ m/e 211.0[M+H+]; ¹H NMR (400 MHz, DMSO-d₆) δ 9.17-8.64 (m, 2H), 4.04 (s, 2H), 3.14 (d, J=13.1 Hz, 2H), 3.01-2.85 (m, 2H), 2.83-2.71 (m, 1H), 1.96-1.83 (m, 2H), 1.83-1.68 (m, 2H), 0.76-0.67 (m, 2H), 0.64-0.55 (m, 2H).

### f) 9-[(4-bromophenyl)sulfonyl]-4-cyclopropyl-1-oxa-4,9-diazaspiro[5.5]undecan-3-one

To a mixture of 4-cyclopropyl-1-oxa-4,9-diazaspiro[5.5]undecan-3-one hydrochloride (8.11 mmol) in dichloromethane (100 mL) was added N,N-diisdopropylethylamine (24.32 mmol). The mixture was stirred for 5 min, then 4-bromobenzenesulfonyl chloride (8.11 mmol) was added, giving a clear solution. After stirring overnight, the reaction was washed with 1M aq hydrochloric acid. The layers were separated and the aqueous layer was extracted with dichloromethane. The combined organics were washed with brine, dried over Na₂SO₄, filtered and concentrated under reduced pressure to a solid. The residue was triturated with ethanol and then collected and washed with ethanol and hexanes to provide the title product (3.0 g, 86% yield) as a white solid. LCMS(ES) m/e 430.0 [M+H+]; ¹H NMR (400 MHz, CDCl₃) δ 7.77-7.68 (m, 2H), 7.68-7.60 (m, 2H), 4.00 (s, 2H), 3.73-3.59 (m, 2H), 3.15 (s, 2H), 2.80-2.67 (m, 1H), 2.57 (td, J=2.7, 12.1 Hz, 2H), 1.94 (dd, J=2.4, 14.3 Hz, 2H), 1.78-1.62 (m, 2H), 0.94-0.83 (m, 2H), 0.69-0.59 (m, 2H).

### g) 9-(4-biphenylylsulfonyl)-4-cyclopropyl-1-oxa-4,9-diazaspiro[5.5]undecan-3-one

In a sealed microwave vial, a mixture of 9-[(4-bromophenyl)sulfonyl]-4-cyclopropyl-1-oxa-4,9-diazaspiro[5.5]undecan-3-one (0.233 mmol), benzeneboronic acid (0.246 mmol), 1,1′-bis(diphenylphosphino)ferrocene-palladium(II)dichloride dichloromethane complex (0.012 mmol) and 2M aq potassium carbonate (0.5 mL) in 1,4-dioxane (2 mL) was stirred at 100° C. for 18 h. The reaction was cooled to room temperature. The reaction was diluted with ethyl acetate (50 mL) and water (50 mL). The layers were separated and the aqueous layer was extracted with ethyl acetate (20 mL). The organic layers were combined, dried over magnesium sulfate and concentrated in vacuo. Purification of the residue by silica gel chromatography (1% methanol/ethyl acetate) provided the title product as a white solid (65 mg, 65%). MS(ES)+ m/e 427.1 [M+H]⁺.

### Example 14

### 4-cyclopropyl-9-{[4-(1H-indol-6-yl)phenyl]sulfonyl}-1-oxa-4,9-diazaspiro[5.5]undecan-3-one

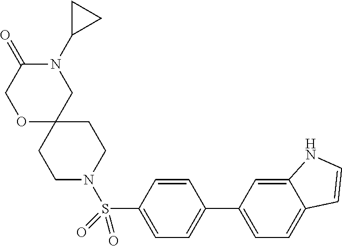 [SMILES: O=C1COC2(CCN(S(=O)(=O)c3ccc(-c4ccc5cc[nH]c5c4)cc3)CC2)CN1C1CC1]

a) A microwave vial was charged with 9-[(4-bromophenyl)sulfonyl]-4-cyclopropyl-1-oxa-4,9-diazaspiro[5.5]undecan-3-one (0.263 mmol), 1H-indol-6-ylboronic acid (0.276 mmol), PdCl₂(dppf) (0.013 mmol), a solution of K₂CO₃ (0.658 mmol) in water (1 mL) and 1,4-dioxane (3 mL). The vial was purged with nitrogen, sealed and irradiated in a microwave reactor for 30 min at 150° C. LCMS analysis of the crude reaction indicated complete and clean conversion to desired product. The reaction mixture was concentrated under reduced pressure and the residue was dissolved in DMSO (3 mL), filtered through a syringe filter, and purified by reverse phase HPLC (10-90% acetonitrile/water+0.1% TFA). The appropriate fractions were concentrated to remove a majority of the acetonitrile, leaving an aqueous suspension of product which was collected by filtration and dried to constant weight to provide the title product (62 mg, 51% yield) as an white solid. MS(ES)+ m/e 466.1 [M+H]⁺. ¹H NMR (400 MHz, DMSO-d₆) δ 11.31 (br s, 1H), 7.96 (d, J=8.34 Hz, 2H), 7.73-7.83 (m, 3H), 7.67 (d, J=8.34 Hz, 1H), 7.36-7.50 (m, 2H), 6.49 (br. s., 1H), 3.86 (s, 2H), 3.52 (d, J=11.37 Hz, 2H), 3.13 (s, 2H), 2.65-2.77 (m, 1H), 2.40-2.48 (m, 2H), 1.84 (d, J=13.64 Hz, 2H), 1.60-1.75 (m, 2H), 0.63-0.74 (m, 2H), 0.52-0.62 (m, 2H).

### Example 15

### 4′-[(4-cyclopropyl-3-oxo-1-oxa-4,9-diazaspiro[5.5]undec-9-yl)sulfonyl]-4-biphenylcarbonitrile

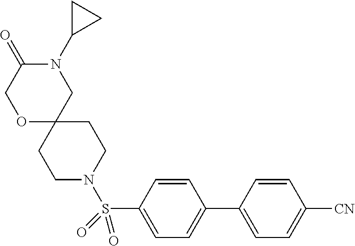 [SMILES: N#Cc1ccc(-c2ccc(S(=O)(=O)N3CCC4(CC3)CN(C3CC3)C(=O)CO4)cc2)cc1]

a) A microwave vial was charged with 9-[(4-bromophenyl)sulfonyl]-4-cyclopropyl-1-oxa-4,9-diazaspiro[5.5]undecan-3-one (0.228 mmol), 4-(4,4,5,5-tetramethyl-1,3,2-dioxaborolan-2-yl)benzonitrile (0.228 mmol), PdCl₂(dppf) (0.011 mmol), a solution of K₂CO₃ (0.571 mmol) in water (1 mL) and 1,4-dioxane (3 mL). The vial was purged with nitrogen, sealed and irradiated in a microwave reactor for 30 min at 150° C. LCMS analysis of the crude reaction indicated complete and clean conversion to desired product. The reaction mixture was concentrated under reduced pressure and the residue was dissolved in DMSO (3 mL), filtered through a syringe filter and purified by reverse phase HPLC (10-90% acetonitrile/water+0.1% TFA). The appropriate fractions were concentrated to remove a majority of the acetonitrile, leaving an aqueous suspension of product which was collected by filtration and dried to constant weight to provide the title product (69 mg, 67% yield) as a white solid. MS(ES)+ m/e 451.9 [M+H]⁺. ¹H NMR (400 MHz, DMSO-d₆) δ 8.04 (d, J=8.34 Hz, 2H), 8.00 (s, 4H), 7.85 (d, J=8.34 Hz, 2H), 3.85 (s, 2H), 3.53 (d, J=11.37 Hz, 2H), 3.12 (s, 2H), 2.66-2.78 (m, J=3.66, 4.04, 7.26, 7.26 Hz, 1H), 2.40-2.48 (m, 2H), 1.83 (d, J=13.14 Hz, 2H), 1.62-1.75 (m, 2H), 0.63-0.73 (m, 2H), 0.50-0.62 (m, 2H).

### Example 16

### 4-cyclopropyl-9-[(4′-fluoro-4-biphenylyl)sulfonyl]-1-oxa-4,9-diazaspiro[5.5]undecan-3-one

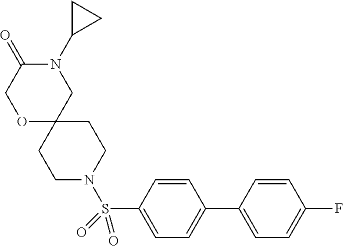 [SMILES: O=C1COC2(CCN(S(=O)(=O)c3ccc(-c4ccc(F)cc4)cc3)CC2)CN1C1CC1]

a) A microwave vial was charged with 9-[(4-bromophenyl)sulfonyl]-4-cyclopropyl-1-oxa-4,9-diazaspiro[5.5]undecan-3-one (0.233 mmol), (4-fluorophenyl)boronic acid (0.233 mmol), PdCl₂(dppf) (0.012 mmol), a solution of K₂CO₃ (0.582 mmol) in water (1 mL) and 1,4-dioxane (3 mL). The vial was purged with nitrogen, sealed and irradiated in a microwave reactor for 30 min at 150° C. LCMS analysis of the crude reaction indicated complete and clean conversion to desired product. The reaction mixture was concentrated under reduced pressure and the residue was dissolved in DMSO (3 mL), filtered through a syringe filter and purified by reverse phase HPLC (10-90% acetonitrile/water+0.1% TFA). The appropriate fractions were concentrated to remove a majority of the acetonitrile, leaving an aqueous suspension of product which was collected by filtration and dried to constant weight to provide the title product (77 mg, 74% yield) as a white solid. MS(ES)+ m/e 445.1 [M+H]⁺. ¹H NMR (400 MHz, DMSO-d₆) δ 7.94 (d, J=8.34 Hz, 2H), 7.75-7.88 (m, 4H), 7.37 (t, J=8.59 Hz, 2H), 3.85 (s, 2H), 3.50 (br. s., 2H), 3.12 (s, 2H), 2.65-2.79 (m, 1H), 2.43 (br. s., 2H), 1.83 (d, J=13.14 Hz, 2H), 1.69 (d, J=9.35 Hz, 2H), 0.63-0.75 (m, 2H), 0.58 (br. s., 2H).

### Example 17

### 4-cyclopropyl-9-{[4-(1H-indazol-6-yl)phenyl]sulfonyl}-1-oxa-4,9-diazaspiro[5.5]undecan-3-one

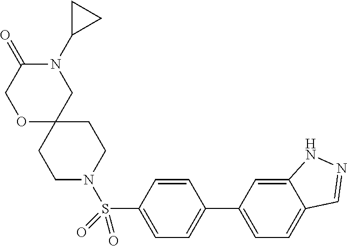 [SMILES: O=C1COC2(CCN(S(=O)(=O)c3ccc(-c4ccc5cn[nH]c5c4)cc3)CC2)CN1C1CC1]

a) A microwave vial was charged with 9-[(4-bromophenyl)sulfonyl]-4-cyclopropyl-1-oxa-4,9-diazaspiro[5.5]undecan-3-one (0.233 mmol), 1H-indazol-6-ylboronic acid (0.280 mmol), PdCl₂(dppf) (0.012 mmol), a solution of K₂CO₃ (0.582 mmol) in water (1 mL) and 1,4-dioxane (3 mL). The vial was purged with nitrogen, sealed and irradiated in a microwave reactor for 30 min at 150° C. LCMS analysis of the crude reaction mixture indicated 86% desired product+14% starting bromide. The reaction mixture was concentrated under reduced pressure and the residue was dissolved in DMSO (3 mL), filtered through a syringe filter and purified by reverse phase HPLC (10-90% acetonitrile/water+0.1% TFA). The appropriate fractions were concentrated to remove a majority of the acetonitrile, leaving an aqueous suspension of product which was collected by filtration and dried to constant weight under high vacuum to provide the title product (39 mg, 36% yield) as a white solid. MS(ES)+ m/e 467.0 [M+H]⁺. ¹H NMR (400 MHz, DMSO-d₆) δ 13.28 (s, 1H), 8.14 (s, 1H), 8.02 (d, J=8.34 Hz, 2H), 7.78-7.94 (m, 4H), 7.52 (d, J=8.34 Hz, 1H), 3.86 (s, 2H), 3.53 (d, J=11.37 Hz, 2H), 3.13 (s, 2H), 2.66-2.80 (m, 1H), 2.39-2.48 (m, 2H), 1.84 (d, J=13.39 Hz, 2H), 1.59-1.77 (m, 2H), 0.62-0.74 (m, 2H), 0.46-0.62 (m, 2H).

### Example 18

### 4-cyclopropyl-9-{[4-(1H-indol-5-yl)phenyl]sulfonyl}-1-oxa-4,9-diazaspiro[5.5]undecan-3-one

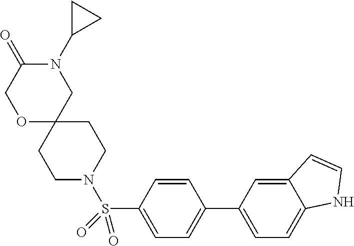 [SMILES: O=C1COC2(CCN(S(=O)(=O)c3ccc(-c4ccc5[nH]ccc5c4)cc3)CC2)CN1C1CC1]

a) A microwave vial was charged with 9-[(4-bromophenyl)sulfonyl]-4-cyclopropyl-1-oxa-4,9-diazaspiro[5.5]undecan-3-one (0.186 mmol), 5-(4,4,5,5-tetramethyl-1,3,2-dioxaborolan-2-yl)-1H-indole (0.196 mmol), PdCl₂(dppf) (9.32 μmol), a solution of K₂CO₃ (0.466 mmol) in water (1 mL) and 1,4-dioxane (3 mL). The vial was purged with nitrogen, sealed and irradiated in a microwave reactor for 30 min at 150° C. LCMS analysis of the crude reaction indicated complete and clean conversion to desired product. The reaction mixture was concentrated under reduced pressure and the residue was dissolved in DMSO (2 mL), filtered through a syringe filter and purified by reverse phase HPLC (10-90% acetonitrile/water+0.1% TFA). The appropriate fractions were concentrated to remove a majority of the acetonitrile, leaving an aqueous suspension of product which was collected by filtration and dried to constant weight to provide the title product (53 mg, 61% yield) as an off-white solid. MS(ES)+ m/e 466.2 [M+H]⁺. ¹H NMR (400 MHz, DMSO-d₆) δ 11.27 (br. s., 1H), 7.95 (d, J=8.84 Hz, 3H), 7.77 (d, J=8.08 Hz, 2H), 7.47-7.57 (m, 2H), 7.43 (br. s., 1H), 6.53 (br. s., 1H), 3.86 (s, 2H), 3.51 (d, J=11.62 Hz, 2H), 3.12 (s, 2H), 2.67-2.78 (m, 1H), 2.39-2.48 (m, 2H), 1.84 (d, J=13.39 Hz, 2H), 1.58-1.75 (m, 2H), 0.64-0.74 (m, 2H), 0.51-0.61 (m, 2H).

### Example 19

### 9-{[4-(1-benzofuran-5-yl)phenyl]sulfonyl}-4-cyclopropyl-1-oxa-4,9-diazaspiro[5.5]undecan-3-one

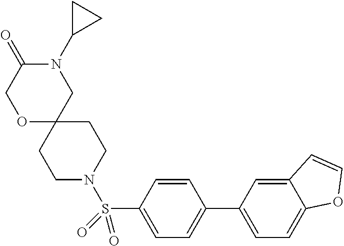 [SMILES: O=C1COC2(CCN(S(=O)(=O)c3ccc(-c4ccc5occc5c4)cc3)CC2)CN1C1CC1]

a) A microwave vial was charged with 9-[(4-bromophenyl)sulfonyl]-4-cyclopropyl-1-oxa-4,9-diazaspiro[5.5]undecan-3-one (0.186 mmol), 5-(4,4,5,5-tetramethyl-1,3,2-dioxaborolan-2-yl)-1-benzofuran (0.196 mmol), PdCl₂(dppf) (9.32 μmol), a solution of K₂CO₃ (0.466 mmol) in water (1 mL) and 1,4-dioxane (3 mL). The vial was purged with nitrogen, sealed and irradiated in a microwave reactor for 30 min at 150° C. LCMS analysis of the crude reaction indicated complete and clean conversion to desired product. The reaction mixture was concentrated under reduced pressure and the residue was dissolved in DMSO (2 mL), filtered through a syringe filter and purified by reverse phase HPLC (10-90% acetonitrile/water+0.1% TFA). The appropriate fractions were concentrated to remove a majority of the acetonitrile, leaving an aqueous suspension of product which was collected by filtration and dried to constant weight to provide the title product (51 mg, 59% yield) as an off-white solid. MS(ES)+ m/e 467.0 [M+H]⁺. ¹H NMR (400 MHz, DMSO-d₆) δ 8.04-8.12 (m, 2H), 7.98 (d, J=8.08 Hz, 2H), 7.81 (d, J=8.08 Hz, 2H), 7.69-7.78 (m, 2H), 7.06 (s, 1H), 3.86 (s, 2H), 3.52 (d, J=11.62 Hz, 2H), 3.13 (s, 2H), 2.73 (dd, J=3.66, 7.45 Hz, 1H), 2.37-2.47 (m, 2H), 1.84 (d, J=13.14 Hz, 2H), 1.60-1.75 (m, 2H), 0.64-0.74 (m, 2H), 0.58 (d, 2H).

### Example 20

### 4′-[(4-cyclopropyl-3-oxo-1-oxa-4,9-diazaspiro[5.5]undec-9-yl)sulfonyl]-3-methyl-4-biphenylcarbonitrile

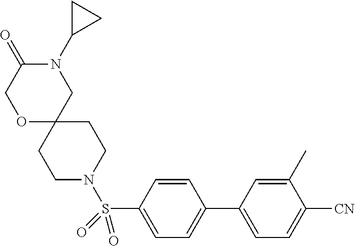 [SMILES: Cc1cc(-c2ccc(S(=O)(=O)N3CCC4(CC3)CN(C3CC3)C(=O)CO4)cc2)ccc1C#N]

### a) 2-methyl-4-(4,4,5,5-tetramethyl-1,3,2-dioxaborolan-2-yl)benzonitrile

A sealable reaction vessel was charged with 4-bromo-2-methylbenzonitrile (5.10 mmol), bis(pinacolato)diboron (5.61 mmol), potassium acetate (15.30 mmol), bis(triphenylphosphine)palladium(II) chloride (0.255 mmol) and 1,4-dioxane (10 mL). The vessel was purged with nitrogen, sealed and heated to 95° C. The reaction mixture turned very dark over the first hour. After stirring overnight (17 h), the reaction mixture was allowed to cool. The reaction mixture was diluted with ethyl acetate and filtered through a pad of silica gel, rinsing with ethyl acetate. The filtrate was concentrated under reduced pressure and purified by silica gel chromatography (15% ethyl acetate in hexanes). The appropriate fractions (product stains on TLC with Hanessian's stain with heat) were concentrated under reduced pressure. The residue was dissolved in hexanes (˜50 mL) and placed in a dry ice bath with scratching to promote crystallization. After standing for 30 min, the resulting solid was collected by filtration, rinsed with minimal very cold (dry ice cooled) hexanes and dried to constant weight to provide the title product (0.493 g, 40% yield) as a white cotton-like solid. LCMS indicates ˜1:1 mixture of boronic acid (M+H⁺=162.2) and boronic ester (M+H⁺=244.4). ¹H NMR (400 MHz, dmso-d₆) indicates exclusive boronic ester.

### b) 4′-[(4-cyclopropyl-3-oxo-1-oxa-4,9-diazaspiro[5.5]undec-9-yl)sulfonyl]-3-methyl-4-biphenylcarbonitrile

A microwave vial was charged with 9-[(4-bromophenyl)sulfonyl]-4-cyclopropyl-1-oxa-4,9-diazaspiro[5.5]undecan-3-one (0.186 mmol), 2-methyl-4-(4,4,5,5-tetramethyl-1,3,2-dioxaborolan-2-yl)benzonitrile (0.186 mmol), PdCl₂(dppf) (9.32 μmol), a solution of K₂CO₃ (0.466 mmol) in water (1 mL) and 1,4-dioxane (3 mL). The vial was purged with nitrogen, sealed and irradiated in a microwave reactor for 30 min at 150° C. LCMS analysis of the crude reaction indicated complete and clean conversion to desired product. The reaction mixture was concentrated under reduced pressure and the residue was dissolved in DMSO (2 mL), filtered through a syringe filter and purified by reverse phase HPLC (10-90% acetonitrile/water+0.1% TFA). The appropriate fractions were concentrated under reduced pressure to remove a majority of the acetonitrile, leaving an aqueous suspension of product which was collected by filtration, suspended in 10% ethyl acetate in hexanes, sonicated and heated to a gentle boil. After cooling to room temperature, the precipitate was collected by filtration and dried to constant weight to provide the title product (49 mg, 56% yield) as a white solid. MS(ES)+ m/e 466.2 [M+H]⁺. ¹H NMR (400 MHz, DMSO-d₆) δ 8.02 (d, J=8.34 Hz, 2H), 7.88-7.97 (m, 2H), 7.85 (d, J=8.08 Hz, 2H), 7.79 (d, J=8.08 Hz, 1H), 3.85 (s, 2H), 3.53 (d, J=11.37 Hz, 2H), 3.12 (s, 2H), 2.66-2.77 (m, 1H), 2.58 (s, 3H), 2.45 (d, J=12.88 Hz, 2H), 1.83 (d, J=13.39 Hz, 2H), 1.58-1.75 (m, 2H), 0.65-0.71 (m, 2H), 0.55-0.60 (m, 2H).

### Example 21

### 4-cyclopropyl-9-{[2-fluoro-4-(1H-indol-6-yl)phenyl]sulfonyl}-1-oxa-4,9-diazaspiro[5.5]undecan-3-one

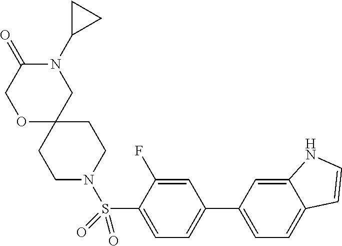 [SMILES: O=C1COC2(CCN(S(=O)(=O)c3ccc(-c4ccc5cc[nH]c5c4)cc3F)CC2)CN1C1CC1]

### a) 9-[(4-bromo-2-fluorophenyl)sulfonyl]-4-cyclopropyl-1-oxa-4,9-diazaspiro[5.5]undecan-3-one

To a mixture of 4-cyclopropyl-1-oxa-4,9-diazaspiro[5.5]undecan-3-one hydrochloride (3.23 mmol) in dichloromethane (DCM) (30 mL) was added triethylamine (8.08 mmol). The mixture was stirred for 5 min, then 4-bromo-2-fluorobenzenesulfonyl chloride (3.88 mmol) was added, giving a clear yellow solution. After stirring overnight (16 h), the reaction mixture was quenched with water. The layers were separated and the aqueous layer was back-extracted with DCM. The combined organics were dried over Na₂SO₄, filtered and concentrated under reduced pressure. The residue was triturated with hot 50% ethyl acetate/hexanes, allowed to stand at room temperature for 1 h, and the resulting precipitate was collected by filtration, rinsed with minimal 50% ethyl acetate/hexanes, and dried to constant weight to provide the title product (1197 mg, 83% yield) as an off-white solid. MS(ES)+ m/e 447.1, 448.9 [M+H]⁺.

### b) 4-cyclopropyl-9-{[2-fluoro-4-(1H-indol-6-yl)phenyl]sulfonyl}-1-oxa-4,9-diazaspiro[5.5]undecan-3-one

A microwave vial was charged with 9-[(4-bromo-2-fluorophenyl)sulfonyl]-4-cyclopropyl-1-oxa-4,9-diazaspiro[5.5]undecan-3-one (0.224 mmol), 1H-indol-6-ylboronic acid (0.235 mmol), PdCl₂(d_{PP} f) (0.011 mmol), a solution of K₂CO₃ (0.559 mmol) in water (1 mL) and 1,4-dioxane (3 mL). The vial was purged with nitrogen, sealed and irradiated in a microwave reactor for 30 min at 150° C. LCMS analysis of the crude reaction mixture indicated complete and clean conversion to desired product. The reaction mixture was concentrated under reduced pressure and the residue was dissolved in DMSO (3 mL), filtered through a syringe filter and purified by reverse phase HPLC (10-90% acetonitrile/water+0.1% TFA). The appropriate fractions were concentrated to remove a majority of the acetonitrile, leaving an aqueous suspension of product which was collected by filtration and dried to constant weight to provide the title product (61 mg, 56% yield) as an off-white solid. MS(ES)+ m/e 484.0 [M+H]⁺. ¹H NMR (400 MHz, DMSO-d₆) δ 11.36 (br. s., 1H), 7.73-7.88 (m, 4H), 7.67 (d, J=8.34 Hz, 1H), 7.40-7.52 (m, 2H), 6.50 (br. s., 1H), 3.92 (s, 2H), 3.55 (d, J=11.62 Hz, 2H), 3.15 (s, 2H), 2.63-2.80 (m, 3H), 1.86 (d, J=13.64 Hz, 2H), 1.58-1.75 (m, 2H), 0.64-0.75 (m, 2H), 0.52-0.63 (m, 2H).

### Example 22

### 4′-[(4-cyclopropyl-3-oxo-1-oxa-4,9-diazaspiro[5.5]undec-9-yl)sulfonyl]-3′-fluoro-4-biphenylcarbonitrile

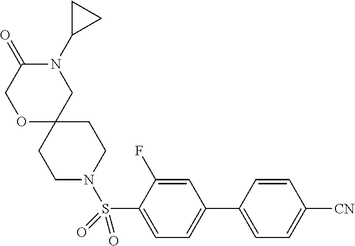 [SMILES: N#Cc1ccc(-c2ccc(S(=O)(=O)N3CCC4(CC3)CN(C3CC3)C(=O)CO4)c(F)c2)cc1]

a) Following the procedure described in Example 21b with 4-(4,4,5,5-tetramethyl-1,3,2-dioxaborolan-2-yl)benzonitrile (0.224 mmol) afforded the title product (50%). MS(ES)+m/e 470.1 [M+H]⁺. ¹H NMR (400 MHz, DMSO-d₆) δ 7.94-8.09 (m, 5H), 7.77-7.93 (m, 2H), 3.92 (s, 2H), 3.56 (d, J=11.87 Hz, 2H), 3.15 (s, 2H), 2.64-2.79 (m, 3H), 1.85 (d, J=13.39 Hz, 2H), 1.57-1.76 (m, 2H), 0.63-0.74 (m, 2H), 0.53-0.63 (m, 2H).

### Example 23

### 4-cyclopropyl-9-[(3,4′-difluoro-4-biphenylyl)sulfonyl]-1-oxa-4,9-diazaspiro[5.5]undecan-3-one

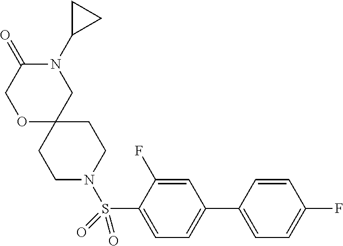 [SMILES: O=C1COC2(CCN(S(=O)(=O)c3ccc(-c4ccc(F)cc4)cc3F)CC2)CN1C1CC1]

a) A microwave vial was charged with 9-[(4-bromo-2-fluorophenyl)sulfonyl]-4-cyclopropyl-1-oxa-4,9-diazaspiro[5.5]undecan-3-one (0.224 mmol), (4-fluorophenyl)boronic acid (0.224 mmol), PdCl₂(dppf) (0.011 mmol), a solution of K₂CO₃ (0.559 mmol) in water (1 mL) and 1,4-dioxane (3 mL). The vial was purged with nitrogen, sealed and irradiated in a microwave reactor for 30 min at 150° C. LCMS of the crude reaction indicated complete and clean conversion to desired product. The reaction mixture was concentrated under reduced pressure and the residue was dissolved in DMSO (3 mL), filtered through a syringe filter and purified by reverse phase HPLC (10-90% acetonitrile/water+0.1% TFA). The appropriate fractions were concentrated under reduced pressure to remove a majority of the acetonitrile and the aqueous mixture was extracted with ethyl acetate. The organic layer was dried (Na₂SO₄), filtered, concentrated under reduced pressure and dried to constant weight under vacuum to provide the title product (66 mg, 64% yield) as an off-white solid. MS(ES)+ m/e 463.2 [M+H]⁺. ¹H NMR (400 MHz, DMSO-d₆) δ 7.79-7.92 (m, 4H), 7.71-7.78 (m, 1H), 7.32-7.42 (m, 2H), 3.91 (s, 2H), 3.55 (d, J=12.13 Hz, 2H), 3.14 (s, 2H), 2.61-2.79 (m, 3H), 1.85 (d, J=13.39 Hz, 2H), 1.58-1.75 (m, 2H), 0.64-0.73 (m, 2H), 0.50-0.63 (m, 2H).

### Example 24

### 4-cyclopropyl-9-{[4-(1-methyl-2-oxo-1,2-dihydro-6-quinolinyl)phenyl]sulfonyl}-1-oxa-4,9-diazaspiro[5.5]undecan-3-one

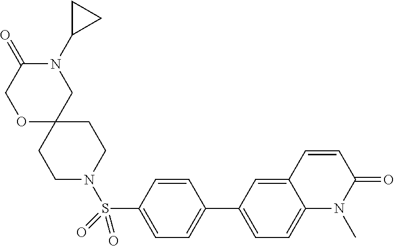 [SMILES: Cn1c(=O)ccc2cc(-c3ccc(S(=O)(=O)N4CCC5(CC4)CN(C4CC4)C(=O)CO5)cc3)ccc21]

### a) 6-bromo-1-methyl-2(1H)-quinolinone

To a stirring solution of 6-bromo-2(1H)-quinolinone (68.0 mmol) in anhydrous N,N-dimethylformamide (100 mL) was slowly added 60% sodium hydride in mineral oil (74.8 mmol). The reaction stirred at room temperature for 10 min then neat iodomethane (71.4 mmol) was added. The reaction was stirred at room temperature for 24 h and then poured into cold water (500 mL). The resulting slurry stirred for 1 h then the solids were collected by filtration, rinsed with water and suction dried overnight. Purification of the residue by silica gel chromatography (50-90% ethyl acetate/hexanes) followed by recrystallization from ethyl acetate/hexanes provided the title compound as pale pink needles (85%). MS(ES)+ m/e 237.7, 239.7 [M+H]⁺, bromine pattern. ¹H NMR (400 MHz, DMSO-d₆) δ ppm 3.59 (s, 3H) 6.67 (d, J=9.35 Hz, 1H) 7.48 (d, J=9.09 Hz, 1H) 7.75 (dd, J=8.97, 2.40 Hz, 1H) 7.88 (d, J=9.35 Hz, 1H) 7.98 (d, J=2.27 Hz, 1H).

### b) 1-methyl-6-(4,4,5,5-tetramethyl-1,3,2-dioxaborolan-2-yl)-2(1H)-quinolinone

A slurry of 6-bromo-1-methyl-2(1H)-quinolinone (15.88 mmol), bis(pinacolato)diboron (17.46 mmol), 1,1′-bis(diphenylphosphino)ferrocene-palladium(II)dichloride dichloromethane complex (0.793 mmol) and potassium acetate (31.8 mmol) in anhydrous 1,4-dioxane (100 mL) was heated at 110° C. for 3 h. The reaction was cooled to room temperature and then treated with silica powder (10 g) and decolorizing (500 mg) charcoal. The slurry was evaporated under reduced pressure to a dry powder then poured onto a short pad of silica powder and rinsed with ethyl acetate. The filtrate was evaporated to a dark oil. Purification of the oil by silica gel chromatography twice (60% ethyl acetate/hexanes and then 1% methanol/dichloromethane) provided the title compound as a white solid (21%). MS(ES)+ m/e 286.1 [M+H]⁺. ¹H NMR (400 MHz, DMSO-d₆) δ ppm 1.31 (s, 12H) 3.61 (s, 3H) 6.62 (d, J=9.35 Hz, 1H) 7.51 (d, J=8.59 Hz, 1H) 7.84 (dd, J=8.34, 1.26 Hz, 1H) 7.99 (d, J=9.60 Hz, 1H) 8.06 (d, J=1.26 Hz, 1H).

### c) 4-cyclopropyl-9-{[4-(1-methyl-2-oxo-1,2-dihydro-6-quinolinyl)phenyl]sulfonyl}-1-oxa-4,9-diazaspiro[5.5]undecan-3-one

Following the procedure described in Example 13g with 1-methyl-6-(4,4,5,5-tetramethyl-1,3,2-dioxaborolan-2-yl)-2(1H)-quinolinone for 1 h provided the title compound as an off-white solid (71%). Additionally, the aqueous layer was neutralized using 1N aq HCl before extraction with ethyl acetate. Silica gel chromatography (0-5% methanol/ethyl acetate) followed by trituration in methanol were utilized to purify this compound. MS(ES)+ m/e 508.0 [M+H]⁺.

### Example 25

### 4-cyclopropyl-9-{[4-(1-methyl-2,3-dihydro-1H-indol-5-yl)phenyl]sulfonyl}-1-oxa-4,9-diazaspiro[5.5]undecan-3-one

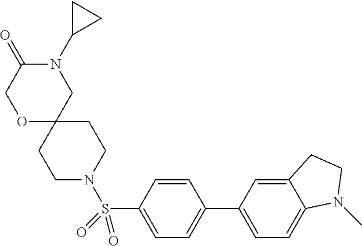 [SMILES: CN1CCc2cc(-c3ccc(S(=O)(=O)N4CCC5(CC4)CN(C4CC4)C(=O)CO5)cc3)ccc21]

a) Following the procedure described in Example 13g with 1-methyl-5-(4,4,5,5-tetramethyl-1,3,2-dioxaborolan-2-yl)-2,3-dihydro-1H-indole for 1 h provided the title compound as a beige solid (45%). Silica gel chromatography (0-2% methanol/ethyl acetate) followed by precipitation from methanol were utilized to purify this compound. MS(ES)+m/z 482.0 [M+H]⁺.

### Example 26

### 9-{[4-(1-benzofuran-2-yl)phenyl]sulfonyl}-4-cyclopropyl-1-oxa-4,9-diazaspiro[5.5]undecan-3-one

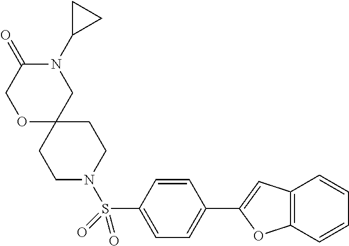 [SMILES: O=C1COC2(CCN(S(=O)(=O)c3ccc(-c4cc5ccccc5o4)cc3)CC2)CN1C1CC1]

a) Following the procedure described in Example 13g with 2-benzofuranboronic acid for 1 h provided the title compound as an off-white solid (35%) after purification by silica gel chromatography (0-2% methanol/ethyl acetate) and subsequent trituration in methanol. MS(ES)+ m/e 467.0 [M+H]⁺.

### Example 27

### 9-{[4-(1-benzothien-2-yl)phenyl]sulfonyl}-4-cyclopropyl-1-oxa-4,9-diazaspiro[5.5]undecan-3-one

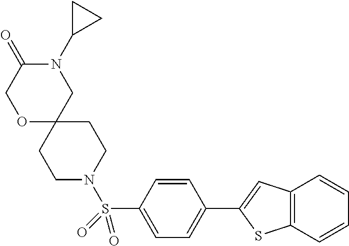 [SMILES: O=C1COC2(CCN(S(=O)(=O)c3ccc(-c4cc5ccccc5s4)cc3)CC2)CN1C1CC1]

a) Following the procedure described in Example 13g with benzo(b)thiophene-2-boronic acid for 1 h provided the title compound as an off-white solid (21%). Trituration of the crude product in methanol was utilized to purify this compound. MS(ES)+ m/e 483.0 [M+H]⁺.

### Example 28

### 9-{[4-(1,3-benzoxazol-2-yl)phenyl]sulfonyl}-4-cyclopropyl-1-oxa-4,9-diazaspiro[5.5]undecan-3-one

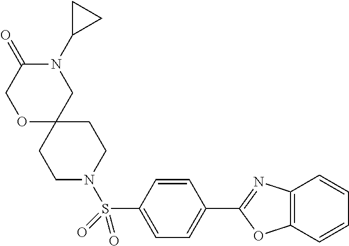 [SMILES: O=C1COC2(CCN(S(=O)(=O)c3ccc(-c4nc5ccccc5o4)cc3)CC2)CN1C1CC1]

a) Following the procedure described in Example 13g with 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one and 2-chlorobenzoxazole provided the title compound as a white solid (9%). Trituration in hot methanol followed by reverse phase HPLC (10-80 acetonitrile/water+0.1% NH₄OH) were utilized to purify this compound. MS(ES)+ m/e 468.1 [M+H]⁺.

### Example 29

### 4-cyclopropyl-9-{[4-(7-quinolinyl)phenyl]sulfonyl}-1-oxa-4,9-diazaspiro[5.5]undecan-3-one

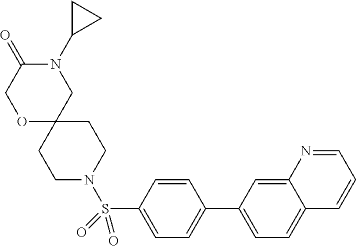 [SMILES: O=C1COC2(CCN(S(=O)(=O)c3ccc(-c4ccc5cccnc5c4)cc3)CC2)CN1C1CC1]

a) A mixture of 9-[(4-bromophenyl)sulfonyl]-4-cyclopropyl-1-oxa-4,9-diazaspiro[5.5]undecan-3-one (0.466 mmol), 7-quinolineboronic acid (0.489 mmol), bis(triphenylphosphino)dichloropalladium(II) (0.035 mmol), and potassium carbonate (1.863 mmol) in ethanol (6.0 mL) and water (1.5 mL) was flushed with nitrogen and then heated at 90° C. for 2 h. The mixture was cooled, diluted with dichloromethane, and washed with water. The aqueous layer was extracted with dichloromethane, and the combined organic extracts were washed with water, dried, and concentrated in vacuo. The residue was taken up in chloroform and silicycle thiol (400 mg) was added and the mixture was stirred at 50° C. overnight. The mixture was filtered hot, washed with chloroform, and evaporated to a foam. Crystallization of the foam from ethyl acetate gave the title product (140 mg, 63% yield). ¹H NMR (400 MHz, DMSO-d₆) δ 8.99 (dd, 1H), 8.45 (dd, J=1.5, 8.3 Hz, 1H), 8.42-8.38 (m, 1H), 8.23-8.11 (m, 3H), 8.05 (dd, J=1.8, 8.6 Hz, 1H), 7.87 (d, J=8.3 Hz, 2H), 7.60 (dd, J=4.2, 8.2 Hz, 1H), 3.86 (s, 2H), 3.54 (d, J=11.6 Hz, 2H), 3.13 (s, 2H), 2.72 (tt, J=3.9, 7.4 Hz, 1H), 2.49-2.39 (m, 2H), 1.93-1.79 (m, 2H), 1.69 (td, J=4.3, 13.1 Hz, 2H), 0.78-0.63 (m, 2H), 0.63-0.49 (m, 2H); LCMS(ES) 477.9 [M+H]⁺.

### Example 30

### 4-cyclopropyl-9-{[4-(7-quinolinyl)phenyl]sulfonyl}-1,4,9-triazaspiro[5.5]undecan-3-one

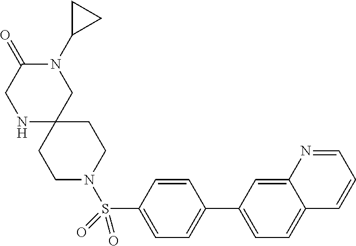 [SMILES: O=C1CNC2(CCN(S(=O)(=O)c3ccc(-c4ccc5cccnc5c4)cc3)CC2)CN1C1CC1]

### a) Methyl 1-[(4-bromophenyl)sulfonyl]-4-({[(1,1-dimethylethyl)oxy]carbonyl}amino)-4-piperidinecarboxylate

To a round-bottom flask, methyl 4-({[(1,1-dimethylethyl)oxy]carbonyl}amino)-4-piperidinecarboxylate (18.97 mmol) was dissolved in dichloromethane (DCM) (80 mL) under nitrogen and cooled to −78° C. 4-Bromobenzenesulfonyl chloride (19.37 mmol) and Hunig's base (57.3 mmol) were added, and the solution was allowed to warm to room temperature and stirred for 1 h. Analysis of the reaction by LCMS indicated formation of the desired product and disappearance of starting material. The solution was diluted with DCM (50 mL) and poured into a separatory funnel containing saturated aq sodium bicarbonate. The aqueous phase was extracted with DCM (3×) and the combined organic layer was washed with brine, dried over Na₂SO₄, and concentrated to dryness. This material was purified by silica gel chromatography (0-20% isopropanol/ethyl acetate), and the desired fractions were combined and concentrated to afford the title product as a white solid (3.75 g, 41%). MS(ES)+ m/e 478.8, 480.0 [M+H]⁺.

### b) 1,1-dimethylethyl [1-[(4-bromophenyl)sulfonyl]-4-(hydroxymethyl)-4-piperidinyl]carbamate

To a round bottom flask, LiBH₄ (18.36 mmol) was added, dissolved in tetrahydrofuran (60 mL) and cooled to −78° C. Methyl 1-[(4-bromophenyl)sulfonyl]-4-({[(1,1-dimethylethyl)oxy]carbonyl}amino)-4-piperidinecarboxylate (5.45 mmol) in 15 mL tetrahydrofuran was added slowly to the cooled solution, which was then allowed to warm to room temperature. After 24 h, the solution was re-cooled to −78° C. and an additional portion of LiBH₄ (9.18 mmol) was added. The solution was allowed to warm to room temperature and stirred for an additional 30 h. Analysis of the reaction mixture by LCMS indicated formation of desired product and disappearance of starting material. The solution was diluted with ethyl acetate (50 mL) and poured into a separatory funnel containing saturated aq sodium bicarbonate. The aqueous phase was extracted with ethyl acetate (3×) and the combined organic layer was washed with brine, dried over Na₂SO₄, and concentrated to a dark tan oil. The crude title product was carried forward without further purification. MS(ES)+ m/e 448.9, 450.7 [M+H]⁺.

### c) 1,1-dimethylethyl {1-[(4-bromophenyl)sulfonyl]-4-formyl-4-piperidinyl}carbamate

To a round-bottom flask, crude 1,1-dimethylethyl[1-[(4-bromophenyl)sulfonyl]-4-(hydroxymethyl)-4-piperidinyl]carbamate (5.39 mmol) was added and dissolved in DCM (40 mL). Dess-Martin periodinane (10.77 mmol) was added to the solution and stirred at room temperature for 20 h. Analysis by LCMS indicated desired product formation. The solution was filtered and the filtrate was partitioned with saturated aq sodium bicarbonate. The aqueous phase was extracted with DCM (3×) and the combined organic layer was washed with brine, dried over Na₂SO₄, and concentrated to dryness. This material was purified by silica gel chromatography (0-15% isopropanol/ethyl acetate), and the desired fractions were combined and concentrated to afford the title product (0.75 g, 31%). MS(ES)+ m/e 447.0, 448.8 [M+H]⁺.

### d) 1,1-dimethylethyl {1-[(4-bromophenyl)sulfonyl]-4-[(cyclopropylamino)methyl]-4-piperidinyl}carbamate

To a round-bottom flask, 1,1-dimethylethyl {1-[(4-bromophenyl)sulfonyl]-4-formyl-4-piperidinyl}carbamate (0.839 mmol) was dissolved in dichloroethane (40 mL) followed by the addition of cyclopropylamine (2.68 mmol) and sodium triacetoxyborohydride (3.35 mmol). The mixture was stirred at room temperature for 16 h. LCMS analysis indicated the presence of desired product. The solution was poured into a separatory funnel containing saturated aq sodium bicarbonate. The aqueous phase was extracted with DCM (3×) and the combined organic layer was washed with brine, dried over Na₂SO₄, and concentrated to dryness. This residue was purified by silica gel chromatography (0-25% isopropanol/ethyl acetate), and the desired fractions were combined and concentrated to afford the title product as a tan residue (350 mg, 43%). MS(ES)+ m/e 488.1, 490.2 [M+H]⁺.

### e) 9-[(4-bromophenyl)sulfonyl]-4-cyclopropyl-1,4,9-triazaspiro[5.5]undecan-3-one

To a round-bottom flask, 1,1-dimethylethyl {1-[(4-bromophenyl)sulfonyl]-4-[(cyclopropylamino)methyl]-4-piperidinyl}carbamate (0.690 mmol) was dissolved in DCM (6000 μL) and cooled to −78° C. under N₂. Chloroacetyl chloride (0.828 mmol) and Hunig's base (1.380 mmol) were added to the solution, which was then allowed to warm to room temperature. After 30 min, LCMS analysis indicated desired intermediate. This solution was cooled to −78° C. and HCl (1552 μL of a 4M solution in dioxane) was added. After 10 min, the mixture was allowed to warm to room temperature and was stirred for 4 h. LCMS analysis indicated desired deprotected intermediate. Hunig's base (2.4 mL) was added and the reaction was stirred for 30 min. LCMS analysis indicated desired product formation. The mixture was poured into a separatory funnel containing saturated aq sodium bicarbonate. The aqueous phase was extracted with DCM (3×) and the combined organic layer was washed with brine, dried over Na₂SO₄, and concentrated to dryness. This residue was purified by silica gel chromatography (0-25% isopropanol/ethyl acetate), and the desired fractions were combined and concentrated to afford the title product as a tan solid (90 mg, 31%). MS(ES)+ m/e 427.7, 429.8 [M+H]⁺.

### f) 4-cyclopropyl-9-{[4-(7-quinolinyl)phenyl]sulfonyl}-1,4,9-triazaspiro[5.5]undecan-3-one

To a microwave vial, 9-[(4-bromophenyl)sulfonyl]-4-cyclopropyl-1,4,9-triazaspiro[5.5]undecan-3-one (0.198 mmol), 7-(4,4,5,5-tetramethyl-1,3,2-dioxaborolan-2-yl)quinoline (0.298 mmol), cesium carbonate (0.595 mmol), and dichloro[1,1′-bis(diphenylphosphino)ferrocene]palladium(II)-dichloromethane adduct (0.02 mmol) were added to a microwave vial and purged with nitrogen. 1,4-Dioxane (1.5 mL) and water (1.0 mL) were added to the mixture, which was heated overnight at 100° C. LCMS analysis indicated desired product formation and consumption of starting material. The mixture was filtered through a syringe filter and poured into a separatory funnel containing brine. The aqueous phase was extracted with ethyl acetate (3×) and the combined organic layer was washed with brine, dried over Na₂SO₄, and concentrated to dryness. This residue was purified by silica gel chromatography (0-40% isopropanol/ethyl acetate), and the desired fractions were combined and concentrated to afford the title product as a tan solid (82 mg, 87%). MS(ES)+ m/e 477.0 [M+H]⁺.

### Example 31

### 4-cyclopropyl-1-methyl-9-{[4-(7-quinolinyl)phenyl]sulfonyl}-1,4,9-triazaspiro[5.5]undecan-3-one

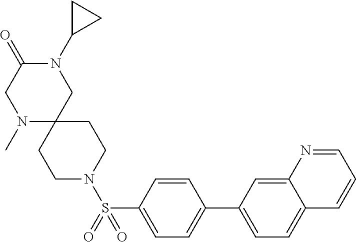 [SMILES: CN1CC(=O)N(C2CC2)CC12CCN(S(=O)(=O)c1ccc(-c3ccc4cccnc4c3)cc1)CC2]

a) To a round-bottom flask, 4-cyclopropyl-9-{[4-(7-quinolinyl)phenyl]sulfonyl}-1,4,9-triazaspiro[5.5]undecan-3-one (0.057 mmol) and potassium acetate (0.227 mmol) were combined in tetrahydrofuran (650 μL) and Hunig's base (0.567 mmol) under N₂. Formaldehyde (1.133 mmol) was added to the mixture, followed by sodium triacetoxyborohydride (0.567 mmol). The solution was stirred at room temperature for 30 min, at which point LCMS analysis indicated complete conversion to desired product. The mixture was diluted with ethyl acetate (5 mL) and partitioned in saturated aq sodium bicarbonate. The aqueous layer was extracted with ethyl acetate (3×), and the combined organic layer was washed with brine, dried over sodium sulfate, and concentrated to a dark tan residue. This residue was purified by silica gel chromatography (0-30% isopropanol/ethyl acetate), and the desired fractions were combined and concentrated to afford the title product as an off-white solid (20 mg, 72%). MS(ES)+ m/e 491.5 [M+H]⁺.

### Example 32

### 4-cyclopropyl-8-methyl-9-{[4-(7-quinolinyl)phenyl]sulfonyl}-1-oxa-4,9-diazaspiro[5.5]undecan-3-one

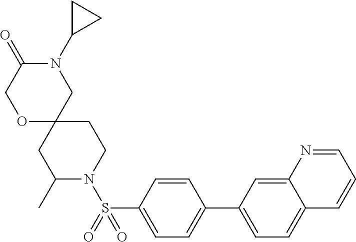 [SMILES: CC1CC2(CCN1S(=O)(=O)c1ccc(-c3ccc4cccnc4c3)cc1)CN(C1CC1)C(=O)CO2]

### a) 1,1-dimethylethyl 5-methyl-1-oxa-6-azaspiro[2.5]octane-6-carboxylate

To a round-bottom flask, trimethylsulfoxonium iodide (7.38 mmol) was dissolved in dimethylsulfoxide (9.58 mL) and cooled to 0° C. Sodium hydride (8.38 mmol of a 60% dispersion in mineral oil) was added to the frozen mixture and allowed to warm to room temperature and stirred for 2 h. This mixture was then re-cooled to 0° C. and 1,1-dimethylethyl 2-methyl-4-oxo-1-piperidinecarboxylate (6.71 mmol) in dimethylsulfoxide (1 mL) was added to the frozen mixture and then allowed to warm to room temperature. This mixture was stirred for 2 h and then determined to be complete by LCMS analysis. Water was slowly added to the reaction, which was then added to a separatory funnel containing DCM. The aqueous phase was extracted with DCM (3×), and the combined organics were washed with brine (2×), dried over Na₂SO₄, filtered, and concentrated to afford the crude title product as a yellow oil (material was carried forward without further purification). MS(ES)+ m/e 228.1 [M+H]⁺.

### b) 1,1-dimethylethyl 4-[(cyclopropylamino)methyl]-4-hydroxy-2-methyl-1-piperidinecarboxylate

To a round-bottom flask, crude 1,1-dimethylethyl 5-methyl-1-oxa-6-azaspiro[2.5]octane-6-carboxylate from Example 32a was added and dissolved in ethanol (20 mL). Cyclopropylamine (42.9 mmol) was added to the solution, which was heated at 65° C. for 5 h. LCMS analysis indicated disappearance of starting material and formation of desired product. The solution was concentrated to afford the title product as a dark tan/brown oil (material was carried forward without further purification). MS(ES)+ m/e 285.0 [M+H]⁺.

### c) 1,1-dimethylethyl 4-{[(chloroacetyl)(cyclopropyl)amino]methyl}-4-hydroxy-2-methyl-1-piperidinecarboxylate

To a round bottom flask, crude 1,1-dimethylethyl 4-[(cyclopropylamino)methyl]-4-hydroxy-2-methyl-1-piperidinecarboxylate from Example 32b was added, dissolved in DCM (40 mL), and cooled to −78° C. Chloroacetyl chloride (6.33 mmol) and Hunig's base (15.82 mmol) were added to the cooled solution, which was immediately allowed to warm to room temperature. After 6 h, LCMS analysis indicated consumption of starting material and desired product formation. The solution was poured into a separatory funnel containing water, and the aqueous layer was adjusted to a pH of 5 with 1N aq HCl. The aqueous layer was extracted with DCM (2×) and ethyl acetate (2×), and the combined organics were dried over Na₂SO₄, filtered, and concentrated to afford the crude title product as a black residue (material was carried forward without further purification). MS(ES)+ m/e 361.7 [M+H]⁺.

### d) 1,1-dimethylethyl 4-cyclopropyl-8-methyl-3-oxo-1-oxa-4,9-diazaspiro[5.5]undecane-9-carboxylate

To a round-bottom flask, crude 1,1-dimethylethyl 4-{[(chloroacetyl)(cyclopropyl)amino]methyl}-4-hydroxy-2-methyl-1-piperidinecarboxylate from Example 32c was dissolved in tetrahydrofuran (50 mL) and NaH (47.5 mmol of a 60% dispersion in mineral oil) was added to the stirring solution in 4 portions over 2 min. The mixture was stirred at room temperature for 30 min. LCMS analysis indicated consumption of starting material and desired product formation. Water was slowly added to the stirred solution until the bubbling disappeared. The mixture was diluted with ethyl acetate (20 mL) and partitioned with saturated aq sodium bicarbonate. The aqueous layer was extracted with ethyl acetate (3×), and the combined organic layer was washed with brine, dried over sodium sulfate, and concentrated to a dark residue. This residue was purified by silica gel chromatography (10-100% ethyl acetate/hexanes), and the desired fractions were combined and concentrated to afford the title product as a yellow oil (1.45 g, 60% over the four steps). MS(ES)+ m/e 325.6 [M+H]⁺.

### e) 9-[(4-bromophenyl)sulfonyl]-4-cyclopropyl-8-methyl-1-oxa-4,9-diazaspiro[5.5]undecan-3-one

To a round-bottom flask, 1,1-dimethylethyl 4-cyclopropyl-8-methyl-3-oxo-1-oxa-4,9-diazaspiro[5.5]undecane-9-carboxylate (1.094 mmol) was suspended in ethyl acetate (5 mL) followed by the addition of HCl (6 mL of a 4M solution in 1,4-dioxane). After 1 h, LCMS analysis indicated the desired deprotected intermediate. The mixture was concentrated in vacuo. Pyridine (5.00 mL) was added, followed by 4-(dimethylamino)pyridine (0.219 mmol) and 4-bromobenzenesulfonyl chloride (2.189 mmol), and the mixture was heated at 70° C. for 2 h. LCMS analysis indicated consumption of starting material and formation of desired product. The mixture was diluted with ethyl acetate (30 mL) and partitioned with saturated aq sodium bicarbonate. The aqueous layer was extracted with ethyl acetate (3×), and the combined organic layer was washed with brine, dried over sodium sulfate, and concentrated to a brown residue. This residue was purified by silica gel chromatography (10-100% ethyl acetate/hexanes), and the desired fractions were combined and concentrated to afford the title product as an off-white solid (350 mg, 72%). MS(ES)+ m/e 443.1, 445.1 [M+H]⁺.

### f) 4-cyclopropyl-8-methyl-9-{[4-(7-quinolinyl)phenyl]sulfonyl}-1-oxa-4,9-diazaspiro[5.5]undecan-3-one

To a microwave vial, 9-[(4-bromophenyl)sulfonyl]-4-cyclopropyl-8-methyl-1-oxa-4,9-diazaspiro[5.5]undecan-3-one (0.316 mmol), 7-(4,4,5,5-tetramethyl-1,3,2-dioxaborolan-2-yl)quinoline (0.411 mmol), Cs₂CO₃ (0.947 mmol), and dichloro[1,1′-bis(diphenylphosphino)ferrocene]palladium(II)-dichloromethane adduct (0.015 mmol) were added to a microwave vial and purged with nitrogen. 1,4-Dioxane (1.5 mL) and water (1.5 mL) were added to the mixture, which was heated for 16 h at 100° C. LCMS analysis indicated desired product formation and consumption of starting material. The mixture was filtered through a syringe filter and purified by reverse phase HPLC (10-90% acetonitrile/water+0.1% TFA). The desired fractions were collected and added to a reparatory funnel containing DCM and saturated aq sodium bicarbonate. The aqueous phase was extracted with DCM (3×) and the combined organics were washed with brine, dried over Na₂SO₄, and concentrated to afford the title product as a white foam and as a single diastereomer (120 mg, 77%). MS(ES)+ m/e 492.2 [M+H]⁺.

### Example 33

### 4-cyclopropyl-7-methyl-9-{[4-(7-quinolinyl)phenyl]sulfonyl}-1-oxa-4,9-diazaspiro[5.5]undecan-3-one

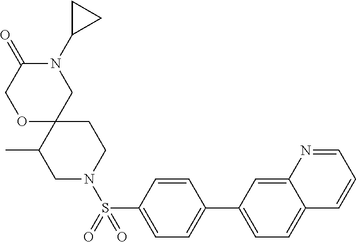 [SMILES: CC1CN(S(=O)(=O)c2ccc(-c3ccc4cccnc4c3)cc2)CCC12CN(C1CC1)C(=O)CO2]

a) Following the procedures described in Example 32a-f with 1,1-dimethylethyl 3-methyl-4-oxo-1-piperidinecarboxylate instead of 1,1-dimethylethyl 2-methyl-4-oxo-1-piperidinecarboxylate provided the title compound as a white solid and as a single diastereomer (138 mg, 89%). MS(ES)+ m/e 492.2 [M+H]⁺.

### Example 34

### 9-[(4-imidazo[1,2-a]pyridin-7-ylphenyl)sulfonyl]-4-(1-methylcyclopropyl)-1-oxa-4,9-diazaspiro[5.5]undecan-3-one

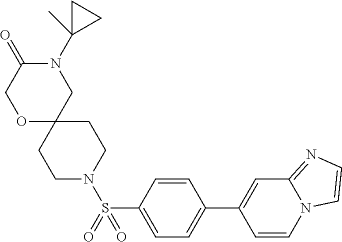 [SMILES: CC1(N2CC3(CCN(S(=O)(=O)c4ccc(-c5ccn6ccnc6c5)cc4)CC3)OCC2=O)CC1]

### a) 1,1-dimethylethyl 4-hydroxy-4-{[(1-methylcyclopropyl)amino]methyl}-1-piperidinecarboxylate

To a round-bottom flask, 1,1-dimethylethyl 5-methyl-1-oxa-6-azaspiro[2.5]octane-6-carboxylate (2.55 mmol) was added and dissolved in ethanol (20 mL). (1-Methylcyclopropyl)amine (8.65 mmol) was added and the reaction was heated at 65° C. for 5 h. LCMS analysis indicated disappearance of starting material and formation of desired product. The solution was concentrated to afford the crude title product as a yellow oil (material was carried forward without further purification). MS(ES)+ m/e 285.0 [M+H]⁺.

### b) 1,1-dimethylethyl 4-{[(chloroacetyl)(1-methylcyclopropyl)amino]methyl}-4-hydroxy-1-piperidinecarboxylate

To a round bottom flask, crude 1,1-dimethylethyl 4-hydroxy-4-{[(1-methylcyclopropyl)amino]methyl}-1-piperidinecarboxylate from Example 34a was added, dissolved in DCM (15 mLl), and cooled to −78° C. Chloroacetyl chloride (2.90 mmol) and Hunig's base (5.73 mmol) were added to the cooled solution which was immediately allowed to warm to room temperature. After 30 min, LCMS analysis indicated consumption of starting material and desired product formation. The solution was poured into a separatory funnel containing water, and the aqueous layer was adjusted to a pH of 5 with 1N aq HCl. The aqueous layer was extracted with DCM (2×) and ethyl acetate (2×), dried over Na₂SO₄, filtered, and concentrated to afford the crude title product as a tan oil (material was carried forward without further purification). MS(ES)+ m/e 361.7 [M+H]⁺.

### c) 1,1-dimethylethyl 4-(1-methylcyclopropyl)-3-oxo-1-oxa-4,9-diazaspiro[5.5]undecane-9-carboxylate

To a round-bottom flask, crude 1,1-dimethylethyl 4-{[(chloroacetyl)(1-methylcyclopropyl)amino]methyl}-4-hydroxy-1-piperidinecarboxylate from Example 34b was dissolved in tetrahydrofuran (15 mL) and NaH (10.5 mmol of a 60% dispersion in mineral oil) was added to the stirring solution in 2 portions. The mixture was stirred at room temperature for 30 min. LCMS analysis indicated consumption of starting material and desired product formation. Water was slowly added to the stirred solution until the bubbling subsided. The mixture was diluted with ethyl acetate (10 mL) and partitioned with saturated aq sodium bicarbonate. The aqueous layer was extracted with ethyl acetate (3×), and the combined organic layer was washed with brine, dried over sodium sulfate, and concentrated to a dark residue. This residue was purified by silica gel chromatography (10-100% ethyl acetate/hexanes), and the desired fractions were combined and concentrated to afford the title product as a yellow oil (600 mg, 73% over the three steps). MS(ES)+ m/e 325.6 [M+H]⁺.

### d) 9-[(4-bromophenyl)sulfonyl]-4-(1-methylcyclopropyl)-1-oxa-4,9-diazaspiro[5.5]undecan-3-one

To a round-bottom flask, 1,1-dimethylethyl 4-(1-methylcyclopropyl)-3-oxo-1-oxa-4,9-diazaspiro[5.5]undecane-9-carboxylate (0.925 mmol) was suspended in ethyl acetate (8 mL) and treated with HCl (3 mL of a 4M solution in 1,4-dioxane). After 2 h, LCMS indicated formation of the desired deprotected intermediate. The mixture was concentrated in vacuo. Pyridine (5.00 mL) was added, followed by 4-(dimethylamino)pyridine (0.231 mmol) and 4-bromobenzenesulfonyl chloride (1.849 mmol), and the mixture was heated at 70° C. for 16 h. LCMS analysis indicated consumption of starting material and formation of desired product. The mixture was diluted with ethyl acetate (30 mL) and partitioned with saturated aq sodium bicarbonate. The aqueous layer was extracted with ethyl acetate (3×), and the combined organic layer was washed with brine, dried over sodium sulfate, and concentrated to a brown residue. This residue was purified by silica gel chromatography (20-100% ethyl acetate/hexanes), and the desired fractions were combined and concentrated to afford the title product as an off-white solid (250 mg, 61%). MS(ES)+ m/e 442.9, 445.0 [M+H]⁺.

### e) 9-[(4-imidazo[1,2-a]pyridin-7-ylphenyl)sulfonyl]-4-(1-methylcyclopropyl)-1-oxa-4,9-diazaspiro[5.5]undecan-3-one

To a microwave vial, 7-bromoimidazo[1,2-a]pyridine (0.508 mmol), 4,4,4′,4′,5,5,5′,5′-octamethyl-2,2′-bi-1,3,2-dioxaborolane (0.584 mmol), potassium acetate (2.03 mmol), and PdCl₂(dppf)-CH₂Cl₂ adduct (0.025 mmol) were added and purged with nitrogen. Dioxane (3.0 mL) was added to the mixture, which was heated at 110° C. for 5 h. 9-[(4-bromophenyl)sulfonyl]-4-(1-methylcyclopropyl)-1-oxa-4,9-diazaspiro[5.5]undecan-3-one (0.180 mmol), cesium carbonate (1.27 mmol), PdCl₂(dppf)-CH₂Cl₂ adduct (0.02 mmol) and water (2.0 mL) were added to the mixture, which was then irradiated in a microwave reactor at 110° C. for 30 min. LCMS analysis indicated desired product formation. The mixture was filtered through a syringe filter and purified by reverse phase HPLC (10-90% acetonitrile/water+0.1% TFA). The desired fractions were collected and added to a separatory funnel containing ethyl acetate and saturated aq NaHCO₃. The aqueous phase was extracted with ethyl acetate (3×) and the combined organic layer was washed with brine, dried over Na₂SO₄, and concentrated to afford the title product as an off-white solid (36 mg, 41%). MS(ES)+ m/e 480.9 [M+H]⁺.

### Example 35

### 4-cyclopropyl-7-fluoro-9-[(4-imidazo[1,2-a]pyridin-7-ylphenyl)sulfonyl]-1-oxa-4,9-diazaspiro[5.5]undecan-3-one (Diastereomer 1)

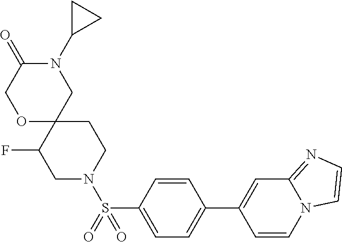 [SMILES: O=C1COC2(CCN(S(=O)(=O)c3ccc(-c4ccn5ccnc5c4)cc3)CC2F)CN1C1CC1]

### a) Phenylmethyl 3-fluoro-4-oxo-1-piperidinecarboxylate

To a round-bottom flask, phenylmethyl 4-oxo-1-piperidinecarboxylate (39.0 mmol) was dissolved in N,N-dimethylformamide (50 mL) followed by the addition of triethylamine (122 mmol) and TMSCl (49.3 mmol) under N₂. This mixture was heated for 16 h at 80° C., cooled to room temperature, and poured into a separatory funnel containing hexanes. The mixture was partitioned with water, washed with brine (3×), dried over sodium sulfate, filtered, and concentrated in vacuo. This residue was dissolved in acetonitrile (200 mL), cooled to 0° C., and treated with Selectfluor® (41.6 mmol) in 3 portions over 3 min. The mixture was allowed to warm to room temperature overnight. Most of the solvent was removed by rotary evaporation and the remainder was partitioned between ethyl acetate and water. The organic layer was washed with brine (3×), dried over sodium sulfate, filtered, and concentrated. The residue was purified by silica gel chromatography (0-70% ethyl acetate/hexanes) and the desired fraction were collected and concentrated to afford the title product as an oil (7.1 g, 72%). MS(ES)+ m/e 252.0 [M+H]⁺.

### b) 1-[(4-bromophenyl)sulfonyl]-3-fluoro-4-piperidinone

To a round-bottom flask, phenylmethyl 3-fluoro-4-oxo-1-piperidinecarboxylate (27.9 mmol) was dissolved in methanol (50 mL) and ethanol (50 mL). A quantity of 10% Pd/C (2.79 mmol) was added to the mixture cautiously, and this mixture was hydrogenated under a hydrogen balloon atmosphere. After 5 h, LCMS analysis indicated disappearance of starting material. The mixture was filtered through Celite and the filtrate was concentrated. This residue was dissolved in dichloromethane (200 mL) and cooled to −78° C. 4-Bromobenzenesulfonyl chloride (33.4 mmol) and Hunig's base (84 mmol) were added and the mixture was allowed to warm to room temperature. After 2 h, LCMS analysis indicated desired product formation. The mixture was poured into a reparatory funnel containing saturated aq sodium bicarbonate. The aqueous layer was extracted with DCM (3×), and the combined organic layer was washed with brine, dried over sodium sulfate, and concentrated to a residue. This residue was purified by silica gel chromatography (10-100% ethyl acetate/hexanes), and the desired fractions were combined and concentrated to afford the title product as an oil (2.17 g, 24%). MS(ES)+m/e 336.0, 338.0 [M+H]⁺.

### c) 6-[(4-bromophenyl)sulfonyl]-4-fluoro-1-oxa-6-azaspiro[2.5]octane (Diastereomer 1 and Diastereomer 2)

To a round-bottom flask, trimethylsulfoxonium iodide (6.45 mmol) was dissolved in dimethylsulfoxide (9.58 mL) and cooled to 0° C. NaH (7.75 mmol of a 60% dispersion in mineral oil) was added to the frozen mixture, which was allowed to warm to room temperature and stirred for 3 h. This mixture was then re-cooled to 0° C. and 1-[(4-bromophenyl)sulfonyl]-3-fluoro-4-piperidinone (6.45 mmol) in dimethylsulfoxide (5 mL) was added to the frozen mixture, which was allowed to warm to room temperature. The reaction was stirred for 2 h and then determined to be complete by LCMS analysis. The reaction mixture was quenched with slow addition of water and was then added to a separatory funnel containing DCM. The aqueous phase was extracted with DCM (3×), and the combined organics were washed with brine, dried over Na₂SO₄, filtered, and concentrated in vacuo. The residue was purified by silica gel chromatography (5-75% ethyl acetate/hexanes) and 2 peaks eluting very closely were separated. Both peaks indicated desired product by LCMS analysis. Each set of fractions was independently combined and concentrated to afford the two diastereomers of the title product as white solids. A combined yield of 37% title product was isolated in the following quantities: Diasteromer 1 (1^{st} product eluted)=500 mg, Diastereomer 2 (2^{nd} product eluted)=320 mg. MS(ES)+ m/e 349.9, 352.0 [M+H]⁺.

### d) 1-[(4-bromophenyl)sulfonyl]-4-[(cyclopropylamino)methyl]-3-fluoro-4-piperidinol (Diastereomer 1)

To a round-bottom flask, 6-[(4-bromophenyl)sulfonyl]-4-fluoro-1-oxa-6-azaspiro[2.5]octane (Diastereomer 1) (1.37 mmol) was added and dissolved in ethanol (8 mL). Cyclopropylamine (5.48 mmol) was added to the solution, which was heated at 75° C. for 15 h. LCMS analysis indicated disappearance of starting material and formation of desired product. The solution was concentrated to afford the title product as a yellow oil (material was carried forward without purification). (ES)+ m/e 407.0, 408.9 [M+H]⁺.

### e) N-({1-[(4-bromophenyl)sulfonyl]-3-fluoro-4-hydroxy-4-piperidinyl}methyl)-2-chloro-N-cyclopropylacetamide (Diastereomer 1)

To a round bottom flask, crude 1-[(4-bromophenyl)sulfonyl]-4-[(cyclopropylamino)methyl]-3-fluoro-4-piperidinol (Diastereomer 1) from Example 35d was added, dissolved in DCM (10 mL), and cooled to −78° C. Chloroacetyl chloride (1.5 mmol) and Hunig's base (2.74 mmol) were added to the cooled solution, which was immediately allowed to warm to room temperature. After 1 h, LCMS analysis indicated consumption of starting material and desired product formation. The solution was poured into a separatory funnel containing saturated aq sodium bicarbonate. The aqueous layer was extracted with DCM (3×), dried over Na₂SO₄, filtered, and concentrated to afford the title product as a yellow oil (material was carried forward without further purification). MS(ES)+ m/e 482.8, 485.0 [M+H]⁺.

### f) 9-[(4-bromophenyl)sulfonyl]-4-cyclopropyl-7-fluoro-1-oxa-4,9-diazaspiro[5.5]undecan-3-one (Diastereomer 1)

To a round-bottom flask, crude N-({1-[(4-bromophenyl)sulfonyl]-3-fluoro-4-hydroxy-4-piperidinyl}methyl)-2-chloro-N-cyclopropylacetamide (Diastereomer 1) from Example 35e was dissolved in tetrahydrofuran (15 mL) and NaH (10.0 mmol of a 60% dispersion in mineral oil) was added to the stirring solution in 2 portions. The mixture was stirred at room temperature for 30 min. LCMS analysis indicated consumption of starting material and desired product formation. Water was slowly added to the stirred solution until the bubbling ceased. The mixture was diluted with ethyl acetate (10 mL) and partitioned in saturated aq sodium bicarbonate. The aqueous layer was extracted with ethyl acetate (3×), and the combined organic layer was washed with brine, dried over sodium sulfate, and concentrated to afford the title product as a yellow solid (420 mg, 70% over the three steps). MS(ES)+ m/e 447.0, 448.9 [M+H]⁺.

### g) 4-cyclopropyl-7-fluoro-9-[(4-imidazo[1,2-a]pyridin-7-ylphenyl)sulfonyl]-1-oxa-4,9-diazaspiro[5.5]undecan-3-one (Diastereomer 1)

Following the procedure described in Example 34e using 9-[(4-bromophenyl)sulfonyl]-4-cyclopropyl-7-fluoro-1-oxa-4,9-diazaspiro[5.5]undecan-3-one (Diastereomer 1) provided the title compound as an off-white solid (6 mg, 14%). MS(ES)+ m/e 485.2 [M+H]⁺.

### Example 36

### 4-cyclopropyl-7-fluoro-9-[(4-imidazo[1,2-a]pyridin-7-ylphenyl)sulfonyl]-1-oxa-4,9-diazaspiro[5.5]undecan-3-one (Diastereomer 2)

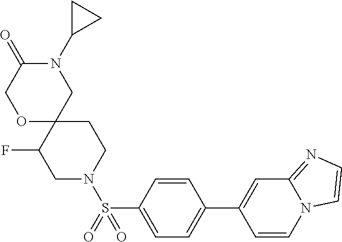 [SMILES: O=C1COC2(CCN(S(=O)(=O)c3ccc(-c4ccn5ccnc5c4)cc3)CC2F)CN1C1CC1]

a) Following the procedure described in Example 35d-g using 6-[(4-bromophenyl)sulfonyl]-4-fluoro-1-oxa-6-azaspiro[2.5]octane (Diasteromer 2) afforded the title compound as an off-white solid (30 mg, 61%). MS(ES)+ m/e 485.2 [M+H]⁺.

### Example 37

### 4-cyclopropyl-7-fluoro-9-{[4-(7-quinolinyl)phenyl]sulfonyl}-1-oxa-4,9-diazaspiro[5.5]undecan-3-one (Diasteromer 1)

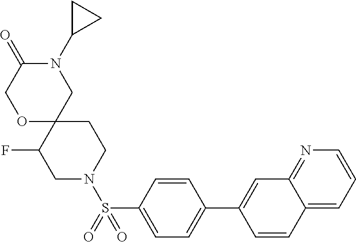 [SMILES: O=C1COC2(CCN(S(=O)(=O)c3ccc(-c4ccc5cccnc5c4)cc3)CC2F)CN1C1CC1]

a) To a microwave vial, 9-[(4-bromophenyl)sulfonyl]-4-cyclopropyl-7-fluoro-1-oxa-4,9-diazaspiro[5.5]undecan-3-one (Diastereomer 1) (0.224 mmol), 7-(4,4,5,5-tetramethyl-1,3,2-dioxaborolan-2-yl)quinoline (0.335 mmol), Cs₂CO₃ (0.671 mmol), and dichloro[1,1′-bis(diphenylphosphino)ferrocene]palladium(II)-dichloromethane adduct (0.010 mmol) were added to a microwave vial and purged with nitrogen. 1,4-Dioxane (1.5 mL) and water (1.5 mL) were added to the mixture, which was heated for 16 h at 100° C. LCMS analysis indicated desired product formation and consumption of starting material. The mixture was filtered through a syringe filter and purified by reverse phase HPLC (10-90% acetonitrile/water+0.1% TFA). The desired fractions were collected and added to a separatory funnel containing DCM and saturated aq sodium bicarbonate. The aqueous phase was extracted with DCM (3×) and the combined organics were washed with brine, dried over Na₂SO₄, and concentrated in vacuo to afford the title product as a white solid (77 mg, 70%). MS(ES)+ m/e 496.0 [M+H]⁺.

### Example 38

### 4-cyclopropyl-7-fluoro-9-{[4-(7-quinolinyl)phenyl]sulfonyl}-1-oxa-4,9-diazaspiro[5.5]undecan-3-one (Diasteromer 2)

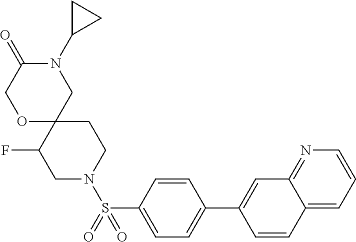 [SMILES: O=C1COC2(CCN(S(=O)(=O)c3ccc(-c4ccc5cccnc5c4)cc3)CC2F)CN1C1CC1]

a) Following the procedure described in Example 37a with 9-[(4-bromophenyl)sulfonyl]-4-cyclopropyl-7-fluoro-1-oxa-4,9-diazaspiro[5.5]undecan-3-one (Diastereomer 2) afforded the title product as a white solid (80 mg, 72%). MS(ES)+ m/e 496.0 [M+H]⁺.

### Example 39

### 4-cyclopropyl-7-fluoro-9-{[4-(7-quinolinyl)phenyl]sulfonyl}-1-oxa-4,9-diazaspiro[5.5]undecan-3-one (Diasteromer 1, enantiomers A and B)

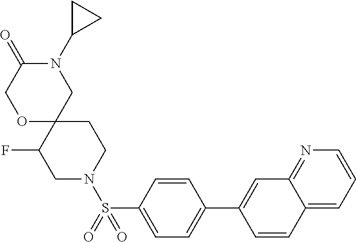 [SMILES: O=C1COC2(CCN(S(=O)(=O)c3ccc(-c4ccc5cccnc5c4)cc3)CC2F)CN1C1CC1]

a) 4-Cyclopropyl-7-fluoro-9-{[4-(7-quinolinyl)phenyl]sulfonyl}-1-oxa-4,9-diazaspiro[5.5]undecan-3-one (Diasteromer 1) (60 mg) was resolved via chiral HPLC (Chiralpak IA, 1:1 acetonitrile:methanol) to afford enantiomers A (27 mg, 100% ee) and B (28 mg, 99.4% ee) as white solids. MS(ES)+ m/e 496.1 [M+H]+ for enantiomer A, 496.1 [M+H]+ for enantiomer B.

### Example 40

### 4-cyclopropyl-7-fluoro-9-{[4-(7-quinolinyl)phenyl]sulfonyl}-1-oxa-4,9-diazaspiro[5.5]undecan-3-one (Diasteromer 2, enantiomers A and B)

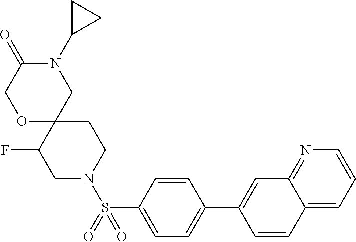 [SMILES: O=C1COC2(CCN(S(=O)(=O)c3ccc(-c4ccc5cccnc5c4)cc3)CC2F)CN1C1CC1]

a) 4-Cyclopropyl-7-fluoro-9-{[4-(7-quinolinyl)phenyl]sulfonyl}-1-oxa-4,9-diazaspiro[5.5]undecan-3-one (Diasteromer 2) (65 mg) was resolved via chiral HPLC (Chiralpak AD-H, 45:45:10 acetonitrile:methanol:isopropanol) to afford enantiomers A (29 mg, 100% ee) and B (30 mg, 100% ee) as white solids. MS(ES)+ m/e 496.1 [M+H]+ for enantiomer A, 496.0 [M+H]+ for enantiomer B.

### Example 41

### 4-cyclopropyl-9-{[3-fluoro-4-(1H-indol-6-yl)phenyl]sulfonyl}-1-oxa-4,9-diazaspiro[5.5]undecan-3-one

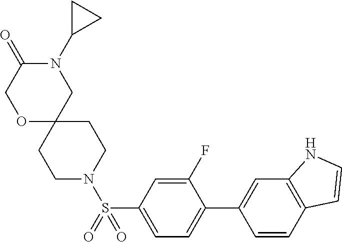 [SMILES: O=C1COC2(CCN(S(=O)(=O)c3ccc(-c4ccc5cc[nH]c5c4)c(F)c3)CC2)CN1C1CC1]

### a) 9-[(4-bromo-3-fluorophenyl)sulfonyl]-4-cyclopropyl-1-oxa-4,9-diazaspiro[5.5]undecan-3-one

To a mixture of 4-cyclopropyl-1-oxa-4,9-diazaspiro[5.5]undecan-3-one hydrochloride (0.951 mmol) in dichloromethane (15 mL) was added N,N-diisopropylethylamine (2.378 mmol). The mixture was stirred for 5 min, then 4-bromo-3-fluorobenzenesulfonyl chloride (0.951 mmol) was added, giving a clear yellow solution. After stirring overnight, the reaction was quenched with water. The layers were separated and the aqueous layer was extracted with dichloromethane. The combined organics were dried over Na₂SO₄, filtered and concentrated under reduced pressure to provide the title product (350 mg, 82% yield) as an off-white solid. MS(ES)+ m/e 448.1 [M+H]⁺; ¹H NMR (400 MHz, DMSO-d₆) δ=8.03 (dd, 1H), 7.74 (dd, J=2.0, 8.1 Hz, 1H), 7.52 (dd, J=1.8, 8.3 Hz, 1H), 3.88 (s, 2H), 3.50 (br. s., 2H), 3.13 (s, 2H), 2.72 (tt, J=3.9, 7.4 Hz, 1H), 2.46 (d, J=2.0 Hz, 2H), 1.87-1.74 (m, 2H), 1.74-1.58 (m, 2H), 0.74-0.63 (m, 2H), 0.63-0.54 (m, 2H).

### b) 4-cyclopropyl-9-{[3-fluoro-4-(1H-indol-6-yl)phenyl]sulfonyl}-1-oxa-4,9-diazaspiro[5.5]undecan-3-one

A mixture of [1,2-bis(diphenylphosphino)ethane]dichloropalladium(II) (0.018 mmol), 1H-indol-6-ylboronic acid (0.179 mmol), potassium carbonate (0.715 mmol) and 9-[(4-bromo-3-fluorophenyl)sulfonyl]-4-cyclopropyl-1-oxa-4,9-diazaspiro[5.5]undecan-3-one (0.179 mmol) in 1,4-dioxane (3 mL) and water (1 mL) was sealed in a microwaveable vessel and heated at 120° C. for 3 h. The reaction mixture was cooled and loaded onto a silica gel pre-column and purified by flash chromatography (dichloromethane to 6% methanol in dichloromethane) to afford the title product (40 mg, 46% yield). MS(ES)+ m/e 484.2 [M+H]⁺; ¹H NMR (400 MHz, CDCl₃) δ=8.37 (br s, 1 H), 7.77 (d, J=8.1 Hz, 1H), 7.75-7.66 (m, 2H), 7.66-7.56 (m, 2H), 7.40-7.32 (m, 2H), 6.64 (br. s., 1H), 4.03 (s, 2H), 3.80-3.61 (m, 2H), 3.17 (s, 2H), 2.83-2.61 (m, 2H), 1.98 (d, J=12.6 Hz, 2H), 1.79-1.64 (m, 2H), 0.88 (q, J=6.8 Hz, 2H), 0.72-0.58 (m, 2H).

### Example 42

### 4-cyclopropyl-9-{[3-fluoro-4-(7-quinolinyl)phenyl]sulfonyl}-1-oxa-4,9-diazaspiro[5.5]undecan-3-one

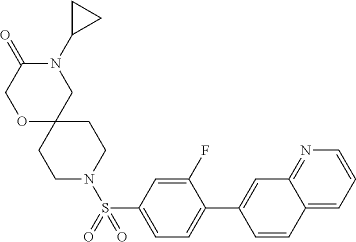 [SMILES: O=C1COC2(CCN(S(=O)(=O)c3ccc(-c4ccc5cccnc5c4)c(F)c3)CC2)CN1C1CC1]

a) Following the procedure described in Example 41b using 7-(4,4,5,5-tetramethyl-1,3,2-dioxaborolan-2-yl)quinoline afforded the title product (38 mg, 43% yield). MS(ES)+ m/e 496.2 [M+H]⁺; ¹H NMR (400 MHz, CDCl₃) δ=9.02 (dd, 1H), 8.35 (s, 1H), 8.25 (d, J=7.6 Hz, 1H), 7.97 (d, J=8.6 Hz, 1H), 7.85-7.76 (m, 2H), 7.73-7.68 (m, 1H), 7.65 (dd, J=1.6, 9.7 Hz, 1H), 7.51 (dd, J=4.3, 8.3 Hz, 1H), 4.04 (s, 2H), 3.81-3.68 (m, 3H), 3.18 (s, 2H), 2.84-2.64 (m, 3H), 1.99 (d, J=12.1 Hz, 2H), 1.74 (td, J=4.5, 13.1 Hz, 2H), 0.94-0.83 (m, 2H), 0.72-0.60 (m, 2H).

### Example 43

### 9-{[3-chloro-4-(1H-indol-6-yl)phenyl]sulfonyl}-4-cyclopropyl-1-oxa-4,9-diazaspiro[5.5]undecan-3-one

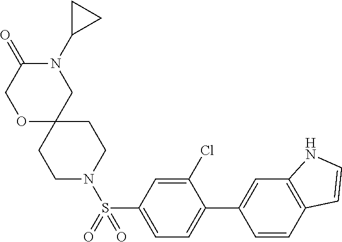 [SMILES: O=C1COC2(CCN(S(=O)(=O)c3ccc(-c4ccc5cc[nH]c5c4)c(Cl)c3)CC2)CN1C1CC1]

### a) 9-[(4-bromo-3-chlorophenyl)sulfonyl]-4-cyclopropyl-1-oxa-4,9-diazaspiro[5.5]undecan-3-one

Following the procedure described in Example 41a using 4-bromo-3-chlorobenzenesulfonyl chloride provided the title product (380 mg, 86% yield). MS(ES)+ m/e 465.0 [M+H]⁺; ¹H NMR (400 MHz, DMSO-d₆) δ=8.08 (d, 1H), 7.91 (d, J=2.0 Hz, 1H), 7.61 (dd, J=2.0, 8.3 Hz, 1H), 3.88 (s, 2H), 3.49 (d, J=11.9 Hz, 2H), 3.13 (s, 2H), 2.72 (tt, J=3.9, 7.4 Hz, 1H), 2.49-2.40 (m, 2H), 1.90-1.74 (m, 2H), 1.74-1.55 (m, 2H), 0.79-0.62 (m, 2H), 0.62-0.52 (m, 2H).

### b) 9-{[3-chloro-4-(1H-indol-6-yl)phenyl]sulfonyl}-4-cyclopropyl-1-oxa-4,9-diazaspiro[5.5]undecan-3-one

Following the procedure described in Example 41b using 9-[(4-bromo-3-chlorophenyl)sulfonyl]-4-cyclopropyl-1-oxa-4,9-diazaspiro[5.5]undecan-3-one provided the title product (38 mg, 44% yield). MS(ES)+ m/e 501.2 [M+H]⁺; ¹H NMR (400 MHz, CDCl₃) δ=8.35 (br. s., 1H), 7.90 (d, J=1.8 Hz, 1H), 7.76 (d, J=8.3 Hz, 1H), 7.70 (dd, J=1.9, 8.0 Hz, 1H), 7.60 (d, J=8.1 Hz, 1H), 7.55 (s, 1H), 7.37-7.32 (m, 1H), 7.24 (dd, J=1.5, 8.3 Hz, 1H), 6.65 (dd, J=1.5, 2.8 Hz, 1H), 4.05 (s, 2H), 3.79-3.66 (m, 2H), 3.18 (s, 2H), 2.83-2.63 (m, 3H), 1.99 (d, J=12.1 Hz, 2H), 1.73 (td, J=4.5, 13.1 Hz, 2H), 0.94-0.83 (m, 2H), 0.73-0.61 (m, 2H).

### Example 44

### 9-{[3-chloro-4-(7-quinolinyl)phenyl]sulfonyl}-4-cyclopropyl-1-oxa-4,9-diazaspiro[5.5]undecan-3-one

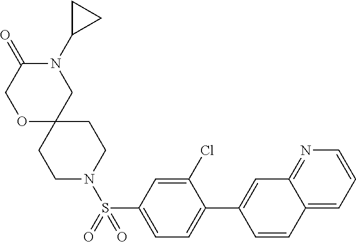 [SMILES: O=C1COC2(CCN(S(=O)(=O)c3ccc(-c4ccc5cccnc5c4)c(Cl)c3)CC2)CN1C1CC1]

a) Following the procedure described in Example 42a using 9-[(4-bromo-3-chlorophenyl)sulfonyl]-4-cyclopropyl-1-oxa-4,9-diazaspiro[5.5]undecan-3-one afforded the title product (50 mg, 57% yield). MS(ES)+ m/e 513.2 [M+H]⁺; ¹H NMR (400 MHz, CDCl₃) δ=9.02 (dd, 1H), 8.26 (dd, J=0.9, 8.5 Hz, 1H), 8.23-8.21 (m, 1H), 7.98-7.94 (m, 2H), 7.78 (dd, J=1.8, 8.1 Hz, 1H), 7.71 (dd, J=1.8, 8.3 Hz, 1H), 7.67 (d, J=8.1 Hz, 1H), 7.53-7.49 (m, 1H), 4.06 (s, 2H), 3.79-3.73 (m, 2H), 3.19 (s, 2H), 2.80-2.70 (m, 3H), 2.00 (d, J=12.1 Hz, 2H), 1.80-1.69 (m, 2H), 0.92-0.86 (m, 2H), 0.70-0.65 (m, 2H).

### Example 45

### 4-cyclopropyl-9-{[4-(1H-indol-6-yl)-3-methylphenyl]sulfonyl}-1-oxa-4,9-diazaspiro[5.5]undecan-3-one

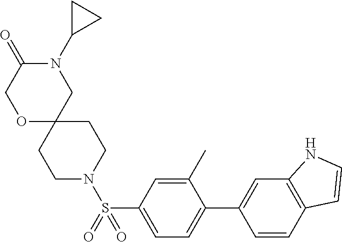 [SMILES: Cc1cc(S(=O)(=O)N2CCC3(CC2)CN(C2CC2)C(=O)CO3)ccc1-c1ccc2cc[nH]c2c1]

### a) 9-[(4-bromo-3-methylphenypsulfonyl]-4-cyclopropyl-1-oxa-4,9-diazaspiro[5.5]undecan-3-one

Following the procedure described in Example 41a using 4-bromo-3-methylbenzenesulfonyl chloride provided the title product (300 mg, 71% yield). MS(ES)+ m/e 444.0 [M+H]⁺; ¹H NMR (400 MHz, DMSO-d₆) δ=7.88 (d, 1H), 7.72 (d, J=1.8 Hz, 1H), 7.47 (dd, J=1.8, 8.3 Hz, 1H), 3.87 (s, 2H), 3.46 (d, J=11.6 Hz, 2H), 3.12 (s, 2H), 2.79-2.68 (m, 1H), 2.46 (s, 3H), 2.44-2.26 (m, 2H), 1.88-1.76 (m, 2H), 1.66 (td, J=4.5, 13.1 Hz, 2H), 0.76-0.63 (m, 2H), 0.62-0.52 (m, 2H).

### b) 4-cyclopropyl-9-{[4-(1H-indol-6-yl)-3-methylphenyl]sulfonyl}-1-oxa-4,9-diazaspiro[5.5]undecan-3-one

Following the procedure described in Example 41b using 9-[(4-bromo-3-methylphenyl)sulfonyl]-4-cyclopropyl-1-oxa-4,9-diazaspiro[5.5]undecan-3-one provided the title product (40 mg, 46% yield). MS(ES)+ m/e 480.0 [M+H]⁺; ¹H NMR (400 MHz, CDCl₃) δ=8.29 (br s, 1H), 7.73 (d, J=8.1 Hz, 1H), 7.68 (d, J=1.8 Hz, 1H), 7.63 (dd, J=1.6, 8.0 Hz, 1H), 7.47 (d, J=7.8 Hz, 1H), 7.39-7.36 (m, 1H), 7.35-7.31 (m, 1H), 7.11 (dd, J=1.5, 8.1 Hz, 1H), 6.65 (ddd, J=0.9, 2.1, 3.1 Hz, 1H), 4.04 (s, 2H), 3.77-3.68 (m, 2H), 3.17 (s, 2H), 2.75 (dq, J=3.6, 7.4 Hz, 1H), 2.67 (td, J=2.0, 11.9 Hz, 2H), 2.40 (s, 3H), 1.97 (d, J=12.4 Hz, 2H), 1.73 (td, J=4.5, 13.1 Hz, 2H), 0.99-0.79 (m, 2H), 0.76-0.56 (m, 2H).

### Example 46

### 4-cyclopropyl-9-{[3-methyl-4-(7-quinolinyl)phenyl]sulfonyl}-1-oxa-4,9-diazaspiro[5.5]undecan-3-one

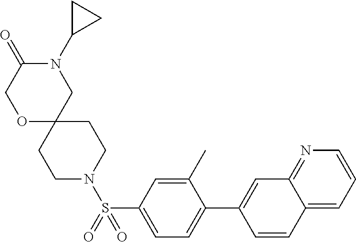 [SMILES: Cc1cc(S(=O)(=O)N2CCC3(CC2)CN(C2CC2)C(=O)CO3)ccc1-c1ccc2cccnc2c1]

a) Following the procedure described in Example 42a using 9-[(4-bromo-3-methylphenyl)sulfonyl]-4-cyclopropyl-1-oxa-4,9-diazaspiro[5.5]undecan-3-one afforded the title product (40 mg, 45% yield). MS(ES)+ m/e 492.2 [M+H]⁺; ¹H NMR (400 MHz, CDCl₃) δ=9.01 (dd, 1H), 8.26 (d, J=7.3 Hz, 1H), 8.10 (s, 1H), 7.94 (d, J=8.3 Hz, 1H), 7.74 (s, 1H), 7.70 (dd, J=1.5, 7.8 Hz, 1H), 7.59-7.46 (m, 3H), 4.05 (s, 2H), 3.81-3.68 (m, 2H), 3.18 (s, 2H), 2.83-2.63 (m, 3H), 2.43 (s, 3H), 1.99 (d, J=12.1 Hz, 2H), 1.74 (td, J=4.4, 13.2 Hz, 2H), 0.94-0.81 (m, 2H), 0.71-0.60 (m, 2H).

### Example 47

### 4-cyclopropyl-9-{[2,5-difluoro-4-(1H-indol-6-yl)phenyl]sulfonyl}-1-oxa-4,9-diazaspiro[5.5]undecan-3-one

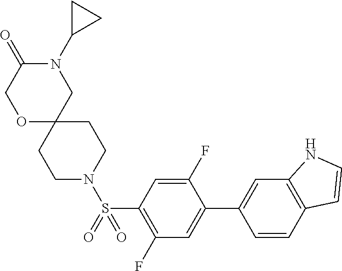 [SMILES: O=C1COC2(CCN(S(=O)(=O)c3cc(F)c(-c4ccc5cc[nH]c5c4)cc3F)CC2)CN1C1CC1]

### a) 9-[(4-bromo-2,5-difluorophenyl)sulfonyl]-4-cyclopropyl-1-oxa-4,9-diazaspiro[5.5]undecan-3-one

Following the procedure described in Example 41a using 4-bromo-2,5-difluorobenzenesulfonyl chloride provided the title product (260 mg, 84% yield). MS(ES)+ m/e 465.1, 466.8 [M+H]⁺; ¹H NMR (400 MHz, CDCl₃) δ=7.63 (s, 1H), 7.54-7.45 (m, 1H), 4.06 (s, 1H), 3.80-3.69 (m, 1H), 3.17 (s, 1H), 2.89 (t, J=12.3 Hz, 1H), 2.75 (tt, J=3.9, 7.3 Hz, 1H), 1.96 (dd, J=2.3, 14.4 Hz, 1H), 1.75-1.63 (m, 1H), 1.55-1.38 (m, 1H), 0.96-0.83 (m, 1H), 0.75-0.58 (m, 1H).

### b) 4-cyclopropyl-9-{[2,5-difluoro-4-(1H-indol-6-yl)phenyl]sulfonyl}-1-oxa-4,9-diazaspiro[5.5]undecan-3-one

Following the procedure described in Example 41b using 9-[(4-bromo-2,5-difluorophenyl)sulfonyl]-4-cyclopropyl-1-oxa-4,9-diazaspiro[5.5]undecan-3-one provided the title product (50 mg, 58% yield). MS(ES)+ m/e 502.0 [M+H]⁺; ¹H NMR (400 MHz, CDCl₃) δ=8.38 (br s, 1H), 7.77 (d, J=8.1 Hz, 1H), 7.72-7.61 (m, 2H), 7.41 (dd, J=5.9, 10.5 Hz, 1H), 7.38-7.30 (m, 2H), 6.70-6.59 (m, 1H), 4.08 (s, 2H), 3.80 (d, J=12.1 Hz, 2H), 3.18 (s, 2H), 2.94 (t, J=12.1 Hz, 2H), 2.76 (tt, J=3.9, 7.3 Hz, 1H), 1.98 (d, J=12.4 Hz, 2H), 1.71 (td, J=4.5, 13.1 Hz, 2H), 0.95-0.81 (m, 2H), 0.71-0.59 (m, 2H).

### Example 48

### 4-cyclopropyl-9-{[2,5-difluoro-4-(7-quinolinyl)phenyl]sulfonyl}-1-oxa-4,9-diazaspiro[5.5]undecan-3-one

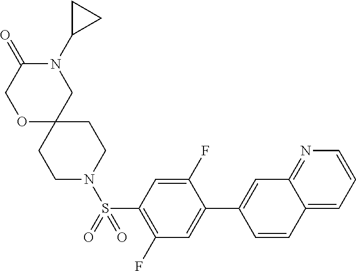 [SMILES: O=C1COC2(CCN(S(=O)(=O)c3cc(F)c(-c4ccc5cccnc5c4)cc3F)CC2)CN1C1CC1]

a) Following the procedure described in Example 42a using 9-[(4-bromo-2,5-difluorophenyl)sulfonyl]-4-cyclopropyl-1-oxa-4,9-diazaspiro[5.5]undecan-3-one afforded the title product (60 mg, 68% yield). MS(ES)+ m/e 514.2 [M+H]⁺; ¹H NMR (400 MHz, CDCl₃) δ=9.02 (dd, 1H), 8.34 (s, 1H), 8.25 (d, J=7.6 Hz, 1H), 7.97 (d, J=8.3 Hz, 1H), 7.83-7.68 (m, 2H), 7.59-7.43 (m, 2H), 4.09 (s, 2H), 3.82 (d, J=12.4 Hz, 2H), 3.20 (s, 2H), 2.98 (t, J=12.1 Hz, 2H), 2.81-2.70 (m, 1H), 2.00 (d, J=12.4 Hz, 2H), 1.73 (td, J=4.7, 13.2 Hz, 2H), 0.95-0.83 (m, 2H), 0.72-0.62 (m, 2H).

### Example 49

### 4-cyclopropyl-9-{[3-(methyloxy)-4-(7-quinolinyl)phenyl]sulfonyl}-1-oxa-4,9-diazaspiro[5.5]undecan-3-one

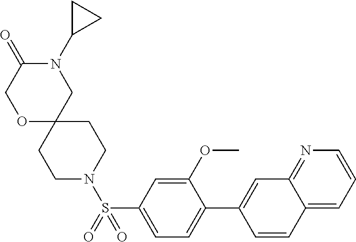 [SMILES: COc1cc(S(=O)(=O)N2CCC3(CC2)CN(C2CC2)C(=O)CO3)ccc1-c1ccc2cccnc2c1]

### a) 4-bromo-3-(methyloxy)benzenesulfonyl chloride

To a 10° C. solution of 4-bromo-3-(methyloxy)aniline (17.32 mmol) in acetic acid (10 mL) and water (5 mL) was added conc. hydrochloric acid (5 mL) and then sodium nitrite (19.05 mmol) in water (5 mL) portionwise, keeping the temperature below 5° C. The mixture was stirred at 10° C. for 1 h. Copper(I) chloride (17.32 mmol) was dissolved in conc. HCl (10 mL) and added to a saturated solution of sulfur dioxide (173 mmol) in acetic acid (30 mL) with a stream of sulfur dioxide passed through the mixture for a further 15 min. The diazonium salt was then added portionwise to the sulfur dioxide mixture, keeping the temperature below 30° C., stirred for 2 h and then quenched by adding to ice and stirring for 20 min. The mixture was extracted with ethyl acetate (3×) and the combined extracts dried and evaporated to give the title product (4 g, 81% yield) as a gum. The crude product was used without purification. ¹H NMR (400 MHz, CDCl₃) δ=7.82 (d, 1H), 7.55 (dd, J=2.0, 8.3 Hz, 1H), 7.48 (d, J=2.0 Hz, 1H), 4.04 (s, 1H).

### b) 9-{[4-bromo-3-(methyloxy)phenyl]sulfonyl}-4-cyclopropyl-1-oxa-4,9-diazaspiro[5.5]undecan-3-one

To a mixture of 4-cyclopropyl-1-oxa-4,9-diazaspiro[5.5]undecan-3-one hydrochloride (0.811 mmol) in dichloromethane (15 mL) was added N,N-diisdopropylethylamine (2.026 mmol). The mixture was stirred for 5 min, then 4-bromo-3-(methyloxy)benzenesulfonyl chloride (0.811 mmol) was added, giving a clear yellow solution. After stirring for 2 h, the reaction was quenched with water. The layers were separated and the aqueous layer was extracted with dichloromethane. The combined organics were dried over Na₂SO₄, filtered and concentrated under reduced pressure. The residue was triturated with ethanol, collected and washed with ethanol and hexanes to afford the title product (120 mg, 32% yield) as an off-white solid. MS(ES)+ m/e 460.2 [M+H]⁺; ¹H NMR (400 MHz, CDCl₃) δ=7.78-7.68 (m, 1H), 7.24 (dq, J=2.1, 4.3 Hz, 2H), 4.01 (s, 2H), 3.98 (s, 2H), 3.66 (d, J=11.4 Hz, 2H), 3.15 (s, 3H), 2.82-2.69 (m, 1H), 2.60 (td, J=2.5, 12.1 Hz, 2H), 1.95 (dd, J=2.3, 14.1 Hz, 2H), 1.78-1.64 (m, 2H), 0.94-0.81 (m, 2H), 0.70-0.60 (m, 2H).

### c) 4-cyclopropyl-9-{[3-(methyloxy)-4-(7-quinolinyl)phenyl]sulfonyl}-1-oxa-4,9-diazaspiro[5.5]undecan-3-one

Following the procedure described in Example 42a using 9-{[4-bromo-3-(methyloxy)phenyl]sulfonyl}-4-cyclopropyl-1-oxa-4,9-diazaspiro[5.5]undecan-3-one afforded the title product (45 mg, 74% yield). MS(ES)+ m/e 508.0 [M+H]⁺; ¹H NMR (400 MHz, CDCl₃) δ=9.06-8.92 (m, 1H), 8.40-8.16 (m, 2H), 7.92 (d, J=8.3 Hz, 1H), 7.79 (d, J=8.6 Hz, 1H), 7.64 (d, J=7.8 Hz, 1H), 7.50 (dd, J=1.8, 7.8 Hz, 2H), 7.40 (d, J=1.8 Hz, 1H), 4.05 (s, 2H), 3.94 (s, 3H), 3.74 (d, J=11.4 Hz, 2H), 3.18 (s, 2H), 2.80-2.67 (m, 3H), 1.99 (d, J=12.1 Hz, 2H), 1.82-1.68 (m, 2H), 0.93-0.84 (m, 2H), 0.70-0.62 (m, 2H).

### Example 50

### 4-cyclopropyl-9-{[4-(1H-indol-6-yl)-3-(methyloxy)phenyl]sulfonyl}-1-oxa-4,9-diazaspiro[5.5]undecan-3-one

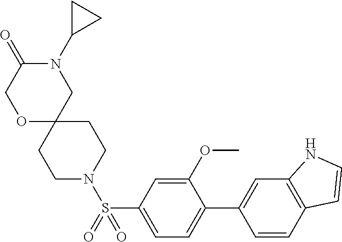 [SMILES: COc1cc(S(=O)(=O)N2CCC3(CC2)CN(C2CC2)C(=O)CO3)ccc1-c1ccc2cc[nH]c2c1]

a) Following the procedure described in Example 41b using 9-{[4-bromo-3-(methyloxy)phenyl]sulfonyl}-4-cyclopropyl-1-oxa-4,9-diaz aspiro[5.5]undecan-3-one provided the title product (35 mg, 59% yield). MS(ES)+ m/e 496.1 [M+H]⁺; ¹H NMR (400 MHz, DMSO-d₆) δ=11.21 (br s, 1H), 7.66-7.56 (m, 3H), 7.45-7.36 (m, 2H), 7.31 (s, 1H), 7.17 (d, J=8.3 Hz, 1H), 6.47 (br. s., 1H), 3.91 (s, 2H), 3.87 (s, 3H), 3.54 (d, J=11.4 Hz, 2H), 3.14 (s, 2H), 2.73 (dt, J=3.5, 7.4 Hz, 1H), 1.85 (d, J=13.4 Hz, 2H), 1.78-1.59 (m, 2H), 0.80-0.64 (m, 2H), 0.63-0.48 (m, 2H).

### Example 51

### 4-cyclopropyl-9-{[5-(7-quinolinyl)-2-pyridinyl]sulfonyl}-1-oxa-4,9-diazaspiro[5.5]undecan-3-one

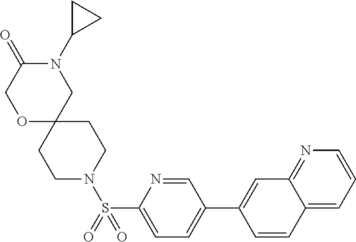 [SMILES: O=C1COC2(CCN(S(=O)(=O)c3ccc(-c4ccc5cccnc5c4)cn3)CC2)CN1C1CC1]

### a) 9-[(5-bromo-2-pyridinyl)sulfonyl]-4-cyclopropyl-1-oxa-4,9-diazaspiro[5.5]undecan-3-one

Following the procedure described in Example 41a using 5-bromo-2-pyridinesulfonyl chloride hydrochloride provided the title product (290 mg, 83% yield). MS(ES)+ m/e 431.2 [M+H]⁺; ¹H NMR (400 MHz, CDCl₃) δ=8.80 (d, 1H), 8.07 (dd, J=2.3, 8.1 Hz, 1H), 7.85 (d, J=8.3 Hz, 1H), 4.06 (s, 2H), 3.88-3.70 (m, 2H), 3.16 (s, 2H), 3.01 (td, J=2.5, 12.4 Hz, 2H), 2.75 (tt, J=3.9, 7.3 Hz, 1H), 1.95 (dd, J=2.3, 14.1 Hz, 2H), 1.68 (td, J=4.7, 13.2 Hz, 2H), 0.96-0.80 (m, 2H), 0.72-0.57 (m, 2H).

### b) 4-cyclopropyl-9-{[5-(7-quinolinyl)-2-pyridinyl]sulfonyl}-1-oxa-4,9-diazaspiro[5.5]undecan-3-one

Following the procedure described in Example 42a using 9-[(5-bromo-2-pyridinyl)sulfonyl]-4-cyclopropyl-1-oxa-4,9-diazaspiro[5.5]undecan-3-one afforded the title product (40 mg, 45% yield). MS(ES)+ m/e 478.8 [M+H+]; ¹H NMR (400 MHz, CDCl₃) δ=9.13 (d, 1H), 9.04 (dd, J=1.6, 4.2 Hz, 1H), 8.43 (s, 1H), 8.34-8.21 (m, 2H), 8.10 (d, J=8.1 Hz, 1H), 8.03 (d, J=8.3 Hz, 1H), 7.84 (dd, J=1.8, 8.6 Hz, 1H), 7.53 (dd, J=4.2, 8.2 Hz, 1H), 4.08 (s, 2H), 3.88 (d, J=12.1 Hz, 2H), 3.18 (s, 2H), 3.10 (td, J=2.4, 12.3 Hz, 2H), 2.76 (tt, J=3.9, 7.3 Hz, 1H), 1.98 (d, J=12.4 Hz, 2H), 1.73 (td, J=4.5, 13.1 Hz, 2H), 0.93-0.82 (m, 2H), 0.71-0.61 (m, 2H).

### Example 52

### 4-cyclopropyl-9-{[5-(1H-indol-6-yl)-2-pyridinyl]sulfonyl}-1-oxa-4,9-diazaspiro[5.5]undecan-3-one

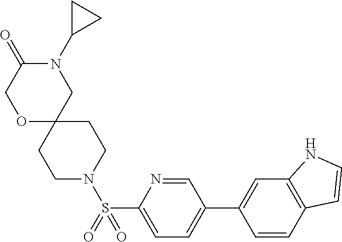 [SMILES: O=C1COC2(CCN(S(=O)(=O)c3ccc(-c4ccc5cc[nH]c5c4)cn3)CC2)CN1C1CC1]

a) Following the procedure described in Example 41b using 9-[(5-bromo-2-pyridinyl)sulfonyl]-4-cyclopropyl-1-oxa-4,9-diazaspiro[5.5]undecan-3-one provided the title product (60 mg, 69% yield). MS(ES)+ m/e 467.0 [M+H]⁺; ¹H NMR (400 MHz, CDCl₃) δ=9.09-8.95 (m, 1H), 8.43 (br s, 1H), 8.21-8.12 (m, 1H), 8.05-7.96 (m, 1H), 7.80 (d, J=8.3 Hz, 1H), 7.72-7.64 (m, 1H), 7.41 (dd, J=1.6, 8.2 Hz, 1H), 7.38-7.33 (m, 1H), 6.66 (t, J=2.1 Hz, 1H), 4.07 (s, 1H), 3.86 (d, J=12.1 Hz, 1H), 3.17 (s, 1H), 3.06 (td, J=2.4, 12.3 Hz, 1H), 2.86-2.67 (m, 1H), 1.95 (br. s., 1H), 1.71 (td, J=4.5, 13.1 Hz, 1H), 1.62 (s, 2H), 0.96-0.81 (m, 2H), 0.71-0.60 (m, 1H).

### Example 53

### 4-cyclopropyl-9-{[4-(7-quinolinyl)-3-(trifluoromethyl)phenyl]sulfonyl}-1-oxa-4,9-diazaspiro[5.5]undecan-3-one

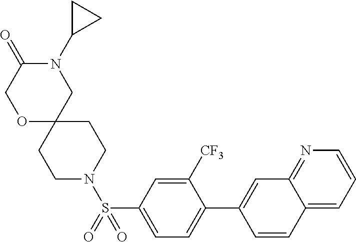 [SMILES: O=C1COC2(CCN(S(=O)(=O)c3ccc(-c4ccc5cccnc5c4)c(C(F)(F)F)c3)CC2)CN1C1CC1]

### a) 9-{[4-bromo-3-(trifluoromethyl)phenyl]sulfonyl}-4-cyclopropyl-1-oxa-4,9-diazaspiro[5.5]undecan-3-one

Following the procedure described in Example 41a using 4-bromo-3-(trifluoromethyl)benzenesulfonyl chloride provided the title product (325 mg, 81% yield). MS(ES)+ m/e 498.0 [M+H]⁺; ¹H NMR (400 MHz, CDCl₃) δ=8.06 (d, 1H), 7.94 (d, J=8.3 Hz, 1H), 7.78 (dd, J=2.0, 8.3 Hz, 1H), 4.02 (s, 2H), 3.73-3.65 (m, 2H), 3.17 (s, 2H), 2.75 (dt, J=3.4, 7.5 Hz, 1H), 2.62 (td, J=2.5, 12.1 Hz, 2H), 1.97 (dd, J=2.3, 14.4 Hz, 1H), 1.76-1.64 (m, 2H), 0.95-0.82 (m, 2H), 0.69-0.61 (m, 2H).

### b) 4-cyclopropyl-9-{[4-(7-quinolinyl)-3-(trifluoromethyl)phenyl]sulfonyl}-1-oxa-4,9-diazaspiro[5.5]undecan-3-one

Following the procedure described in Example 42a using 9-{[4-bromo-3-(trifluoromethyl)phenyl]sulfonyl}-4-cyclopropyl-1-oxa-4,9-diazaspiro[5.5]undecan-3-one afforded the title product (45 mg, 51% yield). MS(ES)+ m/e 546.1 [M+H]⁺; ¹H NMR (400 MHz, CDCl₃) δ=9.03 (dd, 1H), 8.28 (d, J=8.1 Hz, 1H), 8.21 (d, J=1.8 Hz, 1H), 8.14 (s, 1H), 8.02 (dd, J=1.8, 8.1 Hz, 1H), 7.94 (d, J=8.3 Hz, 1H), 7.66 (d, J=7.8 Hz, 1H), 7.61-7.48 (m, 2H), 4.07 (s, 2H), 3.86-3.70 (m, 2H), 3.20 (s, 2H), 2.84-2.68 (m, 3H), 2.01 (d, J=12.1 Hz, 2H), 1.75 (td, J=4.5, 13.1 Hz, 2H), 0.94-0.85 (m, 2H), 0.72-0.63 (m, 2H).

### Example 54

### 4-cyclopropyl-9-{[2-methyl-4-(7-quinolinyl)phenyl]sulfonyl}-1-oxa-4,9-diazaspiro[5.5]undecan-3-one

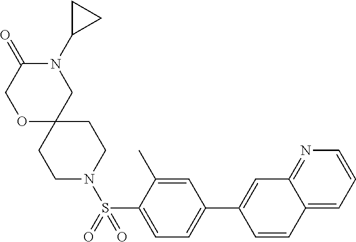 [SMILES: Cc1cc(-c2ccc3cccnc3c2)ccc1S(=O)(=O)N1CCC2(CC1)CN(C1CC1)C(=O)CO2]

### a) 9-[(4-bromo-2-methylphenyl)sulfonyl]-4-cyclopropyl-1-oxa-4,9-diazaspiro[5.5]undecan-3-one

Following the procedure described in Example 41a using 4-bromo-2-methylbenzenesulfonyl chloride provided the title product (280 mg, 78% yield). MS (ES)+ m/e 444.1 [M+H]⁺; ¹H NMR (400 MHz, CDCl₃) δ=7.77 (d, J=8.3 Hz, 1H), 7.56-7.44 (m, 2H), 4.08 (s, 2H), 3.68-3.52 (m, 2H), 3.17 (s, 2H), 2.96 (td, J=2.4, 12.4 Hz, 2H), 2.75 (tt, J=3.9, 7.3 Hz, 1H), 2.60 (s, 3H), 2.00-1.86 (m, 2H), 1.68 (td, J=4.5, 13.1 Hz, 2H), 0.95-0.83 (m, 2H), 0.73-0.61 (m, 2H).

### b) 4-cyclopropyl-9-{[2-methyl-4-(7-quinolinyl)phenyl]sulfonyl}-1-oxa-4,9-diazaspiro[5.5]undecan-3-one

Following the procedure described in Example 42a using 9-[(4-bromo-2-methylphenyl)sulfonyl]-4-cyclopropyl-1-oxa-4,9-diazaspiro[5.5]undecan-3-one afforded the title product (45 mg, 51% yield). MS(ES)+ m/e 492.2 [M+H]⁺; ¹H NMR (400 MHz, DMSO-d₆) δ=8.98 (dd, 1H), 8.49-8.36 (m, 2H), 8.18-8.08 (m, 1H), 8.08-7.98 (m, 2H), 7.93 (s, 2H), 7.59 (dd, J=4.3, 8.3 Hz, 1H), 3.96 (s, 2H), 3.49 (d, J=12.1 Hz, 2H), 3.16 (s, 2H), 2.81 (td, J=2.1, 12.1 Hz, 2H), 2.74 (dt, J=3.4, 7.5 Hz, 1H), 2.69 (s, 3H), 1.84 (d, J=13.1 Hz, 2H), 1.72-1.58 (m, 2H), 0.73-0.63 (m, 2H), 0.60 (sxt, J=4.2 Hz, 2H).

### Example 55

### 4-cyclopropyl-9-{[4-(1H-indol-6-yl)-2-methylphenyl]sulfonyl}-1-oxa-4,9-diazaspiro[5.5]undecan-3-one

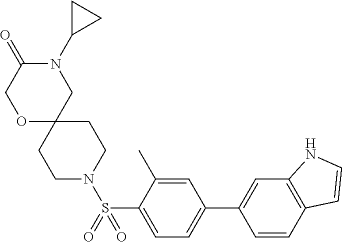 [SMILES: Cc1cc(-c2ccc3cc[nH]c3c2)ccc1S(=O)(=O)N1CCC2(CC1)CN(C1CC1)C(=O)CO2]

a) Following the procedure described in Example 41b using 9-[(4-bromo-2-methylphenyl)sulfonyl]-4-cyclopropyl-1-oxa-4,9-diazaspiro[5.5]undecan-3-one provided the title product (50 mg, 58% yield). MS(ES)+ m/e 480.0 [M+H]⁺; ¹H NMR (400 MHz, DMSO-d₆) δ=11.28 (s, 1H), 7.85 (d, J=8.3 Hz, 1H), 7.78 (s, 1H), 7.76-7.70 (m, 2H), 7.66 (d, J=8.3 Hz, 1H), 7.48-7.43 (m, 1H), 7.40 (dd, J=1.8, 8.3 Hz, 1H), 6.48 (t, J=2.0 Hz, 1H), 3.95 (s, 2H), 3.54-3.43 (m, 2H), 3.15 (s, 2H), 2.85-2.70 (m, 3H), 2.65 (s, 3H), 1.83 (d, J=13.4 Hz, 2H), 1.72-1.56 (m, 2H), 0.74-0.63 (m, 2H), 0.63-0.56 (m, 2H).

### Example 56

### 9-{[2-chloro-4-(1H-indol-6-yl)phenyl]sulfonyl}-4-cyclopropyl-1-oxa-4,9-diazaspiro[5.5]undecan-3-one

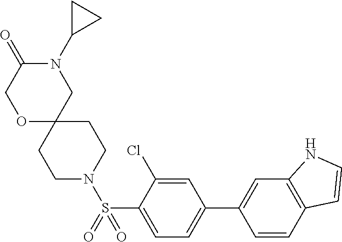 [SMILES: O=C1COC2(CCN(S(=O)(=O)c3ccc(-c4ccc5cc[nH]c5c4)cc3Cl)CC2)CN1C1CC1]

### a) 9-[(4-bromo-2-chlorophenyl)sulfonyl]-4-cyclopropyl-1-oxa-4,9-diazaspiro[5.5]undecan-3-one

Following the procedure described in Example 41a using 4-bromo-2-chlorobenzenesulfonyl chloride provided the title product (330 mg, 88% yield). MS (ES)+ m/e 464.0 [M+H]⁺; ¹H NMR (400 MHz, CDCl₃) δ=7.93 (d, 1H), 7.73 (d, J=2.0 Hz, 1H), 7.56 (dd, J=1.9, 8.5 Hz, 1H), 4.09 (s, 2H), 3.77-3.66 (m, 2H), 3.17 (s, 2H), 3.07 (td, J=2.5, 12.6 Hz, 2H), 2.75 (tt, J=3.9, 7.3 Hz, 1H), 1.93 (dd, J=2.1, 14.3 Hz, 1H), 1.73-1.61 (m, 2H), 0.93-0.84 (m, 2H), 0.70-0.60 (m, 2H).

### b) 9-{[2-chloro-4-(1H-indol-6-yl)phenyl]sulfonyl}-4-cyclopropyl-1-oxa-4,9-diazaspiro[5.5]undecan-3-one

Following the procedure described in Example 41b using 9-[(4-bromo-2-chlorophenyl)sulfonyl]-4-cyclopropyl-1-oxa-4,9-diazaspiro[5.5]undecan-3-one provided the title product (30 mg, 35% yield). MS(ES)+ m/e 501.2 [M+H]⁺; ¹H NMR (400 MHz, DMSO-d₆) δ=11.34 (s, 1H), 8.05-7.96 (m, 2H), 7.88 (dd, J=1.8, 8.3 Hz, 1H), 7.79 (s, 1H), 7.67 (d, J=8.3 Hz, 1H), 7.51-7.46 (m, 1H), 7.43 (dd, J=1.5, 8.3 Hz, 1H), 6.49 (d, J=2.0 Hz, 1H), 3.96 (s, 2H), 3.58 (d, J=12.6 Hz, 2H), 3.16 (s, 2H), 2.92 (td, J=2.4, 12.2 Hz, 2H), 2.74 (tt, J=3.9, 7.4 Hz, 1H), 1.83 (d, J=13.9 Hz, 2H), 1.64 (td, J=4.4, 12.8 Hz, 2H), 0.75-0.63 (m, 2H), 0.60 (dq, J=3.8, 8.0 Hz, 2H).

### Example 57

### 4-cyclopropyl-9-{[5-(1H-indol-6-yl)-2-thienyl]sulfonyl}-1-oxa-4,9-diazaspiro[5.5]undecan-3-one

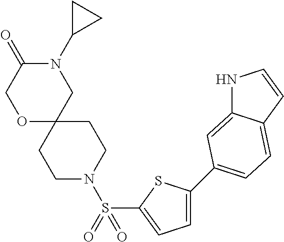 [SMILES: O=C1COC2(CCN(S(=O)(=O)c3ccc(-c4ccc5cc[nH]c5c4)s3)CC2)CN1C1CC1]

### a) 9-[(5-bromo-2-thienyl)sulfonyl]-4-cyclopropyl-1-oxa-4,9-diazaspiro[5.5]undecan-3-one

Following the procedure described in Example 41a using 5-bromo-2-thiophenesulfonyl chloride provided the title product (250 mg, 71% yield). MS(ES)+m/e 434.9 [M+H]⁺; ¹H NMR (400 MHz, CDCl₃) δ=7.31 (d, 1H), 7.16 (d, J=4.0 Hz, 1H), 4.05 (s, 1H), 3.70-3.61 (m, 1H), 3.17 (s, 1H), 2.80-2.73 (m, 1H), 2.69 (td, J=2.7, 12.2 Hz, 1H), 1.98 (dd, J=2.5, 14.4 Hz, 1H), 1.71 (td, J=4.5, 13.3 Hz, 1H), 0.93-0.84 (m, 1H), 0.69-0.63 (m, 1H).

### b) 4-cyclopropyl-9-{[5-(1H-indol-6-yl)-2-thienyl]sulfonyl}-1-oxa-4,9-diazaspiro[5.5]undecan-3-one

Following the procedure described in Example 41b using 9-[(5-bromo-2-thienyl)sulfonyl]-4-cyclopropyl-1-oxa-4,9-diazaspiro[5.5]undecan-3-one provided the title product (40 mg, 46% yield). MS(ES)+ m/e 472.0 [M+H]⁺; ¹H NMR (400 MHz, DMSO-d₆) δ=11.32 (br s, 1H), 7.77-7.72 (m, 1H), 7.66-7.57 (m, 3H), 7.50-7.44 (m, 1H), 7.42-7.35 (m, 1H), 6.49 (td, J=0.9, 2.0 Hz, 1H), 3.90 (s, 2H), 3.50 (d, J=11.6 Hz, 2H), 3.14 (s, 2H), 2.73 (tt, J=3.9, 7.4 Hz, 1H), 2.57 (td, J=2.1, 12.1 Hz, 2H), 1.88 (d, J=12.9 Hz, 2H), 1.77-1.64 (m, 2H), 0.73-0.65 (m, 2H), 0.61-0.55 (m, 2H).

### Example 58

### 9-{[2-chloro-4-(7-quinolinyl)phenyl]sulfonyl}-4-cyclopropyl-1-oxa-4,9-diazaspiro[5.5]undecan-3-one

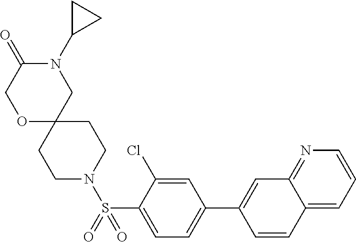 [SMILES: O=C1COC2(CCN(S(=O)(=O)c3ccc(-c4ccc5cccnc5c4)cc3Cl)CC2)CN1C1CC1]

a) Following the procedure described in Example 42a using 9-[(4-bromo-2-chlorophenyl)sulfonyl]-4-cyclopropyl-1-oxa-4,9-diazaspiro[5.5]undecan-3-one afforded the title product (45 mg, 51% yield). MS(ES)+ m/e 513.2 [M+H]⁺; ¹H NMR (400 MHz, DMSO-d₆) δ=8.99 (dd, 1H), 8.49-8.41 (m, 2H), 8.23 (s, 1H), 8.18-8.12 (m, 1H), 8.12-8.03 (m, 3H), 7.61 (dd, J=4.0, 8.3 Hz, 1H), 3.97 (s, 2H), 3.60 (d, J=12.4 Hz, 2H), 3.17 (s, 2H), 2.95 (td, J=2.1, 12.1 Hz, 2H), 2.80-2.70 (m, 1H), 1.84 (d, J=13.4 Hz, 2H), 1.74-1.58 (m, 2H), 0.74-0.63 (m, 2H), 0.60 (dq, J=3.8, 7.9 Hz, 2H).

### Example 59

### 4-cyclopropyl-9-{[2-fluoro-4-(7-quinolinyl)phenyl]sulfonyl}-1-oxa-4,9-diazaspiro[5.5]undecan-3-one

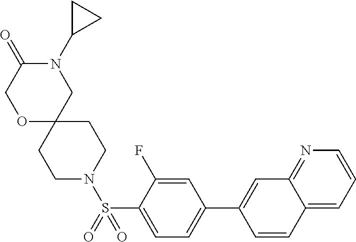 [SMILES: O=C1COC2(CCN(S(=O)(=O)c3ccc(-c4ccc5cccnc5c4)cc3F)CC2)CN1C1CC1]

a) Following the procedure described in Example 42a using 9-[(4-bromo-2-fluorophenyl)sulfonyl]-4-cyclopropyl-1-oxa-4,9-diazaspiro[5.5]undecan-3-one afforded the title product (45 mg, 51% yield). MS(ES)+ m/e 496.1 [M+H]⁺; ¹H NMR (400 MHz, DMSO-d₆) δ=8.99 (dd, 1H), 8.56-8.40 (m, 2H), 8.20-8.13 (m, 1H), 8.13-8.04 (m, 2H), 8.02-7.94 (m, 1H), 7.94-7.84 (m, 1H), 7.67-7.51 (m, 1H), 4.00-3.85 (m, 2H), 3.65-3.52 (m, 2H), 3.16 (s, 2H), 2.84-2.68 (m, 3H), 1.87 (d, J=13.4 Hz, 2H), 1.76-1.61 (m, 2H), 0.75-0.63 (m, 2H), 0.63-0.53 (m, 2H).

### Example 60

### 4-cyclopropyl-9-{[5-(7-quinolinyl)-2-thienyl]sulfonyl}-1-oxa-4,9-diazaspiro[5.5]undecan-3-one

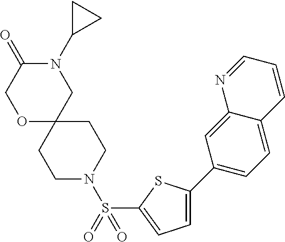 [SMILES: O=C1COC2(CCN(S(=O)(=O)c3ccc(-c4ccc5cccnc5c4)s3)CC2)CN1C1CC1]

a) Following the procedure described in Example 42a using 9-[(5-bromo-2-thienyl)sulfonyl]-4-cyclopropyl-1-oxa-4,9-diazaspiro[5.5]undecan-3-one afforded the title product (10 mg, 11% yield). MS(ES)+ m/e 483.9[M+H]⁺; ¹H NMR (400 MHz, DMSO-d₆) δ=8.97 (dd, 1H), 8.42 (dd, J=1.0, 8.3 Hz, 1H), 8.39 (d, J=2.0 Hz, 1H), 8.17-8.07 (m, 1H), 8.06-8.01 (m, 1H), 7.97 (d, J=4.0 Hz, 1H), 7.71 (d, J=3.8 Hz, 1H), 7.58 (dd, J=4.2, 8.2 Hz, 1H), 3.90 (s, 2H), 3.53 (d, J=11.6 Hz, 2H), 3.15 (s, 2H), 2.78-2.69 (m, 1H), 2.66-2.57 (m, 2H), 1.88 (d, J=13.1 Hz, 2H), 1.72 (td, J=4.4, 13.1 Hz, 2H), 0.74-0.65 (m, 2H), 0.62-0.53 (m, 2H).

### Example 61

### 4-cyclopropyl-9-{[6-(7-quinolinyl)-3-pyridinyl]sulfonyl}-1-oxa-4,9-diazaspiro[5.5]undecan-3-one

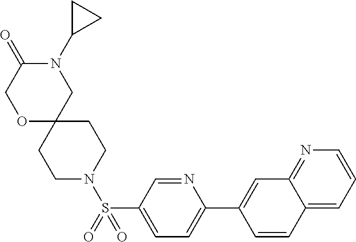 [SMILES: O=C1COC2(CCN(S(=O)(=O)c3ccc(-c4ccc5cccnc5c4)nc3)CC2)CN1C1CC1]

### a) 6-bromo-3-pyridinesulfonyl chloride

To a 10° C. solution of 6-bromo-3-pyridinamine (20.23 mmol) in acetic acid (10 mL) and water (5 mL) was added conc. hydrochloric acid (5 mL) and then sodium nitrite (24.28 mmol) in water (5 mL) portionwise, keeping the temperature below 5° C. The mixture was stirred at 10° C. for 1 h. Copper(I) chloride (20.23 mmol) was dissolved in conc. HCl (10 mL) and added to a saturated solution of sulfur dioxide (202 mmol) in acetic acid (30 mL), with a stream of sulfur dioxide passed through the mixture for a further 15 min. The intermediate diazonium salt was then added portionwise to the sulfur dioxide mixture keeping the temperature below 30° C., stirred for 1 h, then quenched by adding to ice and stirring for 20 min. The mixture was extracted with ethyl acetate (2×) and the combined extracts washed with water (2×), dried, and concentrated in vacuo to a purple gum. The residue was applied to a pad of silica gel and eluted with 40% dichloromethane in hexanes to provide the title product (2.2 g, 42% yield) as a solid. ¹H NMR (400 MHz, CDCl₃) δ=9.10-8.95 (m, 1H), 8.15 (dd, J=2.7, 8.5 Hz, 1H), 7.80 (d, J=8.6 Hz, 1H).

### b) 9-[(6-bromo-3-pyridinyl)sulfonyl]-4-cyclopropyl-1-oxa-4,9-diazaspiro[5.5]undecan-3-one

Following the procedure described in Example 41a using 6-bromo-3-pyridinesulfonyl chloride provided the title product (200 mg, 57% yield). MS(ES)+ m/e 431.0[M+H]⁺; ¹H NMR (400 MHz, CDCl₃) δ=8.82-8.70 (m, 1H), 8.07-7.83 (m, 1H), 7.75-7.51 (m, 1H), 4.01 (s, 2H), 3.77-3.63 (m, 2H), 3.17 (s, 2H), 2.75 (tt, J=3.8, 7.4 Hz, 1H), 2.64 (td, J=2.5, 12.1 Hz, 2H), 1.97 (dd, J=2.3, 14.4 Hz, 2H), 1.70 (td, J=4.7, 13.2 Hz, 2H), 0.94-0.84 (m, 2H), 0.70-0.62 (m, 2H).

### c) 4-cyclopropyl-9-{[6-(7-quinolinyl)-3-pyridinyl]sulfonyl}-1-oxa-4,9-diazaspiro[5.5]undecan-3-one

Following the procedure described in Example 42a using 9-[(6-bromo-3-pyridinyl)sulfonyl]-4-cyclopropyl-1-oxa-4,9-diazaspiro[5.5]undecan-3-one afforded the title product (44 mg, 50% yield). MS(ES)+ m/e 478.8[M+H]⁺; ¹H NMR (400 MHz, DMSO-d₆) δ=9.14-9.04 (m, 1H), 9.01 (dd, J=1.6, 4.2 Hz, 1H), 8.85 (s, 1H), 8.52 (d, J=8.6 Hz, 1H), 8.49-8.41 (m, 2H), 8.28 (dd, J=2.5, 8.3 Hz, 1H), 8.17 (d, J=8.8 Hz, 1H), 7.63 (dd, J=4.2, 8.2 Hz, 1H), 3.87 (s, 2H), 3.58 (d, J=11.9 Hz, 2H), 3.14 (s, 2H), 2.72 (tt, J=3.9, 7.4 Hz, 1H), 2.63-2.53 (m, 2H), 1.90-1.78 (m, 2H), 1.78-1.62 (m, 2H), 0.74-0.63 (m, 2H), 0.62-0.53 (m, 2H).

### Example 62

### 4-cyclopropyl-9-{[6-(1H-indol-6-yl)-3-pyridinyl]sulfonyl}-1-oxa-4,9-diazaspiro[5.5]undecan-3-one

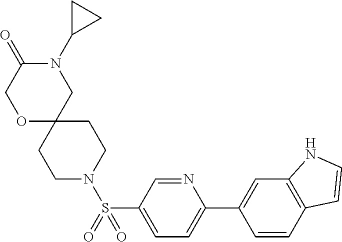 [SMILES: O=C1COC2(CCN(S(=O)(=O)c3ccc(-c4ccc5cc[nH]c5c4)nc3)CC2)CN1C1CC1]

a) Following the procedure described in Example 41b using 9-[(6-bromo-3-pyridinyl)sulfonyl]-4-cyclopropyl-1-oxa-4,9-diazaspiro[5.5]undecan-3-one provided the title product (43 mg, 50% yield). MS(ES)+ m/e 467.0 [M+H]⁺; ¹H NMR (400 MHz, DMSO-d₆) δ=11.42 (br s, 1H), 8.93 (d, J=1.8 Hz, 1H), 8.30 (s, 1H), 8.27-8.19 (m, 1H), 8.14 (dd, J=2.3, 8.6 Hz, 1H), 7.86 (dd, J=1.5, 8.3 Hz, 1H), 7.68 (d, J=8.6 Hz, 1H), 7.52 (t, J=2.8 Hz, 1H), 6.51 (d, J=3.8 Hz, 1H), 3.86 (s, 2H), 3.54 (d, J=11.9 Hz, 2H), 3.14 (s, 2H), 2.77-2.69 (m, 1H), 2.62-2.53 (m, 1H), 1.82 (br. s., 2H), 1.76-1.63 (m, 2H), 0.75-0.63 (m, 2H), 0.62-0.52 (m, 2H).

### Example 63

### 4-cyclopropyl-9-{[2,3-dimethyl-4-(7-quinolinyl)phenyl]sulfonyl}-1-oxa-4,9-diazaspiro[5.5]undecan-3-one

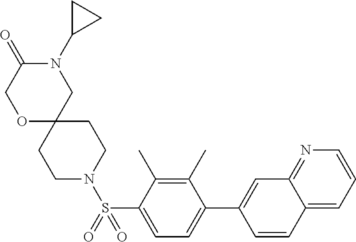 [SMILES: Cc1c(-c2ccc3cccnc3c2)ccc(S(=O)(=O)N2CCC3(CC2)CN(C2CC2)C(=O)CO3)c1C]

### a) 4-bromo-2,3-dimethylbenzenesulfonyl chloride

To a 10° C. suspension of 4-bromo-2,3-dimethylaniline (24.99 mmol) in acetic acid (15 mL) and 6M hydrochloric acid (15 mL) was added sodium nitrite (30.0 mmol) in water (10 mL) portionwise, keeping the temperature below 5° C. The mixture was stirred at 10° C. for 1 h. Copper(I) chloride (24.99 mmol) was dissolved in conc. HCl (15 mL) and added to a saturated solution of sulfur dioxide (250 mmol) in acetic acid (50 mL), with a stream of sulfur dioxide passed through the mixture for a further 15 min. The intermediate diazonium salt was then added portionwise to the sulfur dioxide mixture, keeping the temperature below 30° C., stirred for 3 h, and then quenched by adding to ice and stirring for 20 min. The mixture was extracted with ethyl acetate (2×) and the combined extracts washed with water (2×), dried, and concentrated in vacuo to a pink gum. The residue was applied to a pad of silica gel and eluted with 30% dichloromethane in hexanes to provide the title product (3.4 g, 48% yield) as a pale yellow solid. ¹H NMR (400 MHz, CDCl₃) δ=7.82 (d, 1H), 7.66 (d, J=8.8 Hz, 1H), 2.80 (s, 3H), 2.54 (s, 3H).

### b) 9-[(4-bromo-2,3-dimethylphenyl)sulfonyl]-4-cyclopropyl-1-oxa-4,9-diazaspiro[5.5]undecan-3-one

Following the procedure described in Example 41a using 4-bromo-2,3-dimethylbenzenesulfonyl chloride provided the title product (196 mg, 53% yield). MS (ES)+ m/e 458.2 [M+H]⁺; ¹H NMR (400 MHz, CDCl₃) δ=7.67 (d, 1H), 7.57 (d, J=8.6 Hz, 1H), 4.09 (s, 2H), 3.60 (d, J=12.6 Hz, 2H), 3.18 (s, 2H), 3.01 (td, J=2.8, 12.5 Hz, 2H), 2.75 (tt, J=3.8, 7.4 Hz, 1H), 2.61 (s, 3H), 2.49 (s, 3H), 1.92 (dd, J=2.3, 14.4 Hz, 2H), 1.78-1.63 (m, 2H), 0.99-0.81 (m, 2H), 0.72-0.57 (m, 2H).

### c) 4-cyclopropyl-9-{[2,3-dimethyl-4-(7-quinolinyl)phenyl]sulfonyl}-1-oxa-4,9-diazaspiro[5.5]undecan-3-one

Following the procedure described in Example 42a using 9-[(4-bromo-2,3-dimethylphenyl)sulfonyl]-4-cyclopropyl-1-oxa-4,9-diazaspiro[5.5]undecan-3-one afforded the title product (40 mg, 45% yield). MS(ES)+ m/e 505.9 [M+H]⁺; ¹H NMR (400 MHz, DMSO-d₆) δ=8.97 (dd, 1H), 8.45 (d, J=1.0 Hz, 1H), 8.10 (d, J=8.3 Hz, 1H), 7.95 (s, 1H), 7.80 (d, J=8.1 Hz, 1H), 7.66-7.56 (m, 2H), 7.41 (d, J=8.1 Hz, 1H), 3.99 (s, 2H), 3.49 (d, J=11.6 Hz, 2H), 3.18 (s, 2H), 2.97-2.82 (m, 2H), 2.82-2.71 (m, 1H), 2.59 (s, 3H), 2.25 (s, 3H), 1.85 (d, J=13.1 Hz, 2H), 1.76-1.57 (m, 2H), 0.77-0.64 (m, 2H), 0.61 (m, J=4.0 Hz, 2H).

### Example 64

### 9-{[3-chloro-2-fluoro-4-(7-quinolinyl)phenyl]sulfonyl}-4-cyclopropyl-1-oxa-4,9-diazaspiro[5.5]undecan-3-one

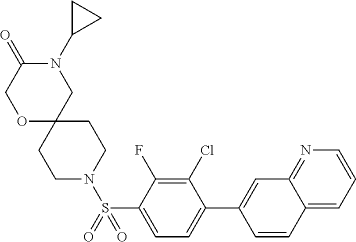 [SMILES: O=C1COC2(CCN(S(=O)(=O)c3ccc(-c4ccc5cccnc5c4)c(Cl)c3F)CC2)CN1C1CC1]

### a) 4-bromo-3-chloro-2-fluorobenzenesulfonyl chloride

To a 0° C. solution of 4-bromo-3-chloro-2-fluoroaniline (17.82 mmol) in acetonitrile (10 mL) was added tetrafluoroboric acid (48% in water, 26.7 mmol) and tert-butyl nitrite (26.7 mmol), which was stirred for 1 h. A suspension of copper(I) chloride (26.7 mmol) in acetic acid (30 mL) was stirred over an ice bath and was saturated with sulfur dioxide (178 mmol) passed through the mixture for 1 h. The intermediate diazonium salt was then added dropwise to the sulfur dioxide mixture, which was allowed to warm to room temperature, stirred for 1 h, and then quenched by adding to ice and stirring for 20 min. The mixture was extracted with diethyl ether (3×) and the combined extracts washed with water (2×) and brine, dried, and concentrated in vacuo to a gum. The residue was absorbed onto silica gel and washed through a pad of silica gel, eluting with hexanes and then with 30% ethyl acetate in hexanes to afford the title product (4.17 g, 76% yield) as red gum. MS(ES)+ m/e 306.8 [M+H]⁺; ¹H NMR (400 MHz, CDCl₃) δ=7.90-7.75 (m, 1H), 7.75-7.64 (m, 1H).

### b) 9-[(4-bromo-3-chloro-2-fluorophenyl)sulfonyl]-4-cyclopropyl-1-oxa-4,9-diazaspiro[5.5]undecan-3-one

Following the procedure described in Example 41a using 4-bromo-3-chloro-2-fluorobenzenesulfonyl chloride provided the title product (315 mg, 81% yield). MS (ES)+ m/e 482.8 [M+H]⁺; ¹H NMR (400 MHz, CDCl₃) δ=7.68-7.62 (m, 1H), 7.62-7.57 (m, 1H), 4.07 (s, 2H), 3.84-3.71 (m, 2H), 3.18 (s, 2H), 2.90 (t, J=12.3 Hz, 2H), 2.75 (t, J=3.8 Hz, 1H), 1.97 (dd, J=2.4, 14.3 Hz, 2H), 1.69 (td, J=4.7, 13.2 Hz, 2H), 0.92-0.84 (m, 2H), 0.70-0.63 (m, 2H).

### c) 9-{[3-chloro-2-fluoro-4-(7-quinolinyl)phenyl]sulfonyl}-4-cyclopropyl-1-oxa-4,9-diazaspiro[5.5]undecan-3-one

Following the procedure described in Example 42a using 9-[(4-bromo-3-chloro-2-fluorophenyl)sulfonyl]-4-cyclopropyl-1-oxa-4,9-diazaspiro[5.5]undecan-3-one afforded the title product (23 mg, 26% yield). MS(ES)+ m/e 531.0 [M+H]⁺; ¹H NMR (400 MHz, DMSO-d₆) δ=9.00 (dd, 1H), 8.52-8.43 (m, 1H), 8.15 (d, J=8.8 Hz, 2H), 7.86 (dd, J=6.9, 8.2 Hz, 1H), 7.77 (dd, J=1.8, 8.3 Hz, 1H), 7.71-7.60 (m, 2H), 3.97 (s, 2H), 3.61 (d, J=12.1 Hz, 2H), 3.18 (s, 2H), 2.85 (t, J=11.9 Hz, 2H), 2.75 (tt, J=3.9, 7.4 Hz, 1H), 1.88 (d, J=13.6 Hz, 2H), 1.80-1.60 (m, 2H), 0.77-0.65 (m, 2H), 0.64-0.52 (m, 2H).

### Example 65

### 9-{[3-chloro-2-fluoro-4-(1H-indol-6-yl)phenyl]sulfonyl}-4-cyclopropyl-1-oxa-4,9-diazaspiro[5.5]undecan-3-one

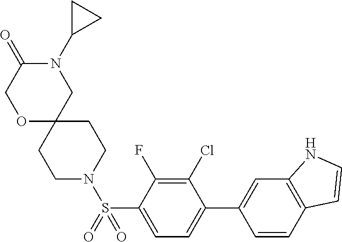 [SMILES: O=C1COC2(CCN(S(=O)(=O)c3ccc(-c4ccc5cc[nH]c5c4)c(Cl)c3F)CC2)CN1C1CC1]

a) Following the procedure described in Example 41b using 9-[(4-bromo-3-chloro-2-fluorophenyl)sulfonyl]-4-cyclopropyl-1-oxa-4,9-diazaspiro[5.5]undecan-3-one provided the title product (47 mg, 55% yield). MS(ES)+ m/e 519.3 [M+H]⁺; ¹H NMR (400 MHz, DMSO-d₆) δ=11.36 (br s, 1H), 7.85-7.75 (m, 1H), 7.67 (d, J=8.3 Hz, 1H), 7.57 (s, 1H), 7.53 (d, J=8.3 Hz, 1H), 7.51-7.45 (m, 1H), 7.14 (dd, J=1.6, 8.2 Hz, 1H), 6.52 (t, J=2.0 Hz, 1H), 3.96 (s, 2H), 3.66-3.53 (m, 2H), 3.17 (s, 2H), 2.87-2.77 (m, 2H), 2.77-2.70 (m, 1H), 1.87 (d, J=13.1 Hz, 2H), 1.78-1.62 (m, 2H), 0.74-0.64 (m, 2H), 0.64-0.56 (m, 2H).

### Example 66

### 4-cyclopropyl-9-{[3,5-difluoro-4-(1H-indol-6-yl)phenyl]sulfonyl}-1-oxa-4,9-diazaspiro[5.5]undecan-3-one

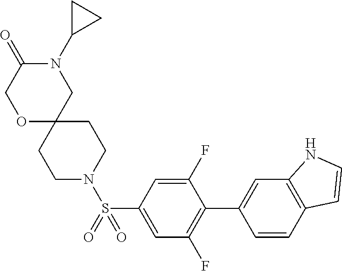 [SMILES: O=C1COC2(CCN(S(=O)(=O)c3cc(F)c(-c4ccc5cc[nH]c5c4)c(F)c3)CC2)CN1C1CC1]

### a) 4-bromo-3,5-difluorobenzenesulfonyl chloride

To a 0° C. solution of 4-bromo-3,5-difluoroaniline (19.23 mmol) in acetonitrile (10 mL) was added tetrafluoroboric acid (48% in water, 28.8 mmol) and tert-butyl nitrite (28.8 mmol) and the resulting mixture was stirred for 1 h. A suspension of copper(I) chloride (28.8 mmol) in acetic acid (30 mL) was stirred over an ice bath and was saturated with sulfur dioxide passed through the mixture for 1 h. The intermediate diazonium salt was then added dropwise to the sulfur dioxide mixture and was allowed to warm to room temperature, was stirred for 2 h, and was quenched by adding to ice and stirring for 5 min. The mixture was extracted with diethyl ether (2×) and the combined extracts were washed with water and brine, dried, and concentrated in vacuo to a pale orange gum that solidified upon standing to afford the title product (3.5 g, 62% yield; material was used without further purification). ¹H NMR (400 MHz, CDCl₃) δ=7.65-7.75 (m, 2H).

### b) 9-[(4-bromo-3,5-difluorophenyl)sulfonyl]-4-cyclopropyl-1-oxa-4,9-diazaspiro[5.5]undecan-3-one

To a mixture of 4-cyclopropyl-1-oxa-4,9-diazaspiro[5.5]undecan-3-one hydrochloride (0.811 mmol) in dichloromethane (15 mL) was added N,N-diisdopropylethylamine (2.432 mmol). The mixture was stirred for 5 min, then 4-bromo-3,5-difluorobenzenesulfonyl chloride (0.973 mmol) was added, giving a clear solution. After stirring for 2 h, the reaction was washed with 1M aq hydrochloric acid. The layers were separated and the aqueous layer was extracted with dichloromethane. The combined organics were dried over Na₂SO₄, filtered, and concentrated under reduced pressure. The residue was triturated with ethanol, collected and washed with ethanol and hexanes to provide the title product (210 mg, 56% yield) as an off-white solid. MS(ES)+m/e 466.0 [M+H]⁺.

### c) 4-cyclopropyl-9-{[3,5-difluoro-4-(1H-indol-6-yl)phenyl]sulfonyl}-1-oxa-4,9-diazaspiro[5.5]undecan-3-one

Following the procedure described in Example 41b using 9-[(4-bromo-3,5-difluorophenyl)sulfonyl]-4-cyclopropyl-1-oxa-4,9-diazaspiro[5.5]undecan-3-one provided the title product (31 mg, 29% yield). MS(ES)+ m/e 502.0 [M+H]⁺; ¹H NMR (400 MHz, DMSO-d₆) δ=11.36 (s, 1H), 7.68 (d, J=8.3 Hz, 1H), 7.64-7.55 (m, 3H), 7.52-7.44 (m, 1H), 7.13 (dd, J=1.4, 8.2 Hz, 1H), 6.52 (d, J=2.0 Hz, 1H), 3.93 (s, 2H), 3.56 (d, J=11.9 Hz, 2H), 3.16 (s, 2H), 2.74 (tt, J=3.9, 7.4 Hz, 1H), 2.70-2.59 (m, 2H), 1.97-1.79 (m, 2H), 1.78-1.56 (m, 2H), 0.75-0.65 (m, 2H), 0.65-0.50 (m, 2H).

### Example 67

### 4-cyclopropyl-9-{[3,5-difluoro-4-(7-quinolinyl)phenyl]sulfonyl}-1-oxa-4,9-diazaspiro[5.5]undecan-3-one

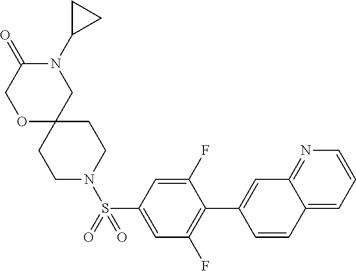 [SMILES: O=C1COC2(CCN(S(=O)(=O)c3cc(F)c(-c4ccc5cccnc5c4)c(F)c3)CC2)CN1C1CC1]

a) Following the procedure described in Example 42a using 9-[(4-bromo-3,5-difluorophenyl)sulfonyl]-4-cyclopropyl-1-oxa-4,9-diazaspiro[5.5]undecan-3-one afforded the title product (45 mg, 41% yield). MS(ES)+ m/e 514.4 [M+H]⁺; ¹H NMR (400 MHz, CDCl₃) δ=9.02 (dd, 1H), 8.30 (s, 1H), 8.28-8.20 (m, 1H), 7.98 (d, J=8.6 Hz, 1H), 7.68 (dd, J=1.6, 8.5 Hz, 1H), 7.57-7.43 (m, 3H), 4.07 (s, 2H), 3.83-3.68 (m, 2H), 3.19 (s, 2H), 2.86-2.69 (m, 3H), 2.01 (d, J=12.1 Hz, 2H), 1.74 (td, J=4.5, 13.1 Hz, 2H), 0.97-0.82 (m, 2H), 0.72-0.61 (m, 2H).

### Example 68

### 4-cyclopropyl-9-{[4-methyl-5-(7-quinolinyl)-2-thienyl]sulfonyl}-1-oxa-4,9-diazaspiro[5.5]undecan-3-one

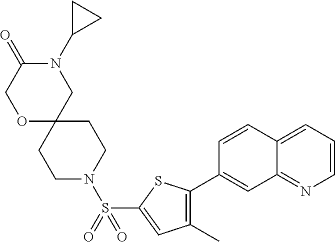 [SMILES: Cc1cc(S(=O)(=O)N2CCC3(CC2)CN(C2CC2)C(=O)CO3)sc1-c1ccc2cccnc2c1]

### a) 5-bromo-4-methyl-2-thiophenesulfonyl chloride

A 0° C. mixture of 2-bromo-3-methylthiophene (5.65 mmol) and chlorosulfonic acid (6.21 mmol) in chloroform (10 mL) was treated with phosphorus pentachloride (6.21 mmol). The mixture was allowed to warm to ambient temperature and stirred for 2 h. The reaction mixture was concentrated in vacuo and azeotroped with toluene (2×). The residue was added to a mixture of phosphorus pentachloride (6.21 mmol) in toluene and heated at 95° C. for 2 h. The mixture was cooled and diluted with ethyl acetate, washed with 5% aq citric acid, washed with saturated aq NaHCO₃ (2×), dried (MgSO₄), concentrated in vacuo, and then evaporated from chloroform to give the title product (1.4 g, 90% yield) as a brown gum (material was used without further purification). ¹H NMR (400 MHz, CDCl₃) δ=7.58 (s, 1H), 2.28 (s, 3H).

### b) 9-[(5-bromo-4-methyl-2-thienyl)sulfonyl]-4-cyclopropyl-1-oxa-4,9-diazaspiro[5.5]undecan-3-one

A mixture of 4-cyclopropyl-1-oxa-4,9-diazaspiro[5.5]undecan-3-one hydrochloride (4.05 mmol) and N,N-diisdopropylethylamine (12.16 mmol) in dichloromethane (10 mL) was treated with 5-bromo-4-methyl-2-thiophenesulfonyl chloride (4.05 mmol) and then stirred for 2 h. The reaction mixture was then washed sequentially with 1M aq HCl and saturated aq NaHCO₃, dried, and concentrated in vacuo to give the title product (1.25 g, 69% yield) as a tan solid from ethanol. MS(ES)+ m/e 449.0, 450.9 [M+H]⁺.

### c) 4-cyclopropyl-9-{[4-methyl-5-(7-quinolinyl)-2-thienyl]sulfonyl}-1-oxa-4,9-diazaspiro[5.5]undecan-3-one

A mixture of bis(triphenylphosphino)dichloropalladium(0) (0.014 mmol), 9-[(5-bromo-4-methyl-2-thienyl)sulfonyl]-4-cyclopropyl-1-oxa-4,9-diazaspiro[5.5]undecan-3-one (0.289 mmol) and 7-(4,4,5,5-tetramethyl-1,3,2-dioxaborolan-2-yl)quinoline (0.347 mmol) in 1,4-dioxane (5 mL) and water (2 mL) was sealed in a pressure vessel and heated at 120° C. for 3 h. The reaction mixture was allowed to cool, diluted with dichloromethane, and filtered through Celite to remove the palladium residue. The filtrate was diluted with water, the layers were separated, and the aqueous layer was extracted with dichloromethane (2×). The combined organic extracts were washed with brine, dried, and concentrated in vacuo. The residue was purified by reverse phase HPLC (20-80% acetonitrile/water+0.1% TFA). The product fractions were evaporated to low volume and partitioned between dichloromethane and dilute aq NaHCO₃, the aqueous layer was extracted with dichloromethane, and the combined organic extracts were dried and concentrated in vacuo to a foam. Trituration of the foam with diethyl ether gave the title product (45 mg, 31% yield). MS(ES)+ m/e 498.0 [M+H]⁺. ¹H NMR (400 MHz, CDCl₃) δ=9.00 (dd, 1H), 8.34-8.17 (m, 1H), 7.94 (d, J=8.6 Hz, 1H), 7.69 (dd, J=1.8, 8.3 Hz, 1H), 7.49 (dd, J=4.3, 8.3 Hz, 1H), 7.43 (s, 1H), 4.07 (s, 2H), 3.84-3.62 (m, 2H), 3.18 (s, 2H), 2.89-2.68 (m, 3H), 2.47 (s, 3H), 2.01 (d, J=12.6 Hz, 2H), 1.75 (td, J=4.5, 13.3 Hz, 2H), 0.96-0.82 (m, 2H), 0.75-0.58 (m, 2H).

### Example 69

### 4-cyclopropyl-9-{[2-(7-quinolinyl)-1,3-thiazol-5-yl]sulfonyl}-1-oxa-4,9-diazaspiro[5.5]undecan-3-one

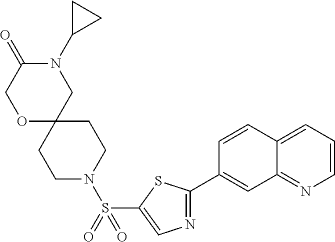 [SMILES: O=C1COC2(CCN(S(=O)(=O)c3cnc(-c4ccc5cccnc5c4)s3)CC2)CN1C1CC1]

### a) 9-[(2-chloro-1,3-thiazol-5-yl)sulfonyl]-4-cyclopropyl-1-oxa-4,9-diazaspiro[5.5]undecan-3-one

2-chloro-1,3-thiazole-5-sulfonyl chloride (4.13 mmol) was stirred in dichloromethane (50 mL) and N,N-diisopropylethylamine (10.32 mmol) was added, followed by 4-cyclopropyl-1-oxa-4,9-diazaspiro[5.5]undecan-3-one hydrochloride (4.13 mmol). The mixture was stirred for 24 h, washed with 1M aq hydrochloric acid and then saturated aq NaHCO₃, dried, and concentrated in vacuo. Trituration of the residue with ethanol gave an off-white solid of the title product (1.1 g, 68% yield) MS(ES)+ m/e 392.2 [M+H]⁺.

### b) 4-cyclopropyl-9-{[2-(7-quinolinyl)-1,3-thiazol-5-yl]sulfonyl}-1-oxa-4,9-diazaspiro[5.5]undecan-3-one

A mixture of 9-[(2-chloro-1,3-thiazol-5-yl)sulfonyl]-4-cyclopropyl-1-oxa-4,9-diazaspiro[5.5]undecan-3-one (0.383 mmol), 7-quinolineboronic acid (0.459 mmol), bis(triphenylphosphino)dichloropalladium(II) (0.029 mmol) and potassium carbonate (1.531 mmol) in a mixture of ethanol (5 mL) and water (2 mL) was sealed in a microwaveable vessel and heated at 150° C. for 20 min. The reaction mixture was cooled and partitioned between dichloromethane and 1M aq hydrochloric acid. The aqueous layer was extracted with dichloromethane and the combined organic extracts were washed with saturated aq NaHCO₃. The organic solution was loaded onto a 20 g silica gel pre-column and subjected to flash chromatography (0-5% methanol/dichloromethane) to give the title product that, when crystallized from ethanol, contains ˜5% w/w ethanol as a solvate (75 mg, 40% yield). MS(ES)+ m/e 485.0 [M+H]⁺. ¹H NMR (400 MHz, DMSO-d₆) δ=9.03 (dd, 1H), 8.66 (d, J=1.8 Hz, 1H), 8.57-8.42 (m, 2H), 8.30-8.22 (m, 1H), 8.22-8.14 (m, 1H), 7.66 (dd, J=4.3, 8.3 Hz, 1H), 3.91 (s, 2H), 3.55 (d, J=11.9 Hz, 2H), 3.16 (s, 2H), 2.83-2.62 (m, 3H), 1.99-1.81 (m, 2H), 1.81-1.65 (m, 2H), 0.76-0.63 (m, 2H), 0.63-0.52 (m, 2H).

### Example 70

### 4-cyclopropyl-9-{[2-fluoro-4-(1H-indol-5-yl)phenyl]sulfonyl}-1-oxa-4,9-diazaspiro[5.5]undecan-3-one

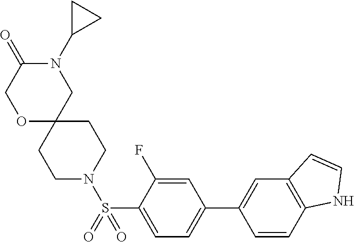 [SMILES: O=C1COC2(CCN(S(=O)(=O)c3ccc(-c4ccc5[nH]ccc5c4)cc3F)CC2)CN1C1CC1]

a) 9-[(4-Bromo-2-fluorophenyl)sulfonyl]-4-cyclopropyl-1-oxa-4,9-diazaspiro[5.5]undecan-3-one (0.179 mmol) and 1H-indol-5-ylboronic acid (0.179 mmol) were dissolved in dioxane (3 mL). To this were added PdCl₂(dppf)-CH₂CL₂ adduct (9.0 mmol) and 2N aq K₂CO₃ (0.30 mL). The vial was capped, flushed with nitrogen, and heated at 135° C. for 1 h. Analysis of the reaction mixture by LCMS showed the starting material gone and a large product peak present. The reaction was cooled and concentrated in vacuo. The residue was taken up in 1 mL of dimethylsulfoxide (DMSO) and 10 mL of dichloromethane and the solution filtered. The dichloromethane was then evaporated to leave a crude DMSO solution which was purified by reverse phase HPLC (5-95% acetonitrile/water containing 0.1% TFA). Product fractions were combined and the acetonitrile evaporated. The remaining water solution was treated with excess saturated aq sodium bicarbonate and the product was extracted into dichloromethane. The dichloromethane was dried with sodium sulfate, filtered, and concentrated in vacuo to yield the title product (32 mg, 36% yield) as a solid. LCMS (ES⁺) m/z 484.4 [M+H]⁺.

### Example 71

### 9-{[4-(1-benzofuran-5-yl)-2-fluorophenyl]sulfonyl}-4-cyclopropyl-1-oxa-4,9-diazaspiro[5.5]undecan-3-one

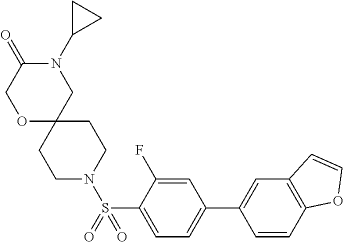 [SMILES: O=C1COC2(CCN(S(=O)(=O)c3ccc(-c4ccc5occc5c4)cc3F)CC2)CN1C1CC1]

a) Following the procedure described in Example 70a using 5-(4,4,5,5-tetramethyl-1,3,2-dioxaborolan-2-yl)-1-benzofuran provided the title product (55 mg, 62%) as a solid. LCMS (ES⁺) m/z 485 [M+H]⁺.

### Example 72

### 4-cyclopropyl-9-{[2-fluoro-4-(1H-indazol-5-yl)phenyl]sulfonyl}-1-oxa-4,9-diazaspiro[5.5]undecan-3-one

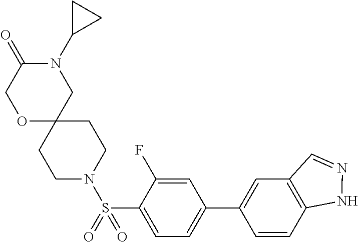 [SMILES: O=C1COC2(CCN(S(=O)(=O)c3ccc(-c4ccc5[nH]ncc5c4)cc3F)CC2)CN1C1CC1]

a) Following the procedure described in Example 70a using indazol-5-ylboronic acid provided the title product (16 mg, 18%) as a solid. LCMS (ES⁺) m/z 485.1 [M+H]⁺.

### Example 73

### 4-cyclopropyl-9-{[2-fluoro-4-(6-quinolinyl)phenyl]sulfonyl}-1-oxa-4,9-diazaspiro[5.5]undecan-3-one

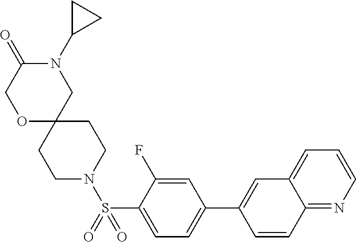 [SMILES: O=C1COC2(CCN(S(=O)(=O)c3ccc(-c4ccc5ncccc5c4)cc3F)CC2)CN1C1CC1]

a) Following the procedure described in Example 70a using 6-(4,4,5,5-tetramethyl-1,3,2-dioxaborolan-2-yl)quinoline provided the title product (52 mg, 57%) as a solid. LCMS (ES⁺) m/z 496.1 [M+H]⁺.

### Example 74

### 4-cyclopropyl-9-[(2′,4′-dichloro-3-fluoro-4-biphenylyl)sulfonyl]-1-oxa-4,9-diazaspiro[5.5]undecan-3-one

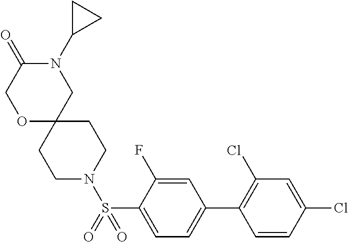 [SMILES: O=C1COC2(CCN(S(=O)(=O)c3ccc(-c4ccc(Cl)cc4Cl)cc3F)CC2)CN1C1CC1]

a) Following the procedure described in Example 70a using (2,4-dichlorophenyl)boronic acid provided the title product (25 mg, 27%) as a solid. LCMS (ES⁺) m/z 513.0 [M+H]⁺.

### Example 75

### 4-cyclopropyl-9-{[3-fluoro-4′-(methyloxy)-4-biphenylyl]sulfonyl}-1-oxa-4,9-diazaspiro[5.5]undecan-3-one

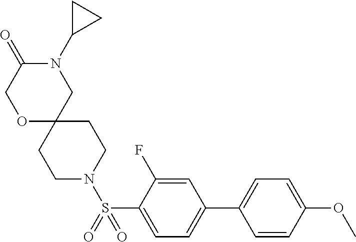 [SMILES: COc1ccc(-c2ccc(S(=O)(=O)N3CCC4(CC3)CN(C3CC3)C(=O)CO4)c(F)c2)cc1]

a) Following the procedure described in Example 70a using (4-methoxyphenyl)boronic acid provided the title product (32 mg, 36%) as a solid. LCMS (ES⁺) m/z 475.0 [M+H]⁺.

### Example 76

### 9-{[4-(1,3-benzothiazol-5-yl)-2-fluorophenyl]sulfonyl}-4-cyclopropyl-1-oxa-4,9-diazaspiro[5.5]undecan-3-one

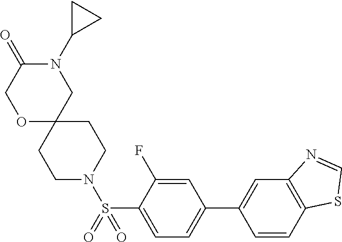 [SMILES: O=C1COC2(CCN(S(=O)(=O)c3ccc(-c4ccc5scnc5c4)cc3F)CC2)CN1C1CC1]

a) Following the procedure described in Example 70a using 5-(4,4,5,5-tetramethyl-1,3,2-dioxaborolan-2-yl)-1,3-benzothiazole provided the title product (45 mg, 49%) as a solid. LCMS (ES⁺) m/z 502.0 [M+H]⁺.

### Example 77

### {4′-[(4-cyclopropyl-3-oxo-1-oxa-4,9-diazaspiro[5.5]undec-9-yl)sulfonyl]-3′-fluoro-4-hydroxy-3-biphenyl}-4 formamide

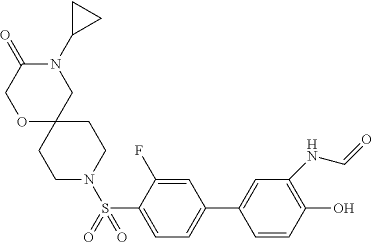 [SMILES: O=CNc1cc(-c2ccc(S(=O)(=O)N3CCC4(CC3)CN(C3CC3)C(=O)CO4)c(F)c2)ccc1O]

a) 9-[(4-Bromo-2-fluorophenyl)sulfonyl]-4-cyclopropyl-1-oxa-4,9-diazaspiro[5.5]undecan-3-one (0.179 mmol) and 5-(4,4,5,5-tetramethyl-1,3,2-dioxaborolan-2-yl)-1,3-benzoxazole (0.232 mmol) were dissolved in dioxane (3 mL). To this were added PdCl₂(dppf)-CH₂CL₂ adduct (9.0 μmol) and 2N aq K₂CO₃ (0.30 mL). The vial was capped, flushed with nitrogen, and heated at 140° C. for 1.1 h. Analysis of the reaction mixture by LCMS showed the starting material gone and a peak corresponding to the benzoxazole product. The reaction was cooled and 1N aq HCl was added to adjust the pH to ˜7.7. After concentrating the reaction in vacuo, the residue was taken up in 1.5 mL of dimethylsulfoxide (DMSO) and 10 mL of dichloromethane and the solution filtered. The dichloromethane was then evaporated to leave a crude DMSO solution which was purified by reverse phase HPLC (5-95% acetonitrile/water containing 0.1% TFA). Product fractions were combined and the acetonitrile evaporated. The remaining water solution was extracted with dichloromethane and concentrated in vacuo to yield the title product (39 mg, 42% yield) as a solid. LCMS (ES⁺) m/z 503.9 [M+H]⁺.

### Example 78

### 4-cyclopropyl-9-{[2-fluoro-4-(5-quinolinyl)phenyl]sulfonyl}-1-oxa-4,9-diazaspiro[5.5]undecan-3-one

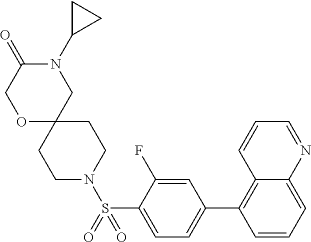 [SMILES: O=C1COC2(CCN(S(=O)(=O)c3ccc(-c4cccc5ncccc45)cc3F)CC2)CN1C1CC1]

a) Following the procedure described in Example 70a using 5-quinolinylboronic acid provided the title product (48 mg, 53%) as a solid. LCMS (ES⁺) m/z 496.0 [M+H]⁺.

### Example 79

### 4-cyclopropyl-9-{[3-fluoro-4′-(1H-pyrazol-1-yl)-4-biphenylyl]sulfonyl}-1-oxa-4,9-diazaspiro[5.5]undecan-3-one

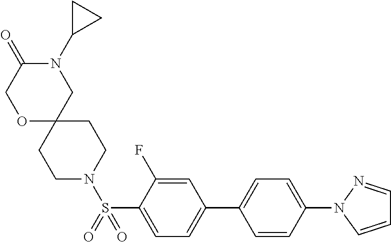 [SMILES: O=C1COC2(CCN(S(=O)(=O)c3ccc(-c4ccc(-n5cccn5)cc4)cc3F)CC2)CN1C1CC1]

a) Following the procedure described in Example 70a using [4-(1H-pyrazol-1-yl)phenyl]boronic acid provided the title product (18 mg, 21%) as a solid. LCMS (ES⁺) m/z 511.2 [M+H]⁺.

### Example 80

### 9-{[4-(1,3-benzoxazol-5-yl)-2-fluorophenyl]sulfonyl}-4-cyclopropyl-1-oxa-4,9-diazaspiro[5.5]undecan-3-one

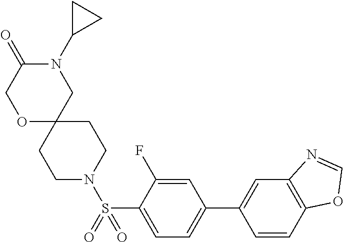 [SMILES: O=C1COC2(CCN(S(=O)(=O)c3ccc(-c4ccc5ocnc5c4)cc3F)CC2)CN1C1CC1]

a) Following the procedure described in Example 77a using except purification of the residue by reverse phase HPLC (5-90% acetonitrile/water with 0.1% ammonium hydroxide) provided the title product (20 mg, 22%) as a solid. LCMS (ES⁺) m/z 486.2 [M+H]⁺.

### Example 81

### 4-cyclopropyl-9-{[3′-(1H-pyrazol-5-yl)-4-biphenylyl]sulfonyl}-1-oxa-4,9-diazaspiro[5.5]undecan-3-one

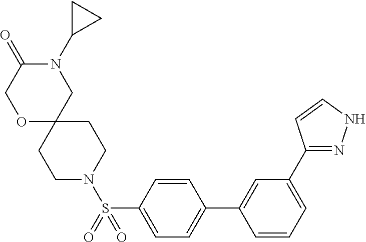 [SMILES: O=C1COC2(CCN(S(=O)(=O)c3ccc(-c4cccc(-c5cc[nH]n5)c4)cc3)CC2)CN1C1CC1]

a) Following the procedure described in Example 70a using [3-(1H-pyrazol-5-yl)phenyl]boronic acid and 9-[(4-bromophenyl)sulfonyl]-4-cyclopropyl-1-oxa-4,9-diazaspiro[5.5]undecan-3-one provided the title product (38 mg, 41%) as a solid. LCMS (ES⁺) m/z 493.0 [M+H]⁺.

### Example 82

### 4-cyclopropyl-9-{[4′-(1H-pyrazol-5-yl)-4-biphenylyl]sulfonyl}-1-oxa-4,9-diazaspiro[5.5]undecan-3-one

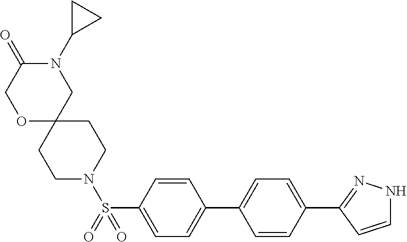 [SMILES: O=C1COC2(CCN(S(=O)(=O)c3ccc(-c4ccc(-c5cc[nH]n5)cc4)cc3)CC2)CN1C1CC1]

a) Following the procedure described in Example 81a using [4-(1H-pyrazol-5-yl)phenyl]boronic acid provided the title product (35 mg, 36%) as a solid. LCMS (ES⁺) m/z 493.1 [M+H]⁺.

### Example 83

### 4-cyclopropyl-9-{[4-(7-isoquinolinyl)phenyl]sulfonyl}-1-oxa-4,9-diazaspiro[5.5]undecan-3-one

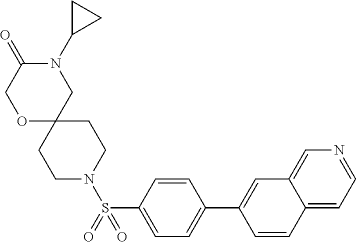 [SMILES: O=C1COC2(CCN(S(=O)(=O)c3ccc(-c4ccc5ccncc5c4)cc3)CC2)CN1C1CC1]

a) Following the procedure described in Example 70a using 7-(4,4,5,5-tetramethyl-1,3,2-dioxaborolan-2-yl)isoquinoline and 9-[(4-bromophenyl)sulfonyl]-4-cyclopropyl-1-oxa-4,9-diazaspiro[5.5]undecan-3-one provided the title product (10 mg, 18%) as a solid. LCMS (ES⁺) m/z 477.8 [M+H]⁺.

### Example 84

### 4-cyclopropyl-9-{[4-(7-quinazolinyl)phenyl]sulfonyl}-1-oxa-4,9-diazaspiro[5.5]undecan-3-one

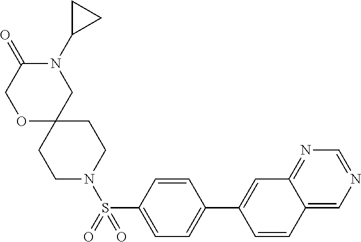 [SMILES: O=C1COC2(CCN(S(=O)(=O)c3ccc(-c4ccc5cncnc5c4)cc3)CC2)CN1C1CC1]

a) Following the procedure described in Example 70a using 7-(4,4,5,5-tetramethyl-1,3,2-dioxaborolan-2-yl)quinazoline and 9-[(4-bromophenyl)sulfonyl]-4-cyclopropyl-1-oxa-4,9-diazaspiro[5.5]undecan-3-one provided the title product (45 mg, 39%) as a solid. LCMS (ES⁺) m/z 479.0 [M+H]⁺.

### Example 85

### N-{4′-[(4-cyclopropyl-3-oxo-1-oxa-4,9-diazaspiro[5.5]undec-9-yl)sulfonyl]-3-biphenylyl}-N,N-dimethylsulfamide

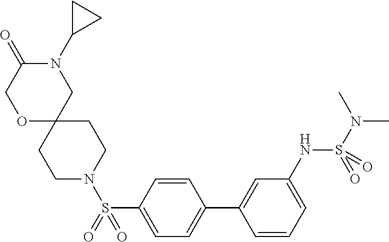 [SMILES: CN(C)S(=O)(=O)Nc1cccc(-c2ccc(S(=O)(=O)N3CCC4(CC3)CN(C3CC3)C(=O)CO4)cc2)c1]

a) Following the procedure described in Example 70a using (3-{[(dimethylamino)sulfonyl]amino}phenyl)boronic acid and 9-[(4-bromophenyl)sulfonyl]-4-cyclopropyl-1-oxa-4,9-diazaspiro[5.5]undecan-3-one provided the title product (45 mg, 34%) as a solid. LCMS (ES⁺) m/z 549.0 [M+H]⁺.

### Example 86

### 4-cyclopropyl-9-{[4-(6-isoquinolinyl)phenyl]sulfonyl}-1-oxa-4,9-diazaspiro[5.5]undecan-3-one

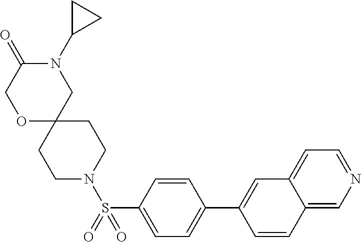 [SMILES: O=C1COC2(CCN(S(=O)(=O)c3ccc(-c4ccc5cnccc5c4)cc3)CC2)CN1C1CC1]

a) Following the procedure described in Example 70a using 6-(4,4,5,5-tetramethyl-1,3,2-dioxaborolan-2-yl)isoquinoline and 9-[(4-bromophenyl)sulfonyl]-4-cyclopropyl-1-oxa-4,9-diazaspiro[5.5]undecan-3-one provided the title product (30 mg, 26%) as a solid. LCMS (ES⁺) m/z 477.9 [M+H]⁺.

### Example 87

### 4-cyclopropyl-9-{[4-(3-quinolinyl)phenyl]sulfonyl}-1-oxa-4,9-diazaspiro[5.5]undecan-3-one

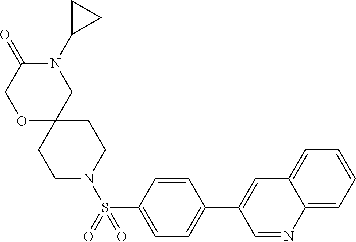 [SMILES: O=C1COC2(CCN(S(=O)(=O)c3ccc(-c4cnc5ccccc5c4)cc3)CC2)CN1C1CC1]

a) Following the procedure described in Example 70a using 3-quinolinylboronic acid and 9-[(4-bromophenyl)sulfonyl]-4-cyclopropyl-1-oxa-4,9-diazaspiro[5.5]undecan-3-one provided the title product (24 mg, 26%) as a solid. LCMS (ES⁺) m/z 477.8 [M+H]⁺.

### Example 88

### 4-cyclopropyl-9-{[4-(2-naphthalenyl)phenyl]sulfonyl}-1-oxa-4,9-diazaspiro[5.5]undecan-3-one

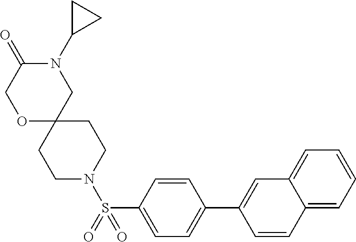 [SMILES: O=C1COC2(CCN(S(=O)(=O)c3ccc(-c4ccc5ccccc5c4)cc3)CC2)CN1C1CC1]

a) Following the procedure described in Example 70a using 4,4,5,5-tetramethyl-2-(2-naphthalenyl)-1,3,2-dioxaborolane and 9-[(4-bromophenyl)sulfonyl]-4-cyclopropyl-1-oxa-4,9-diazaspiro[5.5]undecan-3-one provided the title product (67 mg, 50%) as a solid. LCMS (ES⁺) m/z 477.0 [M+H]⁺.

### Example 89

### 4-cyclopropyl-9-{[4-(2-methyl-1,3-benzothiazol-5-yl)phenyl]sulfonyl}-1-oxa-4,9-diazaspiro[5.5]undecan-3-one

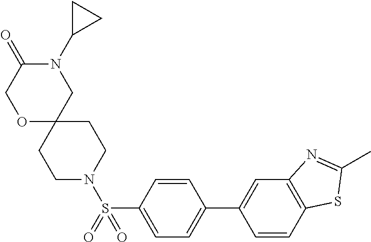 [SMILES: Cc1nc2cc(-c3ccc(S(=O)(=O)N4CCC5(CC4)CN(C4CC4)C(=O)CO5)cc3)ccc2s1]

### a) 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one

To a glass vial were added 9-[(4-bromophenyl)sulfonyl]-4-cyclopropyl-1-oxa-4,9-diazaspiro[5.5]undecan-3-one (2.80 mmol), 4,4,4′,4′,5,5,5′,5′-octamethyl-2,2′-bi-1,3,2-dioxaborolane (3.35 mmol), PdCl₂(dppf)-CH₂Cl₂ adduct (0.140 mmol), potassium acetate (6.99 mmol) and 1,4-dioxane (30 mL). The vial was flushed with nitrogen, capped, stirred, and heated to 140° C. for 1 h. Analysis by LCMS indicated the reaction was completed. The reaction was cooled and 30 mL of dichloromethane was added and the insoluble salts were filtered off. The solution was absorbed onto silica gel, which was then subjected to flash chromatography (40-100% ethyl acetate/hexanes) to give the title compound (960 mg, 72% yield). LCMS (ES⁺) m/z 477.0 [M+H]⁺.

### b) 4-cyclopropyl-9-{[4-(2-methyl-1,3-benzothiazol-5-yl)phenyl]sulfonyl}-1-oxa-4,9-diazaspiro[5.5]undecan-3-one

Following the procedure described in Example 70a using 5-bromo-2-methyl-1,3-benzothiazole and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (70 mg, 45%) as a solid. LCMS (ES⁺) m/z 497.9 [M+H]⁺.

### Example 90

### 4-cyclopropyl-9-({4-[4-(ethyloxy)-7-quinolinyl]phenyl}sulfonyl)-1-oxa-4,9-diazaspiro[5.5]undecan-3-one

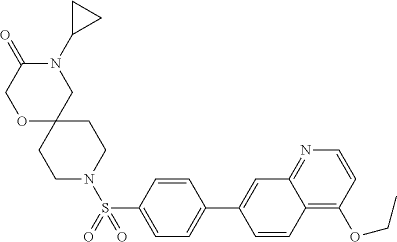 [SMILES: CCOc1ccnc2cc(-c3ccc(S(=O)(=O)N4CCC5(CC4)CN(C4CC4)C(=O)CO5)cc3)ccc12]

### a) 7-bromo-4-ethoxyquinoline

Into a microwaveable vial were placed 7-bromo-4-chloroquinoline (1.237 mmol), sodium ethoxide (4.95 mmol), dioxane (2 mL), and ethanol (2 mL). The vial was capped and heated to 120° C. for 0.5 h. Analysis by LCMS showed the formation of product. The reaction mixture was concentrated in vacuo, and water and dichloromethane were added to the residue. The product was extracted into the dichloromethane, which was separated from the aqueous, dried with sodium sulfate, and concentrated in vacuo. Purification of the residue by flash chromatography (35-75% ethyl acetate in hexanes) gave the title product (72 mg, 20%). LCMS (ES⁺) m/z 253.7 [M+H]⁺.

### b) 4-cyclopropyl-9-({4-[4-(ethyloxy)-7-quinolinyl]phenyl}sulfonyl)-1-oxa-4,9-diazaspiro[5.5]undecan-3-one

Following the procedure described in Example 70a using 7-bromo-4-ethoxyquinoline and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (45 mg, 30%) as a solid. LCMS (ES⁺) m/z 522.0 [M+H]⁺.

### Example 91

### 4-cyclopropyl-9-({4-[4-(methyloxy)-7-quinolinyl]phenyl}sulfonyl)-1-oxa-4,9-diazaspiro[5.5]undecan-3-one

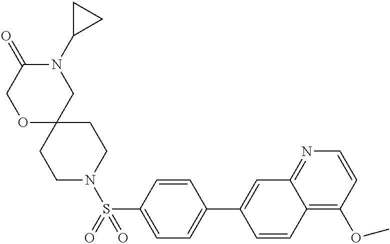 [SMILES: COc1ccnc2cc(-c3ccc(S(=O)(=O)N4CCC5(CC4)CN(C4CC4)C(=O)CO5)cc3)ccc12]

### a) 7-bromo-4-methoxyquinoline

Into a microwaveable vial were placed 7-bromo-4-chloroquinoline (1.237 mmol), sodium methoxide (3.71 mmol), dioxane (2 mL), and methanol (2 mL). The vial was capped and heated to 90° C. for 1 h. Analysis by LCMS showed the formation of product. The reaction mixture was concentrated in vacuo, and water and dichloromethane were added to the residue. The product was extracted into the dichloromethane, which was separated from the aqueous, dried with sodium sulfate, and concentrated in vacuo to provide the title product (250 mg, 78%). LCMS (ES⁺) m/z 237.8, 239.8 [M+H]⁺.

### b) 4-cyclopropyl-9-({4-[4-(ethyloxy)-7-quinolinyl]phenyl}sulfonyl)-1-oxa-4,9-diazaspiro[5.5]undecan-3-one

Following the procedure described in Example 70a using 7-bromo-4-methoxyquinoline and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (30 mg, 26%) as a solid. LCMS (ES⁺) m/z 508.0 [M+H]⁺.

### Example 92

### 4-cyclopropyl-9-[(4-imidazo[1,2-a]pyridin-7-ylphenyl)sulfonyl]-1-oxa-4,9-diazaspiro[5.5]undecan-3-one

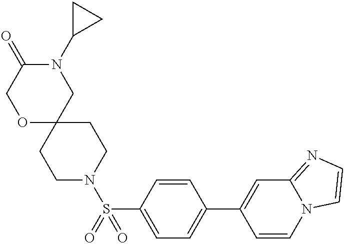 [SMILES: O=C1COC2(CCN(S(=O)(=O)c3ccc(-c4ccn5ccnc5c4)cc3)CC2)CN1C1CC1]

a) Following the procedure described in Example 70a using 7-bromoimidazo[1,2-a]pyridine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (60 mg, 55%) as a solid. LCMS (ES⁺) m/z 467.0 [M+H]⁺.

### Example 93

### 9-{[4-(3-amino-1H-indazol-6-yl)phenyl]sulfonyl}-4-cyclopropyl-1-oxa-4,9-diazaspiro[5.5]undecan-3-one

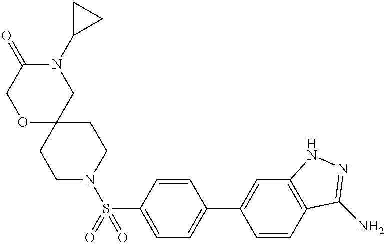 [SMILES: Nc1n[nH]c2cc(-c3ccc(S(=O)(=O)N4CCC5(CC4)CN(C4CC4)C(=O)CO5)cc3)ccc12]

a) Following the procedure described in Example 70a using 6-bromo-1H-indazol-3-amine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (46 mg, 41%) as a solid. LCMS (ES⁺) m/z 481.8 [M+H]⁺.

### Example 94

### 9-{[4-(3-amino-1H-indazol-5-yl)phenyl]sulfonyl}-4-cyclopropyl-1-oxa-4,9-diazaspiro[5.5]undecan-3-one

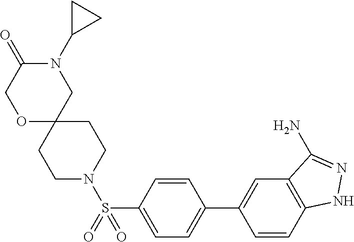 [SMILES: Nc1n[nH]c2ccc(-c3ccc(S(=O)(=O)N4CCC5(CC4)CN(C4CC4)C(=O)CO5)cc3)cc12]

a) Following the procedure described in Example 70a using 5-bromo-1H-indazol-3-amine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (30 mg, 25%) as a solid. LCMS (ES⁺) m/z 481.8 [M+H]⁺.

### Example 95

### 9-{[4-(2-amino-4-pyridinyl)phenyl]sulfonyl}-4-cyclopropyl-1-oxa-4,9-diazaspiro[5.5]undecan-3-one

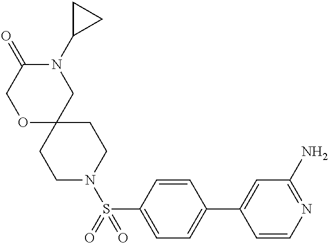 [SMILES: Nc1cc(-c2ccc(S(=O)(=O)N3CCC4(CC3)CN(C3CC3)C(=O)CO4)cc2)ccn1]

a) Following the procedure described in Example 70a using 4-bromo-2-pyridinamine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (44 mg, 25%) as a solid. LCMS (ES⁺) m/z 443.1 [M+H]⁺.

### Example 96

### 4-cyclopropyl-9-{[4-(4-quinolinyl)phenyl]sulfonyl}-1-oxa-4,9-diazaspiro[5.5]undecan-3-one

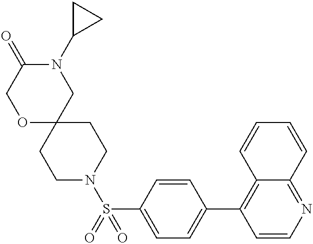 [SMILES: O=C1COC2(CCN(S(=O)(=O)c3ccc(-c4ccnc5ccccc45)cc3)CC2)CN1C1CC1]

a) Following the procedure described in Example 70a using 4-bromoquinoline and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (67 mg, 55%) as a solid. LCMS (ES⁺) m/z 477.9 [M+H]⁺.

### Example 97

### 4-cyclopropyl-9-{[4-(1-methyl-1H-indol-6-yl)phenyl]sulfonyl}-1-oxa-4,9-diazaspiro[5.5]undecan-3-one

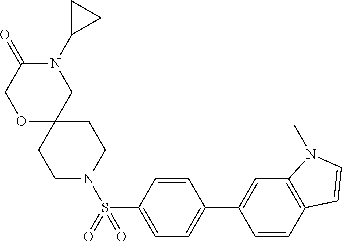 [SMILES: Cn1ccc2ccc(-c3ccc(S(=O)(=O)N4CCC5(CC4)CN(C4CC4)C(=O)CO5)cc3)cc21]

a) Following the procedure described in Example 70a using 6-bromo-1-methyl-1H-indole and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (36 mg, 28%) as a solid. LCMS (ES⁺) m/z 480.0 [M+H]⁺.

### Example 98

### 4-cyclopropyl-9-{[4-(1-methyl-1H-indol-4-yl)phenyl]sulfonyl}-1-oxa-4,9-diazaspiro[5.5]undecan-3-one

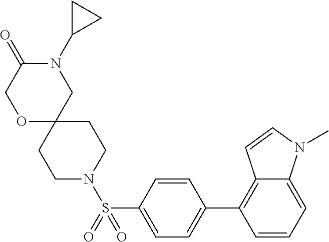 [SMILES: Cn1ccc2c(-c3ccc(S(=O)(=O)N4CCC5(CC4)CN(C4CC4)C(=O)CO5)cc3)cccc21]

a) Following the procedure described in Example 70a using 4-bromo-1-methyl-1H-indole and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (56 mg, 45%) as a solid. LCMS (ES⁺) m/z 480.0 [M+H]⁺.

### Example 99

### 4-cyclopropyl-9-({4-[4-(methylamino)-7-quinolinyl]phenyl}sulfonyl)-1-oxa-4,9-diazaspiro[5.5]undecan-3-one

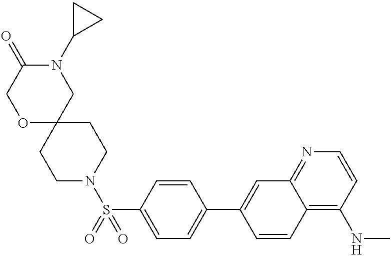 [SMILES: CNc1ccnc2cc(-c3ccc(S(=O)(=O)N4CCC5(CC4)CN(C4CC4)C(=O)CO5)cc3)ccc12]

### a) 7-bromo-N-methyl-4-quinolinamine

Into a microwaveable vial were placed 7-bromo-4-chloroquinoline (1.237 mmol), N-methyl-2-pyrrolidone (1.0 mL), and methylamine in tetrahydrofuran (10 equiv). The vial was capped and heated at 100° C. for 18 h. Excess water was added to the cooled reaction, which was then filtered to remove the insoluble crude product, rinsing with water. The solid was purified by flash chromatography (ethyl acetate/hexanes) to give the title prod (59 mg, 60%) LCMS (ES⁺) m/z 238.9 [M+H]⁺.

### b) 4-cyclopropyl-9-({4-[4-(methylamino)-7-quinolinyl]phenyl}sulfonyl)-1-oxa-4,9-diazaspiro[5.5]undecan-3-one

Following the procedure described in Example 70a using 7-bromo-N-methyl-4-quinolinamine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (35 mg, 27%) as a solid. LCMS (ES⁺) m/z 507.0 [M+H]⁺.

### Example 100

### 4-cyclopropyl-9-{[4-(4-methyl-7-quinolinyl)phenyl]sulfonyl}-1-oxa-4,9-diazaspiro[5.5]undecan-3-one

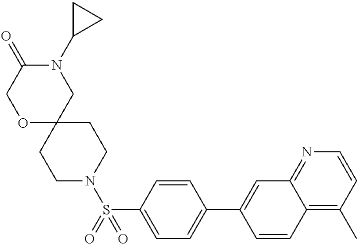 [SMILES: Cc1ccnc2cc(-c3ccc(S(=O)(=O)N4CCC5(CC4)CN(C4CC4)C(=O)CO5)cc3)ccc12]

### a) 7-bromo-4-methylquinoline

Into a microwaveable vial were placed 7-bromo-4-chloroquinoline (1.237 mmol) and 6 mL of dry tetrahydrofuran. The solution was cooled to 0° C. and tetrahydrate Manganese(II) chloride (0.53 mmol) and methylmagnesium chloride (3.71 mmol of a 1M solution in tetrahydrofuran) were added. The vial was capped and the reaction was stirred for 2 h at 0° C. and then 20 h at room temperature. Analysis by LCMS showed the formation of desired product. Excess saturated aq ammonium chloride was added to the cooled reaction, which was then extracted with ethyl acetate (3×). Purification of the residue by flash chromatography (0-15% ethyl acetate/hexanes) gave the title prod (61 mg, 22%) LCMS (ES⁺) m/z 222.2, 224.0 [M+H]⁺.

### b) 4-cyclopropyl-9-{[4-(4-methyl-7-quinolinyl)phenyl]sulfonyl}-1-oxa-4,9-diazaspiro[5.5]undecan-3-one

Following the procedure described in Example 70a using 7-bromo-4-methylquinoline and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (64 mg, 50%) as a solid. LCMS (ES⁺) m/z 492.3 [M+H]⁺.

### Example 101

### 4-cyclopropyl-9-[(4-imidazo[1,2-a]pyridin-6-ylphenyl)sulfonyl]-1-oxa-4,9-diazaspiro[5.5]undecan-3-one

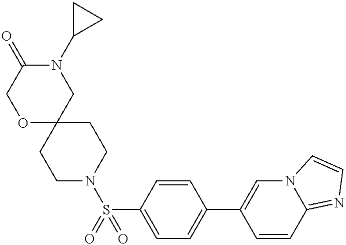 [SMILES: O=C1COC2(CCN(S(=O)(=O)c3ccc(-c4ccc5nccn5c4)cc3)CC2)CN1C1CC1]

a) Following the procedure described in Example 70a using 6-bromoimidazo[1,2-a]pyridine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (18 mg, 15%) as a solid. LCMS (ES⁺) m/z 467.1 [M+H]⁺.

### Example 102

### 9-{[4-(7-cinnolinyl)phenyl]sulfonyl}-4-cyclopropyl-1-oxa-4,9-diazaspiro[5.5]undecan-3-one

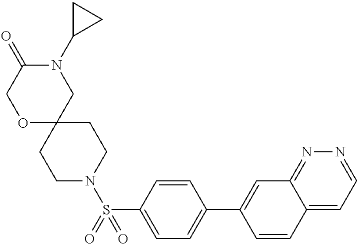 [SMILES: O=C1COC2(CCN(S(=O)(=O)c3ccc(-c4ccc5ccnnc5c4)cc3)CC2)CN1C1CC1]

a) Following the procedure described in Example 70a using 7-bromocinnoline and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (27 mg, 15%) as a solid. LCMS (ES⁺) m/z 479.0 [M+H]⁺.

### Example 103

### 4-cyclopropyl-9-[(4-imidazo[1,2-b]pyridazin-6-ylphenyl)sulfonyl]-1-oxa-4,9-diazaspiro[5.5]undecan-3-one

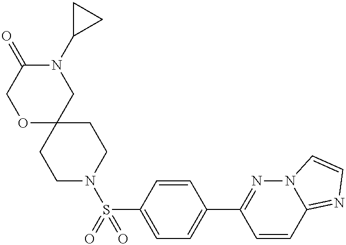 [SMILES: O=C1COC2(CCN(S(=O)(=O)c3ccc(-c4ccc5nccn5n4)cc3)CC2)CN1C1CC1]

### a) 6-bromoimidazo[1,2-b]pyridazine

6-Bromo-3-pyridazinamine (12.8 mmol) was dissolved in isopropanol (25 mL). To this was added chloroacetaldehyde (50% in water, 3.0 mL, 1.5 eq) and sodium bicarbonate (25.6 mmol). The reaction was stirred at 70° C. for 10 h. Analysis by LCMS indicated the reaction was complete. The solvents were evaporated and the residue was extracted with hot ethyl acetate. The ethyl acetate was cooled and filtered through paper. Evaporation of the ethyl acetate gave the crude product, which was purified by silica gel chromatography (75% ethyl acetate/hexanes) to obtain the title product (1.55 g, 61%) LCMS (ES⁺) m/z 197.7 [M+H]⁺.

### b) 4-cyclopropyl-9-[(4-imidazo[1,2-b]pyridazin-6-ylphenyl)sulfonyl]-1-oxa-4,9-diazaspiro[5.5]undecan-3-one

Following the procedure described in Example 70a using 6-bromoimidazo[1,2-b]pyridazine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (28 mg, 23%) as a solid. LCMS (ES⁺) m/z 468.1 [M+H]⁺.

### Example 104

### 9-{[4-(3-amino-1-methyl-1H-indazol-5-yl)phenyl]sulfonyl}-4-cyclopropyl-1-oxa-4,9-diazaspiro[5.5]undecan-3-one

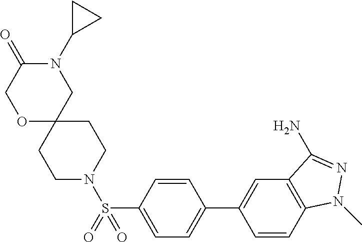 [SMILES: Cn1nc(N)c2cc(-c3ccc(S(=O)(=O)N4CCC5(CC4)CN(C4CC4)C(=O)CO5)cc3)ccc21]

a) Following the procedure described in Example 70a using 5-bromo-1-methyl-1H-indazol-3-amine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (20 mg, 15%) as a solid. LCMS (ES⁺) m/z 496.3 [M+H]⁺.

### Example 105

### N-(5-{4-[(4-cyclopropyl-3-oxo-1-oxa-4,9-diazaspiro[5.5]undec-9-yl)sulfonyl]phenyl}-1-methyl-1H-indazol-3-yl)methanesulfonamide

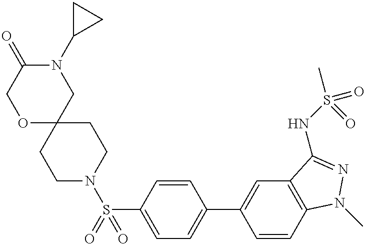 [SMILES: Cn1nc(NS(C)(=O)=O)c2cc(-c3ccc(S(=O)(=O)N4CCC5(CC4)CN(C4CC4)C(=O)CO5)cc3)ccc21]

### a) N-(5-bromo-1-methyl-1H-indazol-3-yl)methanesulfonamide

5-Bromo-1-methyl-1H-indazol-3-amine (1.55 mmol) was dissolved in pyridine (2 mL) and to this was added dropwise methanesulfonyl chloride (3.10 mmol). The reaction was stirred at room temperature for 1 h, at which point additional methanesulfonyl chloride (1.94 mmol) was added. After 2 h, analysis by LCMS indicated the reaction was complete. The reaction was concentrated in vacuo and the residue was taken up in dichloromethane and washed with water (2×) and 0.5N aq HCl, dried over sodium sulfate, and concentrated in vacuo to yield the title product (400 mg, 83%) LCMS (ES⁺) m/z 304.2 [M+H]⁺.

### b) N-(5-{4-[(4-cyclopropyl-3-oxo-1-oxa-4,9-diazaspiro[5.5]undec-9-yl)sulfonyl]phenyl}-1-methyl-1H-indazol-3-yl)methanesulfonamide

Following the procedure described in Example 70a using N-(5-bromo-1-methyl-1H-indazol-3-yl)methanesulfonamide and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (26 mg, 14%) as a solid. LCMS (ES⁺) m/z 574.2 [M+H]⁺.

### Example 106

### 9-{[4-(3-amino-1-methyl-1H-indazol-6-yl)phenyl]sulfonyl}-4-cyclopropyl-1-oxa-4,9-diazaspiro[5.5]undecan-3-one

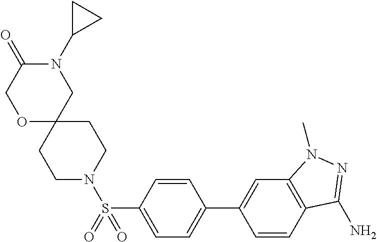 [SMILES: Cn1nc(N)c2ccc(-c3ccc(S(=O)(=O)N4CCC5(CC4)CN(C4CC4)C(=O)CO5)cc3)cc21]

a) Following the procedure described in Example 70a using 6-bromo-1-methyl-1H-indazol-3-amine and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (48 mg, 38%) as a solid. LCMS (ES⁺) m/z 496.3 [M+H]⁺.

### Example 107

### N-(6-{4-[(4-cyclopropyl-3-oxo-1-oxa-4,9-diazaspiro[5.5]undec-9-yl)sulfonyl]phenyl}-1H-indazol-3-yl)-N-methylurea

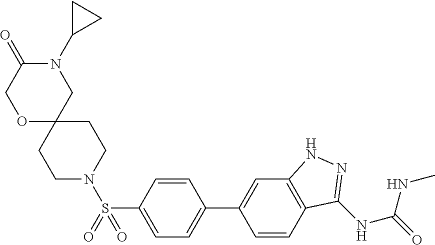 [SMILES: CNC(=O)Nc1n[nH]c2cc(-c3ccc(S(=O)(=O)N4CCC5(CC4)CN(C4CC4)C(=O)CO5)cc3)ccc12]

### a) N-(6-Bromo-1H-indazol-3-yl)-N-methylurea

1,1-Dimethylethyl-3-amino-5-bromo-1H-indazole-1-carboxylate (0.641 mmol) was dissolved in chloroform (3 mL). To this was added diisopropylethylamine (1.28 mmol) and methyl isocyanate (3.2 mmol). The reaction was stirred at room temperature overnight. Analysis by LCMS showed formation of the bis-urea product. The reaction was concentrated in vacuo and the residue was dissolved in tetrahydrofuran (10 mL) and treated with 3.5 mL of 1N aq sodium hydroxide. The reaction was heated to 30° C. for 3 h. Analysis by LCMS showed the desired mono-urea product. The reaction was concentrated in vacuo and the remaining water was neutralized with 1N aq HCl. Ethyl acetate (10 mL) was added with stirring, and an insoluble solid (desired product) was filtered off. The solid was stirred with 4N HCl in dioxane at 36° C. for 4 h during which time the solid slowly went into solution. Analysis by LCMS indicated the Boc group had been completely removed. The reaction was concentrated in vacuo and the residue was taken up in dichloromethane and washed with 10% aq sodium bicarbonate. The organic layer was dried over sodium sulfate, filtered, and concentrated in vacuo to give the title product (110 mg, 57%). LCMS (ES⁺) m/z 269.1 [M+H]⁺.

### b) N-(6-{4-[(4-cyclopropyl-3-oxo-1-oxa-4,9-diazaspiro[5.5]undec-9-yl)sulfonyl]phenyl}-1H-indazol-3-yl)-N-methylurea

Following the procedure described in Example 70a using N-(6-Bromo-1H-indazol-3-yl)-N-methylurea and 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one provided the title product (45 mg, 26%) as a solid. LCMS (ES⁺) m/z 539.2 [M+H]⁺.

### Example 108

### 4-cyclopropyl-9-((4-(8-methylquinolin-7-yl)phenyl)sulfonyl)-1-oxa-4,9-diazaspiro[5.5]undecan-3-one

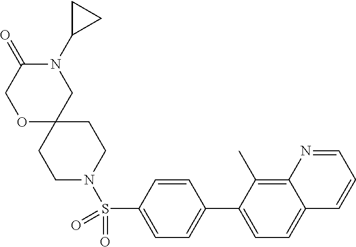 [SMILES: Cc1c(-c2ccc(S(=O)(=O)N3CCC4(CC3)CN(C3CC3)C(=O)CO4)cc2)ccc2cccnc12]

### a) 7-bromo-8-methylquinoline

A 500 mL round bottom flask equipped with a stirbar and condenser was charged with 3-bromo-2-methylaniline (81 mmol), sodium 3-nitrobenzenesulfonate (145 mmol), and propane-1,2,3-triol (glycerol) (227 mmol). The mixture was then taken up in water (14.04 mL) and carefully treated with concentrated sulfuric acid (26.0 mL). The mixture was then heated to 150° C. with stirring for 2 h, then cooled to room temperature and chilled with an ice bath. The mixture was carefully neutralized with a 5N aq solution of sodium hydroxide (˜180 mL), allowed to stir for 20 min, checked for pH, and then diluted with dichloromethane (1000 mL). The entire mixture was then transferred to a 2 L separatory funnel and extracted. The resulting emulsion was treated with ˜200 mL saturated brine and allowed to sit overnight to separate the layers. The organic layer was isolated and the aqueous layer re-extracted with an additional quantity of dichloromethane (˜400 mL). The combined organics were dried over sodium sulfate, filtered, and concentrated to a residue which solidified under high vacuum to afford the desired product as a dark brown solid (17.06 g, 94% yield). LCMS (ES⁺) m/z 221.9, 223.7 [M+H]⁺.

### b) 4-cyclopropyl-9-((4-(8-methylquinolin-7-yl)phenyl)sulfonyl)-1-oxa-4,9-diazaspiro[5.5]undecan-3-one

A 5 mL microwave reaction vial equipped with a stir bar was charged with 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.290 mmol), 7-bromo-8-methylquinoline (0.362 mmol), and PdCl₂(dppf)-CH₂Cl₂ adduct (0.020 mmol). The solids were taken up in 1,4-dioxane (2.61 mL) and treated with 2M aq potassium carbonate (0.290 mL). The mixture was sealed in the vial and subjected to microwave irradiation at 130° C. for 25 min (very high absorption setting). The reaction was then cooled to room temperature and partitioned between 15 mL each of water and dichloromethane. The organic layer was isolated and the aqueous layer re-extracted with an additional 15 mL dichloromethane. The organics were then combined, dried over sodium sulfate, filtered, and concentrated to a residue. The residue was purified on silica gel (0.3-7.5% methanol/dichloromethane). Fractions containing the desired material were combined and concentrated to a residue, which was precipitated from a methanol/diethyl ether mixture to afford the title product as an off-white solid (69 mg, 48%). LCMS (ES⁺) m/z 492.2 [M+H]⁺.

### Example 109

### 4-cyclopropyl-9-((4-(8-fluoroquinolin-7-yl)phenyl)sulfonyl)-1-oxa-4,9-diazaspiro[5.5]undecan-3-one

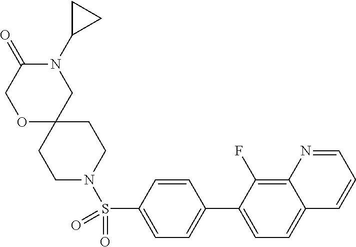 [SMILES: O=C1COC2(CCN(S(=O)(=O)c3ccc(-c4ccc5cccnc5c4F)cc3)CC2)CN1C1CC1]

### a) 7-bromo-8-fluoroquinoline

A 500 mL round bottom flask equipped with a stirbar and condenser was charged with 3-bromo-2-fluoroaniline (52.6 mmol), sodium 3-nitrobenzenesulfonate (94 mmol), and propane-1,2,3-triol (glycerol) (147 mmol). The mixture was then taken up in water (11.38 mL) and then carefully treated with concentrated sulfuric acid (21.06 mL). The reaction mixture was then heated to 150° C. with stirring for 2 h, then cooled to room temperature and chilled with an ice bath. The mixture was then carefully neutralized with a 5N aq solution of sodium hydroxide (˜180 mL), allowed to stir for 20 min, checked for pH, and then diluted with dichloromethane (1000 mL). The entire mixture was then transferred to a 2 L separatory funnel and extracted. The resulting emulsion was treated with ˜200 mL saturated brine and allowed to sit overnight to separate the layers. The organic layer was isolated and the aqueous re-extracted with an additional quantity of dichloromethane (˜400 mL). The combined organics were re-extracted with 400 mL of a 1N aq solution of sodium hydroxide, dried over sodium sulfate, filtered, and concentrated to a residue, which solidified under high vacuum to afford the desired product as a dark brown solid (9.16 g, 75% yield). LCMS (ES⁺) m/z 225.9, 227.8 [M+H]⁺.

### b) 4-cyclopropyl-9-((4-(8-fluoroquinolin-7-yl)phenyl)sulfonyl)-1-oxa-4,9-diazaspiro[5.5]undecan-3-one

Following the procedure described in Example 108 using 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.315 mmol) and 7-bromo-8-fluoroquinoline (0.331 mmol) provided the title product as an off-white solid (123 mg, 77%). LCMS (ES⁺) m/z 496.1 [M+H]⁺.

### Example 110

### 1-(3-oxo-9-{[4-(7-quinolinyl)phenyl]sulfonyl}-1-oxa-4,9-diazaspiro[5.5]undec-4-yl)cyclopropanecarboxamide

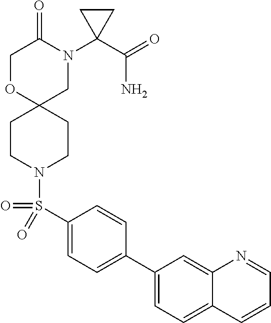 [SMILES: NC(=O)C1(N2CC3(CCN(S(=O)(=O)c4ccc(-c5ccc6cccnc6c5)cc4)CC3)OCC2=O)CC1]

### a) 1,1-dimethylethyl 4-{[(1-cyanocyclopropyl)amino]methyl}-4-hydroxy-1-piperidinecarboxylate

A 20 mL microwave vial was charged with a suspension of 1,1-dimethylethyl 1-oxa-6-azaspiro[2.5]octane-6-carboxylate (1.407 mmol), 1-aminocyclopropanecarbonitrile hydrochloride (2.81 mmol) and 2.0 M aq potassium hydroxide (2.81 mmol) in absolute ethanol (5.0 mL) and sealed with a standard aluminum crimp cap. The vessel was heated on an aluminum block at 80° C. for 3 days then cooled to room temperature. The resulting suspension was evaporated under reduced pressure onto silica powder and then purified by flash chromatography (0-5% methanol in dichloromethane). Product fractions, determined by TLC (5% methanol in dichloromethane, visualizing with eerie ammonium molybdate stain), were combined and concentrated in vacuo to afford the title compound (257 mg, 53% yield) as a colorless residue. MS(ES)+ m/e 296.3 [M+H]⁺.

### b) 1,1-dimethylethyl 4-(1-cyanocyclopropyl)-3-oxo-1-oxa-4,9-diazaspiro[5.5]undecane-9-carboxylate

To a stirring 0° C. solution of 1,1-dimethylethyl 4-{[(1-cyanocyclopropyl)amino]methyl}-4-hydroxy-1-piperidinecarboxylate (0.860 mmol) and triethylamine (3.44 mmol) in anhydrous dichloromethane (15.0 mL) was added neat chloroacetyl chloride (1.286 mmol) in one portion. The ice bath was removed and stirring continued for 30 min whereupon analysis by LCMS indicated complete conversion to the chloroacetamide intermediate. The resulting brown solution was diluted with dichloromethane, washed with saturated aq sodium bicarbonate, dried over sodium sulfate, and evaporated to a crude solid. This intermediate was diluted with anhydrous tetrahydrofuran (15.0 mL), treated with 60% sodium hydride in mineral oil (3.87 mmol) in one portion, and heated at reflux for 18 h. The resulting dark suspension was cooled to room temperature, quenched with water and brine, and extracted with diethyl ether. The combined organics were dried over sodium sulfate, treated with silica powder, and concentrated under reduced pressure. This was purified by flash chromatography (30-100% ethyl acetate in hexanes). The fractions were examined by silica TLC (40% hexanes in ethyl acetate) and visualized with ceric ammonium molybdate stain. Product fractions, determined by TLC (40% hexanes in ethyl acetate, visualizing with ceric ammonium molybdate stain), were combined and concentrated in vacuo to afford the title compound (145 mg, 49% yield) as a colorless residue that solidified upon standing. MS(ES)+ m/e 336.3 [M+H]⁺. ¹H NMR (400 MHz, CDCl₃) δ ppm 1.26-1.39 (m, 2H) 1.47 (s, 9H) 1.48-1.59 (m, 2H) 1.62 (br. s, 2H) 1.78-1.90 (m, 2H) 3.00-3.18 (m, 2H) 3.30 (s, 2H) 3.75-4.01 (m, 2H) 4.17 (s, 2H).

### c) 1-{9-[(4-bromophenyl)sulfonyl]-3-oxo-1-oxa-4,9-diazaspiro[5.5]undec-4-yl}cyclopropanecarboxamide

To a solution of 1,1-dimethylethyl 4-(1-cyanocyclopropyl)-3-oxo-1-oxa-4,9-diazaspiro[5.5]undecane-9-carboxylate (0.432 mmol) in anhydrous dichloromethane (3.0 mL) was added 4N HCl in dioxane (12.00 mmol). The mixture stirred at room temperature for 2 h whereupon analysis by LCMS indicated complete deprotection with concomitant conversion of the nitrile to the amide. The solvents were removed under reduced pressure and the residue was taken into anhydrous dichloromethane (15.0 mL) and treated with triethylamine (2.162 mmol) and 4-bromobenzenesulfonyl chloride (0.648 mmol). The reaction was stirred at room temperature for 3 days and then concentrated under reduced pressure to a residue that was purified by reverse phase HPLC (10-60% acetonitrile w/0.1% TFA/water w/0.1% TFA). Fractions containing the product were combined, treated with saturated aq sodium bicarbonate, concentrated briefly under reduced pressure to remove volatile solvents, and then extracted with dichloromethane. The combined organics were dried over sodium sulfate and concentrated in vacuo to afford the title compound (85 mg, 41% yield) as a white solid. MS(ES)+ m/e 472.0, 473.9 [M+H]⁺. ¹H NMR (400 MHz, DMSO-d6) δ ppm 0.98 (d, J=2.27 Hz, 2H) 1.33 (br. s, 2H) 1.58-1.73 (m, 2H) 1.75-1.88 (m, 2H) 2.42 (t, J=10.99 Hz, 2H) 3.28 (br. s., 2H) 3.45 (d, J=11.62 Hz, 2H) 3.88 (s, 2H) 7.10 (br. s, 2H) 7.63-7.71 (m, 2H) 7.83-7.92 (m, 2H).

### d) 1-(3-oxo-9-{[4-(7-quinolinyl)phenyl]sulfonyl}-1-oxa-4,9-diazaspiro[5.5]undec-4-yl)cyclopropanecarboxamide

A suspension of 1-{9-[(4-bromophenyl)sulfonyl]-3-oxo-1-oxa-4,9-diazaspiro[5.5]undec-4-yl}cyclopropanecarboxamide (0.169 mmol), 7-(4,4,5,5-tetramethyl-1,3,2-dioxaborolan-2-yl)quinoline (0.254 mmol), PdCl₂(dppf)-CH₂Cl₂ adduct (8.47 μmol), and 2.0 M aq potassium carbonate (0.678 mmol) in 1,4-dioxane (10.0 mL) was heated at 100° C. for 4 h. The resulting dark suspension was cooled, treated with silica powder, diluted with methanol, and concentrated under reduced pressure to dryness. Purification of the silica mixture by flash chromatography (ethyl acetate then with 3% methanol in ethyl acetate) afforded the title compound as an off-white solid upon trituration from absolute ethanol (55 mg, 62% yield). MS(ES)+ m/e 521.0 [M+H]⁺. ¹H NMR (400 MHz, DMSO-d6) δ ppm 0.99 (d, J=1.77 Hz, 2H) 1.23-1.44 (m, 2H) 1.60-1.77 (m, 2H) 1.85 (d, J=13.14 Hz, 2H) 2.42-2.49 (m, 2H) 3.30 (br. s, 2H) 3.53 (d, J=11.37 Hz, 2H) 3.88 (br. s, 2H) 7.11 (br. s, 2H) 7.59 (dd, J=8.21, 4.17 Hz, 1H) 7.87 (d, J=8.34 Hz, 2H) 8.05 (dd, J=8.46, 1.89 Hz, 1H) 8.12-8.19 (m, 3H) 8.41 (s, 1H) 8.44 (d, J=7.33 Hz, 1H) 8.98 (dd, J=4.17, 1.64 Hz, 1H).

### Example 111

### 4-(1-methylcyclobutyl)-9-{[4-(7-quinolinyl)phenyl]sulfonyl}-1-oxa-4,9-diazaspiro[5.5]undecan-3-one

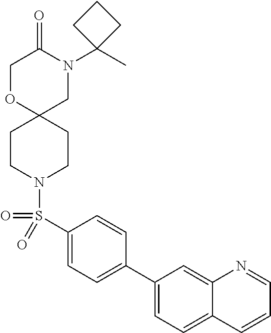 [SMILES: CC1(N2CC3(CCN(S(=O)(=O)c4ccc(-c5ccc6cccnc6c5)cc4)CC3)OCC2=O)CCC1]

### a) 1,1-dimethylethyl 4-hydroxy-4-{[(1-methylcyclobutyl)amino]methyl}-1-piperidinecarboxylate

A 20 mL microwave reaction vial was charged with a mixture of 1,1-dimethylethyl 1-oxa-6-azaspiro[2.5]octane-6-carboxylate (2.119 mmol) and 1-methylcyclobutanamine (5.83 mmol) in absolute ethanol (10.0 mL) and sealed with a standard aluminum crimp cap. The vessel was heated on an aluminum block at 80° C. for 18 h. The resulting clear solution was concentrated under reduced pressure to afford the crude title compound (641 mg, 96% yield) as a colorless residue that solidified upon standing. MS(ES)+ m/e 299.5 [M+H]⁺.

### b) 1,1-dimethylethyl 4-(1-methylcyclobutyl)-3-oxo-1-oxa-4,9-diazaspiro[5.5]undecane-9-carboxylate

To a stirring 0° C. solution of 1,1-dimethylethyl 4-hydroxy-4-{[(1-methylcyclobutyl)amino]methyl}-1-piperidinecarboxylate (2.128 mmol) and triethylamine (5.32 mmol) in anhydrous dichloromethane (15.0 mL) was added neat chloroacetyl chloride (3.20 mmol) in one portion. The ice bath was removed and stirring continued for 30 min whereupon analysis by LCMS indicated complete conversion to the chloroacetamide intermediate. The resulting brown solution was diluted with dichloromethane, washed with saturated aq sodium bicarbonate, dried over sodium sulfate, and concentrated in vacuo to a crude solid. This intermediate was taken into anhydrous tetrahydrofuran (15.0 mL), treated with 60% sodium hydride in mineral oil (9.58 mmol), and heated at reflux for 18 h. The resulting dark suspension was cooled to room temperature, quenched with water and brine, and extracted with diethyl ether. The combined organics were dried over sodium sulfate, treated with silica powder, and concentrated to dryness under reduced pressure. The silica mixture was purified by flash chromatography (15-100% ethyl acetate in hexanes). Product fractions, determined by TLC (50% hexanes in ethyl acetate) and visualized with ceric ammonium molybdate stain, were combined and concentrated under reduced pressure to afford the title compound (551 mg, 76% yield) as a white solid. MS(ES)+ m/e 339.2 [M+H]⁺. ¹H NMR (400 MHz, DMSO-d6) δ ppm 1.32 (s, 3H) 1.39 (s, 9H) 1.43-1.53 (m, 2H) 1.58-1.77 (m, 4H) 1.83-1.96 (m, 2H) 2.14-2.28 (m, 2H) 2.97 (br. s, 2H) 3.06 (s, 2H) 3.61-3.76 (m, 2H) 3.92 (s, 2H).

### c) 9-[(4-bromophenyl)sulfonyl]-4-(1-methylcyclobutyl)-1-oxa-4,9-diazaspiro[5.5]undecan-3-one

To a stirring solution of 1,1-dimethylethyl 4-(1-methylcyclobutyl)-3-oxo-1-oxa-4,9-diazaspiro[5.5]undecane-9-carboxylate (1.610 mmol) in anhydrous dichloromethane (20.0 mL) was added 4N HCl in 1,4-dioxane (30.0 mmol). The reaction was stirred at room temperature overnight, then the solvents were removed under reduced pressure to a crude residue. The residue was taken into anhydrous dichloromethane (20.0 mL) and treated with neat triethylamine (8.05 mmol) and 4-bromobenzene-1-sulfonyl chloride (2.415 mmol). After 1 h at room temperature, the reaction was diluted with dichloromethane, washed with saturated aq sodium bicarbonate and brine, dried over sodium sulfate, and concentrated to dryness in the presence of silica gel. The silica mixture was purified by flash chromatography (30-100% ethyl acetate in hexanes) to provide the title product (550 mg, 73% yield) as a tan solid. MS(ES)+ m/e 456.9, 459.1 [M+H]⁺. ¹H NMR (400 MHz, DMSO-d6) δ ppm 1.30 (s, 3H) 1.53-1.77 (m, 4H) 1.77-1.97 (m, 4H) 2.09-2.27 (m, 2H) 2.33-2.45 (m, 2H) 3.04 (s, 2H) 3.48 (d, J=11.62 Hz, 2H) 3.79 (s, 2H) 7.61-7.72 (m, 2H) 7.79-7.91 (m, 2H).

### d) 4-(1-methylcyclobutyl)-9-{[4-(7-quinolinyl)phenyl]sulfonyl}-1-oxa-4,9-diazaspiro[5.5]undecan-3-one

A suspension of 9-((4-bromophenyl)sulfonyl)-4-(1-methylcyclobutyl)-1-oxa-4,9-diazaspiro[5.5]undecan-3-one (0.437 mmol), 7-(4,4,5,5-tetramethyl-1,3,2-dioxaborolan-2-yl)quinoline (0.656 mmol), PdCl₂(dppf)-CH₂Cl₂ adduct (0.022 mmol), and 2.0 M aq potassium carbonate (1.749 mmol) in 1,4-dioxane (13.1 mL) was heated at 100° C. for 20 h. The resulting dark suspension was treated with silica gel and concentrated under reduced pressure to dryness. Purification of the silica mixture by flash chromatography (50-100% ethyl acetate in hexanes) and subsequent recrystallization of the solid product from ethanol afforded the title compound (121 mg, 54% yield) as a white solid. MS(ES)+m/e 505.9 [M+H]⁺. ¹H NMR (400 MHz, DMSO-d6) δ ppm 1.31 (s, 3H) 1.53-1.78 (m, 4H) 1.79-1.96 (m, 4H) 2.09-2.26 (m, 2H) 2.38-2.48 (m, 2H) 3.05 (s, 2H) 3.56 (d, J=11.62 Hz, 2H) 3.79 (s, 2H) 7.59 (dd, J=8.21, 4.17 Hz, 1H) 7.87 (d, J=8.34 Hz, 2H) 8.05 (dd, J=8.46, 1.89 Hz, 1H) 8.12-8.19 (m, 3H) 8.41 (s, 1H) 8.42-8.47 (m, 1H) 8.98 (dd, J=4.17, 1.64 Hz, 1H).

### Example 112

### 1-(3-oxo-9-{[4-(7-quinolinyl)phenyl]sulfonyl}-1-oxa-4,9-diazaspiro[5.5]undec-4-yl)cyclopropanecarbonitrile

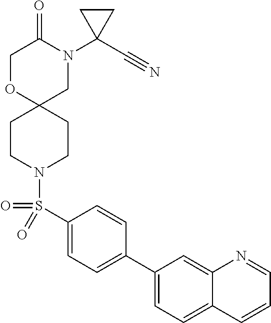 [SMILES: N#CC1(N2CC3(CCN(S(=O)(=O)c4ccc(-c5ccc6cccnc6c5)cc4)CC3)OCC2=O)CC1]

### a) 1-[({1-[(4-bromophenyl)sulfonyl]-4-hydroxy-4-piperidinyl}methyl)amino]cyclopropanecarbonitrile

To a stirring solution of 1-aminocyclopropanecarbonitrile hydrochloride (8.43 mmol) in anhydrous acetonitrile (15 ml) was added triethylamine (16.87 mmol). The resulting suspension was stirred at reflux for 2 h. The resulting clear solution was cooled and concentrated under reduced pressure to a residue that was suspended in 2:1 hexanes/ethyl acetate and then filtered by gravity through a plug of cotton. The filtrate was concentrated under reduced pressure to afford the amine free-base as a light orange-brown oil (603 mg, 87%).

A suspension of the 1-aminocyclopropanecarbonitrile (7.34 mmol) and 6-[(4-bromophenyl)sulfonyl]-1-oxa-6-azaspiro[2.5]octane (2.501 mmol) in absolute ethanol (15 mL) was heated at 95° C. for 20 h. The reaction was cooled and evaporated to a crude oil, which was taken into dichloromethane, treated with silica gel, and concentrated under reduced pressure to dryness. The silica mixture was purified by flash chromatography (20-80% ethyl acetate in hexanes) to afford, after trituration from 3:1 hexanes/ethyl acetate, the title compound (430 mg, 39% yield) as a white solid. MS(ES)+ m/e 414.0, 416.0 [M+H]⁺. ¹H NMR (400 MHz, DMSO-d6) δ ppm 0.83-0.94 (m, 2H) 1.09-1.19 (m, 2H) 1.39-1.59 (m, 4H) 2.41-2.49 (m, 2H) 2.54 (d, J=7.33 Hz, 2H) 3.12 (t, J=7.58 Hz, 1H) 3.38 (d, J=11.12 Hz, 2H) 4.27 (s, 1H) 7.67 (d, J=8.59 Hz, 2H) 7.86 (d, J=8.59 Hz, 2H).

### b) 1-{9-[(4-bromophenyl)sulfonyl]-3-oxo-1-oxa-4,9-diazaspiro[5.5]undec-4-yl}cyclopropanecarbonitrile

To a stirring solution of 1-[({1-[(4-bromophenyl)sulfonyl]-4-hydroxy-4-piperidinyl}methyl)amino]cyclopropanecarbonitrile (1.011 mmol) and N,N-diisopropylethylamine (2.53 mmol) in anhydrous dichloromethane (15 mL) cooled to 0° C. was added chloroacetyl chloride (1.515 mmol). After 30 min, the reaction was diluted with dichloromethane, washed with saturated aq sodium bicarbonate and brine, dried over sodium sulfate, and concentrated under reduced pressure to afford the intermediate chloroacetamide as a brown foam. To a solution of the intermediate in anhydrous tetrahydrofuran (15 mL) was added 60% sodium hydride in mineral oil (4.55 mmol). The reaction was heated at 75° C. for 48 h and then cooled, quenched with water and brine, and extracted with dichloromethane. The combined organic layers were dried over sodium sulfate, treated with silica gel, and concentrated to dryness. Purification of the silica mixture by flash chromatography (50-100% ethyl acetate in hexanes) provided the title compound (132 mg, 27% yield) as a white solid. MS(ES)+ m/e 453.9, 455.8 [M+H]⁺. ¹H NMR (400 MHz, DMSO-d6) δ ppm 1.29-1.39 (m, 2H) 1.52-1.61 (m, 2H) 1.64-1.76 (m, 2H) 1.78-1.90 (m, 2H) 2.41 (d, J=2.02 Hz, 2H) 3.33 (s, 2H) 3.48 (d, J=11.87 Hz, 2H) 3.98 (s, 2H) 7.67 (d, J=8.59 Hz, 2H) 7.84-7.91 (m, 2H).

### c) 1-(3-oxo-9-{[4-(7-quinolinyl)phenyl]sulfonyl}-1-oxa-4,9-diazaspiro[5.5]undec-4-yl)cyclopropanecarbonitrile

A stirring suspension of 1-{9-[(4-bromophenyl)sulfonyl]-3-oxo-1-oxa-4,9-diazaspiro[5.5]undec-4-yl}cyclopropanecarbonitrile (0.275 mmol), 7-(4,4,5,5-tetramethyl-1,3,2-dioxaborolan-2-yl)quinoline (0.413 mmol), PdCl₂(dppf)-CH₂Cl₂ adduct (0.014 mmol), and 2.0 M aq potassium carbonate (1.101 mmol) in anhydrous 1,4-dioxane (5.50 mL) was heated at 95° C. for 20 h. The resulting dark suspension was cooled, treated with silica gel, and concentrated under reduced pressure to dryness. The silica mixture was purified by flash chromatography (50-100% ethyl acetate in hexanes) to afford a light yellow residue that was further purified by reverse phase HPLC (10-60% acetonitrile w/0.1% TFA/water w/0.1% TFA). The product fractions were combined, treated with saturated aq sodium bicarbonate, and extracted with dichloromethane. The combined organic layers were washed with brine, dried over sodium sulfate, and concentrated in vacuo to give a white foam. The residue was taken into acetonitrile (1 mL) and water (1 mL), frozen, and lyophillized to afford the title compound (103 mg, 74% yield) as a white solid. MS(ES)+ m/e 502.9 [M+H]⁺. ¹H NMR (400 MHz, DMSO-d6) δ ppm 1.29-1.39 (m, 2H) 1.53-1.61 (m, 2H) 1.73 (td, J=12.88, 3.79 Hz, 2H) 1.83-1.94 (m, 2H) 2.42-2.49 (m, 2H) 3.35 (s, 2H) 3.55 (d, J=11.62 Hz, 2H) 3.98 (s, 2H) 7.59 (dd, J=8.21, 4.17 Hz, 1H) 7.87 (d, J=8.59 Hz, 2H) 8.05 (dd, J=8.46, 1.89 Hz, 1H) 8.12-8.16 (m, 2H) 8.17 (d, J=6.32 Hz, 1H) 8.41 (s, 1H) 8.44 (d, J=7.33 Hz, 1H) 8.98 (dd, J=4.29, 1.77 Hz, 1H).

### Example 113

### 4-(3-oxetanyl)-9-{[4-(7-quinolinyl)phenyl]sulfonyl}-1-oxa-4,9-diazaspiro[5.5]undecan-3-one

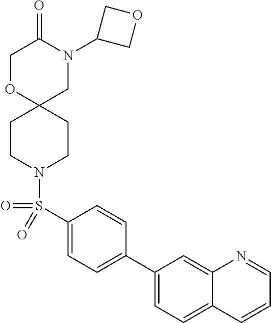 [SMILES: O=C1COC2(CCN(S(=O)(=O)c3ccc(-c4ccc5cccnc5c4)cc3)CC2)CN1C1COC1]

### a) 1-[(4-bromophenyl)sulfonyl]-4-[(3-oxetanylamino)methyl]-4-piperidinol

A 20 mL microwave reaction vial was charged with a suspension of 6-[(4-bromophenyl)sulfonyl]-1-oxa-6-azaspiro[2.5]octane (0.626 mmol) and oxetan-3-amine (2.504 mmol) in absolute ethanol (10.0 mL) and sealed with a standard aluminum crimp cap. The vessel was heated on an aluminum block at 85° C. for 18 h. The resulting solution was cooled to room temperature, treated with silica gel, and concentrated under reduced pressure to dryness. Purification of the crude silica mixture by flash chromatography (0-5% methanol in ethyl acetate) provided the title compound (181 mg, 70% yield) as a white solid. MS(ES)+ m/e 405.1, 407.3 [M+H]⁺. ¹H NMR (400 MHz, DMSO-d6) δ ppm 1.42-1.63 (m, 4H) 2.20 (br. s., 1H) 2.32 (s, 2H) 2.43-2.50 (m, 2H) 3.38 (d, J=11.62 Hz, 2H) 3.80 (quin, J=6.57 Hz, 1H) 4.21 (s, 1H) 4.27 (t, J=6.19 Hz, 2H) 4.57 (t, J=6.57 Hz, 2H) 7.63-7.72 (m, 2H) 7.82-7.90 (m, 2H).

### b) 9-[(4-bromophenyl)sulfonyl]-4-(3-oxetanyl)-1-oxa-4,9-diazaspiro[5.5]undecan-3-one

To a stirring solution of 1-[(4-bromophenyl)sulfonyl]-4-[(3-oxetanylamino)methyl]-4-piperidinol (0.432 mmol) and N,N-diisopropylethylamine (1.079 mmol) in anhydrous dichloromethane (15 mL) cooled to 0° C. was added chloroacetyl chloride (0.651 mmol). After 30 min, the reaction was diluted with dichloromethane, washed with saturated aq sodium bicarbonate, dried over sodium sulfate, and concentrated in vacuo to a brown residue. The chloroacetamide intermediate was taken into anhydrous tetrahydrofuran (15 mL), treated with 60% sodium hydride in mineral oil (4.32 mmol), and heated at 75° C. for 5 h. The reaction was cooled, quenched slowly with water, and extracted with dichloromethane. The combined organic layers were dried over sodium sulfate, treated with silica gel, and concentrated under reduced pressure to dryness. Purification of the crude silica mixture by flash chromatography (ethyl acetate) afforded the title compound (210 mg, 98% yield) at a light tan solid. MS(ES)+ m/e 444.9, 446.9 [M+H]⁺. ¹H NMR (400 MHz, DMSO-d6) δ ppm 1.66-1.82 (m, 2H) 1.82-1.92 (m, 2H) 2.32-2.47 (m, 2H) 3.46 (s, 2H) 3.49 (d, J=11.87 Hz, 2H) 3.92 (s, 2H) 4.62 (quin, J=7.20 Hz, 4H) 5.39 (t, J=7.20 Hz, 1H) 7.67 (d, J=8.59 Hz, 2H) 7.88 (d, J=8.59 Hz, 2H).

### c) 4-(3-oxetanyl)-9-{[4-(7-quinolinyl)phenyl]sulfonyl}-1-oxa-4,9-diazaspiro[5.5]undecan-3-one

A stirring suspension of 9-[(4-bromophenyl)sulfonyl]-4-(3-oxetanyl)-1-oxa-4,9-diazaspiro[5.5]undecan-3-one (0.406 mmol), 7-(4,4,5,5-tetramethyl-1,3,2-dioxaborolan-2-yl)quinoline (0.609 mmol), PdCl₂(dppf)-CH₂Cl₂ adduct (0.020 mmol), and 2.0 M aq potassium carbonate (1.625 mmol) in 1,4-dioxane (8.12 mL) was heated at 95° C. for 18 h. The resulting dark suspension was treated with silica gel and concentrated to dryness under reduced pressure. Purification of the crude silica mixture by flash chromatography (ethyl acetate then 5% methanol in ethyl acetate) and trituration of the resultant light tan residue from ethanol provided the title compound (131 mg, 64% yield) as a white solid. MS(ES)+ m/e 493.9 [M+H]⁺. ¹H NMR (400 MHz, DMSO-d6) δ ppm 1.69-1.86 (m, 2H) 1.86-1.97 (m, 2H) 2.41-2.49 (m, 2H) 3.48 (s, 2H) 3.57 (d, J=11.87 Hz, 2H) 3.92 (s, 2H) 4.55-4.70 (m, 4H) 5.33-5.47 (m, 1H) 7.59 (dd, J=8.34, 4.29 Hz, 1H) 7.88 (d, J=8.59 Hz, 2H) 8.05 (dd, J=8.59, 1.77 Hz, 1H) 8.11-8.20 (m, 3H) 8.41 (d, J=1.77 Hz, 1H) 8.44 (dd, J=8.59, 1.26 Hz, 1H) 8.98 (dd, J=4.29, 1.77 Hz, 1H).

### Example 114

### 4-[1-(hydroxymethyl)cyclopropyl]-9-{[4-(7-quinolinyl)phenyl]sulfonyl}-1-oxa-4,9-diazaspiro[5.5]undecan-3-one

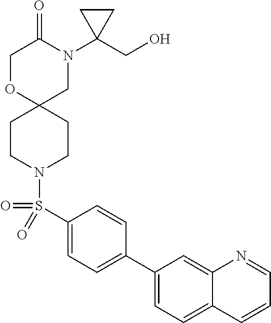 [SMILES: O=C1COC2(CCN(S(=O)(=O)c3ccc(-c4ccc5cccnc5c4)cc3)CC2)CN1C1(CO)CC1]

### a) 1-({[(1,1-dimethylethyl)(dimethyl)silyl]oxy}methyl)cyclopropanamine

To a stirring solution of (1-aminocyclopropyl)methanol (9.69 mmol), 4-(dimethylamino)pyridine (0.484 mmol), and triethylamine (21.32 mmol) in anhydrous dichloromethane (25 mL) was added chloro(1,1-dimethylethyl)dimethylsilane (10.66 mmol). After stirring 20 h, the reaction was quenched with saturated aq ammonium chloride and extracted with dichloromethane. The extracts were washed with water, dried over magnesium sulfate, and concentrated under reduced pressure to afford the crude title compound (1.70 g, 78% yield) as a colorless liquid. MS(ES)+ m/e 202.2 [M+H]⁺.

### b) 1-[(4-bromophenyl)sulfonyl]-4-({[1-({[(1,1-dimethylethyl)(dimethyl)silyl]oxy}methyl)cyclopropyl]amino}methyl)-4-piperidinol

A stirring suspension of 6-[(4-bromophenyl)sulfonyl]-1-oxa-6-azaspiro[2.5]octane (1.505 mmol) and 1-({[(1,1-dimethylethyl)(dimethyl)silyl]oxy}methyl)cyclopropanamine (2.258 mmol) in ethanol (20 mL) was heated at reflux for 24 h. The reaction was cooled, treated with silica gel, and concentrated under reduced pressure to dryness. Purification of the crude silica mixture by flash chromatography (20% ethyl acetate in hexanes) afforded the title compound (520 mg, 64% yield) as a white solid. MS(ES)+ m/e 532.6, 534.8 [M+H]⁺. ¹H NMR (400 MHz, DMSO-d6) δ ppm 0.01 (s, 6H) 0.39 (d, J=4.55 Hz, 4H) 0.85 (s, 9H) 1.39-1.56 (m, 4H) 1.93-2.04 (m, 1H) 2.42-2.48 (m, 2H) 2.52-2.56 (m, 1H) 3.33-3.37 (m, 2H) 3.48 (s, 2H) 4.09 (s, 1H) 7.67 (d, J=8.59 Hz, 2H) 7.86 (d, J=8.59 Hz, 2H).

### c) 9-[(4-bromophenyl)sulfonyl]-4-[1-({[(1,1-dimethylethyl)(dimethyl)silyl]oxy}methyl)cyclopropyl]-1-oxa-4,9-diazaspiro[5.5]undecan-3-one

To a stirring solution of 1-[(4-bromophenyl)sulfonyl]-4-({[1-({[(1,1-dimethylethyl)(dimethyl)silyl]oxy}methyl)cyclopropyl]amino}methyl)-4-piperidinol (0.965 mmol) and N,N-diisopropylethylamine (2.413 mmol) in anhydrous dichloromethane (15 mL) cooled to 0° C. was added chloroacetyl chloride (1.443 mmol). The ice bath was removed and stirring continued for 30 min whereupon analysis by LCMS indicated complete conversion to the intermediate chloroacetamide. The reaction was diluted with dichloromethane, washed with saturated aq sodium bicarbonate, dried over sodium sulfate, and concentrated in vacuo to a tan foam. To a stirring solution of the chloroacetamide intermediate in anhydrous tetrahydrofuran (15 mL) was added 60% sodium hydride in mineral oil (4.83 mmol). The reaction was heated at reflux overnight and then cooled, quenched with water (˜1 mL), treated with silica gel, and concentrated under reduced pressure to dryness. Purification of the crude silica gel mixture by flash chromatography (20-80% ethyl acetate in hexanes) provided the title compound (456 mg, 80% yield) as a white solid. MS(ES)+ m/e 573.0, 574.7 [M+H]⁺. ¹H NMR (400 MHz, DMSO-d6) δ ppm 0.00 (s, 6H) 0.74 (s, 4H) 0.84 (s, 9H) 1.55-1.69 (m, 2H) 1.73-1.83 (m, 2H) 2.36-2.47 (m, 2H) 3.27 (s, 2H) 3.40-3.49 (m, 2H) 3.58-3.65 (m, 2H) 3.86 (s, 2H) 7.66 (d, J=8.84 Hz, 2H) 7.87 (d, J=8.59 Hz, 2H).

### d) 9-[(4-bromophenyl)sulfonyl]-4-[1-(hydroxymethyl)cyclopropyl]-1-oxa-4,9-diazaspiro[5.5]undecan-3-one

To a solution of 9-[(4-bromophenyl)sulfonyl]-4-[1-({[(1,1-dimethylethyl)(dimethyl)silyl]oxy}methyl)cyclopropyl]-1-oxa-4,9-diazaspiro[5.5]undecan-3-one (0.758 mmol) in anhydrous tetrahydrofuran (10.0 mL) was added 1 M tetrabutylammonium fluoride in THF (1.517 mmol). The reaction was stirred at room temperature for 20 h and then silica gel was added and the suspension was concentrated under reduced pressure to dryness. Purification of the crude silica gel mixture by flash chromatography (10% hexanes in ethyl acetate) afforded the title compound (0.610 mmol, 80% yield) as a white solid. MS(ES)+ m/e 459.0, 461.1 [M+H]⁺. ¹H NMR (400 MHz, DMSO-d6) δ ppm 0.65-0.79 (m, 4H) 1.57-1.71 (m, 2H) 1.74-1.84 (m, 2H) 2.36-2.48 (m, 2H) 3.29 (s, 2H) 3.38-3.47 (m, 4H) 3.85 (s, 2H) 4.73 (t, J=5.68 Hz, 1H) 7.62-7.70 (m, 2H) 7.84-7.90 (m, 2H).

### e) 4-[1-(hydroxymethyl)cyclopropyl]-9-{[4-(7-quinolinyl)phenyl]sulfonyl}-1-oxa-4,9-diazaspiro[5.5]undecan-3-one

A 20 mL microwave reaction vial was charged with a suspension of 9-((4-bromophenyl)sulfonyl)-4-(1-(hydroxymethyl)cyclopropyl)-1-oxa-4,9-diazaspiro[5.5]undecan-3-one (0.163 mmol), 7-(4,4,5,5-tetramethyl-1,3,2-dioxaborolan-2-yl)quinoline (0.245 mmol), PdCl₂(dppf)-CH₂Cl₂ adduct (8.16 μmol), and 2.0 M aq potassium carbonate (0.653 mmol) in 1,4-dioxane (3.20 mL) and sealed with a standard aluminum crimp cap. The vessel was heated on an aluminum block at 95° C. for 3 h. The resulting dark solution was cooled, treated with silica gel, and concentrated to dryness. The crude silica mixture was purified by flash chromatography (0-5% methanol in ethyl acetate). The desired product fractions were combined and evaporated to a residue that was taken into acetonitrile (1 mL) and water (1 mL), frozen, and lyophilized to afford the title compound (52 mg, 62% yield) as a white solid. MS(ES)+ m/e 507.8 [M+H]⁺. ¹H NMR (400 MHz, DMSO-d6) δ ppm 0.63-0.81 (m, 4H) 1.59-1.76 (m, 2H) 1.76-1.90 (m, 2H) 2.51-2.56 (m, 2H) 3.31 (s, 2H) 3.43 (d, J=5.81 Hz, 2H) 3.51 (d, J=11.62 Hz, 2H) 3.85 (s, 2H) 4.73 (t, J=5.68 Hz, 1H) 7.59 (dd, J=8.21, 4.17 Hz, 1H) 7.87 (d, J=8.59 Hz, 2H) 8.05 (dd, J=8.59, 2.02 Hz, 1H) 8.11-8.19 (m, 3H) 8.41 (d, J=1.77 Hz, 1H) 8.44 (dd, J=8.59, 1.26 Hz, 1H) 8.98 (dd, J=4.29, 1.77 Hz, 1H).

### Example 115

### 4-{1-[(methyloxy)methyl]cyclopropyl}-9-{[4-(7-quinolinyl)phenyl]sulfonyl}-1-oxa-4,9-diazaspiro[5.5]undecan-3-one

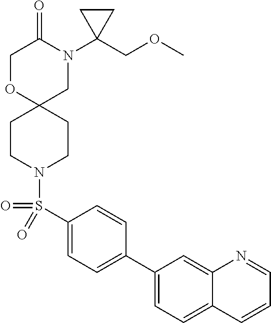 [SMILES: COCC1(N2CC3(CCN(S(=O)(=O)c4ccc(-c5ccc6cccnc6c5)cc4)CC3)OCC2=O)CC1]

### a) 1,1-dimethylethyl 4-hydroxy-4-[({1-[(methyloxy)methyl]cyclopropyl}amino)methyl]-1-piperidinecarboxylate

A 20 mL microwave reaction vial was charged with 1-(methoxymethyl)cyclopropanamine hydrochloride (6.15 mmol) and ethanol (5.0 mL) and treated with 6.0 N aq sodium hydroxide (11.96 mmol). To the resulting suspension was added water (2.0 mL) dropwise until a clear solution resulted, and then 1,1-dimethylethyl 1-oxa-6-azaspiro[2.5]octane-6-carboxylate (4.10 mmol) was added. The vessel was sealed with a standard aluminum crimp cap and heated on an aluminum block at 65° C. overnight. The solution was cooled to room temperature, treated with silica gel, and concentrated under reduced pressure to dryness. The crude silica gel mixture was purified by flash chromatography (30-100% ethyl acetate in hexanes). Fractions containing the desired product, determined by TLC (ethyl acetate) and visualized with ceric ammonium molybdate stain, were combined and concentrated under reduced pressure to afford the title compound (238 mg, 18% yield) as a colorless oil. MS(ES)+m/e 315.0 [M+H]⁺. ¹H NMR (400 MHz, CDCl₃) δ ppm 0.45-0.57 (m, 2H) 0.62-0.70 (m, 2H) 1.34-1.46 (m, 4H) 1.47 (s, 9H) 2.62 (s, 2H) 3.03-3.28 (m, 3H) 3.30 (s, 2H) 3.40 (s, 3H) 3.76-3.96 (m, 2H).

### b) 1,1-dimethylethyl 4-{1-[(methyloxy)methyl]cyclopropyl}-3-oxo-1-oxa-4,9-diazaspiro[5.5]undecane-9-carboxylate

To a stirring solution of 1,1-dimethylethyl 4-hydroxy-4-[({1-[(methyloxy)methyl]cyclopropyl}amino)methyl]-1-piperidinecarboxylate (0.732 mmol) and N,N-diisopropylethylamine (1.829 mmol) in anhydrous dichloromethane (10 mL) cooled to 0° C. was added chloroacetyl chloride (1.099 mmol). The reaction was stirred at 0° C. for 30 min whereupon analysis by LCMS indicated complete conversion to the chloroacetamide intermediate. The reaction was diluted with dichloromethane, washed with saturated aq sodium bicarbonate, dried over sodium sulfate, and concentrated under reduced pressure to a residue. The residue was taken into tetrahydrofuran (10 mL), treated with 60% sodium hydride in mineral oil (3.66 mmol), and heated at 75° C. for 3 days. The resulting dark solution was cooled to room temperature, quenched slowly with water (˜1 mL), treated with silica gel, and concentrated under reduced pressure to dryness. Purification of the crude silica gel mixture by flash chromatography (30-100% ethyl acetate in hexanes) afforded the title compound (195 mg, 74% yield) as a clear, colorless solid. MS(ES)+ m/e 355.4 [M+H]⁺. ¹H NMR (400 MHz, DMSO-d6) δ ppm 0.64-0.89 (m, 4H) 1.39 (s, 9H) 1.45-1.53 (m, 2H) 1.61-1.70 (m, 2H) 3.01 (br. s., 2H) 3.24 (s, 3H) 3.29 (s, 2H) 3.38 (s, 2H) 3.55-3.68 (m, 2H) 3.99 (s, 2H).

### c) 4-{1-[(methyloxy)methyl]cyclopropyl}-1-oxa-4,9-diazaspiro[5.5]undecan-3-one hydrochloride

A stirring solution of 1,1-dimethylethyl 4-{1-[(methyloxy)methyl]cyclopropyl}-3-oxo-1-oxa-4,9-diazaspiro[5.5]undecane-9-carboxylate (0.536 mmol) in anhydrous dichloromethane (8.0 ml) was treated with 4N HCl in 1,4-dioxane (8.00 mmol). After 1 h, the resulting suspension was concentrated under reduced pressure to afford the crude title compound (159 mg, 0.536 mmol, 100% yield) as a white solid. MS(ES)+ m/e 255.2 [M+H]⁺.

### d) 9-[(4-bromophenyl)sulfonyl]-4-{1-[(methyloxy)methyl]cyclopropyl}-1-oxa-4,9-diazaspiro[5.5]undecan-3-one

To a stirring solution of 4-{1-[(methyloxy)methyl]cyclopropyl}-1-oxa-4,9-diazaspiro[5.5]undecan-3-one hydrochloride (0.272 mmol) and N,N-diisopropylethylamine (0.681 mmol) in anhydrous dichloromethane (10 mL) was added 4-bromobenzene-1-sulfonyl chloride (0.408 mmol) in one portion. After 18 h, the reaction was treated with silica gel and concentrated under reduced pressure to dryness. Purification of the crude silica gel mixture by flash chromatography (30-100% ethyl acetate in hexanes) afforded the title compound (116 mg, 86% yield) as a clear, colorless residue that solidified upon standing. MS(ES)+ m/e 473.1, 475.1 [M+H]⁺. ¹H NMR (400 MHz, DMSO-d6) δ ppm 0.67-0.86 (m, 4H) 1.56-1.71 (m, 2H) 1.78 (br. s., 2H) 2.43-2.49 (m, 2H) 3.22 (s, 3H) 3.25 (s, 2H) 3.35 (s, 2H) 3.40 (d, J=11.87 Hz, 2H) 3.86 (s, 2H) 7.62-7.72 (m, 2H) 7.84-7.91 (m, 2H).

### e) 4-{1-[(methyloxy)methyl]cyclopropyl}-9-{[4-(7-quinolinyl)phenyl]sulfonyl}-1-oxa-4,9-diazaspiro[5.5]undecan-3-one

A suspension of 9-[(4-bromophenyl)sulfonyl]-4-{1-[(methyloxy)methyl]cyclopropyl}-1-oxa-4,9-diazaspiro[5.5]undecan-3-one (0.234 mmol), 7-(4,4,5,5-tetramethyl-1,3,2-dioxaborolan-2-yl)quinoline (0.352 mmol), PdCl₂(dppf)-CH₂Cl₂ adduct (0.012 mmol), and 2.0 M aq potassium carbonate (0.938 mmol) in 1,4-dioxane (4.70 mL) was heated at 100° C. for 1 h. The reaction was cooled, treated with silica gel, and concentrated under reduced pressure to dryness. The crude silica gel mixture was purified by flash chromatography (0-5% methanol in ethyl acetate). The desired fractions were combined and concentrated to a yellow residue that was further purified by reverse phase HPLC (10-60% acetonitrile w/0.1% TFA/water w/0.1% TFA). The desired fractions were combined, treated with saturated aq sodium bicarbonate, and concentrated under reduced pressure to remove the volatile solvents. The cloudy solution was extracted with dichloromethane, which was subsequently dried over sodium sulfate and concentrated to a clear, colorless residue that was taken into acetonitrile (2 mL) and water (2 mL), frozen, and lyophilized to afford the title compound (91 mg, 72% yield) as a white solid. MS(ES)+ m/e 522.3 [M+H]⁺. ¹H NMR (400 MHz, DMSO-d6) δ ppm 0.69-0.85 (m, 4H) 1.61-1.75 (m, 2H) 1.76-1.88 (m, 2H) 2.52-2.60 (m, 2H) 3.22 (s, 3H) 3.27 (s, 2H) 3.35 (s, 2H) 3.48 (d, J=11.62 Hz, 2H) 3.87 (s, 2H) 7.60 (dd, J=8.34, 4.29 Hz, 1H) 7.87 (d, J=8.59 Hz, 2H) 8.05 (dd, J=8.59, 2.02 Hz, 1H) 8.13-8.19 (m, 3H) 8.41 (d, J=1.77 Hz, 1H) 8.43-8.48 (m, 1H) 8.99 (dd, J=4.29, 1.77 Hz, 1H).

### Example 116

### 4-[1-(hydroxymethyl)cyclopropyl]-9-({4-[3-(methyloxy)-7-quinolinyl]phenyl}sulfonyl)-1-oxa-4,9-diazaspiro[5.5]undecan-3-one

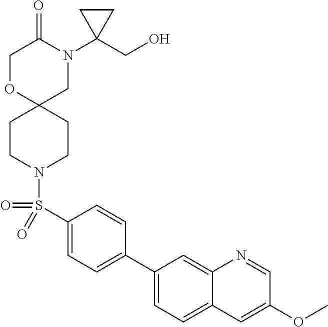 [SMILES: COc1cnc2cc(-c3ccc(S(=O)(=O)N4CCC5(CC4)CN(C4(CO)CC4)C(=O)CO5)cc3)ccc2c1]

### a) 3-(methyloxy)-7-(4,4,5,5-tetramethyl-1,3,2-dioxaborolan-2-yl)quinoline

A 20 mL microwave reaction vial was charged with a suspension of 7-bromo-3-methoxyquinoline (0.328 mmol), bis(pinacolato)diboron (0.360 mmol), tetrakis(triphenylphosphine)palladium(0) (0.016 mmol), and potassium acetate (0.819 mmol) in anhydrous 1,4-dioxane (5.0 mL) and sealed with a standard aluminum crimp cap. The vessel was heated on an aluminum block at 100° C. for 4 h. The resulting orange tan suspension of the title compound was cooled and used without isolation. MS(ES)+ m/e 286.2 [M+H]⁺.

### b) 4-[1-(hydroxymethyl)cyclopropyl]-9-({4-[3-(methyloxy)-7-quinolinyl]phenyl}sulfonyl)-1-oxa-4,9-diazaspiro[5.5]undecan-3-one

A solution of 3-(methyloxy)-7-(4,4,5,5-tetramethyl-1,3,2-dioxaborolan-2-yl)quinoline was transferred to a mixture of 9-[(4-bromophenyl)sulfonyl]-4-[1-(hydroxymethyl)cyclopropyl]-1-oxa-4,9-diazaspiro[5.5]undecan-3-one (0.131 mmol), tetrakis(triphenylphosphine)palladium(0) (6.55 μmol) and 2.0 M aq potassium carbonate (0.655 mmol) in 1,4-dioxane (5.00 mL) and heated at 100° C. for 2 h. The resulting dark suspension was cooled, treated with silica gel, and concentrated under reduced pressure to dryness. The crude silica gel mixture was purified by flash chromatography (0-7% methanol in ethyl acetate). The desired product fractions were combined and concentrated to a residue that was further purified by reverse phase HPLC (10-60% acetonitrile w/0.1% TFA/water w/0.1% TFA). The desired fractions were combined, treated with saturated aq sodium bicarbonate, and extracted with dichloromethane. The combined organic layers were dried over sodium sulfate and concentrated in vacuo to a residue that was taken into acetonitrile (1 mL) and water (0.5 mL), frozen, and lyophilized to afford the title compound (41 mg, 23% yield) as a white solid. MS(ES)+m/e 538.2 [M+H]⁺. ¹H NMR (400 MHz, DMSO-d6) δ ppm 0.72 (d, J=12.88 Hz, 4H) 1.57-1.76 (m, 2H) 1.76-1.89 (m, 2H) 2.44-2.49 (m, 2H) 3.30 (s, 2H) 3.43 (d, J=5.81 Hz, 2H) 3.45-3.55 (m, 2H) 3.85 (s, 2H) 3.96 (s, 3H) 4.72 (t, J=5.56 Hz, 1H) 7.79-7.90 (m, 3H) 7.96-8.09 (m, 2H) 8.12 (d, J=8.59 Hz, 2H) 8.35 (s, 1H) 8.71 (d, J=2.78 Hz, 1H).

### Example 117

### 4-cyclopropyl-9-((4-(8-methoxyquinolin-7-yl)phenyl)sulfonyl)-1-oxa-4,9-diazaspiro[5.5]undecan-3-one

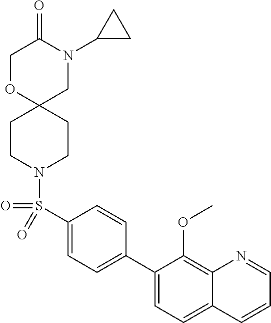 [SMILES: COc1c(-c2ccc(S(=O)(=O)N3CCC4(CC3)CN(C3CC3)C(=O)CO4)cc2)ccc2cccnc12]

### a) 7-bromo-8-methoxyquinoline

In an oven dried round bottom flask under nitrogen, a solution of 7-bromoquinolin-8-ol (1 g, 4.46 mmol) in N,N-dimethylformamide (20 mL) at room temperature was treated with sodium hydride (60% dispersion in mineral oil, 0.268 g, 6.69 mmol) to give a bright yellow mixture and was stirred for 3 min. Iodomethane (0.307 mL, 4.91 mmol) was then added by syringe and the reaction was stirred for 30 min. The reaction was quenched carefully with water (50 mL) and diluted with ethyl acetate (100 mL). The layers were separated and the organic layer was dried over magnesium sulfate and concentrated in vacuo to give a liquid, which solidified to a white solid (1.06 g, quantitative yield) upon standing overnight. MS(ES)+ m/e 237.8, 239.7 [M+H]⁺.

### b) 4-cyclopropyl-9-((4-(8-methoxyquinolin-7-yl)phenyl)sulfonyl)-1-oxa-4,9-diazaspiro[5.5]undecan-3-one

In a sealed microwave vial, a mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (160 mg, 0.336 mmol), 7-bromo-8-methoxyquinoline (80 mg, 0.336 mmol), 1,1′-bis(diphenylphosphino)ferrocene-palladium(II)dichloride dichloromethane complex (13.71 mg, 0.017 mmol) and 2M aq potassium carbonate solution (1 mL, 0.336 mmol) in 1,4-dioxane (3 mL) was stirred at 100° C. for 1 h. The reaction was cooled to room temperature at which point two layers were visible. The reaction mixture was diluted with ethyl acetate (5 mL) and the organic layer was removed by pipette, placed in a flask, dried over magnesium sulfate, and concentrated in vacuo to give a yellow residue. Purification of the residue by silica gel chromatography (0-5% methanol/ethyl acetate) provided the title compound (143 mg, 84%) as a yellow solid. MS(ES)+ m/e 508.2 [M+H]⁺.

### Example 118

### 4-cyclopropyl-9-((4-(8-hydroxyquinolin-7-yl)phenyl)sulfonyl)-1-oxa-4,9-diazaspiro[5.5]undecan-3-one

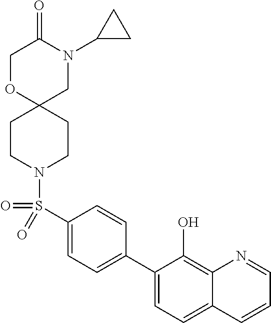 [SMILES: O=C1COC2(CCN(S(=O)(=O)c3ccc(-c4ccc5cccnc5c4O)cc3)CC2)CN1C1CC1]

### a) 7-bromoquinolin-8-yl benzoate

In a round bottom flask under nitrogen, a mixture of 7-bromoquinolin-8-ol (600 mg, 2.68 mmol) in dichloromethane (5 mL) was treated with pyridine (0.325 mL, 4.02 mmol) followed by benzoyl chloride (0.342 mL, 2.95 mmol). The yellow reaction mixture was stirred at room temperature for 48 h during which time a white precipitate formed. The reaction mixture was filtered to collect the precipitate, and the filtrate (which contained additional desired product) was set aside. The precipitate was dried under suction filtration for 12 days to afford the title compound (351 mg, 40%) as a white solid. MS(ES)+ m/e 328.2, 330.2 [M+H]⁺.

### b) 4-cyclopropyl-9-((4-(8-hydroxyquinolin-7-yl)phenyl)sulfonyl)-1-oxa-4,9-diazaspiro[5.5]undecan-3-one

In a sealed microwave vial, a mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (140 mg, 0.294 mmol), 7-bromoquinolin-8-yl benzoate (96 mg, 0.294 mmol), 1,1′-bis(diphenylphosphino)ferrocene-palladium(II)dichloride dichloromethane complex (12 mg, 0.015 mmol) and 2M aq potassium carbonate solution (1 mL, 0.294 mmol) in 1,4-dioxane (2 mL) was stirred at 100° C. for 18 h. The reaction was cooled to room temperature and LCMS analysis revealed completion of the Suzuki reaction and partial progression of the benzoyl deprotection reaction. The reaction mixture was stirred for an additional 52 h at 100° C. and was then cooled to room temperature at which point two layers were visible. The mixture was diluted with ethyl acetate (5 mL) and the organic layer was removed by pipette, placed in a flask, dried over magnesium sulfate, and concentrated in vacuo. Purification of the residue by silica gel chromatography (0-10% methanol/ethyl acetate) followed by reverse phase HPLC (20-90% acetonitrile/water+0.1% NH₄OH) provided the desired product as a light green solid with impurities. The light green solid was purified by reverse phase HPLC (30-90% acetonitrile+0.1% TFA/water+0.1% TFA). The contents of the desired fractions were passed through a conditioned macroporous solid phase extraction plug (PL-HCO₃) to neutralize the compound. The resultant filtrate was concentrated partially to remove all the acetonitrile and lyophilized for 16 h, and then dried in a vacuum oven (50° C.) for 18 h to afford the title compound (17 mg, 12%) as a white solid. MS(ES)+ m/e 494.2 [M+H]⁺.

### Example 119

### 4-cyclopropyl-9-{[4-(7-quinolinyl)phenyl]sulfonyl}-1-oxa-4,9-diazaspiro[5.5]undecan-3-one-d₄

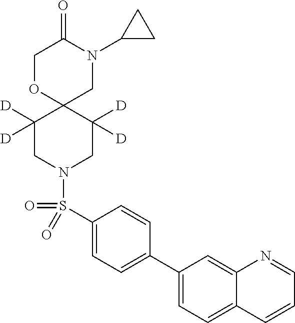 [SMILES: [2H]C1([2H])CN(S(=O)(=O)c2ccc(-c3ccc4cccnc4c3)cc2)CC([2H])([2H])C12CN(C1CC1)C(=O)CO2]

### a) 1-(phenylmethyl)-4-piperidinone-d₄

In a sealed oven-dried 20 mL microwave vial under nitrogen, a mixture of 1-benzyl-4-piperidinone (1.871 mL, 10.57 mmol) and sodium carbonate (4.48 g, 42.3 mmol) in D₂O (10 mL) was stirred at 120° C. for 17 h. The reaction was cooled to room temperature and two layers formed. Analysis by LCMS showed full conversion to the d₄ compound (no isotopic mass peaks detected for d₂ or d₃ compounds). The reaction mixture was diluted with ether (10 mL) and the layers were separated. The organic layer was dried over magnesium sulfate and concentrated in vacuo to give a yellow liquid. To ensure complete conversion and remove any minor undetected side products, the reaction was repeated. In a sealed oven-dried microwave vial under nitrogen, a mixture of the yellow liquid (10.57 mmol) and sodium carbonate (4.48 g, 42.3 mmol) in D₂O (10 mL) was stirred at 120° C. for 66 h. The reaction was cooled to room temperature and two layers formed. The reaction mixture was diluted with ether (10 mL) and the layers were separated. The organic layer was dried over magnesium sulfate and concentrated in vacuo to afford the title compound as a yellow liquid (1.39 g, 68%). MS(ES)+ m/e 193.9 [M+H]⁺.

### b) 6-(phenylmethyl)-1-oxa-6-azaspiro[2.5]octane-d₄

In an oven-dried round bottom flask under nitrogen, a yellow mixture of trimethylsulfoxonium iodide (2.058 g, 9.35 mmol) and anhydrous dimethyl sulfoxide-d₆ (5 mL) was stirred at room temperature for 1 h. The reaction was then cooled to 0° C. and sodium hydride (60% wt dispersion in mineral oil, 0.345 g, 8.63 mmol) was added in small portions over 1 minute. The reaction was allowed to warm to room temperature and stirred for 1.5 h. The resulting white slurry was cooled to 0° C. then treated with a solution of 1-(phenylmethyl)-4-piperidinone-d₄ (1.39 g, 7.19 mmol) in dimethyl sulfoxide-d₆ (2 mL) via cannula. The ice bath was removed and stirring continued at room temperature for 16 h. Water (10 mL) was added to quench the reaction and the mixture was extracted into ether (2×50 mL). The organic extracts were washed with brine, dried over magnesium sulfate, and evaporated under reduced pressure to a yellow oil. To remove residual DMSO contained in the oil, ethyl acetate (40 mL) was added to the oil and the solution was washed with water (40 mL). The layers were separated and the organic layer was dried over magnesium sulfate and concentrated in vacuo to afford the crude title compound as a yellow oil, which was used as is in the next experiment. MS(ES)+ m/e 207.9 [M+H]⁺.

### c) 4-[(cyclopropylamino)methyl]-1-(phenylmethyl)-4-piperidinol-d₄

A round bottom flask equipped with a reflux condenser was charged with 6-(phenylmethyl)-1-oxa-6-azaspiro[2.5]octane-d₄ (7.19 mmol), ethanol (25 mL) and cyclopropylamine (1.52 mL, 21.57 mmol). The reaction was stirred in a 75° C. oil bath giving a clear yellow solution. After stirring for 16 h, the reaction was cooled to room temperature and concentrated under reduced pressure. Purification of the residue by silica gel chromatography (0-10% methanol/ethyl acetate) provided the title product as a pale yellow oil (839 mg, 44%). MS(ES)+ m/e 265.3 [M+H]⁺.

### d) 2-chloro-N-cyclopropyl-N-{[4-hydroxy-1-(phenylmethyl)-4-piperidinyl]methyl}acetamide-d₄

To a cold 0° C. solution of 4-[(cyclopropylamino)methyl]-1-(phenylmethyl)-4-piperidinol-d₄ (839 mg, 3.17 mmol) in dichloromethane (10 mL) was added triethylamine (0.885 mL, 6.35 mmol) followed by chloroacetyl chloride (0.381 mL, 4.76 mmol). The ice bath was removed and the reaction was allowed to warm to room temperature with stirring. After 2 h, the reaction was quenched with water (5 mL) and the pH of the aqueous layer was adjusted to ˜7 with saturated aq sodium bicarbonate. The layers were separated and the aqueous layer was extracted with dichloromethane (10 mL). The combined organic layers were dried over magnesium sulfate and concentrated in vacuo to give the crude title compound as a dark brown gum, which was used as is in the next experiment. MS(ES)+ m/e 341.1 [M+H]⁺.

### e) 4-cyclopropyl-9-(phenylmethyl)-1-oxa-4,9-diazaspiro[5.5]undecan-3-one-d₄

To a mixture of 2-chloro-N-cyclopropyl-N-{[4-hydroxy-1-(phenylmethyl)-4-piperidinyl]methyl}acetamide-d₄ (3.17 mmol) in dry tetrahydrofuran (25 mL) under an atmosphere of nitrogen was added sodium hydride (60% wt dispersion in mineral oil, 571 mg, 14.27 mmol). The reaction mixture was stirred at room temperature for 15 h. Then, the reaction was quenched very carefully with dropwise addition of water (10 mL, note vigorous evolution of H₂ gas) and further diluted with brine (30 mL). The mixture was extracted with EtOAc (2×50 mL) and the extracts were combined, dried over MgSO₄, and concentrated in vacuo. Purification of the residue by silica gel chromatography (0-10% MeOH/EtOAc) provided the title product as a yellow oil (604 mg, 63%). By ¹H NMR weight/weight % calculation, this product contains about 7.8% of combined d₂ and d₃ compounds as impurities, which was carried forward in future experiments. MS(ES)+m/e 305.3 [M+H]⁺.

### f) 4-cyclopropyl-1-oxa-4,9-diazaspiro[5.5]undecan-3-one-d₄ hydrochloride

A solution of 4-cyclopropyl-9-(phenylmethyl)-1-oxa-4,9-diazaspiro[5.5]undecan-3-one-d₄ (136 mg, 0.447 mmol) in ethanol (6 mL) and 1N aq HCl (0.447 mL, 0.447 mmol) was passed through an H-cube containing 20% palladium hydroxide on carbon (cat. cart. 55) one time under hydrogen at 70° C. (1 mL/min, full H₂) and then the collected solution was recycled through the H-cube under hydrogen for 1 h under the same conditions. The resultant solution was concentrated in vacuo and dried further in a vacuum oven (50° C.) for 16 h to afford the title product (50 mg, 54%) as a white solid. MS(ES)+ m/e 215.0 [M+H]⁺.

### g) 9-[(4-bromophenyl)sulfonyl]-4-cyclopropyl-1-oxa-4,9-diazaspiro[5.5]undecan-3-one-d₄

To a mixture of 4-cyclopropyl-1-oxa-4,9-diazaspiro[5.5]undecan-3-one-d₄ hydrochloride (123 mg, 0.491 mmol) in dichloromethane (3 mL) under nitrogen was added triethylamine (0.171 mL, 1.226 mmol). The mixture was stirred for 2 min and then 4-bromobenzenesulfonyl chloride (150 mg, 0.589 mmol) was added. The yellow reaction mixture was stirred at room temperature for 4 h and then diluted with dichloromethane (30 mL) and water (10 mL). The layers were separated and the aqueous layer was extracted with dichloromethane (20 mL). The organic layers were combined, dried over MgSO₄, and concentrated in vacuo to give a white solid. The solid was triturated with hot 50% EtOAc/hexanes, and the solid was collected by filtration, suction dried and further dried in a vacuum oven (50° C.) for 16 h to give a white solid (188 mg). The solid was triturated with hot 50% EtOAc/hexanes, allowed to stand at room temperature for 1 h, and the solid was collected by filtration, rinsed with minimal 50% EtOAc/hexanes and suction dried to afford a white solid. This white solid was dissolved in ethyl acetate (5 mL) and dichloromethane (20 mL) and the solution was transferred to a separatory funnel. Water (10 mL) was added, the mixture was shaken, and then the layers were separated. The aqueous layer was then extracted with dichloromethane (10 mL). The organic layers were combined, dried over magnesium sulfate, and concentrated in vacuo to afford the title compound as a white solid (143 mg, 63%). MS(ES)+ m/e 433.1, 435.0 [M+H]⁺.

### h) 4-cyclopropyl-9-{[4-(7-quinolinyl)phenyl]sulfonyl}-1-oxa-4,9-diazaspiro[5.5]undecan-3-one-d₄

In a sealed microwave vial purged with nitrogen, a mixture of 9-[(4-bromophenyl)sulfonyl]-4-cyclopropyl-1-oxa-4,9-diazaspiro[5.5]undecan-3-one-d₄ (127 mg, 0.293 mmol), 7-(4,4,5,5-tetramethyl-1,3,2-dioxaborolan-2-yl)quinoline (79 mg, 0.308 mmol), and 1,1′-bis(diphenylphosphino)ferrocene-palladium(II)dichloride dichloromethane complex (12 mg, 0.015 mmol) in 1,4-dioxane (2 mL) and 2M aq potassium carbonate solution (1 mL) was stirred at 100° C. in an aluminum block for 1 h. The reaction was cooled to room temperature and two layers formed. The reaction mixture was diluted with ethyl acetate (10 mL) and the organic layer was removed by pipette. The organic layer was dried over magnesium sulfate and concentrated in vacuo to give a yellow solid. The yellow solid was then triturated in ethanol, filtered, and suction dried for 15 h to give a beige solid. The filtrate from the trituration was purified by silica gel chromatography (0-5% methanol/ethyl acetate) and the residue obtained was triturated in ethanol to provide the desired product as an ivory solid (20 mg, 13%). The beige solid was also purified by silica gel chromatography (0-5% methanol/ethyl acetate) and then the isolated solid was dried in a vacuum oven (70-80° C.) for 17 h to afford the title product (51 mg, 35%) as a white solid. MS(ES)+ m/e 481.8 [M+H]⁺.

### Example 120

### 4-cyclopropyl-9-((4-(6-fluoronaphthalen-2-yl)phenyl)sulfonyl)-1-oxa-4,9-diazaspiro[5.5]undecan-3-one

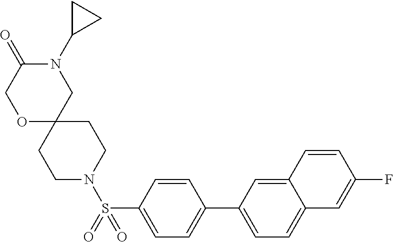 [SMILES: O=C1COC2(CCN(S(=O)(=O)c3ccc(-c4ccc5cc(F)ccc5c4)cc3)CC2)CN1C1CC1]

a) In a 5 mL microwave vial was placed 9-((4-bromophenyl)sulfonyl)-4-cyclopropyl-1-oxa-4,9-diazaspiro[5.5]undecan-3-one (0.233 mmol), 4,4,4′,4′,5,5,5′,5′-octamethyl-2,2′-bi(1,3,2-dioxaborolane) (0.233 mmol), potassium acetate (0.932 mmol), PdCl₂(dppf)-CH₂Cl₂ adduct (0.023 mmol), and 1,4-dioxane (2 mL). The vial was capped and the contents were purged with nitrogen. The solution was stirred at 100° C. for 1 h. Analysis by LCMS revealed complete conversion to the boronic ester intermediate. The reaction was cooled to room temperature and 2-bromo-6-fluoronaphthalene (0.233 mmol) and 2 M aq potassium carbonate (1.00 mL) were added. The vial was recapped, purged with nitrogen, and stirred at 100° C. The solution was cooled to room temperature. The dioxane layer was decanted and filtered through a plug of celite and sodium sulfate. The plug was washed with 1,4-dioxane (2 mL). The combined filtrate was concentrated in vacuo and purified by reverse phase HPLC (40-90% acetonitrile/water+0.1% NH₄OH) to afford the title compound (68 mg, 59%). MS(ES)+ m/e 494.8 [M+H]⁺.

### Example 121

### 4-cyclopropyl-9-((4-(8-fluoronaphthalen-2-yl)phenyl)sulfonyl)-1-oxa-4,9-diazaspiro[5.5]undecan-3-one

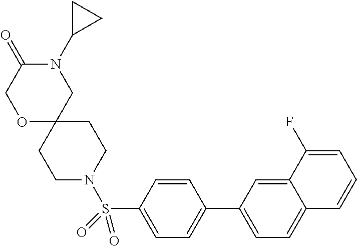 [SMILES: O=C1COC2(CCN(S(=O)(=O)c3ccc(-c4ccc5cccc(F)c5c4)cc3)CC2)CN1C1CC1]

### a) 8-fluoronaphthalen-2-yl trifluoromethanesulfonate

In a 50 mL round bottom flask under nitrogen was placed 8-fluoronaphthalen-2-ol (3.08 mmol). To this was added toluene (5 mL) and a premade 30% by weight solution of potassium phosphate tribasic (9.42 mmol) in water (4.6 mL). The flask was lowered into an ice bath and cooled to 0° C. for 5 min. Triflic anhydride (3.70 mmol) was added dropwise via syrinage and the solution was stirred for 1 hour, allowing the ice bath to warm to room temperature. Analysis by LCMS displayed loss of starting material, with only desired product present. The phases were allowed to separate. The aqueous layer was removed and the organic layer was washed with water (1×5 mL). The toluene layer was separated, dried over sodium sulfate, filtered, and concentrated in vacuo. Purification of the residue by silica gel chromatography (0-10% ethyl acetate/hexanes) afforded the title compound (0.78 g, 86%). ¹H NMR (400 MHz, CDCl₃) δ ppm 7.25-7.36 (m, 1H) 7.46 (dd, J=9.09, 2.53 Hz, 1H) 7.53 (td, J=7.96, 5.31 Hz, 1H) 7.72 (d, J=8.34 Hz, 1H) 7.99 (dd, J=9.09, 1.77 Hz, 1H) 8.02 (d, J=2.53 Hz, 1H).

### b) 2-(8-fluoronaphthalen-2-yl)-4,4,5,5-tetramethyl-1,3,2-dioxaborolane

In a 10 mL microwave vial was placed in succession 8-fluoronaphthalen-2-yl trifluoromethanesulfonate (2.311 mmol), bis(pinacolato)diboron (2.77 mmol), potassium acetate (6.93 mmol), PdCl₂(dppf)-CH₂Cl₂ adduct (0.070 mmol), dppf (0.069 mmol), and 1,4-dioxane (8 mL). The vial was capped and the contents were purged with nitrogen. The vial was stirred at 80° C. for 3 h. The reaction mixture was concentrated to dryness, taken up in ethyl acetate (50 mL), and washed with a 1:1 solution of water:brine. The organic layer was separated, dried over sodium sulfate, filtered, and concentrated in vacuo. The crude title product was used without further purification (0.5 g, 73%). MS(ES)+ m/e 272.8 [M+H]⁺.

### c) 4-cyclopropyl-9-((4-(8-fluoronaphthalen-2-yl)phenyl)sulfonyl)-1-oxa-4,9-diazaspiro[5.5]undecan-3-one

In a 5 ml, microwave vial was placed 9-((4-bromophenyl)sulfonyl)-4-cyclopropyl-1-oxa-4,9-diazaspiro[5.5]undecan-3-one (0.280 mmol) and PdCl₂(dppf)-CH₂Cl₂ adduct (0.024 mmol). A solution of crude 2-(8-fluoronaphthalen-2-yl)-4,4,5,5-tetramethyl-1,3,2-dioxaborolane (0.363 mmol) in 1,4-dioxane (2 mL) was then added, followed by 2 M aq potassium carbonate (1.00 mL). The reaction vial was capped, the contents were purged with nitrogen, and the solution was stirred at 100° C. After 1 hour, LCMS analysis determined the reaction was complete. The reaction was cooled to room temperature and the phases were allowed to separate. The dioxane layer was removed and passed through a plug of celite and sodium sulfate. The plug was washed with dioxane (2 mL). The dioxane filtrate was concentrated in vacuo and purified by reverse phase HPLC (30-90% acetonitrile/water+0.1% NH₄OH) to afford the title compound (67 mg, 48%). MS(ES)+ m/e 495.2 [M+H]⁺.

### Example 122

### 4-ethyl-9-((4-(3-methoxyquinolin-7-yl)phenyl)sulfonyl)-1-oxa-4,9-diazaspiro[5.5]undecan-3-one

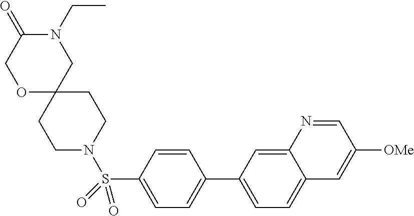 [SMILES: CCN1CC2(CCN(S(=O)(=O)c3ccc(-c4ccc5cc(OC)cnc5c4)cc3)CC2)OCC1=O]

a) In a 5 mL microwave vial was placed in succession 9-((4-bromophenyl)sulfonyl)-4-ethyl-1-oxa-4,9-diazaspiro[5.5]undecan-3-one (0.240 mmol), bis(pinacolato)diboron (0.276 mmol), potassium acetate (1.019 mmol), PdCl₂(dppf)-CH₂Cl₂ adduct (0.037 mmol), and 1,4-dioxane (2 mL). The vial was capped, purged with nitrogen, and stirred at 80° C. Analysis by LCMS indicated the loss of starting material and the presence of the desired boronate intermediate (and the related boronic acid). To the vial was added 7-bromo-3-methoxyquinoline (0.294 mmol) and 2 M aq potassium carbonate (1.00 mL). The vial was capped, purged with nitrogen, and returned to stirring at 80° C. for 1 h. The reaction vessel was set aside to cool to room temperature and to allow the dioxane layer to separate from the aqueous layer. The dioxane layer was decanted with a pipette and absorbed onto a silica pad, which was subjected to flash chromatography (0-10% methanol:dichloromethane). The recovered material was further purified by reverse phase HPLC (20-80% acetonitrile/water+0.1% NH₄OH) to afford the title compound (21 mg, 18%). MS(ES)+ m/e 496.2 [M+H]⁺.

### Example 123

### 4-isopropyl-9-((4-(3-methoxyquinolin-7-yl)phenyl)sulfonyl)-1-oxa-4,9-diazaspiro[5.5]undecan-3-one

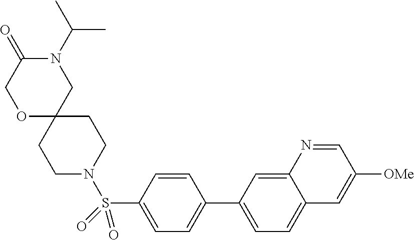 [SMILES: COc1cnc2cc(-c3ccc(S(=O)(=O)N4CCC5(CC4)CN(C(C)C)C(=O)CO5)cc3)ccc2c1]

a) Following the procedure described in Example 122a using 9-((4-bromophenyl)sulfonyl)-4-isopropyl-1-oxa-4,9-diazaspiro[5.5]undecan-3-one and additional purification using reverse phase HPLC (30-60% acetonitrile/water+0.1% TFA) followed by neutralization by partitioning between saturated aq sodium bicarbonate and ethyl acetate afforded the title product upon concentration in vacuo (18 mg, 15%). MS(ES)+ m/e 510.2 [M+H]⁺.

### Example 124

### 4-ethyl-9-((4-(3-fluoroquinolin-7-yl)phenyl)sulfonyl)-1-oxa-4,9-diazaspiro[5.5]undecan-3-one

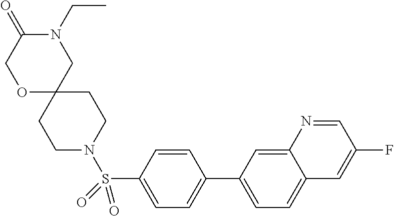 [SMILES: CCN1CC2(CCN(S(=O)(=O)c3ccc(-c4ccc5cc(F)cnc5c4)cc3)CC2)OCC1=O]

### a) 7-bromo-3-fluoroquinoline

Into a 100 mL round bottom flask was placed 7-bromoquinolin-3-amine (8.97 mmol) and chlorobenzene (20 mL). As this solution stirred, borontrifluoride dihydrate (13.61 mmol) was added dropwise via a syringe. This solution stirred under nitrogen as the flask temperature was raised to 50° C. An addition funnel was attached to the flask and into it was placed tert-butyl nitrite (8.97 mmol). This was added slowly portionwise to the warm starting materials over a 15 min period. The flask temperature was raised to 100° C. and the solution stirred under nitrogen for 2 h. The solution was cooled to room temperature and poured into a flask containing ice and saturated aq sodium bicarbonate (˜100 mL). The reaction flask was washed with chloroform and dichloromethane, combining to ˜100 mL. The solution was transferred to a separatory funnel and the organic layer was removed. The aqueous layer was washed with chloroform (3×). The combined organic extractions were washed with brine (1×), dried over sodium sulfate, filtered, and concentrated in vacuo. The residue was purified by silica gel chromatography (20-50% dichloromethane/hexanes) to afford the title compound (0.85 g, 36%). ¹H NMR (400 MHz, DMSO-d6) δ ppm 7.81-7.87 (m, 1H) 7.99 (d, J=8.84 Hz, 1H) 8.27-8.32 (m, 1H) 8.32-8.36 (m, 1H) 9.00 (d, J=2.78 Hz, 1H). MS(ES)+ m/e 226.0, 227.9 [M+H]⁺.

b) Following the procedure described in Example 120a using 9-((4-bromophenyl)sulfonyl)-4-isopropyl-1-oxa-4,9-diazaspiro[5.5]undecan-3-one and 7-bromo-3-fluoroquinoline and an additional purification using reverse phase HPLC (30-60% acetonitrile/water+0.1% TFA) followed by neutralization by passing a solution in acetonitrile through a PL-HCO₃ MP SPE tube (100 mg, 1.18 mmol/g loading) afforded the title product as a white solid upon concentration in vacuo (39 mg, 34%). MS(ES)+m/e 484.2 [M+H]⁺.

### Example 125

### 9-((4-(3-fluoroquinolin-7-yl)phenyl)sulfonyl)-4-isopropyl-1-oxa-4,9-diazaspiro[5.5]undecan-3-one

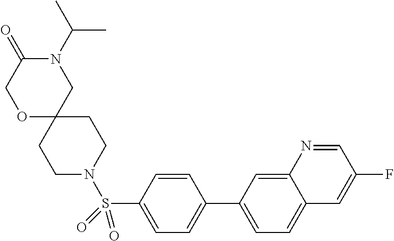 [SMILES: CC(C)N1CC2(CCN(S(=O)(=O)c3ccc(-c4ccc5cc(F)cnc5c4)cc3)CC2)OCC1=O]

a) Following the procedure described in Example 124b using 9-((4-bromophenyl)sulfonyl)-4-isopropyl-1-oxa-4,9-diazaspiro[5.5]undecan-3-one provided the title product (40 mg, 34%) as an off white solid. LCMS (ES⁺) m/z 498.2 [M+H]⁺.

### Example 126

### 9-((4-(3-methoxyquinolin-7-yl)phenyl)sulfonyl)-4-(1-methylcyclopropyl)-1-oxa-4,9-diazaspiro[5.5]undecan-3-one

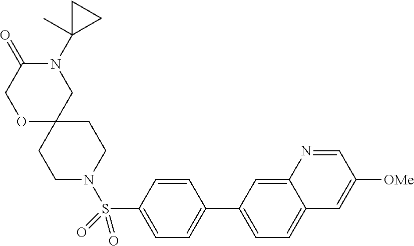 [SMILES: COc1cnc2cc(-c3ccc(S(=O)(=O)N4CCC5(CC4)CN(C4(C)CC4)C(=O)CO5)cc3)ccc2c1]

a) Following the procedure described in Example 120a using 9-((4-bromophenyl)sulfonyl)-4-(1-methylcyclopropyl)-1-oxa-4,9-diazaspiro[5.5]undecan-3-one and 7-bromo-3-methoxyquinoline and an additional purification by reverse phase HPLC (25-55% acetonitrile/water+0.1% TFA) followed by neutralization of the product by passing a solution in acetonitrile through a PL-HCO₃ MP SPE tube (100 mg, 1.18 mmol/g loading) afforded the title product as a white solid upon concentration in vacuo (24 mg, 19%). MS(ES)+ m/e 522.3 [M+H]⁺.

### Example 127

### 9-((4-(3-fluoroquinolin-7-yl)phenyl)sulfonyl)-4-(1-methylcyclopropyl)-1-oxa-4,9-diazaspiro[5.5]undecan-3-one

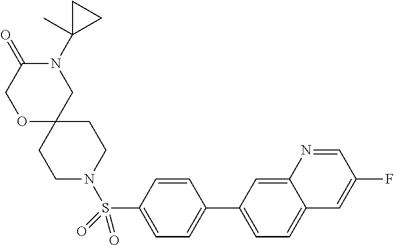 [SMILES: CC1(N2CC3(CCN(S(=O)(=O)c4ccc(-c5ccc6cc(F)cnc6c5)cc4)CC3)OCC2=O)CC1]

a) Following the procedure described in Example 126a using 7-bromo-3-fluoroquinoline provided the title product (30 mg, 24%) as a white solid. LCMS (ES⁺) m/z 510.1 [M+H]⁺.

### Example 128

### 4′-[(4-cyclopropyl-3-oxo-1-oxa-4,9-diazaspiro[5.5]undec-9-yl)sulfonyl]-3-biphenylcarboxylic acid

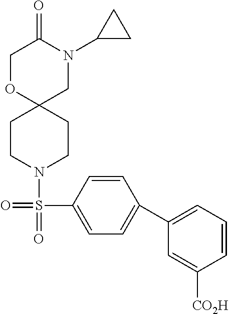 [SMILES: O=C(O)c1cccc(-c2ccc(S(=O)(=O)N3CCC4(CC3)CN(C3CC3)C(=O)CO4)cc2)c1]

a) To a microwave vial (30 mL) was added 9-[(4-bromophenyl)sulfonyl]-4-cyclopropyl-1-oxa-4,9-diazaspiro[5.5]undecan-3-one (0.233 mmol), 3-(dihydroxyboranyl)benzoic acid (0.233 mmol), PdCl₂(dppf)-CH₂Cl₂ adduct (0.012 mmol), 1,4-dioxane (3 mL) and then 2 M aq K₂CO₃ (300 uL). The vial was capped, flushed with nitrogen then heated to 135° C. for 90 min. The reaction mixture was concentrated under reduced pressure and the residue was dissolved in dimethylsulfoxide (3 mL), filtered through a syringe filter, and purified by reverse phase HPLC (10-90% acetonitrile/water+0.1% TFA). The appropriate fractions were concentrated to remove a majority of the acetonitrile, leaving an aqueous suspension of product which was collected by filtration and dried to constant weight to provide the title product (44 mg, 39% yield) as a white solid. MS(ES)+ m/e 486.2 [M+H]⁺.

### Example 129

### 4-cyclopropyl-9-{[4′-(1H-tetrazol-5-yl)-4-biphenylyl]sulfonyl}-1-oxa-4,9-diazaspiro[5.5]undecan-3-one

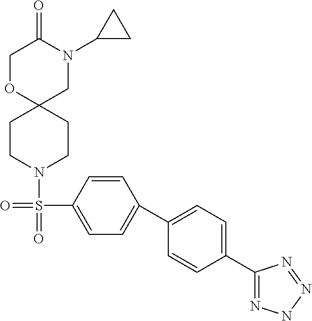 [SMILES: O=C1COC2(CCN(S(=O)(=O)c3ccc(-c4ccc(-c5nn[nH]n5)cc4)cc3)CC2)CN1C1CC1]

a) Following the procedure described in Example 128a with [4-(1H-tetrazol-5-yl)phenyl]boronic acid provided the product. Further purification via reverse phase HPLC (65:35 300 mM aq ammonium formate, pH 4:acetonitrile) afforded the title product (8 mg, 7% yield). MS(ES)+ m/e 495.0 [M+H]⁺.

### Example 130

### {4′-[(4-cyclopropyl-3-oxo-1-oxa-4,9-diazaspiro[5.5]undec-9-yl)sulfonyl]-3-biphenylyl}boronic acid

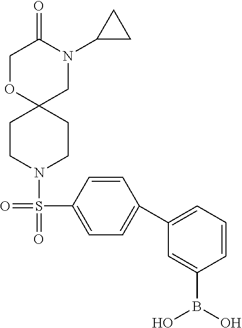 [SMILES: O=C1COC2(CCN(S(=O)(=O)c3ccc(-c4cccc(B(O)O)c4)cc3)CC2)CN1C1CC1]

a) Following the procedure described in Example 128a with benzene-1,3-diyldiboronic acid (3 eq) gave the title product (22 mg, 19% yield). MS(ES)+ m/e 471.1 [M+H]⁺.

### Example 131

### 4′[(4-cyclopropyl-3-oxo-1-oxa-4,9-diazaspiro[5.5]undec-9-yl)sulfonyl]-3-biphenylsulfonic acid

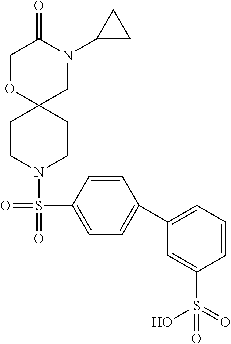 [SMILES: O=C1COC2(CCN(S(=O)(=O)c3ccc(-c4cccc(S(=O)(=O)O)c4)cc3)CC2)CN1C1CC1]

a) To a vial (30 mL) was added 4-cyclopropyl-9-{[4-(4,4,5,5-tetramethyl-1,3,2-dioxaborolan-2-yl)phenyl]sulfonyl}-1-oxa-4,9-diazaspiro[5.5]undecan-3-one (0.315 mmol), pentafluorophenyl 3-bromobenzenesulfonate (0.315 mmol), PdCl₂(dppf)-CH₂Cl₂ adduct (0.016 mmol), 1,4-dioxane (3 mL) and 2M aq sodium bicarbonate (300 μL). The vial was flushed with nitrogen and heated to 125° C. for 60 min. The reaction was cooled, acidified with 1 N aq HCl and the reaction was concentrated. The residue was taken up in dimethylsulfoxide (1.5 mL) and filtered. The resulting solution was chromatographed via reverse phase HPLC (10-90% acetonitrile/water+0.1% TFA). Evaporation of the product fractions gave the title product (50 mg, 31% yield). MS(ES)+ m/e 506.9 [M+H]⁺.

### Example 132

### 2-cyclopropyl-9-{[4-(7-quinolinyl)phenyl]sulfonyl}-2,9-diazaspiro[5.5]undecan-3-one

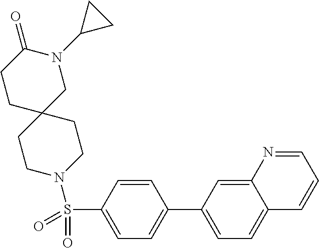 [SMILES: O=C1CCC2(CCN(S(=O)(=O)c3ccc(-c4ccc5cccnc5c4)cc3)CC2)CN1C1CC1]

### a) 1-(1,1-dimethylethyl) 4-ethyl 4-(3-{[(1,1-dimethylethyl)(dimethyl)silyl]oxy}propyl)-1,4-piperidinedicarboxylate

In a round-bottom flask, diisopropylamine (28.2 mmol) in tetrahydrofuran (60 mL) was cooled to −78° C. N-Butyllithium (16.03 mL, 25.6 mmol) was added to the cooled solution, which was allowed to stir for 1 h. In a separate flask, 1-(1,1-dimethylethyl) 4-ethyl 1,4-piperidinedicarboxylate (23.32 mmol) was dissolved in tetrahydrofuran (40 mL) and cooled to −78° C. The prepared LDA was added to the cooled solution via syringe. After 1 h, [(3-bromopropyl)oxy](1,1-dimethylethyl)dimethylsilane (26.8 mmol) was added and the solution was stirred at −78° C. for 1 h and then allowed to gradually warm to room temperature overnight. After 16 h, analysis by LCMS indicated formation of the desired product. Saturated aq NH₄Cl was slowly and carefully added to the reaction flask, which was then transferred to a reparatory funnel containing ether. The organic layer was extracted with ether (3×) and the combined organics were washed with brine, dried over Na₂SO₄, and concentrated in vacuo to afford the crude title product as an oil MS(ES)+m/e 430.1 [M+H]⁺. This material was carried forward without further purification.

### b) 1,1-dimethylethyl 4-(3-{[(1,1-dimethylethyl)(dimethyl)silyl]oxy}propyl)-4-(hydroxymethyl)-1-piperidinecarboxylate

To a round bottom flask, LiBH₄ (93 mmol) was added, dissolved in tetrahydrofuran (100 mL), and cooled to −78° C. In a separate flask, the 1-(1,1-dimethylethyl) 4-ethyl 4-(3-{[(1,1-dimethylethyl)(dimethyl)silyl]oxy}propyl)-1,4-piperidinedicarboxylate from Example 132a was dissolved in tetrahydrofuran (16 mL) and then added to the cooled solution via syringe. The reaction flask was allowed to warm to room temperature and stirred for 18 h. The mixture was then heated to 40° C. for 6 h, until analysis by LCMS indicated complete conversion to desired product. The mixture was cooled to 0° C. and water was slowly and carefully added until evolution of gas subsided. This mixture was poured into a separatory funnel containing ethyl acetate and saturated aq sodium bicarbonate. The organic layer was extracted with ethyl acetate (3×) and the combined organics were washed with brine, dried over Na₂SO₄, and concentrated in vacuo to an oil. Purification of the residue by silica gel chromatography (10-100% ethyl acetate/hexanes) afforded the title product as an oil (4.2 g, 46% over the two steps). MS(ES)+ m/e 388.1 [M+H]⁺.

### c) 1,1-dimethylethyl 4-(3-{[(1,1-dimethylethyl)(dimethyl)silyl]oxy}propyl)-4-formyl-1-piperidinecarboxylate

To a round-bottom flask, 1,1-dimethylethyl 4-(3-{[(1,1-dimethylethyl)(dimethyl)silyl]oxy}propyl)-4-(hydroxymethyl)-1-piperidinecarboxylate (1.548 mmol) was dissolved in dichloromethane (3500 μl) and Hunig's base (1081 μl). In a separate vial, sulfur trioxide pyridine complex (985 mg, 6.19 mmol) was dissolved in dimethyl sulfoxide (3500 μA) and was then added to the reaction mixture via syringe. The reaction was stirred at room temperature for 30 min when analysis by LCMS indicated desired product formation. The solution was cooled to 0° C. and water was slowly and carefully added until evolution of gas subsided. This mixture was poured into a separatory funnel containing dichloromethane and saturated aq sodium bicarbonate. The organic layer was extracted with dichloromethane (3×) and the combined organics were washed with brine, dried over Na₂SO₄, and concentrated in vacuo to afford the crude title product as an oil. MS(ES)+ m/e 386.3 [M+H]⁺. This material was carried forward without further purification.

### d) 1,1-dimethylethyl 4-[(cyclopropylamino)methyl]-4-(3-{[(1,1-dimethylethyl)(dimethyl)silyl]oxy}propyl)-1-piperidinecarboxylate

To a round-bottom flask, the crude 1,1-dimethylethyl 4-(3-{[(1,1-dimethylethyl)(dimethyl)silyl]oxy}propyl)-4-formyl-1-piperidinecarboxylate from example 132c and potassium acetate (5.50 mmol) were added and dissolved in tetrahydrofuran (20 mL). Cyclopropylamine (27.5 mmol) and Hunig's base (13.74 mmol) were added and the solution was stirred at room temperature for 25 h, when analysis by LCMS indicated complete conversion to the desired imine intermediate. Sodium borohydride (13.74 mmol) was added and the mixture was stirred at room temperature for 30 min. Analysis by LCMS indicated desired product formation. This mixture was poured into a separatory funnel containing ethyl acetate and saturated aq sodium bicarbonate. The organic layer was extracted with ethyl acetate (3×) and the combined organics were washed with brine, dried over Na₂SO₄, and concentrated in vacuo to afford the crude title product as an oil. MS(ES)+ m/e 427.0 [M+H]⁺. This material was carried forward without further purification.

### e) 1,1-dimethylethyl 4-[(cyclopropylamino)methyl]-4-(3-hydroxypropyl)-1-piperidinecarboxylate

To a round-bottom flask, the crude 1,1-dimethylethyl 4-[(cyclopropylamino)methyl]-4-(3-{[(1,1-dimethylethyl)(dimethyl)silyl]oxy}propyl)-1-piperidinecarboxylate from example 132d was added and dissolved in tetrahydrofuran (15 mL). Tetrabutylammonium fluoride (5 mL, 5.00 mmol; 1M in THF) was added to the solution, which was then heated to 40° C. for 1 h. Analysis by LCMS indicated conversion to desired product. The mixture was cooled to ambient temperature and poured into a separatory funnel containing ethyl acetate and saturated aq ammonium chloride. The organic layer was extracted with ethyl acetate (3×) and the combined organics were washed with brine, dried over Na₂SO₄, and concentrated in vacuo to afford the crude title product as an oil. MS(ES)+ m/e 313.1 [M+H]⁺. This material was carried forward without further purification.

### f) 3-(4-[(cyclopropyl {[(phenylmethyl)oxy]carbonyl}amino)methyl]-1-{[(1,1-dimethylethyl)oxy]carbonyl}-4-piperidinyl)propanoic acid

To a round-bottom flask, the crude 1,1-dimethylethyl 4-[(cyclopropyl {[(phenylmethyl)oxy]carbonyl}amino)methyl]-4-(3-hydroxypropyl)-1-piperidinecarboxylate from example 132e was added, dissolved in dichloromethane (21.400 mL), and cooled to −78° C. Benzyloxycarbonyl chloride (1.613 mmol) and Hunig's base (1.409 mL) were added to the reaction, which was allowed to warm to room temperature and stirred for 90 min. Analysis by LCMS indicated conversion to the desired protected amine. This solution was concentrated in vacuo, re-dissolved in acetone (20 mL) and cooled to 0° C. The Jones reagent (5 mL, 2.304 mmol; a stock solution was prepared by adding CrO₃ (0.25 mol) and concentrated sulfuric acid (25 mL) to a round bottom flask, cooling the solution to 0° C., and diluting the solution gradually with portions of water (75 mL)) was added to the solution, which was stirred for 5 min. Analysis by LCMS indicated desired product formation. Isopropanol was added to the cooled solution slowly to gradually quench the reaction (color change observed to quench excess jones reagent). A solid began to form, which was filtered off and washed with acetone. The filtrate was concentrated, dissolved in ethyl acetate, and added to a reparatory funnel containing water. The organic layer was extracted with ethyl acetate (3×) and the combined organics were washed with brine, dried over Na₂SO₄, and concentrated in vacuo to a residue. This residue was purified by silica gel chromatography (0-25% isopropanol/ethyl acetate) to afford the title product as a tan solid (275 mg, 38% over the four steps). MS(ES)+ m/e 461.5 [M+H]⁺.

### g) 1,1-dimethylethyl 2-cyclopropyl-3-oxo-2,9-diazaspiro[5.5]undecane-9-carboxylate

To a round-bottom flask, 3-(4-[(cyclopropyl {[(phenylmethyl)oxy]carbonyl}amino)methyl]-1-{[(1,1-dimethylethyl)oxy]carbonyl}-4-piperidinyl)propanoic acid (0.586 mmol) was dissolved in ethanol (6000 μl), and to that was added 10% w/w Pd/C (62.4 mg, 0.059 mmol). The reaction was subjected to a hydrogen atmosphere via balloon and was stirred at room temperature for 1 h. Analysis by LCMS indicated formation of the desired lactam. The palladium was removed via syringe filter and the filtrate was concentrated in vacuo to afford the crude title product as a dark residue. MS(ES)+ m/e 309.3 [M+H]⁺. This material was carried forward without further purification.

### h) 9-((4-bromophenyl)sulfonyl)-2-cyclopropyl-2,9-diazaspiro[5.5]undecan-3-one

To a round-bottom flask, the crude 1,1-dimethylethyl 2-cyclopropyl-3-oxo-2,9-diazaspiro[5.5]undecane-9-carboxylate from Example 132g was added, followed by HCl (1.0 ml, of 4.0M HCl in 1,4-dioxane). This mixture was stirred at room temperature for 3 h. Analysis by LCMS indicated conversion to the desired deprotected intermediate. The solution was concentrated in vacuo and suspended in pyridine (1 mL). 4-Bromobenzene-1-sulfonyl chloride (0.272 mmol) and 4-(dimethylamino)pyridine (0.04 mmol) were added and the mixture was heated at 80° C. for 1 h. Purification of the solution by reverse phase HPLC (10-90% acetonitrile/water+0.1% TFA) afforded the title product as an off-white solid (51 mg, 20% over two steps). MS(ES)+ m/e 427.0, 428.7 [M+H]⁺.

### i) 2-cyclopropyl-9-{[4-(7-quinolinyl)phenyl]sulfonyl}-2,9-diazaspiro[5.5]undecan-3-one

To a microwave vial, 9-((4-bromophenyl)sulfonyl)-2-cyclopropyl-2,9-diazaspiro[5.5]undecan-3-one (0.117 mmol), 7-(4,4,5,5-tetramethyl-1,3,2-dioxaborolan-2-yl)quinoline (0.18 mmol), Cs₂CO₃ (0.522 mmol), and dichloro[1,1′-bis(diphenylphosphino)ferrocene]palladium(II)-dichloromethane adduct (0.015 mmol) were added and the mixture was purged with nitrogen. 1,4-Dioxane (1.1 mL) and water (0.7 mL) were added to the mixture, which was heated for 3 h on a hot plate at 100° C. Analysis by LCMS indicated desired product formation and consumption of starting material. The mixture was filtered through a syringe filter and purified by reverse phase HPLC (10-90% acetonitrile/water+0.1% TFA). The desired fractions were collected and added to a separatory funnel containing ethyl acetate and saturated aq sodium bicarbonate. The aqueous phase was extracted with ethyl acetate (3×) and the combined organics were washed with brine, dried over Na₂SO₄, and concentrated in vacuo to afford the title product as a white solid (34 mg, 61%). MS(ES)+ m/e 476.1 [M+H]⁺.

### Example 133

### ethyl 7-(4-((4-cyclopropyl-3-oxo-1-oxa-4,9-diazaspiro[5.5]undecan-9-yl)sulfonyl)phenyl)quinoline-3-carboxylate

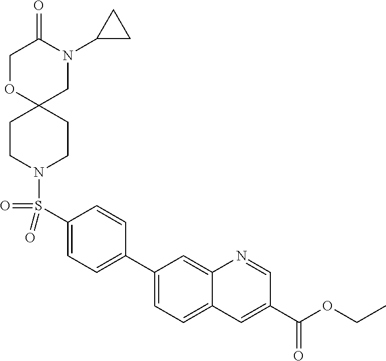 [SMILES: CCOC(=O)c1cnc2cc(-c3ccc(S(=O)(=O)N4CCC5(CC4)CN(C4CC4)C(=O)CO5)cc3)ccc2c1]

### a) ethyl 7-bromo-3-quinolinecarboxylate

A mixture of 2-amino-4-bromobenzaldehyde (25.00 mmol), ethyl (2E)-3-(ethyloxy)-2-propenoate (62.5 mmol) and 4-methylbenzenesulfonic acid (2.500 mmol) in toluene (80 mL) was heated under reflux over an oil bath for 18 h. The mixture was cooled, washed with saturated aq sodium bicarbonate, and concentrated in vacuo. Purification of the residue by flash chromatography (10-25% ethyl acetate/hexanes) gave the title product (3.1 g, 44% yield). MS (ES+) m/e 282.1 [M+H]⁺. ¹H NMR (400 MHz, DMSO-d6) δ=9.33 (d, 1H), 9.06 (d, J=1.8 Hz, 1H), 8.35 (d, J=2.0 Hz, 1H), 8.21 (d, J=8.8 Hz, 1H), 7.90 (dd, J=1.9, 8.7 Hz, 1H), 4.43 (q, J=7.1 Hz, 2H), 1.39 (t, J=7.1 Hz, 3H).

### b) ethyl 7-(4-((4-cyclopropyl-3-oxo-1-oxa-4,9-diazaspiro[5.5]undecan-9-yl)sulfonyl)phenyl)quinoline-3-carboxylate

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.435 mmol), ethyl 7-bromoquinoline-3-carboxylate (0.507 mmol), bis(triphenylphosphino)dichloropalladium(II) (0.022 mmol) and potassium carbonate (0.435 mmol) in ethanol (6.0 mL) and water (1.5 mL) was heated under reflux at 80° C. for 1 h. Analysis by LCMS showed no starting materials present, but partial hydrolysis of the ester was visible. The mixture was cooled, diluted with ethyl acetate and water, the layers separated and the aqueous layer extracted with ethyl acetate. The combined organics were washed with 1 M hydrochloric acid, dried over sodium sulfate, and concentrated in vacuo. Some crystalline material had developed overnight, so the gum was slurried in ethyl acetate and the solid collected by filtration. Purification of the mother liquors by flash chromatography (0-10% methanol in ethyl acetate) and crystallization of the product from ethanol gave title product (56 mg, 23% yield). MS (ES+) m/e 550.1 [M+H]⁺. ¹H NMR (400 MHz, DMSO-d6) δ=9.39 (d, 1H), 9.09 (d, J=1.8 Hz, 1H), 8.50 (s, 1H), 8.39 (d, J=8.6 Hz, 1H), 8.26-8.12 (m, 3H), 7.89 (d, J=8.3 Hz, 2H), 4.45 (q, J=7.1 Hz, 2H), 3.86 (s, 2H), 3.55 (d, J=11.6 Hz, 2H), 3.13 (s, 2H), 2.77-2.69 (m, 1H), 2.48-2.40 (m, 2H), 1.85 (d, J=13.4 Hz, 2H), 1.78-1.61 (m, 2H), 1.41 (t, J=7.1 Hz, 3H), 0.76-0.63 (m, 2H), 0.63-0.53 (m, 2H).

### Example 134

### 7-(4-((4-cyclopropyl-3-oxo-1-oxa-4,9-diazaspiro[5.5]undecan-9-yl)sulfonyl)phenyl)quinoline-3-carboxylic acid

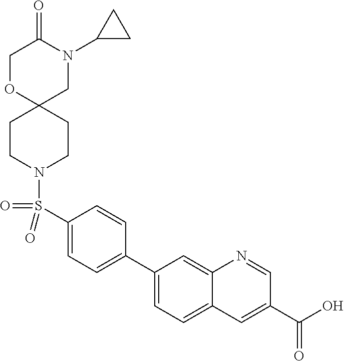 [SMILES: O=C(O)c1cnc2cc(-c3ccc(S(=O)(=O)N4CCC5(CC4)CN(C4CC4)C(=O)CO5)cc3)ccc2c1]

### a) 7-bromoquinoline-3-carboxylic acid

A mixture of ethyl 7-bromoquinoline-3-carboxylate (17.85 mmol) in 1 M aq sodium hydroxide (100 mL) was heated at 70° C. for 4 h. The mixture was cooled, neutralized, saturated with sodium chloride, and extracted into ethyl acetate. The organic solution was dried over sodium sulfate and concentrated in vacuo, and the solid was crystallized from ethanol to give the title product (210 mg, 5% yield). MS (ES+) m/e 253.9 [M+H]⁺. ¹H NMR (400 MHz, DMSO-d6) δ=13.61 (br. s, 1H), 9.33 (d, J=2.0 Hz, 1H), 9.04 (d, J=1.8 Hz, 1H), 8.35 (d, J=1.5 Hz, 1H), 8.20 (d, J=8.8 Hz, 1H), 7.89 (dd, J=1.9, 8.7 Hz, 1H).

### b) 7-(4-((4-cyclopropyl-3-oxo-1-oxa-4,9-diazaspiro[5.5]undecan-9-yl)sulfonyl)phenyl)quinoline-3-carboxylic acid

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.714 mmol), 7-bromoquinoline-3-carboxylic acid (0.595 mmol), bis(triphenylphosphino)dichloropalladium(II) (0.050 mmol) and potassium carbonate (2.380 mmol) in ethanol (10 mL) and water (2.5 mL) was heated under reflux at 80° C. for 2.5 h. The mixture was cooled, diluted with ethyl acetate and water, the layers separated, the aqueous layer was extracted with ethyl acetate, and the combined organic extracts were washed with 1 M aq hydrochloric acid, dried over sodium sulfate, and concentrated in vacuo. Purification of the residue by reverse phase HPLC (10-80% acetonitrile/water+0.1% TFA), followed by evaporation of the desired fractions to low volume, precipitation of the solid, filtration, washing with water, and concentration in vacuo afforded the title product (25 mg, 8% yield). MS (ES+) m/e 522.2 [M+H]⁺. ¹H NMR (400 MHz, DMSO-d6) δ=13.60 (s, 1H), 9.38 (d, J=2.0 Hz, 1H), 9.05 (d, J=1.8 Hz, 1H), 8.49 (s, 1H), 8.36 (d, J=8.6 Hz, 1H), 8.20 (d, J=8.6 Hz, 2H), 8.15 (dd, J=1.8, 8.6 Hz, 1H), 7.89 (d, J=8.3 Hz, 2H), 3.86 (s, 2H), 3.55 (d, J=11.4 Hz, 2H), 3.13 (s, 2H), 2.72 (tt, J=3.9, 7.4 Hz, 1H), 2.49-2.36 (m, 2H), 1.92-1.78 (m, 2H), 1.69 (td, J=4.3, 13.1 Hz, 2H), 0.77-0.63 (m, 2H), 0.63-0.51 (m, 2H).

### Example 135

### 9-((4-(3-aminoquinolin-7-yl)phenyl)sulfonyl)-4-cyclopropyl-1-oxa-4,9-diazaspiro[5.5]undecan-3-one

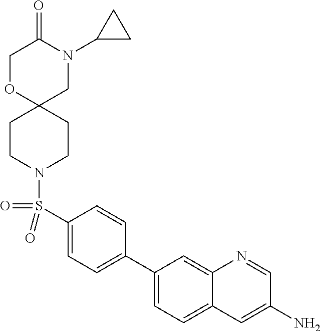 [SMILES: Nc1cnc2cc(-c3ccc(S(=O)(=O)N4CCC5(CC4)CN(C4CC4)C(=O)CO5)cc3)ccc2c1]

### a) 2-(7-bromoquinolin-3-yl)isoindoline-1,3-dione

A mixture of 2-(2,2-diethoxyethyl)isoindoline-1,3-dione (90 mmol), 2-amino-4-bromobenzaldehyde (75.0 mmol) and p-toluenesulfonic acid monohydrate (75.0 mmol) in toluene (300 mL) was heated under reflux using a Dean/Stark head for 4 h. The mixture was cooled and filtered and the solid was washed with toluene and hexanes to give the crude PTSA salt of the product as a brown solid. The solid was stirred with saturated aq sodium bicarbonate and extracted into dichloromethane, ensuring any precipitate was dissolved in additional solvent during separation. However, some solid precipitated, which was found to be mostly desired product. The solid was slurried in ethanol and collected to give desired product. The mother liquors were evaporated onto silica gel and purified by flash chromatography (0-3% methanol in dichloromethane) to afford additional product. The two batches were combined to yield the title product (18.2 g, 69% yield). MS (ES+) m/e 355.1 [M+H]⁺. ¹H NMR (400 MHz, DMSO-d6) δ 7.87 (dd, J=8.59, 2.02 Hz, 1H) 7.93-8.01 (m, 2H) 8.02-8.08 (m, 2H) 8.11 (d, J=8.59 Hz, 1H) 8.35 (d, J=1.77 Hz, 1H) 8.57 (d, J=2.27 Hz, 1H) 9.05 (d, J=2.53 Hz, 1H).

### b) 2-(7-(4-((4-cyclopropyl-3-oxo-1-oxa-4,9-diazaspiro[5.5]undecan-9-yl)sulfonyl)phenyl)quinolin-3-yl)isoindoline-1,3-dione

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (3.27 mmol), 2-(7-bromoquinolin-3-yl)isoindoline-1,3-dione (3.11 mmol), potassium acetate (7.79 mmol) and dichloro[1,1′-bis(diphenylphosphino)ferrocene]palladium(II)-dichloromethane adduct (0.156 mmol) in 1,4-dioxane (60 mL) was heated under reflux in a nitrogen atmosphere for 24 h. The mixture was diluted with water and the solid was collected and washed with water, ethanol, and hexanes. The solid was dissolved in chloroform, silicycle thiol was added, and the mixture was stirred at 50° C. for 48 h. The mixture was filtered hot, washed with chloroform, and concentrated in vacuo to a solid that was slurried in hot ethanol, cooled, and collected to afford the title product (940 mg, 48% yield) as a grey solid. MS (ES+) m/e 623.2 [M+H]⁺. ¹H NMR (400 MHz, DMSO-d6) δ 0.54-0.63 (m, 2H) 0.64-0.73 (m, 2H) 1.62-1.77 (m, 2H) 1.85 (d, J=14.15 Hz, 2H) 2.45 (d, J=1.77 Hz, 2H) 2.69-2.78 (m, 1H) 3.14 (s, 2H) 3.55 (d, J=12.63 Hz, 2H) 3.87 (s, 2H) 7.89 (d, J=8.59 Hz, 2H) 7.94-8.01 (m, 3H) 8.02-8.09 (m, 3H) 8.15 (dd, J=8.59, 1.77 Hz, 1H) 8.22 (d, J=8.59 Hz, 2H) 8.27 (d, J=8.59 Hz, 1H) 8.52 (s, 1H) 8.59 (d, J=2.27 Hz, 1H) 9.08 (d, J=2.27 Hz, 1H).

### c) 9-((4-(3-aminoquinolin-7-yl)phenyl)sulfonyl)-4-cyclopropyl-1-oxa-4,9-diazaspiro[5.5]undecan-3-one

A mixture of 2-(7-(4-((4-cyclopropyl-3-oxo-1-oxa-4,9-diazaspiro[5.5]undecan-9-yl)sulfonyl)phenyl)quinolin-3-yl)isoindoline-1,3-dione (1.494 mmol) in chloroform (50 mL) was treated portionwise (while monitoring the reaction progression) with hydrazine (125 μl, 3.98 mmol) and heated under reflux for 2 h. The solvent was partially concentrated in vacuo and the remaining solution applied to a 10 g silica gel precolumn. Purification by flash chromatography (0-5% methanol in dichloromethane) gave the product as a foam upon concentration in vacuo. Crystallization of the foam from ethanol afforded the title product (480 mg, 65% yield) as an off-white solid. MS (ES+) m/e 493.1 [M+H]⁺. ¹H NMR (400 MHz, DMSO-d6) δ 0.50-0.62 (m, 2H) 0.62-0.74 (m, 2H) 1.57-1.77 (m, 2H) 1.76-1.96 (m, 2H) 2.36-2.47 (m, 2H) 2.71 (br. s, 1H) 3.12 (br. s, 2H) 3.52 (d, J=11.12 Hz, 2H) 3.86 (br. s, 2H) 5.88 (br. s, 2H) 7.16 (br. s, 1H) 7.69-7.78 (m, 1H) 7.81 (d, J=6.57 Hz, 3H) 8.07 (d, J=7.83 Hz, 2H) 8.17 (br. s, 1H) 8.50 (br. s, 1H).

### Example 136

### N-(7-(4-((4-cyclopropyl-3-oxo-1-oxa-4,9-diazaspiro[5.5]undecan-9-yl)sulfonyl)phenyl)quinolin-3-yl)acetamide

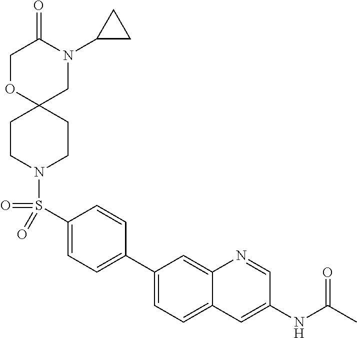 [SMILES: CC(=O)Nc1cnc2cc(-c3ccc(S(=O)(=O)N4CCC5(CC4)CN(C4CC4)C(=O)CO5)cc3)ccc2c1]

a) A solution of 9-((4-(3-aminoquinolin-7-yl)phenyl)sulfonyl)-4-cyclopropyl-1-oxa-4,9-diazaspiro[5.5]undecan-3-one (0.152 mmol) in dichloromethane (6 mL) was treated with N-ethyl-N-isopropylpropan-2-amine (0.228 mmol) and then acetyl chloride (0.183 mmol), and the flask was sealed and the mixture stirred at ambient temperature overnight. The mixture was washed with dilute brine, dried over sodium sulfate, and concentrated in vacuo. Purification of the residue by flash chromatography (0-10% methanol in dichloromethane) and recrystallization from acetonitrile gave the title product (25 mg, 31% yield) as pale yellow crystals. MS (ES+) m/e 535.0 [M+H]⁺. ¹H NMR (400 MHz, DMSO-d6) δ ppm 0.53-0.63 (m, 2H) 0.63-0.72 (m, 2H) 1.62-1.75 (m, 2H) 1.78-1.90 (m, 2H) 2.17 (s, 3H) 2.40-2.49 (m, 2H) 2.72 (tt, J=7.39, 3.85 Hz, 1H) 3.13 (s, 2H) 3.54 (d, J=11.62 Hz, 2H) 3.86 (s, 2H) 7.85 (d, J=8.34 Hz, 2H) 8.00 (dd, J=8.46, 1.89 Hz, 1H) 8.05-8.11 (m, 1H) 8.14 (d, J=8.34 Hz, 2H) 8.33 (s, 1H) 8.77 (d, J=2.27 Hz, 1H) 8.94 (d, J=2.53 Hz, 1H) 10.53 (s, 1H).

### Example 137

### 7-(4-((4-cyclopropyl-3-oxo-1-oxa-4,9-diazaspiro[5.5]undecan-9-yl)sulfonyl)phenyl)quinoline-3-carbonitrile

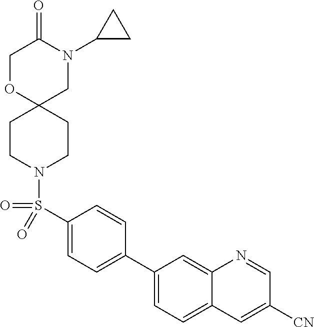 [SMILES: N#Cc1cnc2cc(-c3ccc(S(=O)(=O)N4CCC5(CC4)CN(C4CC4)C(=O)CO5)cc3)ccc2c1]

### a) 7-bromoquinoline-3-carbonitrile

A mixture of 3,3-diethoxypropanenitrile (6.00 mmol), 2-amino-4-bromobenzaldehyde (5.00 mmol), and p-toluenesulfonic acid monohydrate (1.051 mmol) in toluene (30 mL) was heated under reflux using a Dean/Stark head for 1 h. The solvent was concentrated in vacuo and the residue was dissolved in a small amount of N,N-dimethylformamide, diluted with chloroform, and washed with saturated aq sodium bicarbonate. The aqueous layer was extracted with chloroform and the combined organic extracts were washed with brine, dried over sodium sulfate, and concentrated in vacuo. Purification of the residue by flash chromatography (20-80% ethyl acetate/hexanes) gave the title product (775 mg, 66% yield). MS (ES+) m/e 234.8 [M+H]⁺. ¹H NMR (400 MHz, DMSO-d6) δ ppm 7.96 (dd, J=8.84, 2.02 Hz, 1H) 8.09 (d, J=8.84 Hz, 1H) 8.39 (d, J=1.77 Hz, 1H) 9.14 (d, J=2.02 Hz, 1H) 9.22 (d, J=2.02 Hz, 1H).

### b) 7-(4-((4-cyclopropyl-3-oxo-1-oxa-4,9-diazaspiro[5.5]undecan-9-yl)sulfonyl)phenyl)quinoline-3-carbonitrile

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.514 mmol), 7-bromoquinoline-3-carbonitrile (0.472 mmol), potassium carbonate (0.566 mmol), and tetrakis(triphenylphosphine)palladium(0) (0.024 mmol) in 1,4-dioxane (6 mL) and water (2 mL) was heated at 100° C. for 45 min. The mixture was cooled and partitioned between water and dichloromethane. The aqueous layer was extracted with dichloromethane and the combined organic extracts were washed with dilute brine, dried over sodium sulfate, and concentrated in vacuo to a gum. Recrystallization of the residue from ethanol gave the title product (175 mg, 74% yield). MS (ES+) m/e 502.8 [M+H]⁺. ¹H NMR (400 MHz, DMSO-d6) δ ppm 0.53-0.63 (m, 2H) 0.63-0.74 (m, 2H) 1.69 (td, J=13.14, 4.29 Hz, 2H) 1.78-1.95 (m, 2H) 2.37-2.49 (m, 2H) 2.72 (tt, J=7.39, 3.85 Hz, 1H) 3.13 (s, 2H) 3.55 (d, J=11.62 Hz, 2H) 3.86 (s, 2H) 7.89 (d, J=8.34 Hz, 2H) 8.15-8.34 (m, 4H) 8.52 (s, 1H) 9.16 (d, J=2.02 Hz, 1H) 9.25 (d, J=2.02 Hz, 1H).

### Example 138

### 4-cyclopropyl-9-((4-(3-methoxyquinolin-7-yl)phenyl)sulfonyl)-1-oxa-4,9-diazaspiro[5.5]undecan-3-one

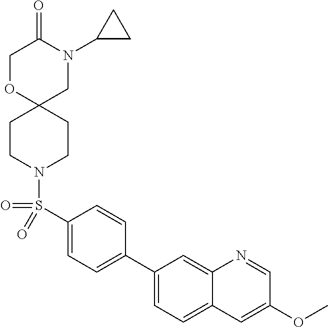 [SMILES: COc1cnc2cc(-c3ccc(S(=O)(=O)N4CCC5(CC4)CN(C4CC4)C(=O)CO5)cc3)ccc2c1]

### a) 7-bromo-3-methoxyquinoline

A mixture of 1,1,2-trimethoxyethane (10.00 mmol), 2-amino-4-bromobenzaldehyde (5.00 mmol), and p-toluenesulfonic acid monohydrate (0.526 mmol) in toluene (30 mL) was heated under reflux using a Dean/Stark head for 1 h. The solvent was concentrated in vacuo and the residue was dissolved in chloroform and washed with saturated aq sodium bicarbonate. The aqueous layer was extracted with chloroform and the combined organic extracts were washed with brine, dried over sodium sulfate, and concentrated in vacuo. Purification of the residue by flash chromatography (0-5% methanol in dichloromethane), concentration of the product fractions, and trituration with diethyl ether afforded the title product (230 mg, 19% yield). Evaporation of the mother liquors, which were rich in product, gave another 500 mg of title product (total yield 730 mg, 61%). ¹H NMR (400 MHz, DMSO-d6) δ ppm 3.94 (s, 3H) 7.72 (dd, J=8.84, 2.02 Hz, 1H) 7.83 (d, J=3.03 Hz, 1H) 7.89 (d, J=8.84 Hz, 1H) 8.17 (d, J=1.77 Hz, 1H) 8.68 (d, J=3.03 Hz, 1H). MS (ES+) m/e 237.9/239.8 Br pattern [M+H]⁺.

### b) 4-cyclopropyl-9-((4-(3-methoxyquinolin-7-yl)phenyl)sulfonyl)-1-oxa-4,9-diazaspiro[5.5]undecan-3-one

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.462 mmol), 7-bromo-3-methoxyquinoline (0.420 mmol), potassium carbonate (0.504 mmol) and tetrakis(triphenylphosphine)palladium(0) (0.021 mmol) in 1,4-dioxane (6 mL) and water (2 mL) was heated at 95° C. for 1 h. The mixture was cooled and partitioned between water and dichloromethane. The aqueous layer was extracted with dichloromethane and the combined organic extracts were washed with dilute brine, dried over sodium sulfate, and concentrated in vacuo to a solid. The solid was taken up in chloroform and silicycle thiol (400 mg) was added and the suspension stirred at 50° C. for 4 h. The mixture was filtered and evaporated to a solid that recrystallized from ethanol to give the title product (132 mg, 62% yield). MS (ES+) m/e 508.0 [M+H]⁺. ¹H NMR (400 MHz, DMSO-d6) δ ppm 0.53-0.63 (m, 2H) 0.63-0.73 (m, 2H) 1.69 (td, J=13.14, 4.29 Hz, 2H) 1.79-1.92 (m, 2H) 2.41-2.48 (m, 2H) 2.72 (tt, J=7.39, 3.85 Hz, 1H) 3.13 (s, 2H) 3.54 (d, J=11.87 Hz, 2H) 3.86 (s, 2H) 3.97 (s, 3H) 7.81-7.91 (m, 3H) 7.98-8.09 (m, 2H) 8.13 (d, J=8.59 Hz, 2H) 8.36 (s, 1H) 8.72 (d, J=3.03 Hz, 1H).

### Example 139

### 4-cyclopropyl-9-((4-(3-methylquinolin-7-yl)phenyl)sulfonyl)-1-oxa-4,9-diazaspiro[5.5]undecan-3-one

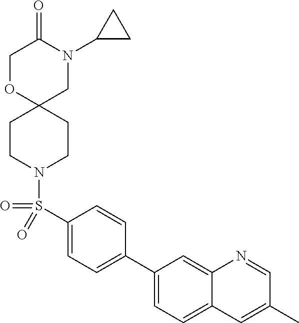 [SMILES: Cc1cnc2cc(-c3ccc(S(=O)(=O)N4CCC5(CC4)CN(C4CC4)C(=O)CO5)cc3)ccc2c1]

### a) 7-bromo-3-methylquinoline

A mixture of (E)-1-ethoxyprop-1-ene (10.00 mmol), 2-amino-4-bromobenzaldehyde (5.00 mmol), and p-toluenesulfonic acid monohydrate (0.526 mmol) in toluene (30 mL) was heated under reflux using a Dean/Stark head for 2 h. The solvent was evaporated and the residue was dissolved in a small amount of N,N-dimethylformamide, diluted with chloroform, and washed with saturate aq sodium bicarbonate. The aqueous layer was extracted with chloroform and the combined organic extracts were washed with brine, dried over sodium sulfate, and concentrated in vacuo. Purification of the residue by flash chromatography (0-20% ethyl acetate in hexanes, then 1:1 ethyl acetate/hexanes) gave the title product (170 mg, 15% yield). MS (ES+) m/e 222. [M+H]⁺. ¹H NMR (400 MHz, DMSO-d6) δ ppm 2.48 (s, 3H) 7.73 (dd, J=8.84, 2.02 Hz, 1H) 7.89 (d, J=8.84 Hz, 1H) 8.16-8.22 (m, 2H) 8.81 (d, J=2.02 Hz, 1H).

### b) 4-cyclopropyl-9-((4-(3-methylquinolin-7-yl)phenyl)sulfonyl)-1-oxa-4,9-diazaspiro[5.5]undecan-3-one

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.495 mmol), 7-bromo-3-methylquinoline (0.450 mmol), potassium carbonate (0.540 mmol), and tetrakis(triphenylphosphine)palladium(0) (0.023 mmol) in 1,4-dioxane (6 mL) and water (2 mL) was heated at 95° C. for 1 h. The mixture was cooled and partitioned between water and dichloromethane. The aqueous layer was extracted with dichloromethane, and the combined organic extracts were washed with dilute brine, dried over sodium sulfate, and concentrated in vacuo to a solid. The solid was taken up in chloroform and silicycle thiol (400 mg) was added and the suspension stirred at 50° C. for 4 h. The mixture was filtered and concentrated to a yellow solid that remained impure. Purification of the solid by flash chromatography (0-5% methanol in ethyl acetate) followed by crystallization of the resultant solid from ethanol gave the title product (110 mg, 50% yield). MS (ES+) m/e 492.3 [M+H]⁺. ¹H NMR (400 MHz, DMSO-d6) δ ppm 0.54-0.63 (m, 2H) 0.63-0.73 (m, 2H) 1.60-1.77 (m, 2H) 1.79-1.92 (m, 2H) 2.41-2.48 (m, 2H) 2.53 (s, 3H) 2.72 (tt, J=7.39, 3.85 Hz, 1H) 3.13 (s, 2H) 3.54 (d, J=11.62 Hz, 2H) 3.86 (s, 2H) 7.86 (d, J=8.59 Hz, 2H) 7.98-8.03 (m, 1H) 8.03-8.09 (m, 1H) 8.15 (d, J=8.34 Hz, 2H) 8.20 (s, 1H) 8.37 (s, 1H) 8.85 (d, J=2.27 Hz, 1H).

### Example 140

### 9-((4-(3-chloroquinolin-7-yl)phenyl)sulfonyl)-4-cyclopropyl-1-oxa-4,9-diazaspiro[5.5]undecan-3-one

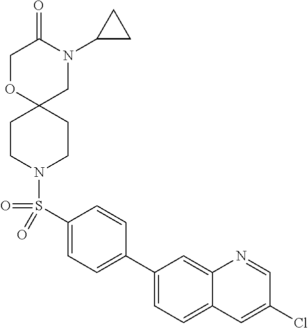 [SMILES: O=C1COC2(CCN(S(=O)(=O)c3ccc(-c4ccc5cc(Cl)cnc5c4)cc3)CC2)CN1C1CC1]

### a) 7-bromo-3-chloroquinoline

A mixture of 2-chloro-1,1-diethoxyethane (10.00 mmol), 2-amino-4-bromobenzaldehyde (5.00 mmol), and p-toluenesulfonic acid monohydrate (0.500 mmol) in toluene (30 mL) was heated at 100° C. for 2 h. The solvent was concentrated in vacuo and the residue partitioned between ethyl acetate and saturated aq sodium bicarbonate. The aqueous layer was extracted with ethyl acetate and the combined organic extracts were concentrated in vacuo. Purification of the residue by flash chromatography (10-20% ethyl acetate/hexanes) afforded the title product (688 mg, 57% yield) as a yellow solid. MS (ES+) m/e 221.8/244.0 Br pattern [M+H]⁺. ¹H NMR (400 MHz, DMSO-d6) δ ppm 7.86 (dd, J=8.72, 1.89 Hz, 1H) 7.98 (d, J=8.84 Hz, 1H) 8.30 (d, J=1.77 Hz, 1H) 8.66 (d, J=2.53 Hz, 1H) 8.94 (d, J=2.53 Hz, 1H).

### b) 9-((4-(3-chloroquinolin-7-yl)phenyl)sulfonyl)-4-cyclopropyl-1-oxa-4,9-diazaspiro[5.5]undecan-3-one

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.340 mmol), 7-bromo-3-chloroquinoline (0.330 mmol), potassium carbonate (0.396 mmol) and tetrakis(triphenylphosphine)palladium(0) (0.016 mmol) in 1,4-dioxane (6 mL) and water (2 mL) was heated at 95° C. for 1 h. The mixture was cooled and partitioned between water and dichloromethane. The aqueous layer was extracted with dichloromethane, and the combined organic extracts were washed with dilute brine, dried over sodium sulfate, and concentrated in vacuo to a solid. The solid was dissolved in chloroform, treated with silicycle thiol (200 mg) and was stirred at 55° C. overnight. The mixture was filtered through Celite and washed with chloroform, and the filtrate was concentrated in vacuo to a solid. The solid was slurried in hot ethanol, cooled, filtered, washed with ethanol and heptane, and dried in vacuo to give the title product (49 mg, 29% yield). MS (ES+) m/e 513.4 [M+H]. ¹H NMR (400 MHz, DMSO-d6) δ ppm 0.52-0.63 (m, 2H) 0.63-0.73 (m, 2H) 1.58-1.77 (m, 2H) 1.79-1.92 (m, 2H) 2.40-2.48 (m, 2H) 2.72 (tt, 1H) 3.13 (s, 2H) 3.54 (d, J=11.62 Hz, 2H) 3.86 (s, 2H) 7.88 (d, J=8.59 Hz, 2H) 8.10-8.23 (m, 4H) 8.45 (s, 1H) 8.67 (d, J=1.77 Hz, 1H) 8.98.

### Example 141

### 4-cyclopropyl-9-((4-(3-hydroxyquinolin-7-yl)phenyl)sulfonyl)-1-oxa-4,9-diazaspiro[5.5]undecan-3-one

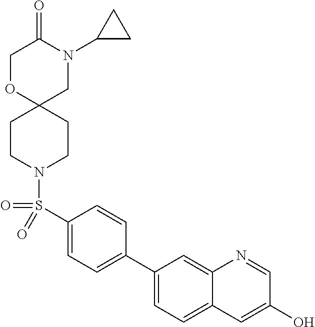 [SMILES: O=C1COC2(CCN(S(=O)(=O)c3ccc(-c4ccc5cc(O)cnc5c4)cc3)CC2)CN1C1CC1]

### a) 7-bromoquinolin-3-ol

A solution of 7-bromo-3-methoxyquinoline (1.470 mmol) in acetic acid (12 mL) was treated with 48% hydrobromic acid (2.0 mL, 36.8 mmol) and heated under reflux at 110° C. for 4 days. Analysis by LCMS then showed the reaction had progressed to about 50% completion. The mixture was poured onto ice and basified with concentrated ammonium hydroxide solution. The aqueous mixture was extracted with diethyl ether (3×) and the combined organic extracts were concentrated in vacuo. Purification of the residue by flash chromatography (10-50% ethyl acetate/hexanes) gave the title product (136 mg, 41% yield) as a tan solid. MS (ES+) m/e 224.0/226.1 Br pattern [M+H]⁺. ¹H NMR (400 MHz, CDCl₃) δ ppm 7.51 (d, J=2.27 Hz, 1H) 7.61 (s, 2H) 8.26 (s, 1H) 8.73 (d, J=2.78 Hz, 1H) (OH not visible).

### b) 4-cyclopropyl-9-((4-(3-hydroxyquinolin-7-yl)phenyl)sulfonyl)-1-oxa-4,9-diazaspiro[5.5]undecan-3-one

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.395 mmol), 7-bromoquinolin-3-ol (0.402 mmol), potassium carbonate (0.482 mmol) and tetrakis(triphenylphosphine)palladium(0) (0.020 mmol) in 1,4-dioxane (6 mL) and water (2 mL) was heated at 95° C. for 2 h. The mixture was cooled and purified by flash chromatography (0-5% methanol in ethyl acetate) to give the title product (35 mg, 18% yield). MS (ES+) m/e 494.2 [M+H]⁺. ¹H NMR (400 MHz, DMSO-d6) δ ppm 0.53-0.63 (m, 2H) 0.63-0.72 (m, 2H) 1.57-1.77 (m, 2H) 1.79-1.91 (m, 2H) 2.38-2.48 (m, 2H) 2.72 (tt, 1H) 3.13 (s, 2H) 3.53 (d, J=11.62 Hz, 2H) 3.86 (s, 2H) 7.56 (d, J=2.53 Hz, 1H) 7.84 (d, J=8.59 Hz, 2H) 7.90-8.00 (m, 2H) 8.11 (d, J=8.34 Hz, 2H) 8.30 (s, 1H) 8.65 (d, J=2.78 Hz, 1H) 10.52 (br. s, 1H).

### Example 142

### 7-(4-((4-cyclopropyl-3-oxo-1-oxa-4,9-diazaspiro[5.5]undecan-9-yl)sulfonyl)phenyl)quinoline-3-carboxamide

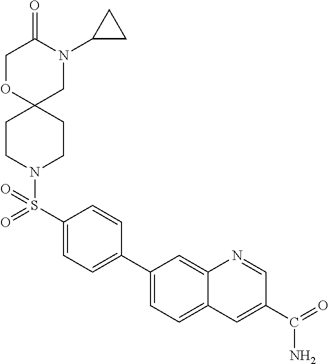 [SMILES: NC(=O)c1cnc2cc(-c3ccc(S(=O)(=O)N4CCC5(CC4)CN(C4CC4)C(=O)CO5)cc3)ccc2c1]

### a) 7-bromoquinoline-3-carboxamide

To an ice cooled solution of 7-bromoquinoline-3-carboxylic acid (1.984 mmol) in tetrahydrofuran (20 mL) was added ethyl chloroformate (4.70 mmol) and triethylamine (5.16 mmol). The reaction mixture was stirred for 10 min. The solid was filtered off and to the filtrate was added 0.5 M ammonia in dioxane (20 mL). The resulting mixture was stirred at room temperature for 1 h. The reaction mixture was then concentrated in vacuo and the white solid was taken up in ethyl acetate and water and the aqueous layer was washed with ethyl acetate. Quite a bit of white solid did not dissolve in either layer, and it was filtered off and air dried as the first batch of product. The combined organic layers were dried over sodium sulfate, filtered, and concentrated in vacuo to afford a second batch of the title product (total yield of 398 mg, 76%). MS (ES+) m/e 250.9, 252.9 [M+H]⁺.

### b) 7-(4-(4-cyclopropyl-3-oxo-1-oxa-4,9-diazaspiro[5.5]undecan-9-yl)sulfonyl)phenyl)quinoline-3-carboxamide

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.378 mmol), 7-bromoquinoline-3-carboxamide (0.378 mmol), potassium carbonate (0.454 mmol) and tetrakis(triphenylphosphine)palladium(0) (0.019 mmol) in 1,4-dioxane (6 mL) and water (2 mL) was heated at 95° C. for 2 h. A solid precipitated upon reaction. The mixture was cooled and the solid was collected and washed with 1:1 ethanol/water, ethanol, and heptane to yield the title product (120 mg, 61% yield). MS (ES+) m/e 521.1 [M+H]⁺. ¹H NMR (400 MHz, DMSO-d6) δ ppm 0.53-0.63 (m, 2H) 0.63-0.73 (m, 2H) 1.62-1.79 (m, 2H) 1.79-1.91 (m, 2H) 2.41-2.49 (m, 2H) 2.69-2.79 (m, 1H) 3.13 (s, 2H) 3.34 (s, 3H) 3.53 (br. s., 2H) 3.87 (s, 2H) 7.75 (s, 1H) 7.89 (d, J=8.59 Hz, 2H) 8.13 (dd, J=8.59, 1.77 Hz, 1H) 8.19 (d, J=8.59 Hz, 2H) 8.24 (d, J=8.59 Hz, 1H) 8.37 (s, 1H) 8.47 (s, 1H) 8.91 (d, J=2.02 Hz, 1H) 9.37 (d, 1H).

### Example 143

### N-(7-(4-((4-cyclopropyl-3-oxo-1-oxa-4,9-diazaspiro[5.5]undecan-9-yl)sulfonyl)phenyl)quinolin-3-yl)cyclopropanecarboxamide

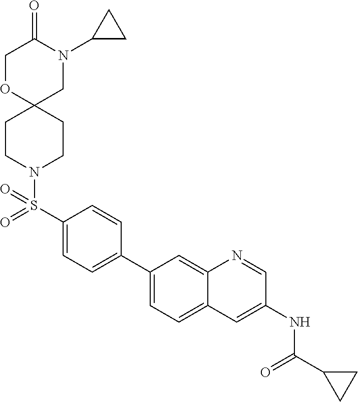 [SMILES: O=C(Nc1cnc2cc(-c3ccc(S(=O)(=O)N4CCC5(CC4)CN(C4CC4)C(=O)CO5)cc3)ccc2c1)C1CC1]

### a) N-(7-bromoquinolin-3-yl)cyclopropanecarboxamide

A solution of 7-bromoquinolin-3-amine (0.897 mmol) in chloroform (10 mL) was cooled over an ice bath and then treated with N-ethyl-N-isopropylpropan-2-amine (1.076 mmol) and cyclopropanecarbonyl chloride (1.076 mmol). The mixture was stirred at ambient temperature for 2 h. The mixture was washed with 50% saturated aq sodium bicarbonate and brine, dried over sodium sulfate, and concentrated in vacuo to a solid that was slurried in a diethyl ether-heptane mix, filtered, washed with heptane, and concentrated in vacuo to give the title product (200 mg, 77% yield). MS (ES+) m/e 290.8/292.7 Br pattern [M+H]⁺. ¹H NMR (400 MHz, CDCl₃) δ ppm 0.93-1.01 (m, 2H) 1.15-1.23 (m, 2H) 1.63-1.70 (m, 1H) 7.59-7.71 (m, 2H) 7.75 (br. s., 1H) 8.23 (s, 1H) 8.74 (d, J=2.53 Hz, 1H) 8.79 (br. s, 1H).

### b) N-(7-(4-((4-cyclopropyl-3-oxo-1-oxa-4,9-diazaspiro[5.5]undecan-9-yl)sulfonyl)phenyl)quinolin-3-yl)cyclopropanecarboxamide

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.378 mmol), N-(7-bromoquinolin-3-yl)cyclopropanecarboxamide (0.343 mmol), potassium carbonate (0.412 mmol) and tetrakis(triphenylphosphine)palladium(0) (0.017 mmol) in 1,4-dioxane (6 mL) and water (2 mL) was heated at 95° C. for 1 h. Analysis by LCMS indicated the reaction was complete, therefore it was cooled and diluted with dichloromethane and washed with 50% saturated aq sodium bicarbonate and brine, dried over sodium sulfate, and concentrated in vacuo. Purification of the residue by flash chromatography (0-5% methanol in dichloromethane) and then by reverse phase HPLC (10-90% acetonitrile/water+0.1% TFA), followed by concentration of the product fractions to low volume, addition of saturated aq sodium bicarbonate, extraction into dichloromethane, and crystallization from ethanol of the resultant solid afforded the title product (47 mg, 24% yield) as a white solid. MS (ES+) m/e 561.3 [M+H]⁺. ¹H NMR (400 MHz, DMSO-d6) δ ppm 0.54-0.63 (m, 2H) 0.63-0.73 (m, 2H) 0.86-0.97 (m, 4H) 1.62-1.76 (m, 2H) 1.79-1.97 (m, 3H) 2.41-2.48 (m, 2H) 2.72 (tt, 1H) 3.13 (s, 2H) 3.54 (d, J=11.87 Hz, 2H) 3.86 (s, 2H) 7.85 (d, J=8.34 Hz, 2H) 7.95-8.03 (m, 1H) 8.03-8.09 (m, 1H) 8.14 (d, J=8.59 Hz, 2H) 8.33 (d, J=1.01 Hz, 1H) 8.76 (d, J=2.27 Hz, 1H) 8.99 (d, J=2.27 Hz, 1H) 10.78 (s, 1H).

### Example 144

### 2-(4-((4-cyclopropyl-3-oxo-1-oxa-4,9-diazaspiro[5.5]undecan-9-yl)sulfonyl)phenyl)thieno[3,2-b]pyridine-6-carboxamide

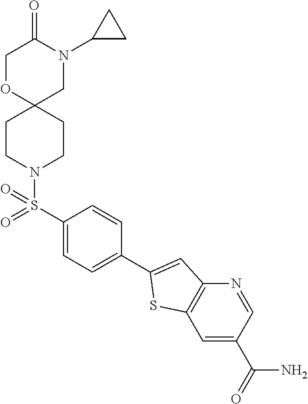 [SMILES: NC(=O)c1cnc2cc(-c3ccc(S(=O)(=O)N4CCC5(CC4)CN(C4CC4)C(=O)CO5)cc3)sc2c1]

### a) diethyl 2-((thiophen-3-ylamino)methylene)malonate

A mixture of methyl 3-aminothiophene-2-carboxylate (1.2 mol), 1 N aq NaOH (1.44 mol) and ethanol (500 mL) was stirred at 90° C. for 3 h. Analysis by TLC and LCMS showed complete conversion of starting material. To this was added acetic acid (83 mL) and the mixture was stirred for 3 h. Then diethyl 2-(ethoxymethylene)malonate (240 mL) was added dropwise and the mixture was continued stirred at room temperature overnight. The mixture was then filtered and the filter cake was collected and washed with ethanol (200 mL) to give the title product as a white solid (190 g, 59%). MS (ES+) m/e 270 [M+H]⁺.

### b) ethyl 7-hydroxythieno[3,2-b]pyridine-6-carboxylate

A mixture of diethyl 2-((thiophen-3-ylamino)methylene)malonate (0.149 mol) in diphenyl ether (200 mL) was stirred at 250° C. for 20 min under nitrogen. The reaction mixture was then cooled and filtered, and the filter cake was collected to give the title product (9.8 g, 30%) as a brown solid. MS (ES+) m/e 224 [M+H]⁺.

### c) 7-hydroxythieno[3,2-b]pyridine-6-carboxylic acid

A mixture of ethyl 7-hydroxythieno[3,2-b]pyridine-6-carboxylate (0.352 mol), 4 N aq NaOH (350 mL) and methanol (300 mL) was stirred at reflux for 16 h. The reaction was cooled to room temperature and the pH of the mixture was adjusted to ˜2 with hydrochloric acid and then filtered. The filter cake was collected and dried to give the title product (65 g, 95%) as a brown solid. MS (ES+) m/e 196 [M+H]⁺.

### d) 7-chlorothieno[3,2-b]pyridine-6-carboxamide

A mixture of 7-hydroxythieno[3,2-b]pyridine-6-carboxylic acid (0.333 mol), thionyl chloride (100 mL) and chloroform (600 mL) was reacted at 70° C. for 48 h. The mixture became clear during this time. The mixture was concentrated in vacuo to afford a residue which was taken up in tetryhydrofuran (200 mL). This solution was added dropwise to an ice-chilled solution of saturated aq ammonium hydroxide (500 mL) in tetrahydrofuran (200 mL) and the mixture was reacted at 0° C. for 1 h. The mixture was then extracted with ethyl acetate (4×300 mL) and the combined organic phases were washed with brine (200 mL), dried, and concentrated in vacuo to give the title product (70 g, 99%). MS (ES+) m/e 213 [M+H]⁺. ¹H NMR (300 MHz, DMSO-d₆) δ ppm 7.71 (d, J=5.4 Hz, 1H), 7.89 (br s, 1H), 8.14 (br s, 1H), 8.35 (d, J=5.4 Hz, 1H), 8.73 (s, 1H).

### e) 7-chlorothieno[3,2-b]pyridine-6-carbonitrile

To a solution of 7-chlorothieno[3,2-b]pyridine-6-carboxamide (0.33 mol) in dry N,N-dimethylformamide (250 mL) was added cyanuric chloride (0.198 mol) in portions. The mixture was reacted for 2 h at room temperature and then poured into ice-water, filtered, and dried to give the title product as a brown solid (62 g, 97%). ¹H NMR (300 MHz, DMSO-d₆) δ ppm 7.82 (d, J=5.4 Hz, 1H), 8.61 (d, J=5.4 Hz, 1H), 9.12 (s, 1H).

### f) 2-bromo-7-chlorothieno[3,2-b]pyridine-6-carbonitrile

To a solution of 7-chlorothieno[3,2-b]pyridine-6-carbonitrile (0.0103 mol) in dry tetrahydrofuran (100 mL) was added 2 N lithium diisopropylamide (6.2 mL). The reaction was stirred for 5 min under nitrogen at −78° C. and then a solution of N-bromosuccinimide (0.0206 mol) in tetrahydrofuran (100 mL) was added over 20 min. The mixture was reacted at room temperature for 20 min, and then water (100 mL) was added and the mixture was extracted with ethyl acetate (2×200 mL). The combined organic phases were washed with brine (2×50 mL), dried over Na₂SO₄, and filtered. Following the same procedure, another 15 experiments on the exact same scale were carried out. The combined organic phases of the 16 batches were combined and concentrated to give a residue, which was purified by silica gel chromatography (1:6 tetrahydrofuran:petroleum ether) to give the title product as a light yellow solid (25 g, 55%). MS (ES+) m/e 273 [M+H]⁺. ¹H NMR (300 MHz, DMSO-d₆) δ ppm 8.11 (s, 1H), 9.11 (s, 1H).

### g) 2-bromo-7-chlorothieno[3,2-b]pyridine-6-carboxamide

A mixture of 2-bromo-7-chlorothieno[3,2-b]pyridine-6-carbonitrile (6 g, 0.0219 mol) in H₂SO₄ (50 mL) was reacted at 30° C. for 12 h and then poured onto ice. Then the mixture was combined with that from another 5 experiments (total amount of 2-bromo-7-chlorothieno[3,2-b]pyridine-6-carbonitrile used in the 5 experiments was 19 g) that were carried out following the same procedure. The combined mixture was extracted with ethyl acetate (100 mL) and the pH of the aqueous layer was adjusted to ˜2 with 5 N aq NaOH. The aqueous layer was filtered and the filter cake was collected, washed with water (100 mL), dried, and washed with 20:1 dichloromethane:methanol (4×100 mL) to give the title product (14.5 g, 55%) as a light brown solid. MS (ES+) m/e 291 [M+H]⁺. ¹H NMR (300 MHz, DMSO-d₆) δ ppm 7.90 (br s, 1H), 7.97 (s, 1H), 8.15 (br s, 1H), 8.72 (s, 1H).

### h) 7-chloro-2-(4-((4-cyclopropyl-3-oxo-1-oxa-4,9-diazaspiro[5.5]undecan-9-yl)sulfonyl)phenyl)thieno[3,2-b]pyridine-6-carboxamide

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (1.029 mmol), 2-bromo-7-chlorothieno[3,2-b]pyridine-6-carboxamide (0.857 mmol), potassium carbonate (1.029 mmol) and tetrakis(triphenylphosphine)palladium(0) (0.043 mmol) in 1,4-dioxane (3 mL) and water (1 mL) was heated at 60° C. in a nitrogen atmosphere for 2.5 h. A solid started to precipitate after ˜1.5 h. The mixture was cooled and the solid was collected and washed with water, ethanol and hexane. Further product precipitated from the mother liquors upon standing to give the title product (340 mg, 71% yield). MS (ES+) m/e 560.7 [M+H]⁺. ¹H NMR (400 MHz, DMSO-d6) δ ppm 0.52-0.63 (m, 2H) 0.63-0.82 (m, 2H) 1.56-1.75 (m, 2H) 1.76-1.92 (m, 2H) 2.39-2.49 (m, 2H) 2.72 (tt, J=7.39, 3.85 Hz, 1H) 3.13 (s, 2H) 3.53 (d, J=11.37 Hz, 2H) 3.86 (s, 2H) 7.86 (d, J=8.59 Hz, 2H) 7.97 (s, 1H) 8.14-8.29 (m, 3H) 8.42 (s, 1H) 8.77 (s, 1H).

### i) 2-(4-((4-cyclopropyl-3-oxo-1-oxa-4,9-diazaspiro[5.5]undecan-9-yl)sulfonyl)phenyl)thieno[3,2-b]pyridine-6-carboxamide

A mixture of 7-chloro-2-(4-((4-cyclopropyl-3-oxo-1-oxa-4,9-diazaspiro[5.5]undecan-9-yl)sulfonyl)phenyl)thieno[3,2-b]pyridine-6-carboxamide (0.535 mmol) and 10% palladium on carbon (2.82 mmol) in N,N-dimethylformamide (20.00 mL) gave a clear solution. The flask was flushed with nitrogen and shaken in a hydrogen atmosphere (50 psi) for 5 h. The catalyst was removed by filtration through celite and the solvent was concentrated in vacuo. The yellow residue was slurried in ethanol and collected by filtration. Purification of the residue by reverse phase HPLC (20-75% acetonitrile/water+0.1% TFA) followed by partial concentration in vacuo of the product fractions, neutralization, extraction with N,N-dimethylformamide, and concentration in vacuo gave a solid that was then slurried in ethanol, collected by filtration, and concentrated in vacuo to afford the title product (8 mg, 3% yield). MS (ES+) m/e 527.0 [M+H]⁺. ¹H NMR (400 MHz, DMSO-d6) δ ppm 0.52-0.62 (m, 2H) 0.63-0.74 (m, 2H) 1.68 (td, J=13.20, 4.42 Hz, 2H) 1.84 (d, J=13.14 Hz, 2H) 2.38-2.48 (m, 2H) 2.69-2.80 (m, 1H) 3.13 (s, 2H) 3.52 (d, J=12.13 Hz, 2H) 3.86 (s, 2H) 7.69 (s, 1H) 7.86 (d, J=8.59 Hz, 2H) 8.19 (d, J=8.34 Hz, 2H) 8.26 (s, 1H) 8.33 (s, 1H) 8.95 (d, J=1.52 Hz, 1H) 9.14 (d, J=2.02 Hz, 1H).

### Example 145

### 4-cyclopropyl-9-((4-(3-fluoroquinolin-7-yl)phenyl)sulfonyl)-1-oxa-4,9-diazaspiro[5.5]undecan-3-one

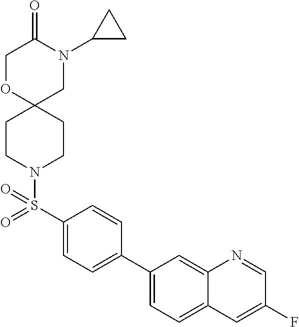 [SMILES: O=C1COC2(CCN(S(=O)(=O)c3ccc(-c4ccc5cc(F)cnc5c4)cc3)CC2)CN1C1CC1]

### a) 7-bromo-3-fluoroquinoline

A solution of 7-bromoquinolin-3-amine (4.48 mmol) in chlorobenzene (10 mL) was added dropwise over 10 min onto boron trifluoride dihydrate (6.72 mmol). The mixture was then heated to 50° C. and t-butyl nitrite (4.48 mmol) was added at this temperature over 20 min. The temperature was then raised to 100° C. and the mixture stirred for 30 min, cooled, and poured onto ice and saturated aq sodium bicarbonate. Material that had solidified onto the flask was suspended in ethanol. The mixture was diluted with more saturated aq sodium bicarbonate and extracted with chloroform (3×). The combined organic extracts were washed with dilute brine, dried over sodium sulfate, and concentrated in vacuo. Purification of the residue by flash chromatography (dichloromethane) gave the title product (350 mg, 34% yield). MS (ES+) m/e 225.9/227.8 [M+H]⁺, Br pattern. ¹H NMR (400 MHz, DMSO-d6) δ ppm 7.84 (dd, J=8.72, 1.39 Hz, 1H) 8.00 (d, J=8.84 Hz, 1H) 8.31 (d, J=2.02 Hz, 1H) 8.34 (dd, J=9.47, 2.91 Hz, H) 9.00 (d, 1H).

### b) 4-cyclopropyl-9-((4-(3-fluoroquinolin-7-yl)phenyl)sulfonyl)-1-oxa-4,9-diazaspiro[5.5]undecan-3-one

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.531 mmol), 7-bromo-3-fluoroquinoline (0.442 mmol), potassium carbonate (0.531 mmol) and tetrakis(triphenylphosphine)palladium(0) (0.022 mmol) in 1,4-dioxane (6 mL) and water (2 mL) was heated at 95° C. for 1 h. The reaction mixture was concentrated in vacuo and the residue purified by flash chromatography (0-5% methanol in dichloromethane). Purification of the resultant white solid by reverse phase HPLC (20-70% acetonitrile/water+0.1% TFA) followed by partial concentration of the product fractions, neutralization with ˜10% aq ammonium hydroxide, extraction with chloroform (3×), and concentration in vacuo of the combined organics gave a foam that recrystallized from ethanol to afford the title product (83 mg, 38% yield) as a white solid. MS (ES+) m/e 496.2 [M+H]⁺. ¹H NMR (400 MHz, DMSO-d6) δ ppm 0.52-0.63 (m, 2H) 0.63-0.72 (m, 2H) 1.69 (td, J=13.07, 4.42 Hz, 2H) 1.77-1.90 (m, 2H) 2.41-2.49 (m, 2H) 2.72 (tt, 1H) 3.13 (s, 2H) 3.54 (d, J=11.62 Hz, 2H) 3.86 (s, 2H) 7.87 (d, J=8.59 Hz, 2H) 8.08-8.14 (m, 1H) 8.17 (d, J=8.59 Hz, 3H) 8.35 (dd, J=9.47, 2.65 Hz, 1H) 8.47 (s, 1H) 9.03 (d, J=2.78 Hz, 1H).

### Example 146

### N-(7-(4-((4-cyclopropyl-3-oxo-1-oxa-4,9-diazaspiro[5.5]undecan-9-yl)sulfonyl)phenyl)quinolin-3-yl)methanesulfonamide

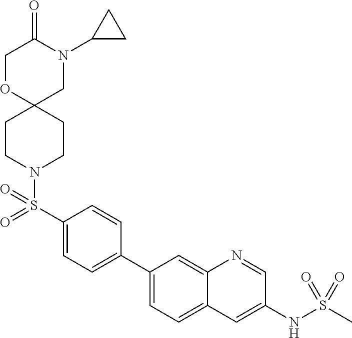 [SMILES: CS(=O)(=O)Nc1cnc2cc(-c3ccc(S(=O)(=O)N4CCC5(CC4)CN(C4CC4)C(=O)CO5)cc3)ccc2c1]

### a) N-(7-bromoquinolin-3-yl)methanesulfonamide

A solution of 7-bromoquinolin-3-amine (1.345 mmol) and pyridine (1.345 mmol) in acetonitrile (20 mL) was treated with pyridine (1.345 mmol) and methanesulfonyl chloride (1.345 mmol), and the mixture was stirred at ambient temperature for 1 h. The reaction was then heated to 40° C. and stirred for 20 h. Analysis by LCMS showed 50-60% conversion. The temperature was raised to 50° C. and the mixture was stirred for an additional 24 h. The mixture was concentrated in vacuo and the residue purified by flash chromatography (0-5% methanol in dichloromethane) to give the title product (186 mg, 46% yield). MS (ES+) m/e 301.1/303.2 Br pattern [M+H]⁺. ¹H NMR (400 MHz, DMSO-d6) δ ppm 3.16 (s, 3H) 7.74 (dd, J=8.84, 2.02 Hz, 1H) 7.96 (d, J=8.84 Hz, 1H) 8.13 (d, J=2.78 Hz, 1H) 8.19 (d, J=2.02 Hz, 1H) 8.76 (d, J=2.78 Hz, 1H) 10.40 (s, 1H).

### b) N-(7-(4-((4-cyclopropyl-3-oxo-1-oxa-4,9-diazaspiro[5.5]undecan-9-yl)sulfonyl)phenyl)quinolin-3-yl)methanesulfonamide

A mixture of 4-cyclopropyl-9-((4-(4,4,5,5-tetramethyl-1,3,2-dioxaborolan-2-yl)phenyl)sulfonyl)-1-oxa-4,9-diazaspiro[5.5]undecan-3-one (0.378 mmol), N-(7-bromoquinolin-3-yl)methanesulfonamide (0.299 mmol), potassium carbonate (0.359 mmol) and tetrakis(triphenylphosphine)palladium(0) (0.015 mmol) in 1,4-dioxane (6 mL) and water (2 mL) was heated at 95° C. for 1 h. The reaction mixture was concentrated in vacuo and purified by flash chromatography (0-5% methanol in dichloromethane) to provide, upon recrystallization from acetonitrile, the title product (100 mg, 59% yield). MS (ES+) m/e 571.3 [M+H]⁺. ¹H NMR (400 MHz, DMSO-d6) δ ppm 0.51-0.62 (m, 2H) 0.62-0.75 (m, 2H) 1.69 (td, J=13.07, 4.42 Hz, 2H) 1.85 (d, J=13.39 Hz, 2H) 2.41-2.49 (m, 2H) 2.72 (tt, J=7.36, 3.88 Hz, 1H) 3.13 (s, 2H) 3.17 (s, 3H) 3.54 (d, J=11.62 Hz, 2H) 3.86 (s, 2H) 7.86 (d, J=8.34 Hz, 2H) 7.99-8.08 (m, 1H) 8.08-8.23 (m, 4H) 8.

### Biological Assays

FAS assay

FAS activity was measured through one of the two following assays.

### Assay #1:

Inhibition of FAS activity can be measured based on the detection of residual NADPH substrate after the FAS assay is quenched. This assay is run as a 10 μL, endpoint assay in 384-well format, where the reaction contains 20 μM malonyl-CoA, 2 μM acetyl-CoA, 30 μM NADPH and 40 nM FAS in 50 mM sodium phosphate, pH 7.0. The assay is run by sequentially dispensing 5 μl of a malonyl-CoA solution, then enzyme solution (containing the acetyl-CoA, and NADPH) into a black, low volume assay plate (Greiner 784076) pre-dispensed with 100 nL compound solutions in DMSO. The reaction is incubated at ambient temperature for 60 minutes, then quenched with 5 μL, of a developing solution composed of 90 μM resazurin, 0.3 IU/ml diaphorase in 50 mM sodium phosphate, pH 7.0. The developed reaction is read on a Molecular Devices Analyst or Acquest (or equivalent) plate reader using a 530 nm excitation wavelength filter, a 580 nm emission filter, and 561 nm dichroic filter. The test compounds are prepared in neat DMSO at a concentration of 10 mM. For inhibition curves, compounds are diluted using a three fold serial dilution and tested at 11 concentrations (e.g. 25 μM-0.42 nM). Curves are analysed using ActivityBase and XLfit, and results are expressed as pIC50 values.

### Assay #2:

Inhibition of FAS can also be quantified based on the detection of the CoA products with a thio-reactive coumarin dye. This assay is run as a 10 μL endpoint assay in 384-well format, where the reaction contains 20 μM malonyl-CoA, 20 μM acetyl-CoA, 40 μM NADPH and 2 nM FAS in 50 mM sodium phosphate, pH 7.0, and 0.04% Tween-20. The assay is run by adding 5 μL enzyme solution to a black, low volume assay plate (Greiner 784076) pre-dispensed with 100 nl compound solutions in DMSO. After 30 minutes, 5 μL substrate is added, and the reaction incubated at ambient temperature for an additional 60 minutes. The reaction is then quenched with 10 μL of 6M guanidine-HCl containing 50 μM CPM (7-diethylamino-3-(4′-maleimidylphenyl)-4-methylcoumarin (CPM; thio-reactive dye), and incubated for 30 minutes. The plate is read on an Envision (PerkinElmer) or equivalent plate reader using a 380 nm excitation wavelength filter, and a 486 nm emission filter. Data fitting and compound preparations are done as described above.

### Lipogenesis Assay

Cultured primary human pre-adipocytes (Zen-Bio, Cat#ASC062801) are plated at confluence (3×10⁴ cells/well) in 96-well plates (Costar, Cat#3598) coated with 0.2% gelatin (Sigma, Cat#G-6650) in DMEM/F12 medium (InvitroGen Cat#11330-032) supplemented with 10% heat inactivated fetal bovine serum (InvitroGen, Cat#16000-044). The following day (day 1) the cell differentiation is induced by replacing the seeding medium with the differentiation medium composed of DMEM/F12 medium supplemented with 10% heat inactivated fetal bovine serum, 200 μM 3-isobutyl-1-methylxanthine (Sigma, Cat#1-5879), 20 nM dexamethasone (Sigma, Cat#D-8893), 20 nM GW1929 (Sigma, Cat#G5668) and 20 nM insulin (InvitroGen, Cat#03-0110SA). On day 7, differentiation medium is replaced by the re-feed medium made of DMEM/F12 supplemented with 10% heat inactivated serum and 20 nM insulin. The appropriate concentration of tested compounds and controls are added into this medium at that time. On day 12, the relative amount of cellular triglyceride is estimated by using a Trinder kit (Sigma, Cat#TR0100). Re-feed medium is aspirated and cells are washed with PBS (InvitroGen, Cat#14190-144) and the assay is performed according the kit manufacturer protocol. Briefly, reconstituted solutions A and B are mixed with 0.01% digitonin (Sigma, Cat#D-5628) prior to performing the assay and added onto the cells; plates are incubated at 37° C. for one hour. The absorbance is read at 540 nm. The data is first normalized using the following equation: 100*((UNK−Control 1)/(Control 2−Control 1)) where Control 1 is the Robust Mean of the 0% response control and Control 2 is the Robust Mean of the 100% response control. When multiple dilutions of compounds are tested, pXC50 are calculated from curves using the 4-parameter curve fitting with the following equation: y=(a−d)/(1+(s/c)̂b)+d and with IRLS (Iterative Re-weighted Least Squares) algorithms to weight outliers (Mosteller, F. & Tukey J. W. (1977) Data Analysis and Regression, pp 353-365, Addison-Wesley).

### Biological Data

Exemplified compounds of the present invention were tested according to the above assays and were found to be inhibitors of FAS. The IC₅₀ values ranged from about 1 nM to about 10 μM. The IC₅₀ values of the more active compounds range from about 1 nM to about 200 nM. The most active compounds are under 10 nM.

Each compound listed below was tested two or more times generally according to the assays described herein, and the average IC₅₀ values are listed in the table below.

| Example No. | IC₅₀ (nM) |
|---|---|
| Example 3 | 251 |
| Example 13 | 1259 |
| Example 14 | 40 |
| Example 26 | 200 |
| Example 29 | 10 |
| Example 59 | 13 |
| Example 70 | 32 |
| Example 76 | 32 |
| Example 86 | 6 |
| Example 105 | 63 |

1. A compound of Formula (I), 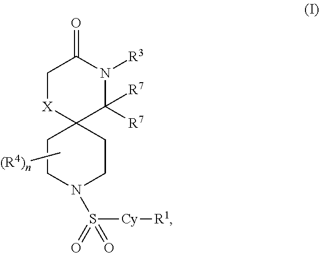
wherein
R³ is selected from the group consisting of: C₁-C₆alkyl, C₃-C₇cycloalkyl, and C₄-C₆heterocycloalkyl, wherein said C₁-C₆alkyl, C₃-C₇cycloalkyl or LC₆heterocycloalkyl is optionally substituted with from 1 to 6 substituents independently selected from the group of: halogen, C₁-C₄alkyl, —C₁-C₄alkylhalogen, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹;
R⁵ is selected from the group consisting of: hydrogen, C₁-C₄alkyl, C₃-C₇cycloalkyl, —C₁-C₃alkylC₃-C₇cycloalkyl, phenyl, and —C₁-C₃alkyl-phenyl;
R⁶ is hydrogen, C₁-C₄alkyl, C₃-C₇cycloalkyl, or —C₁-C₃alkylC₃-C₇cycloalkyl;
or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁵R⁶;
R⁴ is oxo, halogen or C₁-C₆alkyl;
Cy is selected from the group consisting of: phenyl, pyridinyl, and 5- or 6-membered heteroaryl wherein said phenyl, pyridinyl, or 5- or 6-membered heteroaryl is optionally substituted with from one to three R² groups, wherein each R² is independently selected from C₁-C₆alkyl, cyano, C₁-C₄alkoxy, hydroxyl, —CF₃, or halogen;
R¹ is selected from the group consisting of: phenyl, 5- or 6-membered heteroaryl, napthyl, and 9- or 10-membered heterocyclyl, wherein said phenyl, 5- or 6-membered heteroaryl, napthyl, or 9- or 10-membered heterocyclyl, is optionally substituted with from 1 to 4 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹;
each R⁷ is independently H, C₁-C₃alkyl, —C₁-C₄alkylhalogen, halogen, cyano, —CONR⁵R⁶, —C(═O)OC₁-C₄alkyl, hydroxyC₁-C₄alkyl-, and —C(═O)OH;
X is CH₂, NR₆ or O;
n is 0, 1, 2, 3, or 4;
or a pharmaceutically acceptable salt thereof.

2. A compound of claim 1, which compound is represented by Formula (I)(A), 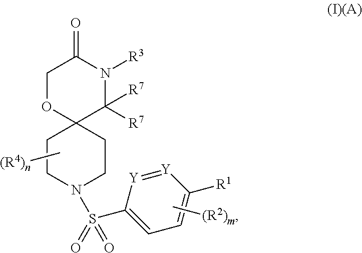
wherein
R³ is selected from the group consisting of: C₁-C₆alkyl, C₃-C₇cycloalkyl, and C₄-C₆heterocycloalkyl, wherein said C₁-C₆alkyl, C₃-C₇cycloalkyl or LC₆heterocycloalkyl is optionally substituted with from 1 to 6 substituents independently selected from the group of: halogen, C₁-C₄alkyl, —C₁-C₄alkylhalogen, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹;
R⁵ is selected from the group consisting of: hydrogen, C₁-C₄alkyl, C₃-C₇cycloalkyl, —C₁-C₃alkylC₃-C₇cycloalkyl, phenyl, and —C₁-C₃alkylphenyl;
R⁶ is hydrogen, C₁-C₄alkyl, C₃-C₇cycloalkyl, or —C₁-C₃alkyl C₃-C₇cycloalkyl;
or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁵R⁶;
R⁴ is oxo, halogen or C₁-C₆alkyl;
R¹ is selected from the group consisting of: phenyl, 5- or 6-membered heteroaryl, napthyl, and 9- or 10-membered heterocyclyl, wherein said phenyl, 5- or 6-membered heteroaryl, napthyl, or 9- or 10-membered heterocyclyl, is optionally substituted with from 1 to 4 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹;
each R² is independently C₁-C₆alkyl, cyano, C₁-C₄alkoxy, hydroxyl, —CF₃, or halogen;
each R⁷ is independently H, C₁-C₃alkyl, —C₁-C₄alkylhalogen, halogen, cyano, —CONR⁵R⁶, —C(═O)OC₁-C₄alkyl, hydroxyC₁-C₄alkyl-, and —C(═O)OH;
n is 0, 1, 2, 3, or 4;
m is 0, 1, 2, or 3;
Y is C or N, provided that when one Y is N the other Y is C;
or a pharmaceutically acceptable salt thereof.

3. A compound of claim 1, wherein said Cy is a phenyl, optionally substituted with from one to three groups selected from the group consisting of: C₁-C₆alkyl, cyano, C₁-C₄alkoxy, hydroxyl, —CF₃, and halogen; or a pharmaceutically acceptable salt thereof.

4. A compound of claim 1, wherein Cy is 5- or 6-membered heteroaryl, optionally substituted with one to two groups selected from the group consisting of: C₁-C₆alkyl, cyano, C₁-C₄alkoxy, hydroxyl, —CF₃, and halogen; or a pharmaceutically acceptable salt thereof.

5. A compound of claim 4, wherein said Cy is 5-membered heteroaryl selected from the group consisting of: 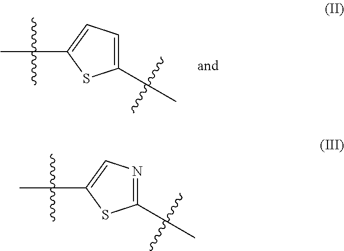 [SMILES: *c1ccc(*)s1 ; *c1cnc(*)s1]
optionally substituted with one to two groups selected from the group consisting of: C₁-C₆alkyl, cyano, C₁-C₄alkoxy, hydroxyl, —CF₃, and halogen; or a pharmaceutically acceptable salt thereof.

6. A compound of claim 1, wherein each R⁷ is H.

7. A compound of claim 1, which compound is represented by Formula (I)(B), 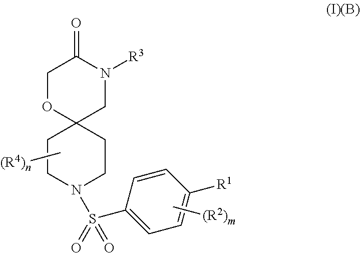
wherein
R³ is selected from the group consisting of: C₁-C₆alkyl, C₃-C₇cycloalkyl, and C₄-C₆heterocycloalkyl, wherein said C₁-C₆alkyl, C₃-C₇cycloalkyl or C₄-C₆heterocycloalkyl is optionally substituted with from 1 to 6 substituents independently selected from the group of: halogen, C₁-C₄alkyl, —C₁-C₄alkylhalogen, —CF₃, C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NHC(O)C₁-C₄alkyl, —NHCONR⁵R⁶, —NHSO₂C₁-C₄alkyl, —NHSO₂NR⁵R⁶, and R⁹;
R⁵ is selected from the group consisting of: hydrogen, C₁-C₄alkyl, C₃-C₇cycloalkyl, —C₁-C₃alkylC₃-C₇cycloalkyl, phenyl, and —C₁-C₃alkylphenyl;
R⁶ is hydrogen, C₁-C₄alkyl, C₃-C₇cycloalkyl, or —C₁-C₃alkyl C₃-C₇cycloalkyl;
or R⁵ and R⁶ taken together with the nitrogen to which they are attached represent a 3- to 7-membered saturated ring optionally containing one other heteroatom which is oxygen, nitrogen, or sulfur, which is optionally substituted 1 or 2 times independently by oxo or C₁-C₄alkyl;
R⁹ is a 5- or 6-membered heteroaryl ring containing 1 to 4 heteroatoms selected from oxygen, nitrogen, and sulfur, which is optionally substituted with 1 or 2 substituents selected from halogen, C₁-C₄alkyl, CF₃, C₁-C₄alkoxy, and —NR⁵R⁶;
R⁴ is oxo, halogen or C₁-C₆alkyl;
R¹ is selected from the group consisting of: phenyl, 5- or 6-membered heteroaryl, napthyl, and 9- or 10-membered heterocyclyl, wherein said phenyl, 5- or 6-membered heteroaryl, napthyl, or 9- or 10-membered heterocyclyl, is optionally substituted with from 1 to 4 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, NR⁶SO₂NR⁵R⁶, —NR⁶C(O)H, tetrazolyl, —B(OH)₂, —SO₃H, and R⁹:
each R² is independently C₁-C₆alkyl, cyano, C₁-C₄alkoxy, hydroxyl, —CF₃, or halogen;
n is 0, 1, 2, 3, or 4;
m is 0, 1, 2, or 3;
or a pharmaceutically acceptable salt thereof.

8. A compound or pharmaceutically acceptable salt according to claim 1, wherein R¹ is phenyl optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹.

9. A compound or pharmaceutically acceptable salt according to claim 1, wherein R¹ is selected from furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl, wherein said furanyl, thienyl, pyrrolyl, imidazolyl, pyrazolyl, triazolyl, tetrazolyl, thiazolyl, oxazolyl, isoxazolyl, oxadiazolyl, thiadiazolyl, isothiazolyl, pyridinyl, pyridazinyl, pyrazinyl, pyrimidinyl, and triazinyl are each optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cyclo alkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹.

10. A compound or pharmaceutically acceptable salt according to claim 1, wherein R¹ is napthyl optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹.

11. A compound or pharmaceutically acceptable salt according to claim 1, wherein R¹ is selected from benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, or pteridinyl, wherein said benzofuranyl, isobenzofuryl, 2,3-dihydrobenzofuryl, 1,3-benzodioxolyl, dihydrobenzodioxinyl, benzothienyl, indolizinyl, indolyl, isoindolyl, indolinyl, isoindolinyl, 1-H-indazolyl, benzimidazolyl, dihydrobenzimidazolyl, benzoxazolyl, dihydrobenzoxazolyl, benzthiazolyl, benzoisothiazolyl, dihydrobenzoisothiazolyl, indazolyl, pyrrolopyridinyl, pyrrolopyrimidinyl, imidazopyridinyl, imidazopyrimidinyl, pyrazolopyridinyl, pyrazolopyrimidinyl, benzoxadiazolyl, benzthiadiazolyl, benzotriazolyl, triazolopyridinyl, purinyl, quinolinyl, tetrahydroquinolinyl, isoquinolinyl, tetrahydroisoquinolinyl, quinoxalinyl, cinnolinyl, phthalazinyl, quinazolinyl, 1,5-naphthyridinyl, 1,6-naphthyridinyl, 1,7-naphthyridinyl, 1,8-naphthyridinyl, and pteridinyl are each optionally substituted with from 1 to 3 substituents independently selected from halogen, C₁-C₄alkylhalogen, optionally substituted C₁-C₄alkyl, —CF₃, —C₃-C₇cycloalkyl, —C(O)C₁-C₄alkyl, —C(O)C₃-C₇cycloalkyl, —CO(phenyl), —C₁-C₄(═O)OH, —C(═O)OC₁-C₄alkyl, —CONR⁵R⁶, phenyl, —SO₂C₁-C₄alkyl, —SO₂NR⁵R⁶, cyano, oxo, hydroxyl, C₁-C₄alkoxy, C₃-C₇cycloalkoxy, hydroxyC₁-C₄alkyl-, C₁-C₄alkoxyC₁-C₄alkyl-, —OCF₃, —NR⁵R⁶, R⁵R⁶NC₁-C₄alkyl-, —NR⁶C(O)C₁-C₄alkyl, —NR⁶C(O)C₃-C₇cycloalkyl, —NR⁶CONR⁵R⁶, —NR⁶SO₂C₁-C₄alkyl, —NR⁶SO₂NR⁵R⁶, —NR⁶C(O)H, tetrazolyl, —B(OH)₂, —SO₃H, and R⁹.

12. A compound according to claim 1, wherein R³ is C₁-C₆alkyl or C₃-C₇cycloalkyl.

13. A compound or pharmaceutically acceptable salt thereof according to claim 1 selected from:
4-methyl-9-{[4-(7-quinolinyl)phenyl]sulfonyl}-1-oxa-4,9-diazaspiro[5.5]undecan-3-one;
9-{[4-(1H-indol-6-yl)phenyl]sulfonyl}-4-methyl-1-oxa-4,9-diazaspiro[5.5]undecan-3-one;
9-{[4-(1-benzofuran-5-yl)phenyl]sulfonyl}-4-ethyl-1-oxa-4,9-diazaspiro[5.5]undecan-3-one;
4-ethyl-9-{[4-(7-quinolinyl)phenyl]sulfonyl}-1-oxa-4,9-diazaspiro[5.5]undecan-3-one;
4-(1-methylethyl)-9-{[4-(7-quinolinyl)phenyl]sulfonyl}-1-oxa-4,9-diazaspiro[5.5]undecan-3-one;
9-{[4-(7-quinolinyl)phenyl]sulfonyl}-4-(2,2,2-trifluoroethyl)-1-oxa-4,9-diazaspiro[5.5]undecan-3-one;
4-(2-furanylmethyl)-9-{[4-(7-quinolinyl)phenyl]sulfonyl}-1-oxa-4,9-diazaspiro[5.5]undecan-3-one;
4-[2-(methyloxy)ethyl]-9-{[4-(7-quinolinyl)phenyl]sulfonyl}-1-oxa-4,9-diazaspiro[5.5]undecan-3-one;
4-(phenylmethyl)-9-{[4-(7-quinolinyl)phenyl]sulfonyl}-1-oxa-4,9-diazaspiro[5.5]undecan-3-one;
4-(1,1-dimethylethyl)-9-{[4-(7-quinolinyl)phenyl]sulfonyl}-1-oxa-4,9-diazaspiro[5.5]undecan-3-one;
4-(1-methylcyclopropyl)-9-{[4-(7-quinolinyl)phenyl]sulfonyl}-1-oxa-4,9-diazaspiro[5.5]undecan-3-one;
4-cyclobutyl-9-{[4-(7-quinolinyl)phenyl]sulfonyl}-1-oxa-4,9-diazaspiro[5.5]undecan-3-one;
9-(4-biphenylylsulfonyl)-4-cyclopropyl-1-oxa-4,9-diazaspiro[5.5]undecan-3-one;
4-cyclopropyl-9-{[4-(1H-indol-6-yl)phenyl]sulfonyl}-1-oxa-4,9-diazaspiro[5.5]undecan-3-one;
4′-[(4-cyclopropyl-3-oxo-1-oxa-4,9-diazaspiro[5.5]undec-9-yl)sulfonyl]-4-biphenylcarbonitrile;
4-cyclopropyl-9-[(4′-fluoro-4-biphenylyl)sulfonyl]-1-oxa-4,9-diazaspiro[5.5]undecan-3-one;
4-cyclopropyl-9-{[4-(1H-indazol-6-yl)phenyl]sulfonyl}-1-oxa-4,9-diazaspiro[5.5]undecan-3-one;
4-cyclopropyl-9-{[4-(1H-indol-5-yl)phenyl]sulfonyl}-1-oxa-4,9-diazaspiro[5.5]undecan-3-one;
9-{[4-(1-benzofuran-5-yl)phenyl]sulfonyl}-4-cyclopropyl-1-oxa-4,9-diazaspiro[5.5]undecan-3-one;
4′-[(4-cyclopropyl-3-oxo-1-oxa-4,9-diazaspiro[5.5]undec-9-yl)sulfonyl]-3-methyl-4-biphenylcarbonitrile;
4-cyclopropyl-9-{[2-fluoro-4-(1H-indol-6-yl)phenyl]sulfonyl}-1-oxa-4,9-diazaspiro[5.5]undecan-3-one;
4′-[(4-cyclopropyl-3-oxo-1-oxa-4,9-diazaspiro[5.5]undec-9-yl)sulfonyl]-3′-fluoro-4-biphenylcarbonitrile;
4-cyclopropyl-9-[(3,4′-difluoro-4-biphenylyl)sulfonyl]-1-oxa-4,9-diazaspiro[5.5]undecan-3-one;
4-cyclopropyl-9-{[4-(1-methyl-2-oxo-1,2-dihydro-6-quinolinyl)phenyl]sulfonyl}-1-oxa-4,9-diazaspiro[5.5]undecan-3-one;
4-cyclopropyl-9-{[4-(1-methyl-2,3-dihydro-1H-indol-5-yl)phenyl]sulfonyl}-1-oxa-4,9-diazaspiro[5.5]undecan-3-one;
9-{[4-(1-benzofuran-2-yl)phenyl]sulfonyl}-4-cyclopropyl-1-oxa-4,9-diazaspiro[5.5]undecan-3-one;
9-{[4-(1-benzothien-2-yl)phenyl]sulfonyl}-4-cyclopropyl-1-oxa-4,9-diazaspiro[5.5]undecan-3-one;
9-{[4-(1,3-benzoxazol-2-yl)phenyl]sulfonyl}-4-cyclopropyl-1-oxa-4,9-diazaspiro[5.5]undecan-3-one;
4-cyclopropyl-9-{[4-(7-quinolinyl)phenyl]sulfonyl}-1-oxa-4,9-diazaspiro[5.5]undecan-3-one;
4-cyclopropyl-9-{[4-(7-quinolinyl)phenyl]sulfonyl}-1,4,9-triazaspiro[5.5]undecan-3-one;
4-cyclopropyl-1-methyl-9-{[4-(7-quinolinyl)phenyl]sulfonyl}-1,4,9-triazaspiro[5.5]undecan-3-one;
4-cyclopropyl-8-methyl-9-{[4-(7-quinolinyl)phenyl]sulfonyl}-1-oxa-4,9-diazaspiro[5.5]undecan-3-one;
4-cyclopropyl-7-methyl-9-{[4-(7-quinolinyl)phenyl]sulfonyl}-1-oxa-4,9-diazaspiro[5.5]undecan-3-one;
9-[(4-imidazo[1,2-a]pyridin-7-ylphenyl)sulfonyl]-4-(1-methylcyclopropyl)-1-oxa-4,9diazaspiro[5.5]undecan-3-one;
4-cyclopropyl-7-fluoro-9-[(4-imidazo[1,2-a]pyridin-7-ylphenyl)sulfonyl]-1-oxa-4,9-diazaspiro[5.5]undecan-3-one;
4-cyclopropyl-7-fluoro-9-{[4-(7-quinolinyl)phenyl]sulfonyl}-1-oxa-4,9-diazaspiro[5.5]undecan-3-one;
4-cyclopropyl-9-{[3-fluoro-4-(1H-indol-6-yl)phenyl]sulfonyl}-1-oxa-4,9-diazaspiro[5.5]undecan-3-one;
4-cyclopropyl-9-{[3-fluoro-4-(7-quinolinyl)phenyl]sulfonyl}-1-oxa-4,9-diazaspiro[5.5]undecan-3-one;
9-{[3-chloro-4-(1H-indol-6-yl)phenyl]sulfonyl}-4-cyclopropyl-1-oxa-4,9-diazaspiro[5.5]undecan-3-one;
9-{[3-chloro-4-(7-quinolinyl)phenyl]sulfonyl}-4-cyclopropyl-1-oxa-4,9-diazaspiro[5.5]undecan-3-one;
4-cyclopropyl-9-{[4-(1H-indol-6-yl)-3-methylphenyl]sulfonyl}-1-oxa-4,9diazaspiro[5.5]undecan-3-one;
4-cyclopropyl-9-{[3-methyl-4-(7-quinolinyl)phenyl]sulfonyl}-1-oxa-4,9-diazaspiro[5.5]undecan-3-one;
4-cyclopropyl-9-{[2,5-difluoro-4-(1H-indol-6-yl)phenyl]sulfonyl}-1-oxa-4,9-diazaspiro[5.5]undecan-3-one;
4-cyclopropyl-9-{[2,5-difluoro-4-(7-quinolinyl)phenyl]sulfonyl}-1-oxa-4,9-diazaspiro[5.5]undecan-3-one;
4-cyclopropyl-9-{[3-(methyloxy)-4-(7-quinolinyl)phenyl]sulfonyl}-1-oxa-4,9-diazaspiro[5.5]undecan-3-one;
4-cyclopropyl-9-{[4-(1H-indol-6-yl)-3-(methyloxy)phenyl]sulfonyl}-1-oxa-4,9-diazaspiro[5.5]undecan-3-one;
4-cyclopropyl-9-{[5-(7-quinolinyl)-2-pyridinyl]sulfonyl}-1-oxa-4,9-diazaspiro[5.5]undecan-3-one;
4-cyclopropyl-9-{[5-(1H-indol-6-yl)-2-pyridinyl]sulfonyl}-1-oxa-4,9-diazaspiro[5.5]undecan-3-one;
4-cyclopropyl-9-{[4-(7-quinolinyl)-3-(trifluoromethyl)phenyl]sulfonyl}-1-oxa-4,9-diazaspiro[5.5]undecan-3-one;
4-cyclopropyl-9-{[2-methyl-4-(7-quinolinyl)phenyl]sulfonyl}-1-oxa-4,9-diazaspiro[5.5]undecan-3-one;
4-cyclopropyl-9-{[4-(1H-indol-6-yl)-2-methylphenyl]sulfonyl}-1-oxa-4,9-diazaspiro[5.5]undecan-3-one;
9-{[2-chloro-4-(1H-indol-6-yl)phenyl]sulfonyl}-4-cyclopropyl-1-oxa-4,9-diazaspiro[5.5]undecan-3-one;
4-cyclopropyl-9-{[5-(1H-indol-6-yl)-2-thienyl]sulfonyl}-1-oxa-4,9-diazaspiro[5.5]undecan-3-one;
9-{[2-chloro-4-(7-quinolinyl)phenyl]sulfonyl}-4-cyclopropyl-1-oxa-4,9-diazaspiro[5.5]undecan-3-one;
4-cyclopropyl-9-{[2-fluoro-4-(7-quinolinyl)phenyl]sulfonyl}-1-oxa-4,9-diazaspiro[5.5]undecan-3-one;
4-cyclopropyl-9-{[5-(7-quinolinyl)-2-thienyl]sulfonyl}-1-oxa-4,9-diazaspiro[5.5]undecan-3-one;
4-cyclopropyl-9-{[6-(7-quinolinyl)-3-pyridinyl]sulfonyl}-1-oxa-4,9-diazaspiro[5.5]undecan-3-one;
4-cyclopropyl-9-{[6-(1H-indol-6-yl)-3-pyridinyl]sulfonyl}-1-oxa-4,9-diazaspiro[5.5]undecan-3-one;
4-cyclopropyl-9-{[2,3-dimethyl-4-(7-quinolinyl)phenyl]sulfonyl}-1-oxa-4,9-diazaspiro[5.5]undecan-3-one;
9-{[3-chloro-2-fluoro-4-(7-quinolinyl)phenyl]sulfonyl}-4-cyclopropyl-1-oxa-4,9-diazaspiro[5.5]undecan-3-one;
9-{[3-chloro-2-fluoro-4-(1H-indol-6-yl)phenyl]sulfonyl}-4-cyclopropyl-1-oxa-4,9-diazaspiro[5.5]undecan-3-one;
4-cyclopropyl-9-{[3,5-difluoro-4-(1H-indol-6-yl)phenyl]sulfonyl}-1-oxa-4,9-diazaspiro[5.5]undecan-3-one;
4-cyclopropyl-9-{[3,5-difluoro-4-(7-quinolinyl)phenyl]sulfonyl}-1-oxa-4,9-diazaspiro[5.5]undecan-3-one;
4-cyclopropyl-9-{[4-methyl-5-(7-quinolinyl)-2-thienyl]sulfonyl}-1-oxa-4,9-diazaspiro[5.5]undecan-3-one;
4-cyclopropyl-9-{[2-(7-quinolinyl)-1,3-thiazol-5-yl]sulfonyl}-1-oxa-4,9-diazaspiro[5.5]undecan-3-one;
4-cyclopropyl-9-{[2-fluoro-4-(1H-indol-5-yl)phenyl]sulfonyl}-1-oxa-4,9-diazaspiro[5.5]undecan-3-one;
9-{[4-(1-benzofuran-5-yl)-2-fluorophenyl]sulfonyl}-4-cyclopropyl-1-oxa-4,9-diazaspiro[5.5]undecan-3-one;
4-cyclopropyl-9-{[2-fluoro-4-(1H-indazol-5-yl)phenyl]sulfonyl}-1-oxa-4,9-diazaspiro[5.5]undecan-3-one;
4-cyclopropyl-9-{[2-fluoro-4-(6-quinolinyl)phenyl]sulfonyl}-1-oxa-4,9-diazaspiro[5.5]undecan-3-on;
4-cyclopropyl-9-[(2′,4′-dichloro-3-fluoro-4-biphenylyl)sulfonyl]-1-oxa-4,9-diazaspiro[5.5]undecan-3-one;
4-cyclopropyl-9-{[3-fluoro-4′-(methyloxy)-4-biphenylyl]sulfonyl}-1-oxa-4,9-diazaspiro[5.5]undecan-3-one;
9-{[4-(1,3-benzothiazol-5-yl)-2-fluorophenyl]sulfonyl}-4-cyclopropyl-1-oxa-4,9-diazaspiro[5.5]undecan-3-one;
{4′-[(4-cyclopropyl-3-oxo-1-oxa-4,9-diazaspiro[5.5]undec-9-yl)sulfonyl]-3′-fluoro-4-hydroxy-3-biphenylyl}formamide;
4-cyclopropyl-9-{[2-fluoro-4-(5-quinolinyl)phenyl]sulfonyl}-1-oxa-4,9-diazaspiro[5.5]undecan-3-one;
4-cyclopropyl-9-{[3-fluoro-4′-(1H-pyrazol-1-yl)-4-biphenylyl]sulfonyl}-1-oxa-4,9-diazaspiro[5.5]undecan-3-one;
9-{[4-(1,3-benzoxazol-5-yl)-2-fluorophenyl]sulfonyl}-4-cyclopropyl-1-oxa-4,9-diazaspiro[5.5]undecan-3-one;
4-cyclopropyl-9-{[3′-(1H-pyrazol-5-yl)-4-biphenylyl]sulfonyl}-1-oxa-4,9-diazaspiro[5.5]undecan-3-one;
4-cyclopropyl-9-{[4′-(1H-pyrazol-5-yl)-4-biphenylyl]sulfonyl}-1-oxa-4,9-diazaspiro[5.5]undecan-3-one;
4-cyclopropyl-9-{[4-(7-isoquinolinyl)phenyl]sulfonyl}-1-oxa-4,9-diazaspiro[5.5]undecan-3-one;
4-cyclopropyl-9-{[4-(7-quinazolinyl)phenyl]sulfonyl}-1-oxa-4,9-diazaspiro[5.5]undecan-3-one;
N-{4′-[(4-cyclopropyl-3-oxo-1-oxa-4,9-diazaspiro[5.5]undec-9-yl)sulfonyl]-3-biphenylyl}-N,N-dimethylsulfamide;
4-cyclopropyl-9-{[4-(6-isoquinolinyl)phenyl]sulfonyl}-1-oxa-4,9-diazaspiro[5.5]undecan-3-one;
4-cyclopropyl-9-{[4-(3-quinolinyl)phenyl]sulfonyl}-1-oxa-4,9-diazaspiro[5.5]undecan-3-one;
4-cyclopropyl-9-{[4-(2-naphthalenyl)phenyl]sulfonyl}-1-oxa-4,9-diazaspiro[5.5]undecan-3-one;
4-cyclopropyl-9-{[4-(2-methyl-1,3-benzothiazol-5-yl)phenyl]sulfonyl}-1-oxa-4,9-diazaspiro[5.5]undecan-3-one;
4-cyclopropyl-94 {4-[4-(ethyloxy)-7-quinolinyl]phenyl}sulfonyl)-1-oxa-4,9-diazaspiro[5.5]undecan-3-one;
4-cyclopropyl-9-({4-[4-(methyloxy)-7-quinolinyl]phenyl}sulfonyl)-1-oxa-4,9-diazaspiro[5.5]undecan-3-one;
4-cyclopropyl-9-[(4-imidazo[1,2-a]pyridin-7-ylphenyl)sulfonyl]-1-oxa-4,9-diazaspiro[5.5]undecan-3-one;
9-{[4-(3-amino-1H-indazol-6-yl)phenyl]sulfonyl}-4-cyclopropyl-1-oxa-4,9-diazaspiro[5.5]undecan-3-one;
9-{[4-(3-amino-1H-indazol-5-yl)phenyl]sulfonyl}-4-cyclopropyl-1-oxa-4,9-diazaspiro[5.5]undecan-3-one;
9-{[4-(2-amino-4-pyridinyl)phenyl]sulfonyl}-4-cyclopropyl-1-oxa-4,9-diazaspiro[5.5]undecan-3-one;
4-cyclopropyl-9-{[4-(4-quinolinyl)phenyl]sulfonyl}-1-oxa-4,9-diazaspiro[5.5]undecan-3-one;
4-cyclopropyl-9-{[4-(1-methyl-1H-indol-6-yl)phenyl]sulfonyl}-1-oxa-4,9-diazaspiro[5.5]undecan-3-one;
4-cyclopropyl-9-{[4-(1-methyl-1H-indol-4-yl)phenyl]sulfonyl}-1-oxa-4,9-diazaspiro[5.5]undecan-3-one;
4-cyclopropyl-9-({4-[4-(methylamino)-7-quinolinyl]phenyl}sulfonyl)-1-oxa-4,9-diazaspiro[5.5]undecan-3-one;
4-cyclopropyl-9-{[4-(4-methyl-7-quinolinyl)phenyl]sulfonyl}-1-oxa-4,9-diazaspiro[5.5]undecan-3-one;
4-cyclopropyl-9-[(4-imidazo[1,2-a]pyridin-6-ylphenyl)sulfonyl]-1-oxa-4,9-diazaspiro[5.5]undecan-3-one;
9-{[4-(7-cinnolinyl)phenyl]sulfonyl}-4-cyclopropyl-1-oxa-4,9-diazaspiro[5.5]undecan-3-one;
4-cyclopropyl-9-[(4-imidazo[1,2-b]pyridazin-6-ylphenyl)sulfonyl]-1-oxa-4,9-diazaspiro[5.5]undecan-3-one;
9-{[4-(3-amino-1-methyl-1H-indazol-5-yl)phenyl]sulfonyl}-4-cyclopropyl-1-oxa-4,9-diazaspiro[5.5]undecan-3-one;
N-(5-{4-[(4-cyclopropyl-3-oxo-1-oxa-4,9-diazaspiro[5.5]undec-9-yl)sulfonyl]phenyl}-1-methyl-1H-indazol-3-yl)methanesulfonamide;
9-{[4-(3-amino-1-methyl-1H-indazol-6-yl)phenyl]sulfonyl}-4-cyclopropyl-1-oxa-4,9-diazaspiro[5.5]undecan-3-one;
N-(6-{4-[(4-cyclopropyl-3-oxo-1-oxa-4,9-diazaspiro[5.5]undec-9-yl)sulfonyl]phenyl}-1H-indazol-3-yl)-N-methylurea;
4-cyclopropyl-9-((4-(8-methylquinolin-7-yl)phenyl)sulfonyl)-1-oxa-4,9-diazaspiro[5.5]undecan-3-one;
4-cyclopropyl-9-((4-(8-fluoroquinolin-7-yl)phenyl)sulfonyl)-1-oxa-4,9-diazaspiro[5.5]undecan-3-one;
1-(3-oxo-9-{[4-(7-quinolinyl)phenyl]sulfonyl}-1-oxa-4,9-diazaspiro[5.5]undec-4-yl)cyclopropanecarboxamide;
4-(1-methylcyclobutyl)-9-{[4-(7-quinolinyl)phenyl]sulfonyl}-1-oxa-4,9-diazaspiro[5.5]undecan-3-one;
1-(3-oxo-9-{[4-(7-quinolinyl)phenyl]sulfonyl}-1-oxa-4,9-diazaspiro[5.5]undec-4-yl)cyclopropanecarbonitrile;
4-(3-oxetanyl)-9-{[4-(7-quinolinyl)phenyl]sulfonyl}-1-oxa-4,9-diazaspiro[5.5]undecan-3-one;
4-[1-(hydroxymethyl)cyclopropyl]-9-{[4-(7-quinolinyl)phenyl]sulfonyl}-1-oxa-4,9-diazaspiro[5.5]undecan-3-one;
4-{1-[(methyloxy)methyl]cyclopropyl}-9-{[4-(7-quinolinyl)phenyl]sulfonyl}-1-oxa-4,9-diazaspiro[5.5]undecan-3-one;
4-[1-(hydroxymethyl)cyclopropyl]-9-({4-[3-(methyloxy)-7-quinolinyl]phenyl}sulfonyl)-1-oxa-4,9-diazaspiro[5.5]undecan-3-one;
4-cyclopropyl-9-((4-(8-methoxyquinolin-7-yl)phenyl)sulfonyl)-1-oxa-4,9-diazaspiro[5.5]undecan-3-one;
4-cyclopropyl-9-((4-(8-hydroxyquinolin-7-yl)phenyl)sulfonyl)-1-oxa-4,9-diazaspiro[5.5]undecan-3-one;
4-cyclopropyl-9-{[4-(7-quinolinyl)phenyl]sulfonyl}-1-oxa-4,9-diazaspiro[5.5]undecan-3-one-d₄, 4-cyclopropyl-9-((4-(6-fluoronaphthalen-2-yl)phenyl)sulfonyl)-1-oxa-4,9-diazaspiro[5.5]undecan-3-one;
4-cyclopropyl-9-((4-(8-fluoronaphthalen-2-yl)phenyl)sulfonyl)-1-oxa-4,9-diazaspiro[5.5]undecan-3-one;
4-ethyl-9-((4-(3-methoxyquinolin-7-yl)phenyl)sulfonyl)-1-oxa-4,9-diazaspiro[5.5]undecan-3-one;
4-isopropyl-9-((4-(3-methoxyquinolin-7-yl)phenyl)sulfonyl)-1-oxa-4,9-diazaspiro[5.5]undecan-3-one;
4-ethyl-9-((4-(3-fluoroquinolin-7-yl)phenyl)sulfonyl)-1-oxa-4,9-diazaspiro[5.5]undecan-3-one;
9-((4-(3-fluoroquinolin-7-yl)phenyl)sulfonyl)-4-isopropyl-1-oxa-4,9-diazaspiro[5.5]undecan-3-one;
9-((4-(3-methoxyquinolin-7-yl)phenyl)sulfonyl)-4-(1-methylcyclopropyl)-1-oxa-4,9-diazaspiro[5.5]undecan-3-one;
9-((4-(3-fluoroquinolin-7-yl)phenyl)sulfonyl)-4-(1-methylcyclopropyl)-1-oxa-4,9-diazaspiro[5.5]undecan-3-one;
4′-[(4-cyclopropyl-3-oxo-1-oxa-4,9-diazaspiro[5.5]undec-9-yl)sulfonyl]-3-biphenylcarboxylic acid;
4-cyclopropyl-9-{[4′-(1H-tetrazol-5-yl)-4-biphenylyl]sulfonyl}-1-oxa-4,9-diazaspiro[5.5]undecan-3-one;
{4′-[(4-cyclopropyl-3-oxo-1-oxa-4,9-diazaspiro[5.5]undec-9-yl)sulfonyl]-3-biphenylyl}boronic acid;
4′-[(4-cyclopropyl-3-oxo-1-oxa-4,9-diazaspiro[5.5]undec-9-yl)sulfonyl]-3-biphenylsulfonic acid;
2-cyclopropyl-9-{[4-(7-quinolinyl)phenyl]sulfonyl}-2,9-diazaspiro[5.5]undecan-3-one;
ethyl 7-(4-((4-cyclopropyl-3-oxo-1-oxa-4,9-diazaspiro[5.5]undecan-9-yl)sulfonyl)phenyl)quinoline-3-carboxylate;
7-(4((4-cyclopropyl-3-oxo-1-oxa-4,9-diazaspiro[5.5]undecan-9-yl)sulfonyl)phenyl)quinoline-3-carboxylic acid;
9-((4-(3-aminoquinolin-7-yl)phenyl)sulfonyl)-4-cyclopropyl-1-oxa-4,9-diazaspiro[5.5]undecan-3-one;
N-(7-(4-((4-cyclopropyl-3-oxo-1-oxa-4,9-diazaspiro[5.5]undecan-9-yl)sulfonyl)phenyl)quinolin-3-yl)acetamide;
7-(4-((4-cyclopropyl-3-oxo-1-oxa-4,9-diazaspiro[5.5]undecan-9-yl)sulfonyl)phenyl)quinoline-3-carbonitrile;
4-cyclopropyl-9-((4-(3-methoxyquinolin-7-yl)phenyl)sulfonyl)-1-oxa-4,9-diazaspiro[5.5]undecan-3-one;
4-cyclopropyl-9-((4-(3-methylquinolin-7-yl)phenyl)sulfonyl)-1-oxa-4,9-diazaspiro[5.5]undecan-3-one;
9-((4-(3-chloroquinolin-7-yl)phenyl)sulfonyl)-4-cyclopropyl-1-oxa-4,9-diazaspiro[5.5]undecan-3-one;
4-cyclopropyl-9-((4-(3-hydroxyquinolin-7-yl)phenyl)sulfonyl)-1-oxa-4,9-diazaspiro[5.5]undecan-3-one;
7-(4-((4-cyclopropyl-3-oxo-1-oxa-4,9-diazaspiro[5.5]undecan-9-yl)sulfonyl)phenyl)quinoline-3-carboxamide;
N-(7-(4-((4-cyclopropyl-3-oxo-1-oxa-4,9-diazaspiro[5.5]undecan-9-yl)sulfonyl)phenyl)quinolin-3-yl)cyclopropanecarboxamide;
2-(4-((4-cyclopropyl-3-oxo-1-oxa-4,9-diazaspiro[5.5]undecan-9-yl)sulfonyl)phenyl)thieno[3,2-b]pyridine-6-carboxamide;
4-cyclopropyl-9-((4-(3-fluoroquinolin-7-yl)phenyl)sulfonyl)-1-oxa-4,9-diazaspiro[5.5]undecan-3-one; and
N-(7-(4-((4-cyclopropyl-3-oxo-1-oxa-4,9-diazaspiro[5.5]undecan-9-yl)sulfonyl)phenyl)quinolin-3-yl)methanesulfonamide.

14. A pharmaceutical composition comprising a compound or salt according to claim 1 and a pharmaceutically acceptable carrier.

15. A method of treating cancer comprising administering to a human in need thereof an effective amount of a compound according to claim 1.

16. A method of treating cancer comprising administering to a human in need thereof an effective amount of a pharmaceutical composition of claim 14.

17. The method claim 15, wherein the cancer is selected from the group consisting of: brain (gliomas), glioblastomas, leukemias, Bannayan-Zonana syndrome, Cowden disease, Lhermitte-Duclos disease, breast, inflammatory breast cancer, Wilm's tumor, Ewing's sarcoma, Rhabdomyosarcoma, ependymoma, medulloblastoma, colon, head and neck, kidney, lung, liver, melanoma, ovarian, pancreatic, prostate, sarcoma, osteosarcoma, giant cell tumor of bone and thyroid.

18. A method of treating cancer in a mammal in need thereof, which comprises: administering to such mammal a therapeutically effective amount of:
a) a compound of Formula (I), as described in claim 1 or a pharmaceutically acceptable salt thereof; and
b) at least one anti-neoplastic agent.
